(19) United States
(12) Patent Application Publication
(10) Pub. No.: US 2025/0326857 A1
(43) Pub. Date: Oct. 23, 2025

(54) BINDING AGENTS TARGETING CD36-EXPRESSING TUMOR CELLS

(71) Applicant: KISOJI BIOTECHNOLOGY INC., Montreal (CA)

(72) Inventors: Shugang YAO, San Diego, CA (US); Hiba ZAHREDDINE, Montreal (CA); Luis DA CRUZ, Montreal (CA); David S. YOUNG, Montreal (CA)

(21) Appl. No.: 19/188,169

(22) Filed: Apr. 24, 2025

Related U.S. Application Data
(63) Continuation of application No. PCT/CA2023/051415, filed on Oct. 25, 2023.
(60) Provisional application No. 63/419,094, filed on Oct. 25, 2022.

(51) Int. Cl. C07K 16/28; A61K 39/00; A61P 35/00

(52) U.S. Cl. CPC C07K 16/2896; A61P 35/00; C07K 16/2803; C07K 16/2818; A61K 2039/505; C07K 2317/31; C07K 2317/569

(57) ABSTRACT

The present disclosure generally relates to binding agents that are capable of targeting tumor cells and/or immune cells and their use for treating cancer. The binding agents of the present disclosure comprise one or more antigen binding domains of single domain antibodies which are capable of binding to Cluster of Differentiation 36 (CD36), to Programmed Cell Death protein 1 (PD-1) and/or to Cluster of Differentiation 47 (CD47). The binding agents of the present disclosure are monospecific or multispecific and may be in the form of monomers or multimers.

### CROSS REFERENCED APPLICATIONS

This application is a Continuation Application of International Application No. PCT/CA2023/051415, filed on Oct. 25, 2023, which claims priority to U.S. Provisional Patent Application Ser. No. 63/419,094, filed on Oct. 25, 2022, the contents of each of which are incorporated by reference herein in entirety for all purposes.

### INCORPORATION OF SEQUENCE LISTING

The instant application contains a Sequence Listing, which has been submitted electronically in XML file format and is hereby incorporated by reference in its entirety. Said XML copy, created on Apr. 2, 2025, is named KJB-009US1_SL.xml and is 425,984 bytes in size.

### TECHNICAL FIELD

The present disclosure generally relates to binding agents that are capable of targeting tumor cells and/or immune cells and their use for treating cancer. The binding agents of the present disclosure comprise one or more antigen binding domains of single domain antibodies which are capable of binding to Cluster of Differentiation 36 (CD36), to Programmed Cell Death protein 1 (PD-1) and/or to Cluster of Differentiation 47 (CD47). The binding agents of the present disclosure are monospecific or multispecific and may be in the form of monomers or multimers.

### BACKGROUND

Camelids and cartilaginous fishes naturally produce antibodies composed of functional homodimeric heavy chain only antibodies (HCAbs) (Hamers-Casterman et al., 1993; Muyldermans and Smider, 2016). The heavy chains of HCAbs lack the first constant domain (CH1) and differs from classical antibodies by only a few amino acids substitutions normally involved in light chain pairing (Muyldermans et al., 1994; Vu et al., 1997). These substitutions (Val37Phe/Tyr, Gly44Glu, Leu45Arg, and Trp47Gly) are present in framework region 2 (FR2). The antigen-binding fragment of HCAbs is referred to as, VHH or nanobody®. VHHs have a molecular weight of around 15 kDa which makes them amenable to applications that require enhanced tissue penetration or rapid clearance, such as radioisotope-based imaging. However, for therapeutic applications, the VHH half-life usually needs to be increased so as to minimize renal clearance and optimize therapeutic efficacy (De Vlieger et al., Antibodies 8(1), 1-22, 2019). Although methods to increase VHH half-life such as PEGylation, N-glycosylation, HSA or other carrier protein fusions have been exploited, such construct can introduce immunogenicity or have limited success.

VHHs have been exploited as building blocks to make bispecific and multispecific antibodies. In some studies, bivalent constructs have been shown to be have increased avidity or affinity compared to the monovalent form (Conrath et al., 2001; Coppieters et al., 2006; Hmila et al., 2008; Simmons et al., 2006 and Hultberg et al., 2011, Jahnichen et al. (2010), Fridy et al., 2014).

A number of VHH-based therapeutics are currently in late investigational stage or have been approved by FDA. These include the bivalent monospecific antibody Caplacizumab against antigen vWF approved for Thrombotic thrombocytopenic purpura (Duggan, 2018). A Trivalent nanobody complex, ALX-0171 against RSV is at late-stage development for Respiratory syncytial virus infection (Detallea et al., 2015). ALX-0061 is a monovalent against antigen IL-6R but attached with HSA nanobody to extend half-life and is at clinical development stage for RA and SLE indications (Van Roy et al., 2015). The investigational drug ALX-0761 contains three nanobodies against antigens IL-17A, IL-17F and HAS and is being developed for Psoriasis (Svecova et., 2019). Anti-RANKL, ALX-0141 is a bivalent for antigen RANKL and attached to HSA to extend half-life (Schoen et al., 2013). Ozoralizumab is bivalent nanobody against antigen TNF(x and attached to HSA to extend half-life (Fleischmann et al., 2012).

CD36 is a transmembrane protein also known as fatty acid translocase (FAT), FAT/CD36, SCARB3, GP88, gpIV and gpIIIb is that plays a major role in lipid metabolism and in other cellular function such as inflammation, immunological recognition and apoptosis (Wang, J et al., Theranostic 9(17), p. 4893-4908, 2019, the entire content of which is incorporated herein by reference). CD36 acts as a receptor for a variety of ligands including that apoptotic cells, thrombospondin-1 (TSP-1), and fatty acids (FAs). CD36 promotes tumor metastasis and treatment resistance by promoting lipid uptake and FA oxidation, inhibits angiogenesis by binding with TSP-1 Chen, Y-J et al., (Journal of Cancer 12(16), p. 4762-4773, 2021, the entire content of which is incorporated herein by reference). CD36 is involved in tumor cell metabolism, anti-angiogenesis, metastasis, therapy resistance and tumor immunoediting.

The amino acid sequence and structure of human CD36 was first disclosed in Armesilla, A et al., (The Journal of Biological Chemistry, 269(29), p. 18895-18991, 1994; GenBank Accession No. CAA83662.1, Uniprot Accession No. P16671-1), the entire contents of which is incorporated herein by reference).

The Applicant has developed novel binding agents that targets tumors cells and/or immune for treatment of cancer.

### SUMMARY

The present disclosure generally relates to binding agents that comprises one or more antigen binding domain and that are capable, amongst other things, of targeting tumor cells and/or immune cells.

In some embodiments, the binding agent of the present disclosure binds to a Cluster of Differentiation 36 (CD36) protein or to a portion thereof.

In some embodiments, the binding agent is a single domain antibody (sdAb) that binds to a CD36 protein such as human CD36 or to a portion thereof.

In some instances, the anti-CD36 single domain antibody of the present disclosure may block binding between CD36 and a CD36 ligand.

In some instances, the anti-CD36 single domain antibody of the present disclosure may modulate lipid metabolism.

In other instances, the anti-CD36 single domain antibody of the present disclosure may inhibit fatty acid oxidation.

In yet other instances, the anti-CD36 single domain antibody of the present disclosure may inhibit fatty acid uptake by tumors.

In other instances, the anti-CD36 single domain antibody of the present disclosure may inhibit the growth of tumor cells or may be used to inhibit the growth of tumor cells.

In yet other instances, the anti-CD36 single domain antibody of the present disclosure may inhibit tumor metastasis or may be used to inhibit tumor metastasis.

In yet other instances, the anti-CD36 single domain antibody of the present disclosure may be used to reduce resistance of tumor cells to chemotherapeutics and/or immune checkpoint inhibitors therapy.

The binding function of sdAbs is conferred by a single polypeptide chain namely, the heavy chain variable domain. This unique property provides formatting flexibility. For example, multiple sdAbs or sdAb antigen binding domains may be fused into a single polypeptide chain conferring multivalence and/or multispecificity. In addition, multiple polypeptide chains may be assembled to increase the diversity or avidity of interactions with CD36. Moreover, the sdAbs antigen binding domains may be fused to any type of polypeptide chain including for example and without limitation, fusion with a heavy chain and/or light chain of a native antibody or antigen binding fragment thereof, with protein scaffold, with immune cell modulating agent and the like.

As such, the present disclosure not only relates to the single domain antibodies disclosed herein but also more generally to binding agents that comprise one or more antigen binding domains of the single domain antibodies disclosed herein.

In accordance with the present disclosure, the binding agent binds to CD36 and also bind to at least one other antigen. For example, the binding agent may bind to CD36 and to an immune checkpoint protein. In another example, the binding agent may bind to CD36 and to a protein expressed at the surface of immune cells.

Accordingly, exemplary embodiments of binding agents of the present disclosure include binding agents that bind to CD36, to an immune checkpoint protein and to a protein expressed at the surface of immune cells.

In some instances, the binding agent is capable of binding to CD36 and to Programmed Cell Death protein 1 (PD-1). Accordingly, in some embodiments, the binding agent comprises at least one antigen binding domain that binds to CD36 and at least one antigen binding domain that binds to PD-1.

In other instances, the binding agent is capable of binding to CD36 and to Cluster of Differentiation 47 (CD47). Accordingly, in some embodiments, the binding agent comprises at least one antigen binding domain that binds to CD36 and at least one antigen binding domain that binds to CD47.

The present disclosure therefore provides binding agents capable of binding to a CD36 protein or a portion thereof, to a Programmed Cell Death protein 1 (PD-1) protein or a portion thereof and to a Cluster of Differentiation 47 (CD47) protein or a portion thereof. In some embodiments, the binding agents may bind to cells expressing the CD36 protein or a portion thereof, to cells expressing the PD-1 protein or a portion thereof and/or to cells expressing the CD47 protein or a portion thereof.

In some aspects, the binding agents of the present disclosure may bind to human CD36 protein, to human PD-1 protein and/or to human CD47 protein and/or to cells expressing human CD36 protein, human PD-1 and/or human CD47 thereof.

The binding agent of the present disclosure comprises one or more antigen binding domains. The binding agent may comprise, for example, at least one antigen binding domain capable of binding to a CD36 protein or a portion thereof or to cells expressing the CD36 protein or a portion thereof, at least one antigen binding domain capable of binding to a PD-1 protein or a portion thereof or to cells expressing the PD-1 protein or a portion thereof and/or at least one antigen binding domain capable of binding to a CD47 protein or a portion thereof or to cells expressing the CD47 protein or a portion thereof.

Accordingly, the binding agents of the present disclosure comprises at least one antigen binding domain capable of binding to the human CD36 protein or a portion thereof, at least one antigen binding domain capable of binding to the human PD-1 protein or a portion thereof and/or at least one antigen binding domain capable of binding to the human CD47 protein or a portion thereof. In accordance with the present disclosure, the binding agent may also comprise additional antigen binding domains.

In some embodiments, the antigen binding domain is from an antibody capable of specifically binding to a CD36 protein or to a portion thereof. As such, the antigen binding domain may be derived from a single domain antibody capable of specifically binding to a CD36 protein or to a portion thereof.

In some embodiments, the antigen binding domain is from an antibody capable of specifically binding to a PD-1 protein or to a portion thereof. As such, the antigen binding domain may be derived from a single domain antibody capable of specifically binding to a PD-1 protein or to a portion thereof.

In some embodiments, the antigen binding domain is from an antibody capable of specifically binding to a CD47 protein or to a portion thereof. As such, the antigen binding domain may be derived from a single domain antibody that is capable of specifically binding to a CD47 protein or to a portion thereof.

The binding agent of the present disclosure may be capable of reducing tumor growth and/or of causing tumor regression. In some embodiments, the binding agent has anti-tumor activity. In some embodiments, the binding agent is able to recruit or redirect immune cells. In some instances, the binding agent of the present disclosure may modulate lipid metabolism.

In other instances, the binding agent of the present disclosure may inhibit fatty acid oxidation.

In yet other instances, the binding agent of the present disclosure may inhibit fatty acid uptake by tumors.

In other instances, the binding agent of the present disclosure may inhibit the growth of tumor cells or may be used to inhibit the growth of tumor cells.

In yet other instances, the binding agent of the present disclosure may inhibit tumor metastasis or may be used to inhibit tumor metastasis.

In yet other instances, the binding agent of the present disclosure may be used to reduce resistance of tumor cells to chemotherapeutics and/or immune checkpoint inhibitors therapy.

In some embodiments, the binding agent comprises antigen binding domains of single domain antibodies that have in vitro and/or in vivo activity. Accordingly, the binding agent comprises one or more antigen binding domains of a single domain antibody that is capable of specifically binding to and inhibiting CD36. In some embodiments, the binding agent comprises more than one antigen binding domain and some of the antigen binding domains may be from same or different single domain antibodies that are capable of specifically binding to and inhibiting CD36.

In an exemplary embodiment, the antigen binding domain(s) is(are) from one or more single domain antibodies capable of specifically binding to CD36 and that has anti-tumor activity.

In another exemplary embodiment, the antigen binding domain(s) is(are) from one or more anti-CD36 single domain antibodies that inhibit the growth tumor cells.

In another exemplary embodiment, the antigen binding domain(s) is(are) from one or more anti-CD36 single domain antibodies that inhibit metastasis of tumor cells.

In another exemplary embodiment, the antigen binding domain(s) is(are) from one or more anti-CD36 single domain antibodies that modulates lipid metabolism of tumors.

In another exemplary embodiment, the antigen binding domain(s) is(are) from one or more anti-CD36 single domain antibodies that inhibit fatty acid oxidation.

In another exemplary embodiment, the antigen binding domain(s) is(are) from one or more anti-CD36 single domain antibodies that inhibits fatty acid uptake by tumors.

In yet other exemplary embodiments, the antigen binding domain(s) is(are) from one or more anti-CD36 single domain antibodies that do not significantly lead to platelet activation.

The binding agent of the present disclosure may also comprise one or more antigen binding domains of antibodies that are capable of specifically binding to an immune checkpoint protein.

For example, the binding agent of the present disclosure may comprise one or more antigen binding domains of a single domain antibody that is capable of specifically binding to and inhibiting PD-1. In some embodiments, the binding agent comprises more than one antigen binding domain and some of the antigen binding domains may be from same or different single domain antibodies that are capable of specifically binding to and inhibiting PD-1.

In some embodiments, the antigen binding domain(s) is(are) from an anti-PD-1 single domain antibody selected for its ability to block the PD-1 and PD-L1 interaction. PD-1/PD-L1 blockade assays are known to a person skilled in the art and include for example, Promega, Cat. No. J1255.

In some embodiments, the antigen binding domain(s) is(are) from an anti-PD-1 single domain antibody selected for its ability to block PD-L1 and/or PD-L2 interaction.

In some embodiments, the antigen binding domain(s) is(are) from an anti-PD-1 single domain antibody selected for its ability to restore T cell effector function.

In some embodiments, the binding agent comprises one or more antigen binding domains of antibodies that are capable of specifically binding to a protein expressed at the surface of immune cells.

For example, the antigen binding domain(s) is(are) from a single domain antibody that is capable of specifically binding to and inhibiting CD47. In some embodiments, the binding agent comprises more than one antigen binding domain and some of the antigen binding domains may be from same or different single domain antibodies that are capable of specifically binding to and inhibiting CD47.

In some embodiments, the antigen binding domain(s) is(are) from an anti-CD47 single domain antibody selected for its ability to block the CD47 and SIRPu interaction. CD47/SIRPu blockade assays are known to a person skilled in the art and include for example, Promega, Cat. No. CS316013. In some embodiments, the antigen binding domain(s) is(are) from an anti-CD47 single domain antibody selected for its ability to block the interaction between CD47 and Thrombospondin-1 (TSP-1).

In some embodiments, the antigen binding domain(s) is(are) from an anti-CD47 single domain antibody selected for its ability to recruit T cells. In some embodiments, the antigen binding domain(s) is(are) from an anti-CD47 single domain antibody selected for its ability to enhance macrophages function.

In some embodiments, the antigen binding domain capable of binding to a CD36 protein or a portion thereof or to cells expressing the CD36 protein or a portion thereof is an antigen binding domain 1 (ABD1) as described herein.

Accordingly, in some embodiments, the binding agent may comprise at least one antigen binding domain 1 (ABD1). In accordance with the present disclosure, ABD1 comprises at least the CDRH3 amino acid sequence of an anti-CD36 single domain antibody disclosed herein. Also in accordance with the present disclosure, ABD1 comprises the CDRH3 amino acid sequence and at least the CDRH1 and/or the CDRH2 amino acid sequence of an anti-CD36 single domain antibody disclosed herein. In some embodiments, ABD1 comprises the CDRH1, CDRH2 and CDRH3 amino acid sequence of an anti-CD36 single domain antibody disclosed herein. In some embodiments, the CDRH1, CDRH2 and CDRH3 amino acid sequence are from a single anti-CD36 single domain antibody. In other embodiments, ABD1 comprises the variable region of an anti-CD36 single domain antibody disclosed herein.

In some embodiment, the binding agent may comprise at least one antigen binding domain 1 (ABD1) and at least one other antigen binding domain. For example, the binding agent may comprise one or more antigen binding domain 1 (ABD1) and one or more antigen binding domains of one or more antibodies.

In some instances, the binding agent may comprise at least one antigen binding domain 1 (ABD1) and may also bind to immune cells. Accordingly, in some embodiments, the binding agent comprises at least one antigen binding domain 1 (ABD1) and at least one antigen binding domain that binds to immune cells.

In some embodiments, the antigen binding domain capable of binding to PD-1 or to cells expressing PD-1 is an antigen binding domain 2 (ABD2) as described herein. In some embodiments, the antigen binding domain capable of binding to PD-1 or to cells expressing PD-1 is not limited to antigen binding domain 2 (ABD2).

Accordingly, in some embodiments, the binding agent may comprise at least one antigen binding domain 2 (ABD2). In accordance with the present disclosure, ABD2 comprises at least the CDRH3 amino acid sequence of an anti-PD-1 single domain antibody disclosed herein. Also in accordance with the present disclosure, ABD2 comprises the CDRH3 amino acid sequence and at least the CDRH1 and/or the CDRH2 amino acid sequence of an anti-PD-1 single domain antibody disclosed herein. In some embodiments, ABD2 comprises the CDRH1, CDRH2 and CDRH3 amino acid sequence of an anti-PD-1 single domain antibody disclosed herein. In some embodiments, the CDRH1, CDRH2 and CDRH3 amino acid sequence are from a single anti-PD-1 single domain antibody. In other embodiments, ABD2 comprises the variable region of an anti-PD-1 single domain antibody disclosed herein.

In some embodiment, the binding agent may comprise at least one antigen binding domain 2 (ABD2) and at least one other antigen binding domain.

In some embodiments, the antigen binding domain capable of binding to CD47 or to cells expressing CD47 is an antigen binding domain 3 (ABD3) as described herein. In some embodiments, the antigen binding domain capable of binding to CD47 or to cells expressing CD47 is not limited to ABD3.

Accordingly, in some embodiments, the binding agent may comprise at least one antigen binding domain 3 (ABD3). In accordance with the present disclosure, ABD3 comprises at least the CDRH3 amino acid sequence of an anti-CD47 single domain antibody disclosed herein. Also in accordance with the present disclosure, ABD3 comprises the CDRH3 amino acid sequence and at least the CDRH1 and/or the CDRH2 amino acid sequence of an anti-CD47 single domain antibody disclosed herein. In some embodiments, ABD3 comprises the CDRH1, CDRH2 and CDRH3 amino acid sequence of an anti-CD47 single domain antibody disclosed herein. In some embodiments, the CDRH1, CDRH2 and CDRH3 amino acid sequence are from a single anti-CD47 single domain antibody. In other embodiments, ABD3 comprises the variable region of an anti-CD47 single domain antibody disclosed herein.

In some embodiment, the binding agent may comprise at least one antigen binding domain 3 (ABD3) and at least one other antigen binding domain.

In some embodiments, the binding agent comprises one or more antigen binding domains, wherein at least one of the antigen binding domains is an antigen binding domain 1 (ABD1).

In some embodiments, the binding agent comprises one or more antigen binding domains, wherein at least one of the antigen binding domains is an antigen binding domain 2 (ABD2).

In some embodiments, the binding agent comprises one or more antigen binding domains, wherein at least one of the antigen binding domains is an antigen binding domain 3 (ABD3).

In some embodiments, the binding agent comprises one or more antigen binding domains, wherein at least one of the antigen binding domains is selected from:
an antigen binding domain 1 (ABD1),
an antigen binding domain 2 (ABD2) and/or,
an antigen binding domain 3 (ABD3).

In some embodiments, the binding agent may comprise an antigen binding domain that targets epitopes other than those recognized by ABD1, ABD2 and ABD3. The binding agent may thus comprise an antigen binding domain that has the same or different specificity as that of ABD1, ABD2 and/or ABD3.

In some embodiments, the binding agent may comprise more than one antigen binding domains.

For example, in some embodiments, the binding agent may comprise two antigen binding domains or more, three antigen binding domains or more, four antigen binding domains or more, five antigen binding domains or more, six antigen binding domains or more, seven antigen binding domains or more, eight antigen binding domains or more, nine antigen binding domains or more, ten antigen binding domains or more.

In some embodiments, the binding agent may comprise between one and twelve antigen binding domains.

In some embodiments, the binding agents of the present disclosure may be monospecific.

In some embodiments, the binding agents of the present disclosure may be multispecific.

In some embodiments, the binding agents of the present disclosure may be monovalent.

In some embodiments, the binding agents of the present disclosure may be multivalent.

In some embodiments, the binding agents of the present disclosure may be in the form of a monomer.

In some embodiments, the binding agents of the present disclosure may be in the form of a dimer or higher order form such as trimer, four-mer, five-mer and the like (e.g., multimer).

In some instances, the antigen binding domain of the binding agent originates from a heavy chain antibody. In some instances, the heavy chain antibodies may be obtained by immunization of camelids or transgenic animals.

The binding agent of the present disclosure is essentially polypeptidic. In some embodiments, the binding agent of the present disclosure is essentially composed of one, two or more than two polypeptide chains. However, the binding agent may comprise non-polypeptidic moieties such as for example, small molecules, sugars, radioactive labels and the like.

In some embodiments, the binding agent comprises one or more polypeptide chains each independently comprising in a N- to C-terminal fashion an antigen binding domain that binds to CD36, an antigen binding domain that binds to PD-1 and an antigen binding domain that binds to CD47.

In some embodiments, the antigen binding domains are on one or more polypeptide chains.

In some embodiments, the antigen binding domains antigen are on same polypeptide chain.

In some embodiments, the binding agent comprises a single polypeptide chain.

In some embodiments, the binding agent comprises two polypeptide chains. Accordingly, the binding agent may be in the form of a dimer.

In some embodiments, the binding agent comprises more than two polypeptide chains, such as three polypeptide chains or more, four polypeptide chains or more, five polypeptide chains or more, six polypeptide chains or more, seven polypeptide chains or more, eight polypeptide chains or more, nine polypeptide chains or more, ten polypeptide chains or more. Accordingly, the binding agent may be in the form of a multimer. For example, a binding agent that comprises three polypeptide chains is referred herein as a trimer, a binding agent that comprises four polypeptide chains is referred herein as a four-mer, and the like.

In some embodiments, the binding agent comprises at least two polypeptide chains that are capable of assembling to form a dimer and wherein each polypeptide chain comprises one or more antigen binding domains.

It is to be understood herein that the polypeptide chains of the present disclosure may alternatively be expressed in tandem.

In some embodiments, the two polypeptide chains are capable of assembling to form a dimer and each polypeptide chain comprises different antigen binding domains.

In some embodiments, the two polypeptide chains are capable of assembling to form a dimer and each polypeptide chain comprises the same antigen binding domains.

In some embodiments, each polypeptide chain comprises identical antigen binding domain 1 (ABD1).

In some embodiments, each polypeptide chain comprises identical antigen binding domain 2 (ABD2).

In some embodiments, each polypeptide chain comprises identical antigen binding domain 3 (ABD3).

In some exemplary embodiments, the binding agent comprises at least two polypeptide chains that assemble to form a dimer and each polypeptide chain comprises identical antigen binding domain 1 (ABD1) and identical antigen binding domain 2 (ABD2).

In other exemplary embodiments, the binding agent comprises at least two polypeptide chains that assemble to form a dimer and each polypeptide chain comprises identical antigen binding domain 1 (ABD1) and identical antigen binding domain 3 (ABD3).

In yet further exemplary embodiments, the binding agent comprises at least two polypeptide chains that assemble to form a dimer and each polypeptide chain comprises identical antigen binding domain 1 (ABD1), identical antigen binding domain 2 (ABD2) and identical antigen binding domain 2 (ABD3).

In some instances, the antigen binding domain 1 (ABD1), antigen binding domain 2 (ABD2) and/or antigen binding domain 2 (ABD3) may occupy the same position on each of the two polypeptide chains.

In some instances, the antigen binding domain 1 (ABD1), antigen binding domain 2 (ABD2) and/or antigen binding domain 2 (ABD3) may occupy different positions on each of the two polypeptide chains.

In some embodiments, each polypeptide chains of the binding agent are the same.

In some embodiments, each polypeptide chains of the binding agent are different.

In some embodiments, the two polypeptide chains are capable of assembling to form a homodimer.

In some embodiments, the two polypeptide chains are capable of assembling to form a heterodimer.

In some embodiments, antigen binding domains are separated by an amino acid sequence.

In some embodiments, the amino acid sequence is a linker.

Binding agents of the present disclosure encompass for example the antigen binding domains disclosed herein, single polypeptide chain disclosed herein, dimers of the polypeptide chains disclosed herein or multimers of the polypeptide chains disclosed herein.

Accordingly, binding agents of the present disclosure may have a format of an antibody and antigen binding fragment thereof, an antibody-like molecule (Fc-, CH3- fusions and the like), a fusion with protein scaffolds, immune cell modulating agents and the like.

Advantageously, the binding agents of the present disclosure may have a format as disclosed in PCT/CA2020/051753 filed on Dec. 18, 2020 (published on Jun. 24, 2021 under No. WO2021/119832 A1 the entire content of which is incorporated herein by reference) as formula Ia, formula Ib, formula Ic, formula II, formula III, formula IV, formula V, formula VI, formula VII or formula VIII and the like or formula I, formula II, formula III, formula IIIa and formula IIIb, formula IV, formula V, formula VI, formula VII or formula VIII disclosed herein.

In some embodiments, the binding agents are composed of polypeptide chains comprising one or more antigen binding domains and a dimerization domain allowing at least two polypeptide chains to form dimers.

In some embodiments, the binding agent of the present disclosure may comprise one or more polypeptide chains each independently comprising in a N- to C-terminal fashion an amino acid sequence of formula I:

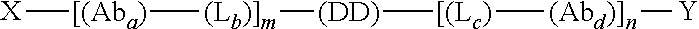

Wherein m is 0, 1, 2 or an integer greater than 2;
Wherein n is 0, 1, 2 or an integer greater than 2;
Wherein m and n are not 0 simultaneously;
Wherein Abₐ, Ab_{d}, each represents an antigen binding domain wherein at least one of the antigen binding domains is an antigen binding domain 1 (ABD1);
Wherein X or Y are independently present or absent and comprises an amino acid sequence;
Wherein L_{b}, L_{c}, each independently comprises one or more linkers; and
Wherein DD represents a dimerization domain.

In some embodiments, when m is 1, 2 or an integer greater than 2 and/or when n is 1, 2 or an integer greater than 2, at least one of Abₐ, or Ab_{d}, is an antigen binding domain 2 (ABD2).

In some embodiments, when m is 1, 2 or an integer greater than 2 and/or when n is 1, 2 or an integer greater than 2, at least one of Abₐ, or Ab_{d}, is an antigen binding domain 3 (ABD3).

In some embodiments, when m is 1, 2 or an integer greater than 2 and/or when n is 1, 2 or an integer greater than 2, at least one of Abₐ, or Ab_{d}, is an antigen binding domain 2 (ABD2) and/or an antigen binding domain 3 (ABD3).

In some embodiments the polypeptide chain comprises two antigen binding domains or more, three antigen binding domains or more, four antigen binding domains or more, five antigen binding domains or more, six antigen binding domains or more, etc.

In some embodiments the polypeptide chain comprises between one and twelve antigen binding domains.

In some embodiments, the binding agent comprises one polypeptide chain.

In some embodiments, the binding agent comprises two polypeptide chains, three polypeptide chains, four polypeptide chains, five polypeptide chains, six polypeptide chains, seven polypeptide chains, eight polypeptide chains, nine polypeptide chains, ten polypeptide chains or more than ten polypeptide chains.

In some embodiment, the polypeptide chains may be covalently linked.

In some embodiment, the polypeptide chains may be non-covalently linked.

In some embodiment, the polypeptide chains may be associated via electrostatic interaction(s).

In some embodiments where m is 2 or an integer greater than 2, the [(Abₐ)-(L_{b})] units is the same or different.

In some embodiments where n is 2 or an integer greater than 2, the [(L_{c})-(Ab_{d})] units are the same or different.

In some embodiments, where m is 2 or an integer greater than 2, each Abₐ is the same or different.

In some embodiments, where n is 2 or an integer greater than 2, each Ab_{d} is the same or different.

In some embodiments, Abₐ represents ABD1, ABD2 or ABD3.

In some embodiments, Ab_{d} represents ABD1, ABD2 or ABD3.

In some embodiments, Ab_{d} represents ABD1, ABD2 or ABD3.

In embodiments, the one or more polypeptide chain further comprises a hinge region of an antibody or antigen binding fragment thereof. For example, in some embodiments L_{b} is a hinge region of an antibody or antigen binding fragment thereof.

In some embodiments, the hinge region is a natural hinge region from an IgG1, IgG2, IgG3 or IgG4.

In some embodiments, the hinge region is a mutated hinge region having at least 70% identity with a natural hinge region from an IgG1, IgG2, IgG3 or IgG4.

In some embodiments each of the one or more linkers independently has at least 5, 10, 15, 20, 25, 30, 35, 40, 45 or 50 amino acid residues in length.

In some embodiments, each of the one or more linkers is independently a flexible linker, a helical linker, or a rigid linker.

In some embodiments, the flexible linker is a GS linker. In some embodiments, the flexible linker comprises one or more units of GGGGS as described herein.

In some embodiments the rigid linker comprises multiple PA repeats as described herein.

In some embodiments, the helical linker comprises one or more units of EAAAK as described herein.

In some embodiments the binding agent comprises one or more polypeptide chains wherein at least one of the polypeptide chains comprises formula II: X-(Abₐ₁)(L_{b1})(DD)-(L_{c1})-(Ab_{d1})Y (formula II).

In some embodiments the binding agent comprises one or more polypeptide chains wherein at least one of the polypeptide chains comprises formula III: X-(Abₐ₁)(L_{b1})(DD)-(L_{c})-(Ab_{d1})-(L_{c2})-(Ab_{d2})-Y (formula III).

In some embodiments the binding agent comprises one or more polypeptide chains wherein at least one of the polypeptide chains comprises formula IV: X-(Abₐ₁)(L_{b2})-(Abₐ₂)-(L_{b1})-(DD)-(L_{c1})-(Ab_{d1})-Y (formula IV).

In some embodiments the binding agent comprises one or more polypeptide chains wherein at least one of the polypeptide chains comprises formula V: X-(Abₐ₁)(L_{b2})-(Abₐ₂)-(L_{b1})-(DD)-(L_{c1})-(Ab_{d1})-(L_{c2})-(Ab_{d2})-Y (formula V).

In some embodiments the binding agent comprises one or more polypeptide chains wherein at least one of the polypeptide chains comprises formula VI: X-(Abₐ₁)-(L_{b2})-(Abₐ₂)-(L_{b1})-(DD)-(L_{c1})-(Ab_{d1})-(L_{c2})-(Ab_{d2})-(L_{c3})-(Ab_{d3})-Y (formula VI).

In some embodiments the binding agent comprises one or more polypeptide chains wherein at least one of the polypeptide chains comprises formula VII: X-(Abₐ₁)-(L_{b3})-(Abₐ₂)-(L_{b2})-(Abₐ₃)-(L_{b1})-(DD)-(L_{c1})-(Ab_{d1})-(L_{c2})-(Ab_{d2})-Y (formula VII).

In some embodiments the binding agent comprises one or more polypeptide chains wherein at least one of the polypeptide chains comprises formula VIII: X-(Abₐ₁)(L_{b3})-(Abₐ₂)-(L_{b2})-(Abₐ₃)-(L_{b1})-(DD)-(L_{c1})-(Ab_{d1})-(L_{c2})-(Ab_{d2})-(L_{c3})-(Ab_{d3})-Y (formula VIII).

In some embodiments Abₐ₁, Abₐ₂, Abₐ₃, Ab_{d1}, Ab_{d2}, or Ab_{d3}, each independently comprises an antigen binding domain.

In some embodiments Abₐ₁, Abₐ₂, Abₐ₃, Ab_{d1}, Ab_{d2}, or Ab_{d3}, each independently represents an antigen binding domain.

In some embodiments, at least one of Abₐ₁, Abₐ₂, Abₐ₃, Ab_{d1}, Ab_{d2}, or Ab_{d3} is an antigen binding domain 1 (ABD1), an antigen binding domain 2 (ABD2) or an antigen binding domain 3 (ABD3).

In some embodiments, at least one of Abₐ₁, Abₐ₂, Abₐ₃, Ab_{d1}, Ab_{d2}, or Ab_{d3} is not an antigen binding domain 1 (ABD1), an antigen binding domain 2 (ABD2) or an antigen binding domain 3 (ABD3).

In some embodiments L_{b1} comprises a linker or linkers and/or a hinge region of an antibody or antigen binding fragment thereof. In some embodiments, the hinge region is a natural hinge region as disclosed herein. In other embodiments, the hinge region is a mutated hinge region as disclosed herein.

In some embodiments L_{b}2, L_{b3} L_{c1}, L_{c2}, and L_{c3} each independently comprise a linker or linkers as disclosed herein.

In some embodiments, the polypeptide chain comprises the antigen binding domain ABD1 as disclosed herein and an antigen binding domain that binds to an immune cell.

In some embodiments, the polypeptide chain comprises the antigen binding domain ABD2 as disclosed herein and an antigen binding domain that binds to a tumor cell.

In some embodiments, the polypeptide chain comprises the antigen binding domain ABD3 as disclosed herein and an antigen binding domain that binds to a tumor cell.

In some embodiments, the polypeptide chain comprises the antigen binding domain ABD3 as disclosed herein and an antigen binding domain that binds to immune cells.

In some embodiments, the polypeptide chain comprises at least one antigen binding domain selected from ABD1, ABD2 and ABD3 as disclosed herein.

In some embodiments, the polypeptide chain comprises at least two antigen binding domains selected from ABD1, ABD2 and ABD3 as disclosed herein.

In some embodiments, the polypeptide chain comprises at least three antigen binding domains selected from ABD1, ABD2 and ABD3 as disclosed herein.

In some embodiments, the dimerization domain comprises an immunoglobulin dimerization domain. Other dimerization domains known to a person skilled in the art are contemplated herein including leucine zippers, etc.

In some embodiments, the dimerization domain comprises an IgG, IgM, IgA, IgD or IgE dimerization domain (from human or animal IgGss, IgM, IgAs, IgDs or IgEs).

In some embodiments, the dimerization domain comprises a CH3 domain of an antibody. The dimerization domain may also comprise a CH2 domain of an antibody.

In some exemplary embodiments, the dimerization domain comprises a natural CH3 domain.

In some exemplary embodiments, the dimerization domain comprises a mutated CH3 domain.

In some embodiments, the dimerization domain comprises a natural CH2 and a natural CH3 domain.

In some embodiments, the dimerization domain comprises a natural CH2 and a mutated CH3 domain.

In some embodiments, the dimerization domain comprises a mutated CH2 and a mutated CH3 domain.

In some embodiments, the dimerization domain comprises a mutated CH2 and a natural CH3 domain.

In some embodiments, the dimerization domain does not comprise a CH1 domain.

In some embodiments, the dimerization domain does not comprise a CH4 domain.

In some embodiments, the dimerization domain comprises a Fc region of an antibody or a portion thereof.

In some embodiments, the polypeptide chain comprises at least two or at least three antigen binding domains and a dimerization domain that allow assembly of two polypeptide chains to form a multivalent and/or multispecific binding agent.

In embodiments, the antigen binding domain comprises or consist of an antigen binding domain of a single domain antibody (sdAb).

In embodiments, the antigen binding domain comprises a heavy chain variable region (VH or VHH).

In some embodiment the VHH is derived from humans, from a mouse, from a rat etc.

In some embodiment, the VHH is from a transgenic mouse or rat capable of expressing camelized mouse or rats VHHs, VHHs from other species (e.g., humans etc.) or camelized VHHs from other species (e.g., camelized human VHH etc.).

In some embodiments, the binding agent does not comprise a light chain variable region (VL or VLL).

In embodiments, the binding agent comprises a light chain variable region (VL or VLL).

In embodiments, the antigen binding domain is a single chain variable fragment (ScFv).

In embodiments, the antigen binding domain is from a V_{NAR} fragment.

In other embodiments, the antigen binding domains of the polypeptide chain comprises a combination of any antigen binding domain of single domain antibodies (sdAbs), heavy chain variable regions (VHs or VHHs), light chain variable regions (VLs or VLLs), single chain variable fragments (ScFvs) and/or V_{NAR} fragments.

In some embodiments, the sdAb or VHH is from a Camelidae antibody.

In embodiments, the Camelidae antibody is from a dromedary, a camel, a llama, an alpaca etc.

In other embodiments, the sdAb or VHH is from a cartilaginous fish antibody.

In embodiments, the cartilaginous fish antibody is a shark antibody.

In some embodiments, each antigen binding domain specifically binds to a different epitope.

In other embodiments, each antigen binding domain specifically binds to a different antigen.

In yet other embodiments, each antigen binding domain specifically binds to a different protein.

It is to be understood herein, that a given antigen binding domain may bind to an epitope that exists in different proteins. As such, in some embodiments, the antigen binding domain, or the binding agent comprising same, may bind to more than one protein. In some embodiments, the antigen binding domain, or the binding agent, may have affinity for more than one protein.

In some embodiments, the binding agent is an antibody or an antigen binding fragment thereof.

In other embodiments, the binding agent comprises an antibody or an antigen binding fragment thereof.

In other embodiments, the binding agent comprises the antigen binding domain of an antibody or an antigen binding fragment thereof.

In some embodiments, the binding agent is a single domain antibody.

In other embodiments, the binding agent comprises a single domain antibody.

In other embodiments, the binding agent comprises the antigen binding domain of a single domain antibody.

In some embodiments, the binding agent is a single chain antibody (e.g., scFv).

In other embodiments, the binding agent comprises a single chain antibody (e.g., scFv).

In some embodiments, the binding agent comprises at least two polypeptide chains which are capable of forming dimers.

In some embodiments, the two polypeptide chains are the same.

In some embodiments, the two polypeptide chains are different.

In some embodiments, the polypeptide chains are the same, and the binding agent is a homodimer.

In some embodiments, the polypeptide chains are different, and the binding agent is a heterodimer.

In some embodiments, the binding agent is multispecific.

In some embodiments, the binding agent is bispecific, trispecific or tetra specific.

In some embodiments, the binding agent comprises one or more polypeptide chains that are multispecific.

In some embodiments, the binding agent comprises one or more polypeptide chains that are bispecific, trispecific or tetra specific.

In some embodiments, the one or more polypeptide chains have the same valency and specificity.

In some embodiments, the one or more polypeptide chains have different valency and specificity.

In some embodiments, the one or more polypeptide chains each is an antibody heavy chain.

In some embodiments, the binding agent is an antibody or an antigen binding fragment thereof.

In some embodiments, the binding agent is a bispecific antibody.

In some embodiments, the bispecific antibody further comprises a first antibody light chain and a second antibody light.

In some embodiments, one or more of the antigen binding domains is humanized.

In some embodiments, one or more of the antigen binding domains is partially humanized.

In some embodiments, the antigen binding domains comprise one or more human frameworks.

In some embodiments, X or Y are independently selected from a linker, a cytokine, a chemokine, a tag, a masking domain, a phage coat protein (pIII, pVI, pV, pVII or pIX), an antigen binding domain or combination thereof.

In some embodiments, the polypeptide chain is conjugated to a therapeutic moiety.

In some embodiments, the polypeptide chain is conjugated to a detectable moiety.

In some embodiments, the polypeptide chain is conjugated to a protein allowing an extended half-life.

In some embodiments, the polypeptide chain is attached to nanoparticles.

Other aspects and embodiments of the present disclosure relate to a composition comprising at least one of the binding agents disclosed herein.

In some embodiments, the composition comprises monomers, dimers and mixture thereof.

In some embodiments, greater than 70%, 75%, 80%, 85%, 90%, 95%, 96%, 97%, 98%, or 99% of the first and second polypeptide chains exist as dimers in the composition.

In some embodiments, greater than 70%, 75%, 80%, 85%, 90%, 95%, 96%, 97%, 98%, or 99% of the first and second polypeptide chains exist as homodimers in the composition.

In some embodiments, greater than 70%, 75%, 80%, 85%, 90%, 95%, 96%, 97%, 98%, or 99% of the first and second polypeptide chains exist as heterodimers in the composition.

Other aspects and embodiments of the present disclosure relate to a pharmaceutical composition comprising a binding agent disclosed herein and a pharmaceutically acceptable carrier.

Yet other aspects and embodiments of the present disclosure relate to a nucleic acid or set of nucleic acids encoding the polypeptide chains and/or the binding agent disclosed herein.

The nucleic acid may be in the form of DNA segments as disclosed herein.

Additional aspects and embodiments of the present disclosure relate to a vector comprising a nucleic disclosed herein or a set of vectors each comprising a nucleic acid disclosed herein Further aspects and embodiments of the present disclosure relate to a cell expressing the polypeptide chains or binding agents disclosed herein.

Additional aspects and embodiments of the present disclosure relate to a cell comprising the nucleic acid or the vector disclosed herein.

Further aspects and embodiments of the present disclosure relate to a kit comprising the binding agent disclosed herein.

Yet further aspects and embodiments of the present disclosure relate to a kit comprising the nucleic acid disclosed herein.

Yet further aspects and embodiments of the present disclosure relate to a kit comprising the vector disclosed herein.

Yet further aspects and embodiments of the present disclosure relate to a kit comprising the cells disclosed herein.

In an additional aspect and embodiments, the present disclosure relates to a method of treating or preventing a disease or disorder associated with CD36, CD36 expression, CD36 overexpression or CD36 upregulation.

Exemplary embodiments of disease or disorder include cancer, metabolic diseases such as atherosclerosis, diabetes, non-alcoholic fatty liver disease, thrombosis, or inflammation.

In an additional aspect and embodiments, the present disclosure relates to a method of treating cancer comprising administering the binding agent disclosed herein to a subject in need.

In further aspects and embodiments, the present disclosure relates to a method of treating cancer comprising administering the composition disclosed herein to a subject in need.

In further aspects and embodiments, the present disclosure relates to a method of treating cancer comprising administering the pharmaceutical composition disclosed herein to a subject in need.

In some embodiments, the cancer is metastatic.

In some embodiments, the cancer is leukemia including without limitation acute myeloid leukemia.

In some embodiments, the cancer is a solid tumor.

In some embodiments, the cancer is selected from endometrial cancer, breast cancer (e.g., triple negative breast cancer), liver cancer (e.g., hepatocellular carcinoma), lung cancer, prostate cancer, renal cancer e.g., (renal clear cell cancer), ovarian cancer, cervical cancer, pancreatic cancer, gastric cancer, bladder cancer (e.g., bladder urothelial carcinoma), brain cancer, oral cancer, head and neck cancer, glioblastoma (e.g., glioblastoma multiform), adrenocortical carcinoma, colon cancer and colorectal cancer.

In some embodiments, the cancer originates from fat-rich (adipose) tissue.

In some embodiments, the cancer is associated with increased lipid metabolism. For example, the subject's tumor may show increased fatty acid uptake and/or increased fatty acid oxidation.

For example, the binding agent may be used to reduce and/or prevent the growth of tumor in a subject in need. In other example, the binding agent may be used to reduce and/or prevent tumor metastasis. In yet other examples, the binding agent may be used for reducing resistance to chemotherapy and/or immune checkpoint inhibitor therapy.

The binding agent may thus be administered to a subject having cancer selected from leukemia (e.g., acute myeloid leukemia), endometrial cancer, breast cancer (e.g., triple negative breast cancer), liver cancer (e.g., hepatocellular carcinoma), lung cancer, prostate cancer, renal cancer e.g., (renal clear cell cancer), ovarian cancer, cervical cancer, pancreatic cancer, gastric cancer, bladder cancer (e.g., bladder urothelial carcinoma), oral cancer, head and neck cancer, glioblastoma (e.g., glioblastoma multiform), adrenocortical carcinoma and colorectal cancer.

In some embodiments, the binding agent is administered to a subject that has failed prior treatment with chemotherapy (chemotherapeutics) and/or immune checkpoint therapy.

In some embodiments, the binding agent is administered to a subject that has relapsed leukemia. In some embodiments, the binding agent is administered to a subject that has relapsed acute myeloid leukemia. In some embodiments, the binding agent is administered to a subject having cancer with increased lipid metabolism such as for example and without limitations, increased fatty acid uptake and/or increased fatty acid oxidation.

In some embodiments, the binding agent is administered to a subject having cancer for reducing and/or preventing tumor growth.

In some embodiments, the binding agent is administered to a subject having cancer for reducing and/or preventing tumor metastasis.

In some embodiments, the binding agent is administered to a subject having cancer for reducing resistance to chemotherapy and/or immune checkpoint inhibitor therapy.

In other aspects and embodiments, the present disclosure relates to a method of making the binding agent disclosed herein, the method comprising transforming cells with one or more vectors comprising the nucleic acid disclosed herein.

In some embodiments, the method may further comprise isolating and/or purifying the binding agent from impurities.

In other embodiments, the method may further comprise isolating and/or purifying heterodimers from monomers and/or homodimers.

In other embodiments, the method may further comprise isolating and/or purifying homodimers from monomers and/or heterodimers.

In some embodiments, the method further comprises conjugating the binding agent with a therapeutic moiety, detectable moiety, or a protein allowing an extended half-life or to nanoparticles.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### BRIEF DESCRIPTION OF THE DRAWINGS

FIG. 1: Bar graph illustrating the binding of CD36 antibodies to human CD36 protein by ELISA at 10 μg/ml.

FIG. 2: Bar graph showing the binding of CD36 antibodies to OCI-AML3 cells (CD36+) and OCI-AML4 cells (CD36-).

FIG. 3: Graph illustrating the inhibitory effect of CD36 antibodies on fatty acid oxidation in OCI-AML3 cells at 350 nM concentration.

FIG. 4: Graph showing the effect of anti-CD36 antibodies on platelet activation at 50 μg/ml.

FIG. 5A-5B: schematic illustrating the configuration of monospecific single domain antibodies of Table 6 (FIG. 5A) and multivalent, multispecific binding agents of Table 7 (FIG. 5B).

FIG. 6: Graph showing the tumor regression of OCI-AML3 xenograft tumors in the I/O model by the anti-CD36 KisoBody KF061.

FIGS. 7A-7B: Alignment of anti-CD36 VHHs of Table 3 with IMGT CDRs (A) and Kabat CDRs (B) highlighted.

FIGS. 8A-8D: Alignment of selected anti-CD36 VHHs of Table 3.

FIG. 9: Alignment of selected anti-PD-1 VHHs of Table 4.

### DETAILED DESCRIPTION

### Definitions

Unless indicated otherwise, the amino acid numbering indicated for the dimerization domain are in accordance with the EU numbering system.

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “treatment” for purposes of this disclosure refers to both therapeutic treatment and prophylactic or tumor prevention measures. Subject in need of treatment include those already having cancer as well as those prone to have cancer or those in whom cancer is to be managed.

As used herein the term “CD36” refers to a CD36 protein or to a polypeptide comprising the CD36 amino acid sequence. The term “CD36” encompasses the human CD36 protein, CD36 antigen and any protein having at least 80% identity with the human CD36 protein.

The term “anti-CD36 antibody” refers to an antibody of any format that binds to the CD36 protein, CD36 antigen, or to a portion thereof and include without limitations “anti-CD36 single domain antibody”.

As used herein the term “PD-1” refers to a PD-1 protein or to a polypeptide comprising the PD-1 amino acid sequence. The term “PD-1” encompasses the human PD-1 protein, PD-1 antigen and any protein having at least 80% identity with the human PD-1 protein.

The term “anti-PD-1 antibody” refers to an antibody of any format that binds to the PD-1 protein, PD-1 antigen, or to a portion thereof and include without limitations “anti-PD-17 single domain antibody”.

As used herein the term “CD47” refers to a CD47 protein or to a polypeptide comprising the CD47 amino acid sequence. The term “CD47” encompasses the human CD47 protein, CD47 antigen and any protein having at least 80% identity with the human CD47 protein.

The term “anti-CD47 antibody” refers to an antibody of any format that binds to the CD47 protein, CD47 antigen, or to a portion thereof and include without limitations “anti-CD47 single domain antibody”.

The term “antibody” is used in the broadest sense and encompasses various antibody formats and structures, including any immunoglobulin, monoclonal antibody, polyclonal antibody, bivalent antibody, monovalent antibody, bispecific antibody, multiple specific (multi-specific) antibody, conventional antibody, single domain antibody, heavy chain only antibody, nanobody, full-length antibody, humanized antibody, chimeric antibody, that binds to a specific antigen, and any antigen binding fragment that exhibits the desired antigen binding activity. An antibody can be naturally occurring (native) or the results or recombination technologies.

The term “subject in need” as used herein refer to a subject having, suspected of having a disorder or disease associated with CD36, CD36 expression, CD36 overexpression or CD36 upregulation that may benefit from treatment with a binding agent targeting CD36. A “subject in need” include a subject having cancer or suspected of having cancer.

As used herein the expressions “single domain antibody” and “heavy chain only antibody” are used interchangeably. The term “single domain antibody” includes naturally occurring single domain antibody as well as single domain antibody in which the naturally occurring constant region is replaced by another dimerization domain such as for example a constant region of a human immunoglobulin (e.g., IgG) as well as variants such as humanized or chimeric single domain antibodies.

A “naturally occurring single domain antibody” includes antibodies produced by camelids (camelid antibodies) or by shark. “naturally occurring single domain antibody(ies)” also encompass antibody(ies) that are produced by transgenic animals modified to express heavy chain only antibodies. Exemplary embodiments of transgenic animals are provided in international application No. PCT/CA2021/050951 filed on Jul. 21, 2021 and published on Jan. 20, 2022 under No. WO2022/011457, the entire content of which is incorporated herein by reference. Although, naturally occurring single domain antibodies do not usually include light chain, the present disclosure does not exclude single domain antibodies comprising a light chain. It is to be understood herein that the term “naturally occurring antibody(ies)” or “naturally occurring single domain antibody(ies)” refers to antibody(ies) or single domain antibody(ies) isolated and/or purified from an animal generally upon immunization. The term “naturally occurring” is not intended to encompass products that exist in nature without human or machine intervention.

The term “antigen binding domain” relates to the portion of an antibody that is involved in antigen binding and comprises for example, one or more CDRs, one or more framework regions (FR) or the entire variable region. The term “antigen binding domain” in the context of a single domain antibody thereof relates to the portion of single domain antibody that is involved in antigen binding and comprises for example, one or more of CDR1 (CDRH1), CDR2 (CDRH2) or CDR3 (CDRH3), one or more framework regions FR1, FR2, FR3, FR4 or the entire variable region (VH or VHH). The term “antigen binding domain” in the context of a native antibody thereof relates to the portion of a native antibody that is involved in antigen binding and comprises for example, one or more of CDRH1, CDRH2, CDRH3, CDRL1, CDRL2 or CDRL3, one or more light chain or heavy chain framework regions FR1, FR2, FR3, FR4 or one or both entire variable regions (heavy chain variable domain (VH) and/or light chain variable domain (VL)).

The term “antigen binding fragment(s)” as used in the expression “antibody or antigen binding fragment thereof” or “single domain antibody or antigen binding fragment thereof” refers to a fragment of the antibody or single antibody that encompasses the antigen binding domain and that may incorporate or not, other portion(s) of the antibody or single domain antibody such as for example amino acid residues of the hinge region, amino acid residues of a constant region, portion of a Fc region. Regardless of structure, an antigen binding fragment binds to the same antigen that is recognized by the complete antibody (e.g., single domain antibody).

The term “binding agent” refers to a molecule that comprises a polypeptidic portion (such as polypeptide chain(s)) and that is capable of specifically binding to an antigen or that comprises a domain such as an antigen binding domain that is capable of specifically binding an antigen.

As used herein, the term “humanized” means that the antigen binding domain comprises human antibody amino acid residues. A “humanized antigen binding domain” will generally have CDRs derived from non-human animal antibodies, FR amino acid residues derived from human antibodies, and when applicable, a constant region derived from human antibodies. In some instances however, a “humanized antigen binding domain” may also comprise CDR amino acid residues derived from human antibodies. The term “humanized” encompasses “fully humanized” and “partially humanized”.

As used herein the expression “fully humanized” means that the amino acid sequence of all framework regions of a given antigen binding domain is identical to that of a human antibody such as a germline human antibody variable region. As such, the term “fully humanized antigen binding domain” encompasses an antigen binding domain with CDRs derived from non-human animal antibodies, all FR amino acid residues derived from human antibodies, and when applicable, a constant region derived from human antibodies. In some instances, a “fully humanized antigen binding domain” may also comprise CDR amino acid residues derived from human antibodies.

As used herein the expression “partially humanized” means that the amino acid sequence of one or more or all framework regions of a given antigen binding domain is not totally identical to that of a human antibody such as a germline human antibody variable region.

The expression “partially humanized” also applies to antigen binding domain that comprises human framework region comprising back-mutations reintroducing one or more original non-human amino acid residues in a human framework. As such, the term “partially humanized antigen binding domain” encompasses an antigen binding domain with CDRs derived from non-human animal antibodies, some FR amino acid residues derived from human antibodies, and when applicable, a constant region derived from human antibodies. In some instances, a “partially humanized antigen binding domain” may also comprise CDR amino acid residues derived from human antibodies.

The term “specific binding” or “specifically binds” as used herein refers to a non-random binding reaction between two molecules, such as for example between an antibody and an antigen. Specific binding can be characterized in binding affinity. A K_{D} value of ≤10⁻⁶M (e.g., ≤5×10⁻⁷M, ≤2×10⁻⁷M, ≤10⁻⁷M, ≤5×10⁻⁸M, ≤2×10⁻⁸M, ≤10⁻⁸M, ≤5×10⁻⁹M, ≤4×10⁻⁹M, ≤3×10⁻⁹M, ≤2×10⁻⁹M, or ≤10⁻⁹M and any value≤10⁻⁶) can indicate specific binding between a binding agent (e.g., antibody such as single domain antibody) and CD36 (e.g., human CD36).

As used herein, the term “affinity” refers to the strength of non-covalent interaction between a binding agent or antigen binding domain(s) thereof and an antigen. Affinity is represented for example, by K_{D} value, i.e., the ratio of dissociation rate to association rate (k_{off}/kₒₙ or kₐₛₛ/k_{d1ss}) when the binding between the antigen and the binding agent (e.g., antibody such as single domain antibody) reaches equilibrium. The affinity may be determined by using any conventional method known in the art, including but are not limited to, surface plasmon resonance method, microscale thermophoresis method, HPLC-MS method and flow cytometry (such as FACS) method. An antibody generally has a K_{D} value of ≤10⁻⁶M (e.g., ≤5×10⁻⁷M,≤2×10⁻⁷M,≤10⁻⁷M,≤5×10⁻⁸M, 2×10⁻⁸M,≤10⁻⁸M,≤5×10⁻⁹M,≤4×10⁻⁹M, ≤3×10⁻⁹M, ≤2×10⁻⁹M, or ≤10⁻⁹M and any value≤10⁻⁶). Preferablly, an antibody has a K_{D} value in the nanomolar range or lower (e.g., ≤9×10⁻⁹M, ≤8×10⁻⁹M, ≤7×10⁻⁹M, ≤6×10-9M, ≤5×10⁻⁹M, ≤4×10⁻⁹M, ≤3×10⁻⁹M, ≤2×10⁻⁹M, ≤1×10⁻⁹M or lower e.g., ≤1×10⁻¹⁰M, ≤1×10⁻¹¹ M, ≤1×10⁻¹²M). Even more preferably, an antibody has a K_{D} value in the picomolar range or lower (e.g., ≤9×10⁻¹²M, ≤8×10⁻¹²M, ≤7×10⁻¹²M, 6×10⁻¹²M, ≤5×10⁻¹²M, ≤4×10⁻¹²M, ≤3×10⁻¹²M, ≤2×10⁻¹²M, ≤1×10⁻¹²M or lower).

As used herein, the term “competing for binding”, “compete for binding”, “compete(s) with” or “competing with” refers to the ability of a first antibody or antigen-binding fragment thereof (e.g., single domain antibody or antigen binding fragment thereof) to inhibit the binding interaction between CD36 and a second antibody to the same CD36, between PD-1 and a second anti-PD-1 antibody, between CD47 and a second anti-CD47 antibody (e.g., a single domain antibody or antigen binding fragment thereof) to any detectable degree.

As used herein the term “epitope” as used herein refers to the specific group of atoms or amino acid residues on an antigen to which an antibody (e.g., single domain antibody) binds. Two antibodies may bind the same or a closely related epitope within an antigen if they exhibit competitive binding for the antigen. An epitope can be linear or conformational (i.e., including amino acid residues spaced apart). For example, if an antibody or antigen binding fragment blocks binding of a reference antibody (e.g., single domain antibody) to the antigen by at least 85%, or at least 90%, or at least 95%, then the antibody or antigen-binding fragment may be considered to bind the same/closely related epitope as the reference antibody. For example, a monoclonal, chimeric, human or humanized antibody or an antigen binding fragment thereof may compete with a single domain antibody of the present disclosure for binding to human DR2.

As used herein, the term “sequence identity” of the present invention indicates the degree of identity between two nucleic acid or two amino acid sequences when best compared and compared when a mutation such as substitution, insertion or deletion is appropriate. The sequence identity can be at least 85%, 90% or 95%, preferably at least 95%. Non-limiting examples include 85%, 86%, 87%, 88%, 89%, 90%, 91%, 92%, 93%, 94%, 95%, 95%, 96%, 97%, 98%, 99%, and 100%. In some embodiments, the framework regions are generally not considered as being part of the antigen binding domain. However, some amino acid residues of one or more framework region may contribute to antigen binding.

The term “functionally active” with reference to an antigen binding domain means that the antigen binding domain is capable of binding to its target and optionally that the antigen binding domain possesses one or more biological activities.

As used herein the term “flexible linker” refers to peptide comprising at least a portion composed of flexible amino acid residues that allow adjacent modules to move relative to one another.

As used herein the term “rigid linker” refers to peptide comprising at least a portion composed of amino acids that exhibit a rigid structure and that keeps a distance between two modules.

As used herein the term “helical linker” means a linker that is composed of amino acid residues that adopt a a-helical conformation.

As used herein the term “cleavable linker” refers to peptides that comprise an enzymatic cleavage site that is sensitive to proteases selected from ADAMS, ADAMTS, aspartate proteases, caspases, cysteine cathepsins, cysteine proteinases, metalloproteinases, serine proteases, coagulation factor proteases, Type II Transmembrane Serine Proteases (TTSPs) and combination thereof.

As used herein the term “monospecific” with respect to binding agents refers to binding agents that bind to a single antigen or epitope. A monospecific binding agents may thus have one antigen binding domain or more than one binding domains (which are the same or different) having the same specificity towards a given antigen or epitope.

As used herein the term “multivalent” with respect to binding agents refers to binding agents that bind to more than one antigen or epitope.

The term “multivalent” encompasses “bivalent “trivalent”, “tetravalent”, “pentavalent”, “hexavalent” and the like.

As used herein the term “multispecific” with respect to binding agents refers to binding agents that bind to at least two different antigens or epitopes.

The term “multispecific” encompasses “bispecific”, “trispecific”, “tetraspecific”, “pentaspecific”, “hexaspecific” and the like.

As used herein in the context of binding agents the term “bispecific” designates a binding agent that binds to two different antigens or proteins or to two epitopes of the same antigen or protein.

As used herein, the expression “at least two polypeptide chains” and the like such as in the expression “binding agent comprises at least two polypeptide chains” and the like refers to a number of polypeptide chain species and not to an absolute numerical value.

It is to be understood herein, that expressions referring to ranges of values in the format such as “from A to B”, include each individual value and any sub-range comprised and including such ranges. For example, the expression “from 1 to 10” includes sub-ranges such as and without limitations, “from 2 to 10”, “from 2 to 9”, “from 3 to 6”, “from 5 to 7” and any individual values comprised between and including 1 and 10, i.e., 1, 2, 3, 4, 5, 6, 7, 8, 9 or 10.

It is to be understood herein that the term “at least” with respect to a given value intends to include the value and superior values. For example, the term “at least 80%” include “at least 81%”, “at least 82%”, “at least 83%”, “at least 84%”, “at least 85%”, “at least 86%”, “at least 87%”, “at least 88%”,“at least 89%”, “at least 90%”, “at least 91%”, “at least 92%”, “at least 93%”, “at least 94%”, “at least 95%”, “at least 96%”, “at least 97%”, “at least 98%”, “at least 99%”, “at least 99.1%”, “at least 99.2%”, at least 99.3%”, at least 99.4%”, at least 99.5%”, at least 99.6%”, at least 99.7%”, at least 99.8%”, at least 99.9%”, and 100%.

The term “about” or “approximately” with respect to a given value means that variation in the value is contemplated. In some embodiments, the term “about” or “approximately” shall generally mean a range within +/−10 percent, within +/−5 percent, within +/−4 percent, within +/−3 percent, within +/−2 percent or within +/−1 percent of a given value or range.

The term “codon-optimized” refers to a sequence for which a codon has been changed for another codon encoding the same amino acid but that is preferred or that performs better in a given organism (increases expression, minimize secondary structures in RNA etc.). “Codon-optimized” sequences may be obtained, using publicly available softwares or via service providers including GenScript (OptimumGene™, U.S. Pat. No. 8,326,547).

The term “single chain format” with respect to antigen binding domain means that the domain necessary for antigen binding is on a single polypeptide chain.

It is to be understood herein that any of the terms “KF023”, “KF024”, “KF025”, “KF026”, “KF027”, “KF028”, “KF029”, “KF030”, “KF031”, “KF032”, “KF033”, “KF034”, “KF035”, “KF036”, “KF037”, “KF038”, “KF039”, “KF040”, “KF041”, “KF042”, “KF043”, “KF044”, “KF045”, “KF046”, “KF047”, “KF048”, “KF049”, “KF050”, “KF051”, “KF052”, “KF053”, “KF054”, “KF055”, “KF056”, “KF057”, “KF058”, “KF059”, or “KF061” refer to a binding agent that comprises two chains, each having the corresponding amino acid sequence set forth in Table 6 or Table 7 or as described herein.

### Antigen Binding Domains (Abs)

As disclosed herein, the binding agent may comprise one or more antigen binding domains.

For example, the binding agent of the present disclosure comprises one or more antigen binding domain capable of binding to CD36. The binding agent may also comprise one or more antigen binding domain that binds to one or more other antigens.

In some instances, the antigen binding domain may, for example, bind to an immune checkpoint protein.

In other instances, the antigen binding domain may, for example, bind to a protein expressed at the surface of immune cells.

The antigen binding domains of the present disclosure may be selected for their ability to bind specific targets. The antigen binding domains may also be selected for their in vivo and/or in vitro functional properties or biological effects including, for example, their ability to modulate cellular processes such as gene expression, signal transduction, cell growth, cell viability and the like.

For example, the binding agents of the present disclosure comprise one or more antigen binding domains each independently comprising one or more complementarity determining region(s) (CDRs) of an antibody.

The specificity of the binding agents of the present disclosure may thus be conferred by their antigen binding domains.

The binding agent of the present disclosure may comprise an antigen binding domain capable of binding to CD36 or to cells expressing CD36, an antigen binding domain capable of binding to PD-1 or to cells expressing PD-1 and/or an antigen binding domain capable of binding to CD47 or to cells expressing CD47.

In some embodiments, the antigen binding domain is an antigen binding domain capable of binding to CD36 or to cells expressing CD36 and includes for example, antigen binding domain 1 (ABD1). In some embodiments, the cells expressing CD36 comprise tumor cells.

In some embodiments, the antigen binding domain is an antigen binding domain capable of binding to PD-1 or to cells expressing PD-1 and includes for example, antigen binding domain 2 (ABD2). In some embodiments, the cells expressing PD-1 comprises immune cells.

In some embodiments, the antigen binding domain is an antigen binding domain capable of binding to CD47 or to cells expressing CD47 and includes for example, antigen binding domain 3 (ABD3). In some embodiments, the cells expressing CD47 comprises immune cells or tumor cells.

In some embodiments, the antigen binding domain targets the same epitope or antigen as ABD1, ABD2 and/or ABD3. In some embodiments, the antigen binding domain competes with ABD1, ABD2 and/or ABD3 for binding to their respective epitope or antigen.

In some embodiments, the antigen binding domain binds to at least one antigen that is not selected amongst CD36, PD-1 or CD47.

In some embodiments, the binding agent may comprise more than one antigen binding domains.

For example, in some embodiments, the binding agent may comprise two antigen binding domains or more.

In some embodiments, the binding agent may comprise three antigen binding domains or more.

In some embodiments, the binding agent may comprise four antigen binding domains or more.

In some embodiments, the binding agent may comprise five antigen binding domains or more.

In some embodiments, the binding agent may comprise six antigen binding domains or more.

In some embodiments, the binding agent may comprise between one and twelve antigen binding domains, such as between one and two, between one and three, between one and four, between one and five, between one and six, between one and seven, between one and eight, between one and nine, between one and ten, between one and eleven, between one and twelve, between two and three, between two and four, between two and five, between two and six, between two and seven, between two and eight, between two and nine, between two and ten, between two and eleven, between two and twelve, between three and four, between three and five, between three and six, between three and seven, between three and eight, between three and nine, between three and ten, between three and eleven, between three and twelve, between four and five, between four and six, between four and seven, between four and eight, between four and nine, between four and ten, between four and eleven, between four and twelve, between five and six, between five and seven, between five and eight, between five and nine, between five and ten, between five and eleven, between five and twelve, between six and seven, between six and eight, between six and nine, between six and ten, between six and eleven, between six and twelve, between seven and eight, between seven and nine, between seven and ten, between seven and eleven, between seven and twelve, between eight and nine, between eight and ten, between eight and eleven, between eight and twelve, between nine and ten, between nine and eleven, between nine and twelve, between ten and eleven, between ten and twelve, or between eleven and twelve.

In some embodiments, the binding agents of the present disclosure may comprise one or more antigen binding domains and at least one of the antigen binding domains is capable of binding to tumor cells.

In some embodiments, the binding agent is capable of binding to tumor cells. In other embodiments, the binding agent is capable of binding to a protein expressed at the surface of tumor cells.

In some embodiments, the binding agents of the present disclosure may comprise one or more antigen binding domains and at least one of the antigen binding domains is capable of binding to an immunomodulator. In some embodiments, at least one of the antigen binding domains is capable of binding to an immune checkpoint protein. In some embodiments, the immune checkpoint protein is PD-1.

In some embodiments, the binding agents of the present disclosure may comprise one or more antigen binding domains and at least one of the antigen binding domains is capable of binding to immune cells. In some embodiments, at least one of the antigen binding domains is capable of binding to a protein expressed at the surface of immune cells (e.g., T cells, NK-cells, monocytes, macrophages etc.).

In some embodiments, the binding agent is capable of binding to immune cells. In other embodiments, the binding agent is capable of binding to a protein expressed at the surface of immune cells.

In some embodiments, the binding agents of the present disclosure may be multivalent and may comprise at least one antigen binding domain that binds tumor cells and at least one antigen binding domain that binds immune cells.

In some embodiments, the protein expressed at the surface of immune cells is selected from CD47 or CD3.

In some embodiments, the protein expressed at the surface of immune cells is CD47.

In some embodiments, the protein expressed at the surface of immune cells is CD3.

The antigen binding domains may be derived from a natural antibody (of human or animal origin) or from a synthetic antibody.

In some embodiments, antigen binding domains of a natural antibody are engineered so as to form a single chain.

In some embodiments, antigen binding domains may be obtained from IgGs such as IgG1, IgG2, IgG3 or IgG4. In particular embodiments, antigen binding domains are derived from a human IgG heavy chain.

In some embodiments, the antigen binding domains may be obtained from heavy chain only antibodies (HCAbs).

Exemplary embodiments of antigen binding domains include for example and without limitation a single domain antibody (sdAb), a heavy chain variable region (VH or VHH), a light chain variable region (VL or VLL), a single chain variable fragment (scFv), a V_{NAR} fragment, and combinations thereof.

In some embodiments the antigen binding domain of the binding agents disclosed herein is a VHH.

In some embodiments the antigen binding domain of the binding agents disclosed herein is a VHH that comprises the complementarity determining regions of the VHH disclosed herein.

In some embodiments the complementarity determining regions (CDRs) of the VHH disclosed herein correspond to Kabat CDRs.

In some embodiments the complementarity determining regions (CDRs) of the VHH disclosed herein correspond to IMGT CDRs.

In a particular embodiment, the binding agents of the present disclosure may comprise an antigen binding domain VHH derived from humans or a mouse or rat or from a transgenic mouse or rat wherein a mouse or rat VHH has been camelized, a human VHH, a human VHH which has been camelized, of an IgG1, IgG2a, IgG2b, IgG2c or IgG3 or combination thereof.

The antibodies may be obtained by immunizing a mouse or a rat or a transgenic mouse or rat which is lacking a functional CH1 domain in any of its heavy chains, IgG1, IgG2a, IgG2b, IgG2c or IgG3 or combination thereof, or a combination of the VHH described above, with an antigen of interest.

In a particular embodiment, the polypeptide chains of the present disclosure may comprise an antigen binding domain of a camelid antibody such as VHH of an IgG2 or IgG3.

The camelid antibodies may be obtained by immunizing a dromedary, a camel, a llama or an alpaca with an antigen of interest.

In some embodiments, the camelid antibodies may originate from the so-called old-world camelids such as Camelus bactrianus, Camelus dromaderus or from new-world camelids such as Lama pacos, Lama glama and Lama vicugna.

In another particular embodiment, the polypeptide chains of the present disclosure may comprise an antigen binding domain of a cartilaginous fish such as a V_{NAR} fragment of IgNAR. The V_{NAR} fragment may originate from shark antibodies.

If desired, the antigen binding domain of a non-human antibody may be humanized. For example, the framework region of non-human VH, VHH or HCAbs may be modified so as to render them more human-like. Humanization of camelid antibodies is discussed for example in Vincke C. et al. (J.Biol Chem. 2009, 284(5):3273-3284), the entire content of which is incorporated herein by reference. Humanized camelid antibodies may be obtained, for example, by CDR grating onto a universal humanized nanobody scaffold (e.g., h-NbBcIIOFG_{L}A disclosed in Vincke C. et al.). V_{NAR} antibodies can be humanized by converting non-CDR residues to those of human germline V□1 sequence DPK9 as discussed in Kovalenko OV et al. (J Biol Chem. 2013, 288:17408-17419) the entire content of which is incorporated herein by reference. The polypeptide chains of the present disclosure therefore encompass humanized antigen binding domains.

In yet another particular embodiment, the antigen binding domain may comprise a human VH (modified or not). Human VH may be obtained for example, from synthetic human VH libraries. Modified human VH include those in which some amino acid residues have been modified to render them more camel-like (i.e., by camelization).

A person skilled in the art will understand that the antigen binding domains may be incorporated into an antibody, antigen binding fragment or an antibody-like molecule including without limitations, single domain antibodies (sdAb), VHH, conventional antibodies or antigen binding fragments thereof, bispecific antibodies, single chain Fv-CH3 (scFv-CH3) fusion, tandem-scFv-CH3 (TaFv-CH3) fusion, diabody-CH3 (Db-CH3) fusion, tandem Db-CH3 (TaDb-CH3) fusion, single chain Db-CH3 fusion (scDb-CH3), Fab-CH3 fusion, single chain Fab-CH3 fusion, Fab-scFv-CH3 fusion, dual affinity retargeting (DART)-CH3 fusion, Fab-DART-CH3 fusion, single chain Fv- Fc (scFv-Fc) fusion, tandem-scFv-Fc (TaFv-Fc) fusion, diabody-Fc (Db-Fc) fusion, tandem Db-Fc (TaDb-Fc) fusion, single chain Db-Fc fusion (scDb-Fc), Fab-Fc fusion, single chain Fab-Fc fusion, Fab-scFv-Fc fusion, dual affinity retargeting (DART)-Fc fusion, Fab-DART-Fc fusion etc. These antibody formats may have natural CH3, mutated CH3 domains, natural CH2-CH3 domains or mutated CH2-CH3 domains disclosed herein.

### Exemplary Embodiments of ABD1

Antigen binding domain 1 (ABD1) may be selected, for example, from antigen binding domains that bind CD36, some non-limiting exemplary embodiments of which is provided herein.

In some instances, ABD1 is capable of binding human CD36 or a portion thereof or cells expressing human CD36 or a portion thereof.

In some exemplary embodiments, the binding agent of the present disclosure may comprise one or more antigen binding domains, wherein at least one of the antigen binding domains is an antigen binding domain 1 (ABD1) and comprises a heavy chain complementarity determining region 1 (CDRH1) having the amino acid sequence set forth in SEQ ID NO:1, a heavy chain complementarity determining region 2 (CDRH2) having the amino acid sequence set forth in SEQ ID NO:2 and a heavy chain complementarity determining region 3 (CDRH3) having the amino acid sequence set forth in SEQ ID NO:3.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:4, a CDRH2 having the amino acid sequence set forth in SEQ ID NO:5 and a CDRH3 having the amino acid sequence set forth in SEQ ID NO:6.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:8, a CDRH2 having the amino acid sequence set forth in SEQ ID NO:9 and a CDRH3 having the amino acid sequence set forth in SEQ ID NO:10.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:11, a CDRH2 having the amino acid sequence set forth in SEQ ID NO:12 and a CDRH3 having the amino acid sequence set forth in SEQ ID NO:13.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:15, a CDRH2 having the amino acid sequence set forth in SEQ ID NO:16 and a CDRH3 having the amino acid sequence set forth in SEQ ID NO:17.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:18, a CDRH2 having the amino acid sequence set forth in SEQ ID NO:19 and a CDRH3 having the amino acid sequence set forth in SEQ ID NO:20.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:22, a CDRH2 having the amino acid sequence set forth in SEQ ID NO:23 and a CDRH3 having the amino acid sequence set forth in SEQ ID NO:24.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:25, a CDRH2 having the amino acid sequence set forth in SEQ ID NO:26 and a CDRH3 having the amino acid sequence set forth in SEQ ID NO:27.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:29, a CDRH2 having the amino acid sequence set forth in SEQ ID NO:30 and a CDRH3 having the amino acid sequence set forth in SEQ ID NO:31.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:32, a CDRH2 having the amino acid sequence set forth in SEQ ID NO:33 and a CDRH3 having the amino acid sequence set forth in SEQ ID NO:34.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:36, a CDRH2 having the amino acid sequence set forth in SEQ ID NO:37 and a CDRH3 having the amino acid sequence set forth in SEQ ID NO:38.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:39, a CDRH2 having the amino acid sequence set forth in SEQ ID NO:40 and a CDRH3 having the amino acid sequence set forth in SEQ ID NO:41.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:43, a CDRH2 having the amino acid sequence set forth in SEQ ID NO:44 and a CDRH3 having the amino acid sequence set forth in SEQ ID NO:45.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:46, a CDRH2 having the amino acid sequence set forth in SEQ ID NO:47 and a CDRH3 having the amino acid sequence set forth in SEQ ID NO:48.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:50, a CDRH2 having the amino acid sequence set forth in SEQ ID NO:51 and a CDRH3 having the amino acid sequence set forth in SEQ ID NO:52.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:53, a CDRH2 having the amino acid sequence set forth in SEQ ID NO:54 and a CDRH3 having the amino acid sequence set forth in SEQ ID NO:55.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:71, a CDRH2 having the amino acid sequence set forth in SEQ ID NO:72 and a CDRH3 having the amino acid sequence set forth in SEQ ID NO:73.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:74, a CDRH2 having the amino acid sequence set forth in SEQ ID NO:75 and a CDRH3 having the amino acid sequence set forth in SEQ ID NO:76.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:78, a CDRH2 having the amino acid sequence set forth in SEQ ID NO:79 and a CDRH3 having the amino acid sequence set forth in SEQ ID NO:80.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:81, a CDRH2 having the amino acid sequence set forth in SEQ ID NO:82 and a CDRH3 having the amino acid sequence set forth in SEQ ID NO:83.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:85, a CDRH2 having the amino acid sequence set forth in SEQ ID NO:86 and a CDRH3 having the amino acid sequence set forth in SEQ ID NO:87.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:88, a CDRH2 having the amino acid sequence set forth in SEQ ID NO:89 and a CDRH3 having the amino acid sequence set forth in SEQ ID NO:90.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:92, a CDRH2 having the amino acid sequence set forth in SEQ ID NO:93 and a CDRH3 having the amino acid sequence set forth in SEQ ID NO:94.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:95, a CDRH2 having the amino acid sequence set forth in SEQ ID NO:96 and a CDRH3 having the amino acid sequence set forth in SEQ ID NO:97.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:99, a CDRH2 having the amino acid sequence set forth in SEQ ID NO:100 and a CDRH3 having the amino acid sequence set forth in SEQ ID NO:101.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:102, a CDRH2 having the amino acid sequence set forth in SEQ ID NO:103 and a CDRH3 having the amino acid sequence set forth in SEQ ID NO:104.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:106, a CDRH2 having the amino acid sequence set forth in SEQ ID NO:107 and a CDRH3 having the amino acid sequence set forth in SEQ ID NO:108.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:109, a CDRH2 having the amino acid sequence set forth in SEQ ID NO:110 and a CDRH3 having the amino acid sequence set forth in SEQ ID NO:111.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:113, a CDRH2 having the amino acid sequence set forth in SEQ ID NO: 114 and a CDRH3 having the amino acid sequence set forth in SEQ ID NO:15.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:116, a CDRH2 having the amino acid sequence set forth in SEQ ID NO:117 and a CDRH3 having the amino acid sequence set forth in SEQ ID NO:118.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:120, a CDRH2 having the amino acid sequence set forth in SEQ ID NO:121 and a CDRH3 having the amino acid sequence set forth in SEQ ID NO:122.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:123, a CDRH2 having the amino acid sequence set forth in SEQ ID NO:124 and a CDRH3 having the amino acid sequence set forth in SEQ ID NO:125.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:127, a CDRH2 having the amino acid sequence set forth in SEQ ID NO:128 and a CDRH3 having the amino acid sequence set forth in SEQ ID NO:129.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:130, a CDRH2 having the amino acid sequence set forth in SEQ ID NO:131 and a CDRH3 having the amino acid sequence set forth in SEQ ID NO:132.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:134, a CDRH2 having the amino acid sequence set forth in SEQ ID NO:135 and a CDRH3 having the amino acid sequence set forth in SEQ ID NO:136.

In some exemplary embodiments, the binding agent of the present disclosure may comprise one or more antigen binding domains, wherein at least one of the antigen binding domains is an antigen binding domain 1 (ABD1) and comprises a CDRH1 having the amino acid sequence set forth in SEQ ID NO:137, a CDRH2 having the amino acid sequence set forth in SEQ ID NO:138 and a CDRH3 having the amino acid sequence set forth in SEQ ID NO:139.

In some embodiments, the binding agent of the present disclosure may have the CDRH1, CDRH2, CDRH3 combination of ABD1 with one, two, three, four or five amino acid substitutions in any of the CDRH1, CDRH2 or CDRH3. In some embodiments, the total number of amino acid substitutions in any of the CDRH1, CDRH2 or CDRH3 is ten amino acid substitutions or less (total of nine, eight, seven, six, five, four, three, two or one). In some embodiments, the total number of amino acid substitutions in CDRH3 is three or less (one, two or three).

In some exemplary embodiments, the binding agent of the present disclosure may comprise one or more antigen binding domains, wherein at least one of the antigen binding domains is an antigen binding domain 1 (ABD1) and comprises the amino acid sequence set forth in SEQ ID NO:380.

(SEQ ID NO: 380)
QVQLQESGGGLX₂ₐQPGGSLRLSCAASGX_{2b}X_{2c}X_{2d}SX₂ₑYAMSWYRQX_{2f}
PGKERELVALITX_{2g}X₂ₕGGX₂ᵢTDYADSVKGRFTIX₂ⱼRDDAEKTVSLQ
MNSLKPEDTAVYYCSAWRAX₂ₖLX₂ₗX₂ₘPYWGQGTQVTVSS

wherein any one of X₂ₐ to X₂ₘ is any (e.g., natural) amino acid residue or wherein X₂ₐ is A or V, wherein X_{2b} is L or F, wherein X_{2c} is A, T or S, wherein X_{2d} is F or V, wherein X₂ₑ is T or S, wherein X_{2f} is A or P, wherein X_{2g} is S or T, wherein X₂ₕ is A or T, wherein X₂ᵢ is I or T, wherein X₂ⱼ is A or S, wherein X₂ₖ is S or T, wherein X₂ₗ is F, Y or L and/or wherein X₂ₘ is E or K.

In some embodiments, CDR1, CDR2, and/or CDR3 of SEQ ID NO:380 may independently correspond to the CDR1, CDR2, and/or CDR3 of any one of SEQ ID NOs.7, 28, 98, or 119 and combination thereof.

In some embodiments, the framework region of SEQ ID NO:380 may be modified to include amino acid substitutions, additions and/or deletions. In some embodiments, the framework region of SEQ ID NO:380 may comprise amino acid residues of a human antibody (e.g., human germline antibody sequence or consensus thereof).

In some exemplary embodiments, the binding agent of the present disclosure may comprise one or more antigen binding domains, wherein at least one of the antigen binding domains is an antigen binding domain 1 (ABD1) and comprises the amino acid sequence set forth in SEQ ID NO:381.

(SEQ ID NO: 381)
QVQLQESGGGLVQPGX₃ₐSLRLSCX_{3b}ASGX_{3c}X_{3d}X₃ₑX_{3f}FTFSX_{3g}YAM
SWYRQX₃ₕPGKERELVAAX₃ᵢX₃ⱼX₃ₖX₃ₗGX₃ₘX₃ₙX₃ₒX₃ₚYX_{3q}DSVKGRF
X₃ᵣISRDX₃ₛAKNTVYLX₃ₜMNSLKPEDTAX₃ᵤYYCNAVTLQGWX₃ᵥEYD
YWGQGTQVTVSS

wherein any one of X₃ₐ to X₃ᵥ is any (e.g., natural) amino acid residue or wherein X₃ₐ is G or E, wherein X_{3b} is A or T, wherein X_{3c} is absent or F, wherein X_{3d} is absent or T, wherein X₃ₑ is absent or S, wherein X_{3f} is absent or G, wherein X_{3g} is S or N, wherein X₃ₕ is A or T, wherein X₃ᵢ is M or I, wherein X₃ⱼ is Q, P or T, wherein X₃ₖ is S, H or N, wherein X₃ₗ is T, A or D, wherein X₃ₘ is D or G, wherein X₃ₙ is S or T, wherein X₃ₒ is T or I, wherein X₃ₚis Y or H, wherein X_{3q} is A or V, wherein X₃ᵣ is S or T, wherein X₃ₛ is D or N, wherein X₃ₜ is Q or L, wherein X₃ᵤ is V or A and/or wherein X₃ᵥ is S or R.

In some embodiments, X_{3c}, X_{3d}, X₃ₑ and X_{3f} are absent.

In some embodiments, CDR1, CDR2, and/or CDR3 of SEQ ID NO:381 may independently correspond to the CDR1, CDR2, and/or CDR3 of any one of SEQ ID NOs.14, 21, 35 or 49 and combination thereof.

In some embodiments, the framework region of SEQ ID NO:381 may be modified to include amino acid substitutions, additions and/or deletions. In some embodiments, the framework region of SEQ ID NO:381 may comprise amino acid residues of a human antibody (e.g., human germline antibody sequence or consensus thereof).

In some exemplary embodiments, the binding agent of the present disclosure may comprise one or more antigen binding domains, wherein at least one of the antigen binding domains is an antigen binding domain 1 (ABD1) and comprises the amino acid sequence set forth in SEQ ID NO:382.

(SEQ ID NO: 382)
QVQLQESGGGLVQPGGSLRLSCAASGFRFSX₄ₐSIMX_{4b}WYRQAPX_{4c}K
ERELVASISSDSX_{4d}RTNYAESVKGRFTISRDNEQX₄ₑX_{4f}X_{4g}FLEMN
X₄ₕLKPVDTAVYFCTRPX₄ᵢIRGRDWGQGTQVTVSS

wherein any one of X₄ₐ to X₄ᵢ is any (e.g., natural) amino acid residue or wherein X₄ₐ is S or T, wherein X_{4b} is S or T, wherein X_{4c} is E or G, wherein X_{4d} is V or R, wherein X₄ₑ is R or S, wherein X_{4f} is M or T, wherein X_{4g} is V or L, wherein X₄ₕ is S or N, wherein X₄, is S or N.

In some embodiments, CDR1, CDR2, and/or CDR3 of SEQ ID NO:382 may independently correspond to the CDR1, CDR2, and/or CDR3 of any one of SEQ ID NOs.56 or 84 and combination thereof.

In some embodiments, the framework region of SEQ ID NO:382 may be modified to include amino acid substitutions, additions and/or deletions. In some embodiments, the framework region of SEQ ID NO:382 may comprise amino acid residues of a human antibody (e.g., human germline antibody sequence or consensus thereof).

In some exemplary embodiments, the binding agent of the present disclosure may comprise one or more antigen binding domains, wherein at least one of the antigen binding domains is an antigen binding domain 1 (ABD1) and comprises the amino acid sequence set forth in SEQ ID NO:383.

(SEQ ID NO: 383)
QVQLQESGGGLVQPGGSLRLSCAASGRTFSSYPMGWFRQAPGKEREFVAA
IDWSGDITYYADSVKGRFAISRDNAKNMVYLQMNSLKPEDTAVYYCNAGP
LDSWERNDFPSWX₅ₐQGTQVTVSS

wherein X₅ₐ is any (e.g., natural) amino acid residue or wherein X₅ₐ is G or D.

In some embodiments, CDR1, CDR2, and/or CDR3 of SEQ ID NO:383 may independently correspond to the CDR1, CDR2, and/or CDR3 of any one of SEQ ID NOs.105 or 112 and combination thereof.

In some embodiments, the framework region of SEQ ID NO:383 may be modified to include amino acid substitutions, additions and/or deletions. In some embodiments, the framework region of SEQ ID NO:383 may comprise amino acid residues of a human antibody (e.g., human germline antibody sequence or consensus thereof).

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:7.

In some embodiments, the antigen binding domain 1 (ABD1) comprises an amino acid sequence having from one to ten amino acid substitutions, additions and/or deletions in the amino acid sequence set forth in SEQ ID NO:7.

In some embodiments, the amino acid variation may be located in one or more framework regions of SEQ ID NO:7.

In some embodiment, the antigen binding domain 1 (ABD1) comprises an amino acid sequence at least 75%, 80% 85%, 90%, 95% identical to the amino acid sequence set forth in SEQ ID NO:7 and CDRs identical to the Kabat CDRs of SEQ ID NO:7.

In some embodiment, the antigen binding domain 1 (ABD1)comprises an amino acid sequence at least 75%, 80% 85%, 90%, 95% identical to the amino acid sequence set forth in SEQ ID NO:7 and CDRs identical to the IMGT CDRs of SEQ ID NO:7.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:7.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:14.

In some embodiments, the antigen binding domain 1 (ABD1) comprises an amino acid sequence having from one to ten amino acid substitutions, additions and/or deletions in the amino acid sequence set forth in SEQ ID NO:14.

In some embodiments, the amino acid variation may be located in one or more framework regions of SEQ ID NO:14.

In some embodiment, the antigen binding domain 1 (ABD1) comprises an amino acid sequence at least 75%, 80% 85%, 90%, 95% identical to the amino acid sequence set forth in SEQ ID NO:14 and CDRs identical to the Kabat CDRs of SEQ ID NO:14.

In some embodiment, the antigen binding domain 1 (ABD1) comprises an amino acid sequence at least 75%, 80% 85%, 90%, 95% identical to the amino acid sequence set forth in SEQ ID NO:14 and CDRs identical to the IMGT CDRs of SEQ ID NO:14.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:14.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:21.

In some embodiments, the antigen binding domain 1 (ABD1) comprises an amino acid sequence having from one to ten amino acid substitutions, additions and/or deletions in the amino acid sequence set forth in SEQ ID NO:21.

In some embodiments, the amino acid variation may be located in one or more framework regions of SEQ ID NO:21.

In some embodiment, the antigen binding domain 1 (ABD1) comprises an amino acid sequence at least 75%, 80% 85%, 90%, 95% identical to the amino acid sequence set forth in SEQ ID NO:21 and CDRs identical to the Kabat CDRs of SEQ ID NO:21.

In some embodiment, the antigen binding domain 1 (ABD1) comprises an amino acid sequence at least 75%, 80% 85%, 90%, 95% identical to the amino acid sequence set forth in SEQ ID NO:21 and CDRs identical to the IMGT CDRs of SEQ ID NO:21.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:21.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:28.

In some embodiments, the antigen binding domain 1 (ABD1) comprises an amino acid sequence having from one to ten amino acid substitutions, additions and/or deletions in the amino acid sequence set forth in SEQ ID NO:28.

In some embodiments, the amino acid variation may be located in one or more framework regions of SEQ ID NO:28.

In some embodiment, the antigen binding domain 1 (ABD1) comprises an amino acid sequence at least 75%, 80% 85%, 90%, 95% identical to the amino acid sequence set forth in SEQ ID NO:28 and CDRs identical to the Kabat CDRs of SEQ ID NO:28.

In some embodiment, the antigen binding domain 1 (ABD1) comprises an amino acid sequence at least 75%, 80% 85%, 90%, 95% identical to the amino acid sequence set forth in SEQ ID NO:28 and CDRs identical to the IMGT CDRs of SEQ ID NO:28.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:28.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:35.

In some embodiments, the antigen binding domain 1 (ABD1) comprises an amino acid sequence having from one to ten amino acid substitutions, additions and/or deletions in the amino acid sequence set forth in SEQ ID NO:35.

In some embodiments, the amino acid variation may be located in one or more framework regions of SEQ ID NO:35.

In some embodiment, the antigen binding domain 1 (ABD1) comprises an amino acid sequence at least 75%, 80% 85%, 90%, 95% identical to the amino acid sequence set forth in SEQ ID NO:35 and CDRs identical to the Kabat CDRs of SEQ ID NO:35.

In some embodiment, the antigen binding domain 1 (ABD1) comprises an amino acid sequence at least 75%, 80% 85%, 90%, 95% identical to the amino acid sequence set forth in SEQ ID NO:35 and CDRs identical to the IMGT CDRs of SEQ ID NO:35.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:35.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:42.

In some embodiments, the antigen binding domain 1 (ABD1) comprises an amino acid sequence having from one to ten amino acid substitutions, additions and/or deletions in the amino acid sequence set forth in SEQ ID NO:42.

In some embodiments, the amino acid variation may be located in one or more framework regions of SEQ ID NO:42.

In some embodiment, the antigen binding domain 1 (ABD1) comprises an amino acid sequence at least 75%, 80% 85%, 90%, 95% identical to the amino acid sequence set forth in SEQ ID NO:42 and CDRs identical to the Kabat CDRs of SEQ ID NO:42.

In some embodiment, the antigen binding domain 1 (ABD1) comprises an amino acid sequence at least 75%, 80% 85%, 90%, 95% identical to the amino acid sequence set forth in SEQ ID NO:42 and CDRs identical to the IMGT CDRs of SEQ ID NO:42.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:42.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:49.

In some embodiments, the antigen binding domain 1 (ABD1) comprises an amino acid sequence having from one to ten amino acid substitutions, additions and/or deletions in the amino acid sequence set forth in SEQ ID NO:49.

In some embodiments, the amino acid variation may be located in one or more framework regions of SEQ ID NO:49.

In some embodiment, the antigen binding domain 1 (ABD1) comprises an amino acid sequence at least 75%, 80% 85%, 90%, 95% identical to the amino acid sequence set forth in SEQ ID NO:49 and CDRs identical to the Kabat CDRs of SEQ ID NO:49.

In some embodiment, the antigen binding domain 1 (ABD1) comprises an amino acid sequence at least 75%, 80% 85%, 90%, 95% identical to the amino acid sequence set forth in SEQ ID NO:49 and CDRs identical to the IMGT CDRs of SEQ ID NO:49.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:49.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:56.

In some embodiments, the antigen binding domain 1 (ABD1) comprises an amino acid sequence having from one to ten amino acid substitutions, additions and/or deletions in the amino acid sequence set forth in SEQ ID NO:56.

In some embodiments, the amino acid variation may be located in one or more framework regions of SEQ ID NO:56.

In some embodiment, the antigen binding domain 1 (ABD1) comprises an amino acid sequence at least 75%, 80% 85%, 90%, 95% identical to the amino acid sequence set forth in SEQ ID NO:56 and CDRs identical to the Kabat CDRs of SEQ ID NO:56.

In some embodiment, the antigen binding domain 1 (ABD1) comprises an amino acid sequence at least 75%, 80% 85%, 90%, 95% identical to the amino acid sequence set forth in SEQ ID NO:56 and CDRs identical to the IMGT CDRs of SEQ ID NO:56.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:56.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:77.

In some embodiments, the antigen binding domain 1 (ABD1) comprises an amino acid sequence having from one to ten amino acid substitutions, additions and/or deletions in the amino acid sequence set forth in SEQ ID NO:77.

In some embodiments, the amino acid variation may be located in one or more framework regions of SEQ ID NO:77.

In some embodiment, the antigen binding domain 1 (ABD1) comprises an amino acid sequence at least 75%, 80% 85%, 90%, 95% identical to the amino acid sequence set forth in SEQ ID NO:77 and CDRs identical to the Kabat CDRs of SEQ ID NO:77.

In some embodiment, the antigen binding domain 1 (ABD1) comprises an amino acid sequence at least 75%, 80% 85%, 90%, 95% identical to the amino acid sequence set forth in SEQ ID NO:77 and CDRs identical to the IMGT CDRs of SEQ ID NO:77.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:77.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:84.

In some embodiments, the antigen binding domain 1 (ABD1) comprises an amino acid sequence having from one to ten amino acid substitutions, additions and/or deletions in the amino acid sequence set forth in SEQ ID NO:84.

In some embodiments, the amino acid variation may be located in one or more framework regions of SEQ ID NO:84.

In some embodiment, the antigen binding domain 1 (ABD1) comprises an amino acid sequence at least 75%, 80% 85%, 90%, 95% identical to the amino acid sequence set forth in SEQ ID NO:84 and CDRs identical to the Kabat CDRs of SEQ ID NO:84.

In some embodiment, the antigen binding domain 1 (ABD1) comprises an amino acid sequence at least 75%, 80% 85%, 90%, 95% identical to the amino acid sequence set forth in SEQ ID NO:84 and CDRs identical to the IMGT CDRs of SEQ ID NO:84.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:84.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:91.

In some embodiments, the antigen binding domain 1 (ABD1) comprises an amino acid sequence having from one to ten amino acid substitutions, additions and/or deletions in the amino acid sequence set forth in SEQ ID NO:91.

In some embodiments, the amino acid variation may be located in one or more framework regions of SEQ ID NO:91.

In some embodiment, the antigen binding domain 1 (ABD1) comprises an amino acid sequence at least 75%, 80% 85%, 90%, 95% identical to the amino acid sequence set forth in SEQ ID NO:91 and CDRs identical to the Kabat CDRs of SEQ ID NO:91.

In some embodiment, the antigen binding domain 1 (ABD1) comprises an amino acid sequence at least 75%, 80% 85%, 90%, 95% identical to the amino acid sequence set forth in SEQ ID NO:91 and CDRs identical to the IMGT CDRs of SEQ ID NO:91.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:91.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:98.

In some embodiments, the antigen binding domain 1 (ABD1) comprises an amino acid sequence having from one to ten amino acid substitutions, additions and/or deletions in the amino acid sequence set forth in SEQ ID NO:98.

In some embodiments, the amino acid variation may be located in one or more framework regions of SEQ ID NO:98.

In some embodiment, the antigen binding domain 1 (ABD1) comprises an amino acid sequence at least 75%, 80% 85%, 90%, 95% identical to the amino acid sequence set forth in SEQ ID NO:98 and CDRs identical to the Kabat CDRs of SEQ ID NO:98.

In some embodiment, the antigen binding domain 1 (ABD1) comprises an amino acid sequence at least 75%, 80% 85%, 90%, 95% identical to the amino acid sequence set forth in SEQ ID NO:98 and CDRs identical to the IMGT CDRs of SEQ ID NO:98.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:98.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:105.

In some embodiments, the antigen binding domain 1 (ABD1) comprises an amino acid sequence having from one to ten amino acid substitutions, additions and/or deletions in the amino acid sequence set forth in SEQ ID NO:105.

In some embodiments, the amino acid variation may be located in one or more framework regions of SEQ ID NO:105.

In some embodiment, the antigen binding domain 1 (ABD1) comprises an amino acid sequence at least 75%, 80% 85%, 90%, 95% identical to the amino acid sequence set forth in SEQ ID NO:105 and CDRs identical to the Kabat CDRs of SEQ ID NO:105.

In some embodiment, the antigen binding domain 1 (ABD1) comprises an amino acid sequence at least 75%, 80% 85%, 90%, 95% identical to the amino acid sequence set forth in SEQ ID NO:105 and CDRs identical to the IMGT CDRs of SEQ ID NO:105.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:105.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:112.

In some embodiments, the antigen binding domain 1 (ABD1) comprises an amino acid sequence having from one to ten amino acid substitutions, additions and/or deletions in the amino acid sequence set forth in SEQ ID NO:112.

In some embodiments, the amino acid variation may be located in one or more framework regions of SEQ ID NO:112.

In some embodiment, the antigen binding domain 1 (ABD1) comprises an amino acid sequence at least 75%, 80% 85%, 90%, 95% identical to the amino acid sequence set forth in SEQ ID NO:112 and CDRs identical to the Kabat CDRs of SEQ ID NO:112.

In some embodiment, the antigen binding domain 1 (ABD1) comprises an amino acid sequence at least 75%, 80% 85%, 90%, 95% identical to the amino acid sequence set forth in SEQ ID NO:112 and CDRs identical to the IMGT CDRs of SEQ ID NO:112.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:112.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:119.

In some embodiments, the antigen binding domain 1 (ABD1) comprises an amino acid sequence having from one to ten amino acid substitutions, additions and/or deletions in the amino acid sequence set forth in SEQ ID NO:119.

In some embodiments, the amino acid variation may be located in one or more framework regions of SEQ ID NO:119.

In some embodiment, the antigen binding domain 1 (ABD1) comprises an amino acid sequence at least 75%, 80% 85%, 90%, 95% identical to the amino acid sequence set forth in SEQ ID NO:119 and CDRs identical to the Kabat CDRs of SEQ ID NO:119.

In some embodiment, the antigen binding domain 1 (ABD1) comprises an amino acid sequence at least 75%, 80% 85%, 90%, 95% identical to the amino acid sequence set forth in SEQ ID NO: 119 and CDRs identical to the IMGT CDRs of SEQ ID NO: 119.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:119.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:126.

In some embodiments, the antigen binding domain 1 (ABD1) comprises an amino acid sequence having from one to ten amino acid substitutions, additions and/or deletions in the amino acid sequence set forth in SEQ ID NO:126.

In some embodiments, the amino acid variation may be located in one or more framework regions of SEQ ID NO:126.

In some embodiment, the antigen binding domain 1 (ABD1) comprises an amino acid sequence at least 75%, 80% 85%, 90%, 95% identical to the amino acid sequence set forth in SEQ ID NO:126 and CDRs identical to the Kabat CDRs of SEQ ID NO:126.

In some embodiment, the antigen binding domain 1 (ABD1) comprises an amino acid sequence at least 75%, 80% 85%, 90%, 95% identical to the amino acid sequence set forth in SEQ ID NO:126 and CDRs identical to the IMGT CDRs of SEQ ID NO:126.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:126.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:133.

In some embodiments, the antigen binding domain 1 (ABD1) comprises an amino acid sequence having from one to ten amino acid substitutions, additions and/or deletions in the amino acid sequence set forth in SEQ ID NO:133.

In some embodiments, the amino acid variation may be located in one or more framework regions of SEQ ID NO:133.

In some embodiment, the antigen binding domain 1 (ABD1) comprises an amino acid sequence at least 75%, 80% 85%, 90%, 95% identical to the amino acid sequence set forth in SEQ ID NO:133 and CDRs identical to the Kabat CDRs of SEQ ID NO:133.

In some embodiment, the antigen binding domain 1 (ABD1) comprises an amino acid sequence at least 75%, 80% 85%, 90%, 95% identical to the amino acid sequence set forth in SEQ ID NO:133 and CDRs identical to the IMGT CDRs of SEQ ID NO:133.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:133.

In some exemplary embodiments, the binding agent of the present disclosure may comprise one or more antigen binding domains, wherein at least one of the antigen binding domains is an antigen binding domain 1 (ABD1) and comprises an amino acid sequence at least 75%, 80% 85%, 90%, 95% identical to the amino acid sequence set forth in SEQ ID NO:140.

In some embodiments, the antigen binding domain 1 (ABD1) comprises an amino acid sequence having from one to ten amino acid substitutions, additions and/or deletions in the amino acid sequence set forth in SEQ ID NO:140.

In some embodiments, the amino acid variation may be located in one or more framework regions of SEQ ID NO:140.

In some embodiment, the antigen binding domain 1 (ABD1) comprises an amino acid sequence at least 75%, 80% 85%, 90%, 95% identical to the amino acid sequence set forth in SEQ ID NO:140 and CDRs identical to the Kabat CDRs of SEQ ID NO:140.

In some embodiment, the antigen binding domain 1 (ABD1) comprises an amino acid sequence at least 75%, 80% 85%, 90%, 95% identical to the amino acid sequence set forth in SEQ ID NO:140 and CDRs identical to the IMGT CDRs of SEQ ID NO:140.

In some exemplary embodiments, the binding agent of the present disclosure may comprise one or more antigen binding domains, wherein at least one of the antigen binding domains is an antigen binding domain 1 (ABD1) and comprises an amino acid sequence as set forth in SEQ ID NO:140.

### Exemplary Embodiments of ABD2

Antigen binding domain 2 (ABD2) may be selected, for example, from antigen binding domains that bind PD-1, some non-limiting exemplary embodiments of which is provided herein.

In some instances, ABD2 is capable of binding human PD-1 or a portion thereof or cells expressing human PD-1 or a portion thereof.

In some exemplary embodiments, the binding agent of the present disclosure may comprise one or more antigen binding domains, wherein at least one of the antigen binding domains is an antigen binding domain 2 (ABD2) and comprises a heavy chain complementarity determining region 1 (CDRH1) having the amino acid sequence set forth in SEQ ID NO:57, a heavy chain complementarity determining region 2 (CDRH2) having the amino acid sequence set forth in SEQ ID NO:58 and a heavy chain complementarity determining region 3 (CDRH3) having the amino acid sequence set forth in SEQ ID NO:59.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:60, a CDRH2 having the amino acid sequence set forth in SEQ ID NO:61 and a CDRH3 having the amino acid sequence set forth in SEQ ID NO:62.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:275, a CDRH2 having the amino acid sequence set forth in SEQ ID NO:276 and a CDRH3 having the amino acid sequence set forth in SEQ ID NO:277.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:278, a CDRH2 having the amino acid sequence set forth in SEQ ID NO:279 and a CDRH3 having the amino acid sequence set forth in SEQ ID NO:280.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:282, a CDRH2 having the amino acid sequence set forth in SEQ ID NO:283 and a CDRH3 having the amino acid sequence set forth in SEQ ID NO:284.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:285, a CDRH2 having the amino acid sequence set forth in SEQ ID NO:286 and a CDRH3 having the amino acid sequence set forth in SEQ ID NO:287.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:289, a CDRH2 having the amino acid sequence set forth in SEQ ID NO:290 and a CDRH3 having the amino acid sequence set forth in SEQ ID NO:291.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:292, a CDRH2 having the amino acid sequence set forth in SEQ ID NO:293 and a CDRH3 having the amino acid sequence set forth in SEQ ID NO:294.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:296, a CDRH2 having the amino acid sequence set forth in SEQ ID NO:297 and a CDRH3 having the amino acid sequence set forth in SEQ ID NO:298.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:299, a CDRH2 having the amino acid sequence set forth in SEQ ID NO:300 and a CDRH3 having the amino acid sequence set forth in SEQ ID NO:301.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:303, a CDRH2 having the amino acid sequence set forth in SEQ ID NO:304 and a CDRH3 having the amino acid sequence set forth in SEQ ID NO:305.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:306, a CDRH2 having the amino acid sequence set forth in SEQ ID NO:307 and a CDRH3 having the amino acid sequence set forth in SEQ ID NO:308.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:310, a CDRH2 having the amino acid sequence set forth in SEQ ID NO:311 and a CDRH3 having the amino acid sequence set forth in SEQ ID NO:312.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:313, a CDRH2 having the amino acid sequence set forth in SEQ ID NO:314 and a CDRH3 having the amino acid sequence set forth in SEQ ID NO:315.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:317, a CDRH2 having the amino acid sequence set forth in SEQ ID NO:318 and a CDRH3 having the amino acid sequence set forth in SEQ ID NO:319.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:320, a CDRH2 having the amino acid sequence set forth in SEQ ID NO:321 and a CDRH3 having the amino acid sequence set forth in SEQ ID NO:322.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:324, a CDRH2 having the amino acid sequence set forth in SEQ ID NO:325 and a CDRH3 having the amino acid sequence set forth in SEQ ID NO:326.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:327, a CDRH2 having the amino acid sequence set forth in SEQ ID NO:328 and a CDRH3 having the amino acid sequence set forth in SEQ ID NO:329.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:331, a CDRH2 having the amino acid sequence set forth in SEQ ID NO:332 and a CDRH3 having the amino acid sequence set forth in SEQ ID NO:333.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:334, a CDRH2 having the amino acid sequence set forth in SEQ ID NO:335 and a CDRH3 having the amino acid sequence set forth in SEQ ID NO:336.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:338, a CDRH2 having the amino acid sequence set forth in SEQ ID NO:339 and a CDRH3 having the amino acid sequence set forth in SEQ ID NO:340.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:341, a CDRH2 having the amino acid sequence set forth in SEQ ID NO:342 and a CDRH3 having the amino acid sequence set forth in SEQ ID NO:343.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:345, a CDRH2 having the amino acid sequence set forth in SEQ ID NO:346 and a CDRH3 having the amino acid sequence set forth in SEQ ID NO:347.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:348, a CDRH2 having the amino acid sequence set forth in SEQ ID NO:349 and a CDRH3 having the amino acid sequence set forth in SEQ ID NO:350.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:352, a CDRH2 having the amino acid sequence set forth in SEQ ID NO:353 and a CDRH3 having the amino acid sequence set forth in SEQ ID NO:354.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:355, a CDRH2 having the amino acid sequence set forth in SEQ ID NO:356 and a CDRH3 having the amino acid sequence set forth in SEQ ID NO:357.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:359, a CDRH2 having the amino acid sequence set forth in SEQ ID NO:360 and a CDRH3 having the amino acid sequence set forth in SEQ ID NO:361.

In some exemplary embodiments, the binding agent of the present disclosure may comprise one or more antigen binding domains, wherein at least one of the antigen binding domains is an antigen binding domain 2 (ABD2) and comprises a CDRH1 having the amino acid sequence set forth in SEQ ID NO:362, a CDRH2 having the amino acid sequence set forth in SEQ ID NO:363 and a CDRH3 having the amino acid sequence set forth in SEQ ID NO:364.

In some embodiments, the binding agent of the present disclosure may have the CDRH1, CDRH2, CDRH3 combination of ABD2 with one, two, three, four or five amino acid substitutions in any of the CDRH1, CDRH2 or CDRH3. In some embodiments, the total number of amino acid substitutions in any of the CDRH1, CDRH2 or CDRH3 is ten amino acid substitutions or less (total of nine, eight, seven, six, five, four, three, two or one). In some embodiments, the total number of amino acid substitutions in CDRH3 is three or less (one, two or three).

In some exemplary embodiments, the binding agent of the present disclosure may comprise one or more antigen binding domains, wherein at least one of the antigen binding domains is an antigen binding domain 2 (ABD2) and comprises the amino acid sequence set forth in SEQ ID NO:379, wherein any one of X₁ₐ to X₁ₖ is any (e.g., natural) amino acid residue or wherein X₁ₐ is L or E, wherein X_{1b} is A or P, wherein X_{1c} is D or G, wherein X₁ₐ is N or S, wherein X₁ₑ is S or T, wherein X_{1f} is K or R, wherein X_{1g} is D or G, wherein X₁ₕ is K or Q, wherein X₁ⱼ is T or A and/or wherein X₁ₖ is L or V.

(SEQ ID NO: 379)
QVQLQESGGGX₁ₐVQX_{1b}GX_{1c}SLRLSCAASGFTFSX_{1d}YGMSWVRQAPG
EGLEWVSSIDSXᵢₑGGTTX_{1f}YAX_{1g}SVX₁ₕGRFTISRDNAKNX₁ⱼX₁ₖYL
QMNSLKPEDTAVYYCAKDFLSWMPRGQGTQVTVSS

In some embodiments, CDR1, CDR2, and/or CDR3 of SEQ ID NO:379 may independently correspond to the CDR1, CDR2, and/or CDR3 of any one of SEQ ID NOs. 288, 295, 302, 309, 323, 330, 337, 344, 351, or 358 and combination thereof.

In some embodiments, the framework region of SEQ ID NO:379 may be modified to include amino acid substitutions, additions and/or deletions. In some embodiments, the framework region of SEQ ID NO:379 may comprise amino acid residues of a human antibody (e.g., human germline antibody sequence or consensus thereof).

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:63.

In some embodiments, the antigen binding domain 2 (ABD2) comprises an amino acid sequence having from one to ten amino acid substitutions, additions and/or deletions in the amino acid sequence set forth in SEQ ID NO:63.

In some embodiments, the amino acid variation may be located in one or more framework regions of SEQ ID NO:63.

In some embodiment, the antigen binding domain 2 (ABD2) comprises an amino acid sequence at least 75%, 80% 85%, 90%, 95% identical to the amino acid sequence set forth in SEQ ID NO:63 and CDRs identical to the Kabat CDRs of SEQ ID NO:63.

In some embodiment, the antigen binding domain 2 (ABD2) comprises an amino acid sequence at least 75%, 80% 85%, 90%, 95% identical to the amino acid sequence set forth in SEQ ID NO:63 and CDRs identical to the IMGT CDRs of SEQ ID NO:63.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:63.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:281.

In some embodiments, the antigen binding domain 2 (ABD2) comprises an amino acid sequence having from one to ten amino acid substitutions, additions and/or deletions in the amino acid sequence set forth in SEQ ID NO:281.

In some embodiments, the amino acid variation may be located in one or more framework regions of SEQ ID NO:281.

In some embodiment, the antigen binding domain 2 (ABD2) comprises an amino acid sequence at least 75%, 80% 85%, 90%, 95% identical to the amino acid sequence set forth in SEQ ID NO:281 and CDRs identical to the Kabat CDRs of SEQ ID NO:281.

In some embodiment, the antigen binding domain 2 (ABD2) comprises an amino acid sequence at least 75%, 80% 85%, 90%, 95% identical to the amino acid sequence set forth in SEQ ID NO:281 and CDRs identical to the IMGT CDRs of SEQ ID NO:281.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:281.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:288.

In some embodiments, the antigen binding domain 2 (ABD2) comprises an amino acid sequence having from one to ten amino acid substitutions, additions and/or deletions in the amino acid sequence set forth in SEQ ID NO:288.

In some embodiments, the amino acid variation may be located in one or more framework regions of SEQ ID NO:288.

In some embodiment, the antigen binding domain 2 (ABD2) comprises an amino acid sequence at least 75%, 80% 85%, 90%, 95% identical to the amino acid sequence set forth in SEQ ID NO:288 and CDRs identical to the Kabat CDRs of SEQ ID NO:288.

In some embodiment, the antigen binding domain 2 (ABD2) comprises an amino acid sequence at least 75%, 80% 85%, 90%, 95% identical to the amino acid sequence set forth in SEQ ID NO:288 and CDRs identical to the IMGT CDRs of SEQ ID NO:288.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:288.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:295.

In some embodiments, the antigen binding domain 2 (ABD2) comprises an amino acid sequence having from one to ten amino acid substitutions, additions and/or deletions in the amino acid sequence set forth in SEQ ID NO:295.

In some embodiments, the amino acid variation may be located in one or more framework regions of SEQ ID NO:295.

In some embodiment, the antigen binding domain 2 (ABD2) comprises an amino acid sequence at least 75%, 80% 85%, 90%, 95% identical to the amino acid sequence set forth in SEQ ID NO:295 and CDRs identical to the Kabat CDRs of SEQ ID NO:295.

In some embodiment, the antigen binding domain 2 (ABD2) comprises an amino acid sequence at least 75%, 80% 85%, 90%, 95% identical to the amino acid sequence set forth in SEQ ID NO:295 and CDRs identical to the IMGT CDRs of SEQ ID NO:295.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:295.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:302.

In some embodiments, the antigen binding domain 2 (ABD2) comprises an amino acid sequence having from one to ten amino acid substitutions, additions and/or deletions in the amino acid sequence set forth in SEQ ID NO:302.

In some embodiments, the amino acid variation may be located in one or more framework regions of SEQ ID NO:302.

In some embodiment, the antigen binding domain 2 (ABD2) comprises an amino acid sequence at least 75%, 80% 85%, 90%, 95% identical to the amino acid sequence set forth in SEQ ID NO:302 and CDRs identical to the Kabat CDRs of SEQ ID NO:302.

In some embodiment, the antigen binding domain 2 (ABD2) comprises an amino acid sequence at least 75%, 80% 85%, 90%, 95% identical to the amino acid sequence set forth in SEQ ID NO:302 and CDRs identical to the IMGT CDRs of SEQ ID NO:302.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:302.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:309.

In some embodiments, the antigen binding domain 2 (ABD2) comprises an amino acid sequence having from one to ten amino acid substitutions, additions and/or deletions in the amino acid sequence set forth in SEQ ID NO:309.

In some embodiments, the amino acid variation may be located in one or more framework regions of SEQ ID NO:309.

In some embodiment, the antigen binding domain 2 (ABD2) comprises an amino acid sequence at least 75%, 80% 85%, 90%, 95% identical to the amino acid sequence set forth in SEQ ID NO:309 and CDRs identical to the Kabat CDRs of SEQ ID NO:309.

In some embodiment, the antigen binding domain 2 (ABD2) comprises an amino acid sequence at least 75%, 80% 85%, 90%, 95% identical to the amino acid sequence set forth in SEQ ID NO:309 and CDRs identical to the IMGT CDRs of SEQ ID NO:309.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:309.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:316.

In some embodiments, the antigen binding domain 2 (ABD2) comprises an amino acid sequence having from one to ten amino acid substitutions, additions and/or deletions in the amino acid sequence set forth in SEQ ID NO:316.

In some embodiments, the amino acid variation may be located in one or more framework regions of SEQ ID NO:316.

In some embodiment, the antigen binding domain 2 (ABD2) comprises an amino acid sequence at least 75%, 80% 85%, 90%, 95% identical to the amino acid sequence set forth in SEQ ID NO:316 and CDRs identical to the Kabat CDRs of SEQ ID NO:316.

In some embodiment, the antigen binding domain 2 (ABD2) comprises an amino acid sequence at least 75%, 80% 85%, 90%, 95% identical to the amino acid sequence set forth in SEQ ID NO:316 and CDRs identical to the IMGT CDRs of SEQ ID NO:316.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:316.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:323.

In some embodiments, the antigen binding domain 2 (ABD2) comprises an amino acid sequence having from one to ten amino acid substitutions, additions and/or deletions in the amino acid sequence set forth in SEQ ID NO:323.

In some embodiments, the amino acid variation may be located in one or more framework regions of SEQ ID NO:323.

In some embodiment, the antigen binding domain 2 (ABD2) comprises an amino acid sequence at least 75%, 80% 85%, 90%, 95% identical to the amino acid sequence set forth in SEQ ID NO:323 and CDRs identical to the Kabat CDRs of SEQ ID NO:323.

In some embodiment, the antigen binding domain 2 (ABD2) comprises an amino acid sequence at least 75%, 80% 85%, 90%, 95% identical to the amino acid sequence set forth in SEQ ID NO:323 and CDRs identical to the IMGT CDRs of SEQ ID NO:323.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:323.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:330.

In some embodiments, the antigen binding domain 2 (ABD2) comprises an amino acid sequence having from one to ten amino acid substitutions, additions and/or deletions in the amino acid sequence set forth in SEQ ID NO:330.

In some embodiments, the amino acid variation may be located in one or more framework regions of SEQ ID NO:330.

In some embodiment, the antigen binding domain 2 (ABD2) comprises an amino acid sequence at least 75%, 80% 85%, 90%, 95% identical to the amino acid sequence set forth in SEQ ID NO:330 and CDRs identical to the Kabat CDRs of SEQ ID NO:330.

In some embodiment, the antigen binding domain 2 (ABD2) comprises an amino acid sequence at least 75%, 80% 85%, 90%, 95% identical to the amino acid sequence set forth in SEQ ID NO:330 and CDRs identical to the IMGT CDRs of SEQ ID NO:330.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:330.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:337.

In some embodiments, the antigen binding domain 2 (ABD2) comprises an amino acid sequence having from one to ten amino acid substitutions, additions and/or deletions in the amino acid sequence set forth in SEQ ID NO:337.

In some embodiments, the amino acid variation may be located in one or more framework regions of SEQ ID NO:337.

In some embodiment, the antigen binding domain 2 (ABD2) comprises an amino acid sequence at least 75%, 80% 85%, 90%, 95% identical to the amino acid sequence set forth in SEQ ID NO:337 and CDRs identical to the Kabat CDRs of SEQ ID NO:337.

In some embodiment, the antigen binding domain 2 (ABD2) comprises an amino acid sequence at least 75%, 80% 85%, 90%, 95% identical to the amino acid sequence set forth in SEQ ID NO:337 and CDRs identical to the IMGT CDRs of SEQ ID NO:337.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:337.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:344.

In some embodiments, the antigen binding domain 2 (ABD2) comprises an amino acid sequence having from one to ten amino acid substitutions, additions and/or deletions in the amino acid sequence set forth in SEQ ID NO:344.

In some embodiments, the amino acid variation may be located in one or more framework regions of SEQ ID NO:344.

In some embodiment, the antigen binding domain 2 (ABD2) comprises an amino acid sequence at least 75%, 80% 85%, 90%, 95% identical to the amino acid sequence set forth in SEQ ID NO:344 and CDRs identical to the Kabat CDRs of SEQ ID NO:344.

In some embodiment, the antigen binding domain 2 (ABD2) comprises an amino acid sequence at least 75%, 80% 85%, 90%, 95% identical to the amino acid sequence set forth in SEQ ID NO:344 and CDRs identical to the IMGT CDRs of SEQ ID NO:344.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:344.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:351.

In some embodiments, the antigen binding domain 2 (ABD2) comprises an amino acid sequence having from one to ten amino acid substitutions, additions and/or deletions in the amino acid sequence set forth in SEQ ID NO:351.

In some embodiments, the amino acid variation may be located in one or more framework regions of SEQ ID NO:351.

In some embodiment, the antigen binding domain 2 (ABD2) comprises an amino acid sequence at least 75%, 80% 85%, 90%, 95% identical to the amino acid sequence set forth in SEQ ID NO:351 and CDRs identical to the Kabat CDRs of SEQ ID NO:351.

In some embodiment, the antigen binding domain 2 (ABD2) comprises an amino acid sequence at least 75%, 80% 85%, 90%, 95% identical to the amino acid sequence set forth in SEQ ID NO:351 and CDRs identical to the IMGT CDRs of SEQ ID NO:351.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:351.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:358.

In some embodiments, the antigen binding domain 2 (ABD2) comprises an amino acid sequence having from one to ten amino acid substitutions, additions and/or deletions in the amino acid sequence set forth in SEQ ID NO:358.

In some embodiments, the amino acid variation may be located in one or more framework regions of SEQ ID NO:358.

In some embodiment, the antigen binding domain 2 (ABD2) comprises an amino acid sequence at least 75%, 80% 85%, 90%, 95% identical to the amino acid sequence set forth in SEQ ID NO:358 and CDRs identical to the Kabat CDRs of SEQ ID NO:358.

In some embodiment, the antigen binding domain 2 (ABD2) comprises an amino acid sequence at least 75%, 80% 85%, 90%, 95% identical to the amino acid sequence set forth in SEQ ID NO:358 and CDRs identical to the IMGT CDRs of SEQ ID NO:358.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:358.

In some exemplary embodiments, the binding agent of the present disclosure may comprise one or more antigen binding domains, wherein at least one of the antigen binding domains is an antigen binding domain 2 (ABD2) and comprises an amino acid sequence at least 75%, 80% 85%, 90%, 95% identical to the amino acid sequence set forth in SEQ ID NO:365.

In some embodiments, the antigen binding domain 2 (ABD2) comprises an amino acid sequence having from one to ten amino acid substitutions, additions and/or deletions in the amino acid sequence set forth in SEQ ID NO:365.

In some embodiments, the amino acid variation may be located in one or more framework regions of SEQ ID NO:365.

In some embodiment, the antigen binding domain 2 (ABD2) comprises an amino acid sequence at least 75%, 80% 85%, 90%, 95% identical to the amino acid sequence set forth in SEQ ID NO:365 and CDRs identical to the Kabat CDRs of SEQ ID NO:365.

In some embodiment, the antigen binding domain 2 (ABD2) comprises an amino acid sequence at least 75%, 80% 85%, 90%, 95% identical to the amino acid sequence set forth in SEQ ID NO:63 and CDRs identical to the IMGT CDRs of SEQ ID NO:365.

In some exemplary embodiments, the binding agent of the present disclosure may comprise one or more antigen binding domains, wherein at least one of the antigen binding domains is an antigen binding domain 2 (ABD2) and comprises an amino acid sequence as set forth in SEQ ID NO:365.

### Exemplary Embodiments of ABD3

Antigen binding domain 3 (ABD3) may be selected, for example, from antigen binding domains that bind CD47, some non-limiting exemplary embodiments of which is provided herein.

In some instances, ABD3 is capable of binding human CD47 or a portion thereof or cells expressing human CD47 or a portion thereof.

In some exemplary embodiments, the binding agent of the present disclosure may comprise one or more antigen binding domains, wherein at least one of the antigen binding domains is an antigen binding domain 3 (ABD3) and comprises a heavy chain complementarity determining region 1 (CDRH1) having the amino acid sequence set forth in SEQ ID NO:64, a heavy chain complementarity determining region 2 (CDRH2) having the amino acid sequence set forth in SEQ ID NO:65 and a heavy chain complementarity determining region 3 (CDRH3) having the amino acid sequence set forth in SEQ ID NO:66.

In some exemplary embodiments, the binding agent of the present disclosure may comprise one or more antigen binding domains, wherein at least one of the antigen binding domains is an antigen binding domain 3 (ABD3) and comprises a CDRH1 having the amino acid sequence set forth in SEQ ID NO:67, a CDRH2 having the amino acid sequence set forth in SEQ ID NO:68 and a CDRH3 having the amino acid sequence set forth in SEQ ID NO:69.

In some embodiments, the binding agent of the present disclosure may have the CDRH1, CDRH2, CDRH3 combination of ABD3 with one, two, three, four or five amino acid substitutions in any of the CDRH1, CDRH2 or CDRH3. In some embodiments, the total number of amino acid substitutions in any of the CDRH1, CDRH2 or CDRH3 is ten amino acid substitutions or less (total of nine, eight, seven, six, five, four, three, two or one). In some embodiments, the total number of amino acid substitutions in CDRH3 is three or less (one, two or three).

In some exemplary embodiments, the binding agent of the present disclosure may comprise one or more antigen binding domains, wherein at least one of the antigen binding domains is an antigen binding domain 3 (ABD3) and comprises an amino acid sequence at least 75%, 80% 85%, 90%, 95% identical to the amino acid sequence set forth in SEQ ID NO:70.

In some embodiments, the antigen binding domain 3 (ABD3) comprises an amino acid sequence having from one to ten amino acid substitutions, additions and/or deletions in the amino acid sequence set forth in SEQ ID NO:70.

In some embodiments, the amino acid variation may be located in one or more framework regions of SEQ ID NO:70.

In some embodiment, the antigen binding domain 3 (ABD3) comprises an amino acid sequence at least 75%, 80% 85%, 90%, 95% identical to the amino acid sequence set forth in SEQ ID NO:70 and CDRs identical to the Kabat CDRs of SEQ ID NO:70.

In some embodiment, the antigen binding domain 3 (ABD3) comprises an amino acid sequence at least 75%, 80% 85%, 90%, 95% identical to the amino acid sequence set forth in SEQ ID NO:70 and CDRs identical to the IMGT CDRs of SEQ ID NO:70.

In some exemplary embodiments, the binding agent of the present disclosure may comprise one or more antigen binding domains, wherein at least one of the antigen binding domains is an antigen binding domain 3 (ABD3) and comprises an amino acid sequence as set forth in SEQ ID NO:70.

### Exemplary Embodiments of Other Antigen Binding Domains

In some embodiments, the binding agent comprises one or more antigen binding domains, wherein at least one of the antigen binding domains is an antigen binding domain 1 (ABD1), an antigen binding domain 2 (ABD2) or an antigen binding domain 3 (ABD3) and wherein at least one antigen binding domains binds to a different antigen.

In some embodiments, the binding agents may thus comprise an antigen binding domain that binds to CD3. Such antigen binding domains include those known to a person skilled in the art. An exemplary embodiment of an antigen binding domain that binds to CD3 is provided in SEQ ID NO:197.

Accordingly, in some embodiments, the binding agents may comprise an antigen binding domain that binds to CD36, PD-1 and/or CD3. In other embodiments, the binding agents may comprise an antigen binding domain that binds to CD36 and/or CD3. In yet other embodiments, the binding agents may comprise an antigen binding domain that binds to PD-1 and/or CD3.

In some embodiments, the binding agent comprises one or more antigen binding domains, wherein at least one of the antigen binding domains is an antigen binding domain 1 (ABD1) and wherein at least one of the antigen binding domains is selected, for example and without limitations, from antigen binding domains that specifically bind to CD3, CD36, DRD1, DRD2, DRD3, DRD4, DRD5, TROP2, CD147, MCT1, ILIRAP, AMIGO2, PTK7, MCT2, MCT4, NHE1, H+/K+-ATPase, LAP, HLA-I A2, CD73, CD98, CEACAM5/6, ICAM-1, MCSP, fibronectin, Beta 1 Integrin, Tetraspanin 8, CD164, CD59, CD63, CD44, CD166, cWF, TNF, IL-17A, IL17-F, IL-6R, BCMA, TNF, RANKL, ADAMTS5, VEGF, Ang2, CX₃CR1, CXCR4, TfR1 (CD71), CXCR2, CD3, PD1, PDL-1, CTLA-4, CD8, LAG-3, OX40, CD27, CD122/IL2RB, TLR8/CD288, TIM-3, ICOS/CD278, NKG2A, A2AR, B7-H3, B7-H4, GITR/TNFRSF18, 4-IBB/CD137, KIR2DL1, KIR3DL2, SIRPa, CD47, VISTA, CD40, CD112, CD96, TOGOT, BTLA, TIGIT, CD4, VEGFR2, CD19, IGFR1, EpCAM, EGFR, DLL3, CGRP, CD79b, CD28, CCR5, ErbB3, ErbB2, TGFβ1, TGFβ2, TGFβ3, TGFβR1, TGFβR2, IDO1, IDO2, TLR-4, TLR-7, TLR-8, TLR-9, NOX2, or SIGLEC-7.

### Polypeptide Chain(s)

The binding agents of the present disclosures comprise one or more polypeptide chains.

Segments of DNA encoding desired polypeptide chain sequences may be synthesized in vitro. The different DNA modules are assembled into a single piece in an organized and directional manner which is then cloned into an expression vector. The resulting polypeptide chains are therefore composed of different modules forming a single chain.

The polypeptide chains of the present disclosure include, for example and without limitation, antigen binding domains, linkers and a dimerization domain that promote assembly of at least two polypeptide chains.

In some embodiments, the polypeptide chain of the present disclosure may be monospecific.

In some embodiments, the polypeptide chain of the present disclosure may be multispecific.

In some embodiments, the polypeptide chain of the present disclosure may be monovalent.

In some embodiments, the polypeptide chain of the present disclosure may be multivalent.

In some instances, the polypeptide chain does not comprise a dimerization domain.

In some instances, the polypeptide chain comprises a dimerization domain.

In some aspects of the disclosure, the polypeptide chains may be monospecific.

An exemplary embodiment of a monospecific polypeptide chain includes a polypeptide chain that comprise one antigen binding domain. Another exemplary embodiment of a monospecific polypeptide chain includes a polypeptide chain that comprise more than one antigen binding domains, but the antigen binding domains have identical CDRs and framework regions. Yet another exemplary embodiment of a monospecific polypeptide chain includes a polypeptide chain that comprise more than one antigen binding domains, but the antigen binding domains have identical CDRs and different framework regions. A further exemplary embodiment of a monospecific polypeptide chain includes a polypeptide chain that comprise antigen binding domains that differ in the amino acid sequence of one or more of their CDRs (e.g., conservative substitution in one or more CDRs) without affecting their ability to bind to the same antigen or epitope.

In some aspects of the disclosure, the polypeptide chains may be multispecific. The polypeptide chains may encompass for example, bispecific polypeptide chains, trispecific polypeptide chains, tetraspecific polypeptide chains, pentaspecific polypeptide chains, hexaspecific polypeptide chains, biparatopic polypeptide chains, multiparatopic polypeptide chains and the like.

In an exemplary configuration, one or more antigen binding domains may be located at the N-terminus, at the C-terminus or on each side of the dimerization domain.

In another exemplary configuration, the polypeptide chains may comprise at least one antigen binding domain at the N-terminus of the dimerization domain and at least one antigen binding domain at the C-terminus of the dimerization domain.

In a further exemplary configuration, the polypeptide chains may comprise one antigen binding domain at the N-terminus of the dimerization domain and at least two antigen binding domains at the C-terminus of the dimerization domain.

In yet a further exemplary configuration, the polypeptide chains may comprise two antigen binding domains at the N-terminus of the dimerization domain and two antigen binding domains at the C-terminus of the dimerization domain.

The polypeptide chains may comprise in a N- to C-terminal fashion an amino acid sequence of formula I:

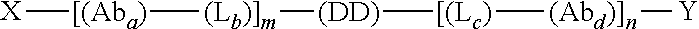

Wherein m is 0, 1, 2 or an integer greater than 2;
Wherein n is 0, 1, 2 or an integer greater than 2;
Wherein m and n are not 0 simultaneously;
Wherein Abₐ, Ab_{d}, each represents an antigen binding domain wherein at least one of the antigen binding domains is an antigen binding domain 1 (ABD1), an antigen binding domain 2 (ABD2) or an antigen binding domain 3 (ABD3);
Wherein X or Y are independently present or absent and comprises an amino acid sequence;
Wherein L_{b}, L_{c}, each independently comprises one or more linkers; and
Wherein DD represents a dimerization domain.

In some embodiments L_{b} may be absent.

In some embodiments L_{c} may be absent.

In some embodiments both L_{b} and L_{c} may be absent.

In some embodiments the polypeptide chain comprises more than one antigen binding domains.

In some embodiments where m is 2 or an integer greater than 2, the [(Abₐ)-(L_{b})] units is the same.

In other embodiments where m is 2 or an integer greater than 2, the [(Abₐ)-(L_{b})] units of the polypeptide chain or binding agent is different.

In other embodiments where m is an integer greater than 2, the [(Abₐ)-(L_{b})] units of the polypeptide chain or binding agent may comprise the same and different units.

In some embodiments where n is 2 or an integer greater than 2, the [(L_{c})-(Ab_{d})] units are the same.

In other embodiments where n is 2 or an integer greater than 2, the [(L_{c})-(Ab_{d})] units are different.

In other embodiments where n is 2 or an integer greater than 2, the [(L_{c})-(Ab_{d})] units comprise the same and different units.

In some embodiments, where m is 2 or an integer greater than 2, each Abₐ is the same or different.

In some embodiments, where n is 2 or an integer greater than 2, each Ab_{d} is the same or different.

In some embodiments, Abₐ represents ABD1.

In some embodiments, Ab_{d} represents ABD2.

In some embodiments, Ab_{d} represents ABD3.

In some embodiments, m is 1 and Abₐ represents ABD1.

In some embodiments, n is 2 and one of Ab_{d} represents ABD2.

In some embodiments, n is 2 and one of Ab_{d} represents ABD3.

In some embodiments, n is 2 and one of Ab_{d} represents ABD2 and the other Ab_{d}represents ABD3.

In some embodiments, m is 2, 3, 4, 5 or an integer greater than 5.

In some embodiments m is 2.

In other embodiments m is 3.

In yet other embodiments m is 4.

In further embodiments m is 5 In other embodiments m is an integer greater than 5.

In some embodiments, n is 2, 3, 4, 5 or an integer greater than 5.

In some embodiments n is 2.

In other embodiments n is 3.

In additional embodiments n is 4.

In further embodiments n is 5.

In other embodiments n is an integer greater than 5.

In embodiments, the one or more polypeptide chain further comprises a hinge region of an antibody or antigen binding fragment thereof.

In some embodiments, the hinge region is at the N-terminus of the dimerization domain.

In some embodiments L_{b} is a hinge region of an antibody or antigen binding fragment thereof.

In some embodiments, the hinge region is a natural human IgG1 hinge region. In other embodiments the hinge region is a mutated human IgG1 hinge region.

In other embodiments, the hinge region is a natural human IgG2 hinge region. In other embodiments, the hinge region is a mutated human IgG2 hinge region.

In other embodiments, the hinge region is a natural human IgG3 hinge region. In other embodiments, the hinge region is a mutated human IgG3 hinge region.

In yet other embodiments, the hinge region is a natural human IgG4 hinge region. In other embodiments, the hinge region is a mutated human IgG4 hinge region.

In some embodiments each of the one or more linkers independently has at least 5, 10, 15, 20, 25, 30, 35, 40, 45 or 50 amino acid residues in length.

In some embodiments, each of the one or more linkers is independently a flexible linker, a helical linker, or a rigid linker.

In some embodiments, the linker L_{c} is a rigid linker.

In some embodiments, L_{c} comprises a non-cleavable linker. In other embodiments, L_{c} consists of a non-cleavable linker.

In some embodiments the one or more linkers comprise a flexible linker.

In some embodiments the one or more linkers comprise a rigid linker.

In some embodiments, the flexible linker is a GS linker.

In some embodiments, the flexible linker comprises one or more units of GGGGS.

In some embodiments, the flexible linker comprises at least 2, 3, 4, 5, or more units of GGGGS.

In some embodiments the rigid linker comprises multiple PA repeats.

In some embodiments, the rigid linker is selected from PAPAPKA (SEQ ID NO:187); APAPAPAPAPKA (SEQ ID NO:188); APAPAPAPAPAPAPAPAPAPKA (SEQ ID NO:189); or combinations thereof.

In some embodiments, the helical linker comprises one or more units of EAAAK.

In some embodiments, the helical linker is selected from AEAAAKEAAAKA (SEQ ID NO:191); AEAAAKEAAAKEAAAKA (SEQ ID NO:192); AEAAAKEAAAKEAAAKEAAAKEAAAKA (SEQ ID NO:193); or combinations thereof.

In some embodiments, the dimerization domain comprises an amino acid sequence at least 80%, 85%, 90%, 95%, 96%, 97%, 98%, or 99% identical to SEQ ID NO:198.

In some embodiments the dimerization domain further comprises an amino acid sequence at least 80%, 85%, 90%, 95%, 96%, 97%, 98%, or 99% identical to SEQ ID NO:199.

In other exemplary embodiments, the polypeptide chain comprises formula II:

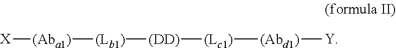

In yet other exemplary embodiments, the polypeptide chain comprises formula III:

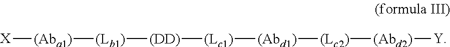

In further exemplary embodiments, the polypeptide chain comprises formula IV:

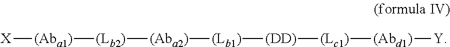

In additional exemplary embodiments the polypeptide chain comprises formula V:

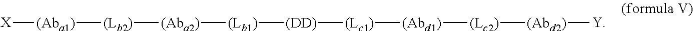

In further exemplary embodiments, the polypeptide chain comprises formula VI:

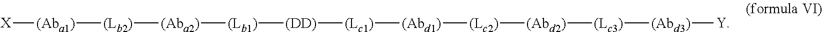

In yet further exemplary embodiments, the polypeptide chain comprises formula VII:

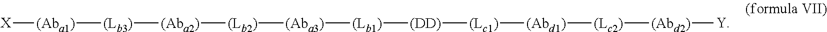

In other exemplary embodiments, the polypeptide chain comprises formula VIII:

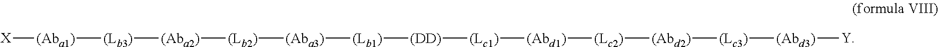

In some embodiments Abₐ₁, Abₐ₂, Abₐ₃, Ab_{d1}, Ab_{d2}, Ab_{d3}, each independently comprises an antigen binding domain.

In some embodiments Abₐ₁, Abₐ₂, Abₐ₃, Ab_{d1}, Ab_{d2}, Abₐ₃, each independently represents an antigen binding domain.

In some embodiments, at least one of the antigen binding domains is an antigen binding domain 1 (ABD1).

In some embodiments, the polypeptide chain also comprises an antigen binding domain 2 (ABD2) or an antigen binding domain 3 (ABD3).

In some embodiments, the polypeptide chain also comprises at least one antigen binding domain 2 (ABD2) and at least one antigen binding domain 3 (ABD3).

In some embodiments L_{b1} comprises a linker or linkers and/or a hinge region of an antibody or antigen binding fragment thereof. In some embodiments, L_{b1} is L_{b}.

In some embodiments L_{b}2, L_{b3} L_{c1}, L_{c2}, and L_{c3} each independently comprise a linker or linkers.

In some embodiments one or more of L_{b1}, L_{b2} and/or L_{b3} may be absent.

In some embodiments L_{b}2 and/or L_{b3} may be absent.

In some embodiments, one or more of L_{c1}, L_{c2}, and/or L_{c3} may be absent.

In some embodiments L_{c2} and/or L_{c3} may be absent.

In some embodiments, L_{c1} is a rigid linker.

In some embodiments, L_{c2} is a rigid linker.

In some embodiments, L_{c3} is a rigid linker.

In some embodiments, L_{c1}, and L_{c2} are rigid linkers.

In some embodiments, L_{c1}, L_{c2} and L_{c3} are rigid linkers.

In some embodiments L_{c1}, L_{c2} and/or L_{c3} may be absent.

In some embodiments L_{c2}, and/or L_{c3} may be absent.

In some embodiments L_{c1}, and/or L_{c3} may be absent.

In some embodiments L_{c2} and/or L_{c3} may be absent.

In some embodiments, L_{c1}, L_{c2}, and L_{c3} may be absent.

The polypeptide chains of the present disclosure comprise antigen binding domains that are functionally active either as a single chain or when part of the binding agent disclosed herein.

For example, the antigen binding domain of the polypeptide chains may bind to its target and may biologically active.

In some embodiments, the biological activity of an antigen binding domain includes, for example and without limitation, blocking binding of a target to its natural receptor or ligand. Alternatively, the biological activity of an antigen binding domain includes its ability to sequester a target. Moreover, the biological activity of an antigen binding domain includes its ability to induce signalling.

A polypeptide chain that comprises more than one antigen binding domain is characterized as being multivalent.

The polypeptide chain of the present disclosure may comprise an additional amino acid sequence at its N- or C- terminus or at both ends (defined by X and Y respectively in the formulas disclosed herein).

In some embodiments, the amino acid sequence at the N-terminus (defined by X) may include a signal peptide, an exemplary embodiment of which is provided in SEQ ID NO:215.

In some embodiments, the amino acid sequence at the N-terminus (defined by X) or C-terminus (defined by Y) may independently include a linker, a cytokine, a chemokine, a tag (e.g., His tag (e.g. SEQ ID NO:216), a masking domain, aphage coat protein, an antigen binding domain or combination thereof.

An exemplary embodiment of a multispecific polypeptide chain include a polypeptide chain that comprises at least two antigen binding domains that differ in the amino acid sequence of one or more of their CDRs leading to different binding specificities.

A polypeptide chain may more particularly be characterized as being bispecific when it binds to two different epitopes or antigens. A polypeptide chain may be characterized as being trispecific when it binds to three different epitopes or antigens. A polypeptide chain may be characterized as being tetraspecific when it binds to four different epitopes or antigens. A polypeptide chain may be characterized as being pentaspecific when it binds to five different epitopes or antigens. A polypeptide chain may be characterized as being hexaspecific when it binds to six different epitopes or antigens.

A polypeptide chain comprising two antigen binding domains that bind to two non-overlapping epitopes on the same target is characterized as being biparatopic. A polypeptide chain comprising antigen binding domains that bind to three, four or more epitopes on the same target is characterized as being multiparatopic.

The antigen binding domains of a given polypeptide chain will be selected based on the intended use such as detection, diagnostic and/or therapeutic use. Each of the antigen binding domains of a particular polypeptide chain may be selected so as to generate an additive or synergic effect.

For example, polypeptide chains of the present disclosure may comprise at least one antigen binding domain that specifically binds to an antigen expressed by tumor cells or by the tumor cell environment (i.e., tumor-specific antigen binding domains).

In other aspects and embodiments of the disclosure the polypeptide chains may comprise at least one antigen binding domain that specifically binds to an immunomodulator.

For example, the polypeptide chain may comprise one or more antigen binding domains that bind an immune checkpoint protein, a cytokine, a chemokine or an immune receptor or coreceptor etc. (e.g., immune-specific antigen binding domains).

In some embodiments, the polypeptide chain comprises in a N- to C-terminal fashion an antigen binding domain that binds to CD36, an antigen binding domain that binds to PD-1 and an antigen binding domain that binds to CD47.

In some exemplary embodiment, the antigen binding domain may bind to CD36.

In some exemplary embodiment, the antigen binding domain may bind to PD1.

In some exemplary embodiment, the antigen binding domain may bind to CD47.

In an exemplary embodiment, the polypeptide chain of the present disclosure may comprise at least one tumor-specific antigen binding domain and at least one immune-specific antigen binding domain.

In some embodiments, the tumor-specific antigen binding domain(s) may be located at the N-terminus of the dimerization domain.

In some embodiments, the tumor-specific antigen binding domain(s) may be located at the C-terminus of the dimerization domain.

In some embodiments, the tumor-specific antigen binding domains may be located at both the N- and C-terminus of the dimerization domain.

In some embodiments, the more immune-specific antigen binding domain(s) may be located at the N-terminus of the dimerization domain.

In some embodiments, the immune-specific antigen binding domain(s) may be located at the C-terminus of the dimerization domain.

In some embodiments, the immune-specific antigen binding domains may be located at both the N- and C-terminus of the dimerization domain.

In exemplary and non-limiting embodiments, the polypeptide chain or binding agent may comprise two immune-specific antigen binding domains at the C-terminus of the dimerization domain. In some embodiments, the immune-specific antigen binding domain that is immediately adjacent to the C-terminal part of the dimerization domain may be linked via a non-cleavable linker.

### Dimerization Domain (DD)

In some embodiments, the polypeptide chains of the present disclosure comprise a dimerization domain. As such, two polypeptide chains may assemble to form a binding agent. Exemplary embodiments of binding agent include homodimers and heterodimers.

The dimerization domain may comprise, for example and without limitation, constant regions of an immunoglobulin, including for example a Fc, CH2 and/or CH3 domain of a heavy chain immunoglobulin. A constant region of an immunoglobulin heavy chain is particularly contemplated.

In certain embodiments and aspects of the present disclosure the dimerization domain may have a sequence identical to that of a natural IgG1, IgG2, IgG3 or IgG4 constant region or with their corresponding CH2 and/or CH3 domains.

Particularly encompassed by the present disclosure dimerization domains having a sequence identical to that of a natural human antibody. Exemplary embodiments of dimerization domains include for example a CH2-CH3 domain of a natural human heavy chain.

Accordingly, in some embodiments, the dimerization domain comprises a natural constant region of an antibody, such as for example, a natural human IgG1 constant region, a natural human IgG2 constant region, a natural human IgG3 constant region or a natural human IgG4 constant region.

Accordingly, in some embodiments, the dimerization domain comprises a natural CH3 domain.

In exemplary embodiments, the dimerization domain comprises a natural human CH3 domain.

In some embodiments, the dimerization domain comprises a natural CH2 domain and a natural CH3 domain.

In some embodiments, the natural CH3 domain is a natural IgG1 CH3 domain. In some embodiments, the natural CH3 domain is a natural human IgG1 CH3 (e.g., SEQ ID NO:198).

In some embodiments, the natural CH3 domain is a natural IgG2 CH3 domain. In other embodiments, the natural CH3 domain is a natural human IgG2 CH3 domain.

In some embodiments, the natural CH3 domain is a natural IgG3 CH3 domain. In other embodiments, the natural CH3 domain is a natural human IgG3 CH3 domain.

In some embodiments, the natural CH3 domain is a natural IgG4 CH3 domain. In some embodiments, the natural CH3 domain is a natural human IgG4 CH3 domain.

When the two polypeptide chains of the binding agent are composed of the same amino acid sequence, the binding agent will form a homodimer. However, co-expression of polypeptide chains having a CH2-CH3 domain of a natural antibody, but different amino acid sequence may result in a mixture of homodimers and heterodimers. The different binding agents present in a mixture may be separated by methods known in the art and including for example, size-exclusion chromatography.

Exemplary heterodimers of the present disclosure therefore include those having a CH3 domain or a CH2-CH3 domain of a natural antibody and that are formed by two polypeptide chains having different sequences or configurations.

In some embodiments, the polypeptide chains may have a mutated dimerization domain that comprises, for example, from 1 to 30, from 1 to 20, from 1 to 15, from 1 to 10, from 1 to 9, from 1 to 8, from 1 to 7, from 1 to 6, from 1 to 5, from 1 to 4, from 1 to 3 amino acid substitutions in comparison with a natural or wild type sequence.

In exemplary embodiments, mutated dimerization domains may comprise 1, 2, 3, 4, 5, 6, 7, 8, 9 or 10 amino acid substitutions. Amino acid substitutions may be conservatives or non-conservatives as outlined in Table 2.

In exemplary embodiments, the polypeptide chains may have a mutated dimerization domain having a sequence which is from 80% to 99% identical with that of a natural IgG1, IgG2, IgG3 or IgG4 constant region or with a CH2 and/or CH3 domain. Polypeptide chains encompassed by the present disclosure include those comprising a mutated dimerization domain that is from 85% to 99% identical, from 90% to 99% identical, from 95% to 99% identical with that of a natural IgG1, IgG2, IgG3 or IgG4 constant region or with a CH2 and/or CH3 domain.

In some embodiments, the polypeptide chains of the present disclosure may comprise a mutated dimerization domain comprising amino acid substitutions that favorize heterodimer formation. Heterodimers of the present disclosure may therefore be formed by polypeptide chains comprising such mutations.

Accordingly, in some embodiments, the dimerization domain comprises a mutated constant region of an antibody, such as for example, a mutated human IgG1 constant region, a mutated human IgG2 constant region, a mutated human IgG3 constant region or a mutated human IgG4 constant region. A mutated constant region may have one or more amino acid substitutions, amino acid insertion or amino acid deletion in comparison with a natural constant region.

Accordingly, in some embodiments, the dimerization domain comprises a mutated CH3 domain. A mutated CH3 domain may have one or more amino acid substitutions, amino acid insertion or amino acid deletion in comparison with a natural CH3 domain.

In some embodiments, the dimerization domain comprises a natural CH2 and a mutated CH3 domain.

In some embodiments, the dimerization domain comprises a mutated CH2 and a mutated CH3 domain. A mutated CH2 domain may have one or more amino acid substitutions, amino acid insertion or amino acid deletion in comparison with a natural CH2 domain.

In some embodiments, the mutated CH3 domain is a mutated IgG1 CH3 domain. In other embodiments, the mutated CH3 domain is a mutated human IgG1 CH3 domain.

In some embodiments, the mutated CH3 domain is a mutated IgG2 CH3 domain. In other embodiments, the mutated CH3 domain is a mutated human IgG2 CH3 domain.

In some embodiments, the mutated CH3 domain is a mutated IgG3 CH3 domain. In other embodiments, the mutated CH3 domain is a mutated human IgG3 CH3 domain.

In some embodiments, the mutated CH3 domain is a mutated IgG4 CH3 domain. In other embodiments, the mutated CH3 domain is a mutated human IgG4 CH3 domain.

In some embodiments, the mutated CH2 domain is a mutated IgG1 CH2 domain. In other embodiments, the mutated CH2 domain is a mutated human IgG1 CH2 domain.

In some embodiments, the mutated CH2 domain is a mutated IgG2 CH2 domain. In other embodiments, the mutated CH2 domain is a mutated human IgG2 CH2 domain.

In some embodiments, the mutated CH2 domain is a mutated IgG3 CH2 domain. In other embodiments, the mutated CH2 domain is a mutated human IgG3 CH2 domain.

In some embodiments, the mutated CH2 domain is a mutated IgG4 CH2 domain. In other embodiments, the mutated CH2 domain is a mutated human IgG4 CH2 domain.

In some embodiments, the Fc region may be modified so as to prevent glycosylation, to extend its half-life, to modulate receptor binding or effector function. Exemplary mutations are discussed in Saunders K.O. (Front. Immunol. 10:1296, 2019 the entire content of which is incorporated herein by reference) and include for example mutation of asparagine 297 (e.g., N297).

In some embodiments, the dimerization domain comprises a Fc region of an antibody or a portion thereof.

In some embodiments, the dimerization domain comprises a Fc region or a portion thereof that comprises Fc modification(s) or not.

In other embodiments, the dimerization domain comprises an altered or mutated Fc region or portion thereof. For example, the Fc region or portion thereof may be altered or mutated so as to modify one or more characteristics of the binding agent. In exemplary embodiments, the Fc region or portion thereof is unglycosylated. In other exemplary embodiments, the Fc region or portion thereof comprises one or more of the Fc modification(s) of Table 1.

In other exemplary embodiments, the Fc region or portion thereof is altered or mutated so as to increase ADCC activity. For example, afucosylation (e.g., N297 in accordance with the EU numbering system) may result in an increased FcRgII binding on NK cells and may potently increase ADCC. Accordingly, in some embodiments, the Fc region or portion thereof may have a reduced number of fucose residues. In other embodiments, the binding agent comprises an afucosylated Fc region or portion thereof. In yet other embodiments, the Fc region or portion thereof of the binding agent lacks fucose residues. In additional embodiments, the Fc region or portion thereof of the binding agent lacks core fucose residues. In further embodiments, the Fc region or portion thereof of the binding agent fully lacks core fucose residues.

Exemplary and non-limiting embodiments of mutations in the constant region (e.g., Fc region) that improves one or more effector function are encompassed by the present disclosure, exemplary embodiments of which, are provided in the Table 1 (List of Mutations Antibodies (Basel). 2020 Nov. 17; 9(4):64, the entire content of which is incorporated herein by reference).

TABLE 1

| | Effector Function vs. WT | | | |
|---|---|---|---|---|
| Fc Modification(s) | ID | ADCC | ADCP | Reference |
| S298A/E333A/K334A | AAA | + | n.d. | R. J. Biol. Chem. 2001, 276, 6591- |
| | | | | 6604. |
| S239D/I332E | DE | ++ | + | Proc. Natl. Acad. Sci. USA 2006, |
| | | | | 103, 4005-401 |
| S239D/A330L/I332E | DLE | +++ | + | Proc. Natl. Acad. Sci. USA 2006, |
| | | | | 103, 4005-401 |
| G236A | G236A | − | + | Mol. Cancer Ther. 2008, 7, 2517- |
| | | | | 2527. |
| G236A/S239D/I332E | ADE | + | + | Mol. Cancer Ther. 2008, 7, 2517- |
| | | | | 2527. |
| F243L/R292P/Y300L/ | LPLIL | ++ | n.d. | Cancer Res. 2007, 67, 8882-8890. |
| V305I/P396L | | | | |
| L235V/F243L/R292P/ | VLPLL | + | n.d. | Breast Cancer Res. 2011, 13, R123. |
| Y300L/P396L | | | | |
| P247I/A339Q | | ++ | | Immunol. Cell Biol. 2020, 98, 287- |
| | | | | 304. |
| Afucosylation (Potelligent)* | Potelligent | ++ | n.d. | Biotechnol. Bioeng. 2004, 87, 614- |
| | | | | 622. |

*POTELLIGENT Technology involves the reduction of the amount of fucose in the carbohydrate structure of an antibody using a proprietary fucosyl transferase-knockout CHO cell line.
(−): no change; (+): 2 fold increase; (++): 2-9.99 fold increase; (+++):10-99.99; (nd): no data.

In accordance with the present disclosure, the mutated CH3 domain comprises one or more mutations in comparison with a natural CH3 domain. Accordingly, in some embodiments, the dimerization domain comprises a mutated CH3 domain comprising one or more mutations in comparison with a natural CH3 domain.

In some embodiments, the dimerization domain comprises a natural CH2 domain and a mutated CH3 domain. Exemplary embodiments of mutated CH3 domain are provided in PCT/CA2020/051753 filed on Dec. 18, 2020, and published on Jun. 24, 2021 under No. WO2021/119832A1, the entire content of which is incorporated herein by reference. For example, a heterodimer may be formed by co-transfection of a nucleic acid sequences encoding a polypeptide chain comprising a mutated CH3 domain as set forth in SEQ ID NO:218 (chain A) and a nucleic acid sequences encoding a polypeptide chain comprising a mutated CH3 domain as set forth in SEQ ID NO:219 (chain B).

### Linkers (L)

The different modules of the polypeptide chains disclosed herein may be associated to each other via linkers.

In some embodiments, the linkers used to join one or more modules of the polypeptide chain are not cleavable linkers.

In an exemplary embodiment, the linker located immediately adjacent to the C-terminal end of the dimerization domain (L_{c}) does not comprise a cleavable linker.

In another exemplary embodiment, at least one of the linkers located between two antigen binding domains do not comprise a cleavable linker.

In other embodiments the linkers used to join one or more modules of the polypeptide chain may include non-cleavable linkers.

In an exemplary embodiment, the linker located immediately adjacent to the C-terminal end of the dimerization domain is a non-cleavable linker.

In another exemplary embodiment, at least one of the linkers located between two antigen binding domains is a non-cleavable linker.

In a further exemplary embodiment, the linker located immediately adjacent to the C-terminal end of the dimerization domain and the linker joining the first two antigen binding domains located at the C-terminal end of the dimerization domain are non-cleavable linkers.

In some embodiment, the linker immediately adjacent to the N-terminal end of the dimerization domain may preferably comprise hinge region of an antibody.

In some embodiments, the hinge region is natural hinge region.

In some embodiments, the natural hinge region is a natural IgG1 hinge region. In some embodiments, the natural hinge region is a natural human IgG1 hinge region.

In some embodiments, the natural hinge region is a natural IgG2 hinge region. In other embodiments, the natural hinge region is a natural human IgG2 hinge region.

In some embodiments, the natural hinge region is a natural IgG3 hinge region. In other embodiments, the natural hinge region is a natural human IgG3 hinge region.

In some embodiments, the natural hinge region is a natural IgG4 hinge region. In some embodiments, the natural hinge region is a natural human IgG4 hinge region.

In some embodiments, the hinge region is a mutated hinge region.

In some embodiments, the mutated hinge region is a mutated IgG1 hinge region. In some embodiments, the mutated hinge region is a mutated human IgG1 hinge region.

In some embodiments, the mutated hinge region is a mutated IgG2 hinge region. In other embodiments, the mutated hinge region is a mutated human IgG2 hinge region.

In some embodiments, the mutated hinge region is a mutated IgG3 hinge region. In other embodiments, the mutated hinge region is a mutated human IgG3 hinge region.

In some embodiments, the mutated hinge region is a mutated IgG4 hinge region. In some embodiments, the mutated hinge region is a mutated human IgG4 hinge region.

In some embodiments, all modules of the polypeptide chain are linked via non-cleavable linkers.

Exemplary embodiments of non-cleavable linkers include those that remains substantially intact during protein expression or during manufacturing process. As used herein “substantially intact” means that linker cleavage occurs in 20% or less, in 15% or less, in 10% or less, in 7.5% or less, in 5% or less, in 4% or less, in 3% or less, in 2% or less, in 1% or less of the total polypeptide content of a given solution or composition.

Other exemplary embodiments of non-cleavable linkers also include linkers that do not comprise a specific cleavage site for one or more proteases present in human or animal blood or serum.

Additional exemplary embodiments of non-cleavable linkers further include linkers that retain their integrity for at least one, two, three, four, five, six, twelve, twenty-four, forty-eight hours or more after administration upon administration of the binding agent in individuals.

In further exemplary embodiments, a linker comprises both non-cleavable linkers and cleavable linkers.

In some embodiments, a linker is not cleavable.

In some instance cleavable linkers may be used for in vivo release of drugs (e.g., cytostatic molecules, cytotoxic molecules, chemotherapeutics etc.) or labels attached to the polypeptide chain of the present disclosure.

Exemplary embodiments of cleavable linkers are provided for example in 1US2019/0010242 and include linkers that are sensitive to cleavage by a protease, usually an extracellular protease, such as a protease that is produced by a tumor or an activated immune effector cell and include those having a site for specific cleavage by proteases selected from ADAMS, ADAMTS, e.g. ADAMS; ADAMS; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; cysteine proteinases, e.g., Cruzipain; Legumain; Otubain-2; KLKs, e.g., KLK4, KLK5, KLK6, KLK7, KLK8, KLK10, KLK11, KLK13, or KLK14; metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, granzyme B, Guanidinobenzoatase, HtrAl, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, or TMPRSS4; and any combination thereof. In some embodiments the polypeptide chains of the present disclosure do not include such linkers at position corresponding to L_{c}.

Exemplary embodiments of linkers include flexible linkers, rigid linkers, helical linkers and combination thereof. Linkers are discussed for example, in Chen X et al. (Adv Drug Deliv Rev. 2013; 65(10):1357-1369) the entire content of which is incorporated herein by reference.

In some embodiments, a hinge region or a portion thereof may be used to link a module to the dimerization domain and is considered herein as a linker. The hinge region may be derived from a natural antibody (of human or animal origin) or from a synthetic antibody.

Hinge regions may be obtained, for example, from IgGs such as IgG1, IgG2, IgG3 or IgG4.

Exemplary embodiments of hinge regions are provided in SEQ ID NO:180, SEQ ID NO:203, SEQ ID NO:207 and SEQ ID NO:211.

In some instances, the hinge region may have one or more amino acid substitutions, amino acid insertion and/or amino acid deletions in comparison with a natural hinge region.

Mutated hinge region includes, for example, a sequence that is from 80% to 99% identical with that of a natural IgG1, IgG2, IgG3 or IgG4 hinge region (mutated hinge region). An exemplary and non-limiting embodiment of a mutated hinge region includes a hinge region of an IgG4 in which S228 is replaced with P (EU numbering) (Angal, S. et al., Mol Immunol 30, 105-108, 1993). Other exemplary embodiments of mutated hinge are provided in SEQ ID NOs: 118-120, 122-124, 126-128 and 130-132.

Flexible linkers are usually composed of small polar amino acids such as tlhreonine or serine and glycine. Exemplary and non-limiting embodiments of flexible linkers include GS linkers (glycine/serine repeats) such as for example, (GGGS)n(GGGGS)m_(SEQ ID NO:271), (GS)ₙ, (GGS)ₙ, (GGGS)ₙ(SEQ ID NO:272), (GGGGS)ₙ(SEQ ID NO:186), (GGSG)ₙ(SEQ ID NO:273), (GGGSS)ₙ(SEQ ID NO:274) wherein n and m may independently be an integer such as 1, 2, 3, 4, 5, 6, 7, 8, 9, or 10 or more, such as 15, 20 or 25.

It is to be understood that SEQ ID NO:186 may be represented by formula (GGGGS)ₙ, wherein n is an integer selected from 1 to 10 or alternatively by formula GGGGSX₁, wherein X₁ is absent or, if present is from 1 to 9 repeats of amino acid residues 1 to 5 of SEQ ID NO:186.

It is to be understood that SEQ ID NO:271 may be represented by formula (GGGS)ₙ(GGGGS)ₘ, wherein n and/or m is independently an integer selected from 1 to 10 or alternatively by formula GGGSX₁GGGGSX₂, wherein X₁ is absent or, if present is from 1 to 9 repeats of amino acid residues 1 to 4 of SEQ ID NO:271 and wherein X₂ is absent or, if present is from 1 to 9 repeats of amino acid residues 6 to 10 of SEQ ID NO:271.

It is to be understood that SEQ ID NO:272 may be represented by formula (GGGS)ₙ, wherein n is an integer selected from 1 to 10 or alternatively by formula GGGSX₁, wherein X₁ is absent or, if present is from 1 to 9 repeats of amino acid residues 1 to 4 of SEQ ID NO:272.

It is to be understood that SEQ ID NO:273 may be represented by formula (GGSG)ₙ, wherein n is an integer selected from 1 to 10 or alternatively by formula GGSGX₁, wherein X₁ is absent or, if present is from 1 to 9 repeats of amino acid residues 1 to 4 of SEQ ID NO:273.

It is to be understood that SEQ ID NO:274 may be represented by formula (GGGSS)ₙ, wherein n is an integer selected from 1 to 10 or alternatively by formula GGGSSX₁, wherein X₁ is absent or, if present is from 1 to 9 repeats of amino acid residues 1 to 5 of SEQ ID NO:274.

In accordance with the present disclosure n may be an integer such as 1, 2, 3, 4, 5, 6, 7, 8, 9, 10 or more, such as 15, 20 or 25.

In accordance with the present disclosure m may be an integer such as 1, 2, 3, 4, 5, 6, 7, 8, 9, 10 or more, such as 15, 20 or 25.

Specific exemplary and non-limiting embodiments of flexible linkers include those comprising of consisting of the amino acid sequence set forth in SEQ ID NO:182, SEQ ID NO:183, SEQ ID NO:184, SEQ ID NO: 185 or SEQ ID NO: 186.

Other exemplary and non-limiting embodiments of linkers include those comprising of consisting of the amino acid sequence set forth in SEQ ID NO:271, 272, 273 or 274.

It is to be understood that SEQ ID NO:186 may be represented by formula (GGGGS)ₙ wherein n is an integer selected from 1 to 10 or alternatively by formula GGGGSX₁ wherein X₁ is absent or, if present is from 1 to 9 repeats of amino acid residues 1 to 5 of SEQ ID NO:186.

Rigid linkers of the present disclosure are usually composed of proline-rich sequences (XP)ₙ, wherein X designate any amino acid, preferably Ala, Lys or Glu and n is an integer such as 1, 2, 3, 4, 5, 6, 7, 8, 9, 10 etc. (Chen X el at., 2013).

Specific exemplary and non-limiting embodiments of rigid linkers include those comprising of consisting of the amino acid sequence set forth in SEQ ID NO:187, SEQ ID NO:188, SEQ ID NO:189 or SEQ ID NO:190.

It is to be understood that SEQ ID NO:190 may be represented by formula (X(PAPAP))ₙKA wherein n is an integer selected from 1 to 10, wherein X is present or absent and, if present, is A or, alternatively, SEQ ID NO:190 may be represented by formula (XPAPAP)X₂KA wherein X may be present or absent and, if present, is A; and wherein X₂ is absent or, if present, is from 1 to 9 repeats of amino acid residues 1 to 6 of SEQ ID NO:190.

Helical linkers may sometimes be characterized as rigid but are herein being separated into a distinct linker family. Exemplary embodiments of helical linkers are discussed in Chen X e at., 2013 and comprise, for example, repeats of alanine residues flanked by a positively charged- and a negatively charged amino acid residue.

Specific exemplary and non-limiting embodiments of helical linkers include those comprising of consisting of the amino acid sequence set forth in SEQ ID NO:191, SEQ ID NO:192, SEQ ID NO:193 and SEQ ID NO:194.

It is to be understood that SEQ ID NO:194 may be represented by formula X(EAAAK)ₙX₂ wherein n is an integer selected from 1 to 10, more preferably 2-5 wherein X and X₂ are independently present or absent and, if present, is preferably A. Alternatively, SEQ ID NO:194 may be represented by formula X(EAAAK)X₃X₂, wherein X and X₂ are independently present or absent and, if present, is preferably A; and X₃ is absent or, if present, is from 1 to 9 repeats of amino acid residues 2 to 6 of SEQ ID NO:194.

In an exemplary embodiment the linker immediately adjacent to the C-terminal end of the dimerization domain (identified as L_{c1} in formulas II to VIII) may comprise either a flexible linker, a rigid linker or a helical linker. Linkers that may be particularly selected to occupy this position include for example and without limitations, a linker comprising or consisting of the amino acid sequence set forth in SEQ ID NO:182-191, SEQ ID NO:193 or in SEQ ID NO:194 wherein n is 1.

In an exemplary embodiment the linker joining the first two antigen binding domains located at the C-terminal end of the dimerization domain (identified as L_{c2} in formulas II to VIII) may comprise either a flexible linker, a rigid linker or a helical linker. Linkers that may be particularly selected to occupy this position include for example and without limitations, a linker comprising or consisting of the amino acid sequence set forth in SEQ ID NO:182-192, or in SEQ ID NO:194 wherein n is 1.

The present disclosure also provides linkers having an addition of from 1 to 10 amino acids (and any range or value comprised within 1 and 10 such as for example, from 1 to 5) at one or both the N- or C-terminus of any of SEQ ID NOs: 182 to 194. These additional amino acid residues may each independently be selected from any amino acid residues. These additional amino acid residues preferably form a non-cleavable sequence.

The present disclosure also provides linkers having a deletion of from 1, 2, 3, 4 or 5 amino acids (and any value comprised within 1 and 5) at one or both the N- or C-terminus of any of SEQ ID NOs: 182 to 194.

Suitable linkers may comprise, for example, an amino acid sequence comprising from about 3 to about 50, from about 3 to about 40, from about 3 to about 30, from about 3 to about 25, from about 3 to about 20, from about 3 to about 15, from about 3 to about 10 amino acid residues.

In exemplary embodiments, the length of each linker may independently range from about 5 to about 50 amino acid residues, including for example, from about 5 to about 40 amino acid residues, from about 10 to about 40 amino acid residues, from about 20 to about 40 amino acid residues, from about 20 to about 35 amino acid residues, from about 25 to about 30 amino acid residues and any sub-range comprised and including such ranges.

In some embodiments linkers that comprise the amino acid sequence set forth in SEQ ID NO:186, SEQ ID NO:190 or SEQ ID NO:194 may have a “n” value preferably from 1 to 10, more preferably from 2-5 including 2, 3, 4 or 5.

### Binding Agents

Binding agents of the present disclosure encompass for example the antigen binding domains disclosed herein.

Binding agents of the present disclosure encompass for example the polypeptide chains disclosed herein. Accordingly in some embodiments the binding agent may be composed of a single polypeptide chain. In some embodiments, the binding agent is a polypepdide chain.

Binding agents of the present disclosure encompass for example dimers of polypeptide chains disclosed herein.

Binding agents of the present disclosure encompass for example multimers of polypeptide chains disclosed herein.

The binding agent of the present disclosure is capable of binding to a CD36 protein or a portion thereof. In some embodiments, the binding agent comprises one or more antigen binding domains that are capable of binding to a CD36 protein or a portion thereof or to cells expressing a CD36 protein or a portion thereof. In accordance with the present disclosure, the one or more antigen binding domains are from an anti-CD36 antibody or antigen binding fragment thereof. Also in accordance with the present disclosure, the one or more antigen binding domains are from an anti-CD36 single domain antibody.

Exemplary and non-limiting embodiments of antigen binding domains are provided in Table 3, Table 4 and Table 5.

The binding agent of the present disclosure may also be capable of binding to an immune checkpoint protein and/or to a protein expressed at the surface of immune cells. The binding agent may thus comprise one or more antigen binding domains that are capable of binding to an immune checkpoint protein and/or one or more antigen binding domains that are capable of binding to to a protein expressed at the surface of immune cells.

In some embodiments, the binding agent of the present disclosure may comprise an antigen binding domain that binds CD36, an antigen binding domain that binds PD1 and an antigen binding domain that binds to CD47. In some embodiments, the binding agent of the present disclosure may be capable of bringing T cells in proximity to CD36-expressing tumor cells, may be capable of restoring T cell function and/or may be capable of enhancing macrophage function via blocking of SIRPa/CD47 interaction. In some embodiments of the present disclosure, the binding agent may exhert its anti-tumor activity via antibody-dependent cell-mediated cellular cytotoxicity (ADCC). In some embodiments of the present disclosure, the binding agent may exert its anti-tumor activity via antibody-dependent cellular phagocytosis (ADCP) activity.

As such, in some exemplary embodiments, the binding agent may comprise one or more antigen binding domains that are capable of binding to a PD-1 protein or a portion thereof or to cells expressing a PD-1 protein or a portion thereof. In other exemplary embodiments, the binding agent may comprise one or more antigen binding domains that are capable of binding to a CD47 protein or a portion thereof or to cells expressing a CD47 protein or a portion thereof.

In some embodiments, the binding agent of the present disclosure may be capable of reducing tumor growth and/or of causing tumor regression. In some embodiments, the binding agent has anti-tumor activity. In some embodiments, the binding agent is able to recruit or redirect immune cells.

In some instances, the binding agent of the present disclosure may modulate lipid metabolism.

In other instances, the binding agent of the present disclosure may inhibit fatty acid oxidation.

In yet other instances, the binding agent of the present disclosure may inhibit fatty acid uptake by tumors.

In other instances, the binding agent of the present disclosure may be able to inhibit the growth of tumor cells.

In yet other instances, the binding agent of the present disclosure may be able to inhibit tumor metastasis.

In yet other instances, the binding agent of the present disclosure may be able to reduce resistance of tumor cells to chemotherapeutics and/or immune checkpoint inhibitors therapy.

Binding agents of the present disclosure may have a format of an antibody and antigen binding fragment thereof, an antibody-like molecule (Fc-, CH3- fusions and the like), a fusion with protein scaffolds, immune cell modulating agents and the like.

In some embodiments, the binding agent comprises an antibody or an antigen binding fragment thereof. In some embodiments, the binding agent is an antibody or an antigen binding fragment thereof.

In some embodiments, the binding agent comprises an antibody-like molecule. In some embodiments, the binding agent is an antibody-like molecule.

In some embodiments, the binding agent may be fused with protein scaffold.

In some embodiments, the binding agent comprises an immune cell modulating agent.

In some embodiments, the binding agent is an immune cell modulating agent.

Accordingly, in some embodiments, binding agents include antibodies and antigen-binding fragments thereof such as, for example and without limitations, single domain antibodies from camelids or sharks, human antibodies including IgGs (including human IgG1, human IgG2, human IgG3, human IgG4), human IgMs, human IgAs (including human IgA1 and human IgA2), human IgEs, human IgDs, animal antibodies including for example, IgGs (IgG1, IgG2a, IgG2b, IgG2c, IgG3, IgG4), IgMs, IgAs, IgEs and IgDs.

In some embodiments, the binding agent comprises the antigen binding domain of a single domain antibody. In yet other embodiments, the binding agent comprises the antigen binding domain of one or more single domain antibodies.

For example, the binding agent may comprise the CDRs of one or more single domain antibodies. In another example, the binding agent may comprise the variable region (VH or VHH) of one or more single domain antibodies.

In some embodiments, the binding agent comprises a single domain antibody.

In some embodiments, the binding agent is a single domain antibody.

In other embodiments, binding agents include antigen-binding fragments such as, for example and without limitations, Fab, Fab′, F(ab′)₂, complementarity determining regions, variable regions including VHs, VHHs, VLs, and the like.

Binding agents also include immune cell modulating agents such as for example, dual-affinity retargeting molecules (DARTs), chimeric antigen receptors (CAR) constructs, bispecific T cell engagers construct (BiTEs), bispecific killer cell engagers (BiKEs), trispecific killer cell engagers (TriKEs) containing scFvs or VHHs.

Binding agents also encompass fusion with protein scaffolds, including ankyrin repeat proteins, Z-domain of Staphylococcus protein-A, Type-III fibronetin, knottin and the like.

Exemplary embodiments of binding agents include monospecific-, bispecifics (symmetric or asymmetric) trispecific-, or multispecific antibodies as well as monovalent, bivalent, trivalent or multivalent antibodies, single chain FVs (scFVs) and derivatives such diabody, triabody, tetrabody, tandem di-scFvs, tandem tri-scFvs, scFV-Fc, minibody (scFV-CH3), tandem diabody, di-diabody, bibody and the like, VH or VHHs and derivatives such as, tandem bispecific or multispecific VHH, bivalent VHH-Fc fusions, VHH-hinge-CH2-CH3 fusions, bivalent CH3 fusions, VHH pentabody, decabody and the like.

In some embodiments, the binding agents of the present disclosure may have a format as disclosed in PCT/CA2020/051753 filed on Dec. 18, 2020 (published on Jun. 24, 2021 under No. WO2021/119832 A1) as formula Ia, formula Ib, formula Ic, formula II, formula III, formula IV, formula V, formula VI, formula VII or formula VIII and the like or formula I, formula II, formula III, formula IIIa and formula IIIb, formula IV, formula V, formula VI, formula VII or formula VIII disclosed herein.

The binding agents of the present disclosure may be formed by the assembly of two polypeptide chains having the same configuration (with same or different amino acid sequence) or having different configurations where the same or different configurations include the configuration set forth in formula I, formula II, formula III, formula IIIa and formula IIIb, formula IV, formula V, formula VI, formula VII or formula VIII disclosed herein.

The binding agent of the present disclosure may comprise for example, one or more polypeptide chains independently comprising in a N- to C-terminal fashion an amino acid sequence of formula I:

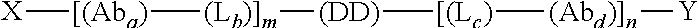

Wherein m is 0, 1, 2 or an integer greater than 2;
Wherein n is 0, 1, 2 or an integer greater than 2;
Wherein m and n are not 0 simultaneously;
Wherein Abₐ, Ab_{d}, each represents an antigen binding domain wherein at least one of the antigen binding domains is an antigen binding domain 1 (ABD1);
Wherein X or Y are independently present or absent and comprises an amino acid sequence;
Wherein L_{b}, L_{c}, each independently comprises one or more linkers; and
Wherein DD represents a dimerization domain.

In some embodiments, when m is 1, 2 or an integer greater than 2, one of Abₐ, or Ab_{d}is an antigen binding domain 1 (ABD1) and the other of Abₐ, or Ab_{d} is selected from an antigen binding domain 2 (ABD2) and/or an antigen binding domain 3 (ABD3).

In some embodiments, when n is 1, 2 or an integer greater than 2, one of Abₐ, or Ab_{d}is an antigen binding domain 1 (ABD1) and the other of Abₐ, or Ab_{d} is selected from an antigen binding domain 2 (ABD2) and/or an antigen binding domain 3 (ABD3).

In some embodiments, when m is 1, 2 or an integer greater than 2 and/or when n is 1, 2 or an integer greater than 2, at least one of Abₐ, or Ab_{d} is an antigen binding domain 1 (ABD1), at least one Abₐ, or Ab_{d} is an antigen binding domain 2 (ABD2) and and at least one Abₐ, or Ab_{d} is an antigen binding domain 3 (ABD3).

In some embodiments L_{b} may be absent. In some embodiments L_{b}2 and/or L_{b3} may be absent.

In some embodiments L_{c} may be absent. In some embodiments, L_{c1}, L_{c2}, and/or L_{c3} may be absent.

In some embodiments both L_{b} and L_{c} may be absent.

In some embodiments the polypeptide chain comprises more than one antigen binding domains.

In some embodiments the polypeptide chain comprises two antigen binding domains or more.

In some embodiments the polypeptide chain comprises three antigen binding domains or more.

In some embodiments the polypeptide chain comprises four antigen binding domains or more.

In some embodiments the polypeptide chain comprises five antigen binding domains or more.

In some embodiments the polypeptide chain comprises six antigen binding domains or more.

In some embodiments the polypeptide chain comprises between one and twelve antigen binding domains.

In some embodiments, the binding agent may comprise between one and twelve antigen binding domains, such as between one and two, between one and three, between one and four, between one and five, between one and six, between one and seven, between one and eight, between one and nine, between one and ten, between one and eleven, between one and twelve, between two and three, between two and four, between two and five, between two and six, between two and seven, between two and eight, between two and nine, between two and ten, between two and eleven, between two and twelve, between three and four, between three and five, between three and six, between three and seven, between three and eight, between three and nine, between three and ten, between three and eleven, between three and twelve, between four and five, between four and six, between four and seven, between four and eight, between four and nine, between four and ten, between four and eleven, between four and twelve, between five and six, between five and seven, between five and eight, between five and nine, between five and ten, between five and eleven, between five and twelve, between six and seven, between six and eight, between six and nine, between six and ten, between six and eleven, between six and twelve, between seven and eight, between seven and nine, between seven and ten, between seven and eleven, between seven and twelve, between eight and nine, between eight and ten, between eight and eleven, between eight and twelve, between nine and ten, between nine and eleven, between nine and twelve, between ten and eleven, between ten and twelve, or between eleven and twelve.

In some embodiments, the binding agent comprises one polypeptide chain.

In some embodiments, the binding agent comprises two polypeptide chains.

In some embodiments, the binding agent comprises three polypeptide chains.

In some embodiments, the binding agent comprises four polypeptide chains.

In some embodiments, the binding agent comprises five polypeptide chains.

In some embodiments, the binding agent comprises six polypeptide chains.

In some embodiments, the binding agent comprises seven polypeptide chains.

In some embodiments, the binding agent comprises eight polypeptide chains.

In some embodiments, the binding agent comprises nine polypeptide chains.

In some embodiments, the binding agent comprises ten polypeptide chains.

In some embodiments, the binding agent comprises more than ten polypeptide chains.

In some embodiments the binding agent comprises at least one polypeptide chain having formula II: X-(Abₐ₁)-(L_{b1})-(DD)-(L_{c1})-(Abₐ₁)-Y (formula II).

In some embodiments the binding agent comprises at least one polypeptide chain having formula III: X-(Abₐ₁)-(L_{b1})-(DD)-(L_{c1})-(Abₐ₁)-(L_{c2})-(Abₐ₂)-Y (formula III).

In some embodiments the binding agent comprises at least one polypeptide chain having formula IV: X-(Abₐ₁)-(L_{b2})-(Abₐ₂)-(L_{b1})-(DD)-(L_{c1})-(Ab_{d1})-Y (formula IV).

In some embodiments the binding agent comprises at least one polypeptide chain having formula V: X-(Abₐ₁)-(L_{b2})-(Abₐ₂)-(L_{b1})-(DD)-(L_{c1})-(Ab_{d1})-(L_{c2})-(Ab_{d2})-Y (formula V).

In some embodiments the binding agent comprises at least one polypeptide chain having formula VI: X-(Abₐ₁)-(L_{b2})-(Abₐ₂)-(L_{b1})-(DD)-(L_{c1})-(Ab_{d1})-(L_{c2})-(Abₐ₂)-(L_{c3})-(Ab_{d3})-Y (formula VI).

In some embodiments the binding agent comprises at least one polypeptide chain having formula VII: X-(Abₐ₁)-(L_{b3})-(Abₐ₂)-(L_{b2})-(Abₐ₃)-(L_{b1})-(DD)-(L_{c1})-(Ab_{d1})-(L_{c2})-(Ab_{d2})-Y (formula VII).

In some embodiments the binding agent comprises at least one polypeptide chain having formula VIII: X-(Abₐ₁)-(L_{b3})-(Abₐ₂)-(L_{b2})-(Abₐ₃)-(L_{b1})-(DD)-(L_{c})-(Ab_{d1})-(L_{c2})-(Ab_{d2})-(L_{c3})-(Ab_{d3})-Y (formula VIII).

In some embodiments, the binding agent comprises one or more polypeptide chains each independently comprising in a N- to C-terminal fashion an amino acid sequence of formula III

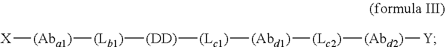

wherein one of Abₐ₁, Ab_{d1} or Ab_{d2} represents the antigen binding domain 1 (ABD1).

In some embodiments, the binding agent comprises one or more polypeptide chains each independently comprising in a N- to C-terminal fashion an amino acid sequence of formula III

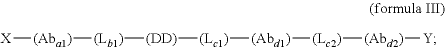

wherein one of Abₐ₁, Ab_{d1} or Ab_{d2} represents the antigen binding domain 1 (ABD1), one of Abₐ₁, Ab_{d1} or Ab_{d2} represents the antigen binding domain 2 (ABD2) and one of Abₐ₁, Ab_{d1} or Ab_{d}2 represents the antigen binding domain 3 (ABD3).

In some embodiments, the binding agent comprises one or more polypeptide chains each independently comprising in a N- to C-terminal fashion an amino acid sequence of formula III

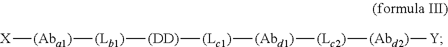

wherein Abₐ₁ represents the antigen binding domain 1 (ABD1), Ab_{d1} represents the antigen binding domain 2 (ABD2) and Abₐ₂ represents the antigen binding domain 3 (ABD3).

In some embodiments, the binding agent comprises one or more polypeptide chains each independently comprising in a N- to C-terminal fashion an antigen binding domain that binds to CD36, an antigen binding domain that binds to PD-1 and an antigen binding domain that binds to CD47.

In some embodiments, the binding agent comprises one or more polypeptide chains each independently comprising in a N- to C-terminal fashion an amino acid sequence of formula III, wherein Abₐ₁ is an antigen binding domain that binds to CD36, Ab_{d1} is an antigen binding domain that binds to PD-1 and Abₐ₂ is an antigen binding domain that binds to CD47.

In some embodiments, the binding agent comprises one or more polypeptide chains each independently comprising in a N- to C-terminal fashion an amino acid sequence of formula III, wherein Abₐ₁ is an antigen binding domain that binds to CD36, Ab_{d1} is an antigen binding domain that binds to CD47 and Ab_{d2} is an antigen binding domain that binds to PD-1.

In some embodiments, the binding agent comprises one or more polypeptide chains each independently comprising in a N- to C-terminal fashion an amino acid sequence of formula IIIa:

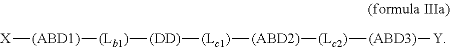

In some embodiments, the binding agent comprises one or more polypeptide chains each independently comprising in a N- to C-terminal fashion an amino acid sequence of formula IIIb:

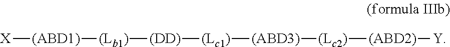

In some embodiments, the binding agent comprises two identical polypeptide chains.

In some embodiments, both polypeptide chains of the binding agent may have the configuration set forth in formula II (with same or different amino acid sequence).

In some embodiments, both polypeptide chains of the binding agent may have the configuration set forth in formula III (with same or different amino acid sequence).

In some embodiments, both polypeptide chains of the binding agent may have the configuration set forth in formula IIIa (with same or different amino acid sequence).

In some embodiments, both polypeptide chains of the binding agent may have the configuration set forth in formula IIIb (with same or different amino acid sequence).

In some embodiments, one polypeptide chain may have the configuration set forth in formula IIIa one polypeptide chain may have the configuration set forth in formula IIIb (with antigen binding domains having same or different amino acid sequence).

In some embodiments, one of the polypeptide chains may have the configuration set forth in formula II, while the other may have the configuration set forth in formula III.

In some embodiments, one of the polypeptide chains may have the configuration set forth in formula II, while the other has the configuration set forth in formula IV.

In some embodiments, one of the polypeptide chains may have the configuration set forth in formula III, while the other has the configuration set forth in formula IV.

In some embodiments, one of the polypeptide chains may have the configuration set forth in formula IV, while the other has the configuration set forth in formula IV.

In some embodiments, the binding agents of the present disclosure is monospecific or multispecific.

Monospecific binding agents encompass binding agents that are specific for a single epitope of a given antigen.

An exemplary embodiment of a monospecific binding agent includes a binding agent that comprise one antigen binding domain. Another exemplary embodiment of a monospecific binding agent includes a binding agent that comprise more than one antigen binding domains, but the antigen binding domains have identical CDRs and framework regions. Yet another exemplary embodiment of a binding agent chain includes a binding agent that comprise more than one antigen binding domains, but the antigen binding domains have identical CDRs and different framework regions. A further exemplary embodiment of a monospecific binding agent includes a binding agent that comprise antigen binding domains that differ in the amino acid sequence of one or more of their CDRs (e.g., conservative substitution in one or more CDRs) without affecting their ability to bind to the same antigen or epitope.

Multispecific binding agents encompass binding agents that are specific for more than one epitope (of the same antigen or different antigens) or to more than one antigen. For example, a multipecific polypeptide chain or binding agent may thus have more than one antigen binding domains, at least two of which bind to different antigens or epitopes.

The binding agents of the present disclosure may thus be bispecific, trispecific, tetraspecific, pentaspecific, hexaspecific etc. In some embodiments, each antigen binding domain may be specific for a given antigen. In some embodiments, two or more antigen binding domains of a given binding agent may be specific for the same or different antigens. In some embodiments, three or more antigen binding domains of a given binding agent may be specific for the same or different antigens. In some embodiments, four or more antigen binding domains of a given binding agent may be specific for the same or different antigens. In some embodiments, five or more antigen binding domains of a given binding agent may be specific for the same or different antigens. In some embodiments, six or more antigen binding domains of a given binding agent may be specific for the same or different antigens. The specificity may depend on the number of antigen binding domains present in a given binding agent.

Exemplary non-limiting embodiments of multispecific binding agent include those composed of multispecific polypeptide chains. Other exemplary non-limiting embodiments of multispecific binding agent include those having two antigen binding domains of different specificities. Yet other exemplary non-limiting embodiments of multispecific binding agent include those having more than two antigen binding domains that bind to two different antigens, proteins or to two different epitopes on the same antigens or proteins.

In some embodiments, the binding agents of the present disclosure is monovalent or multivalent.

Exemplary non-limiting embodiments of multivalent binding agents include binding agents composed of multivalent polypeptide chains. Other non-limiting exemplary embodiments of multivalent binding agent include binding agents composed of more than one monovalent polypeptide chain.

In accordance with the present disclosure, a bispecific binding agent may be bivalent or multivalent depending on the number of antigen binding domains that it contains. Exemplary non-limiting embodiments of bispecific binding agent include those comprising two identical bispecific polypeptide chains that form a dimer.

Exemplary and non-limiting embodiments of binding agents are provided in Table 6 and Table 7.

Other exemplary embodiments of binding agent, include those comprising the amino acid sequence set forth in Table 3 in which the amino acid sequence set forth in SEQ ID NO:178 is fused at the C-terminal end.

Yet other exemplary embodiments of binding agents include those comprising the amino acid sequence set forth in Table 3 and comprising linkers, dimerization domains and optionally one or more antigen binding domains having different amino acid sequences.

Other exemplary embodiments of binding agent, include those comprising the amino acid sequence set forth in Table 6 in which the amino acid sequence set forth in SEQ ID NO:179 is fused at the C-terminal end.

### Competing Binding Agents

Binding agents of the present disclosure also encompass those that compete with a binding agent disclosed herewith, or with a single domain antibody having the amino acid sequence disclosed herewith.

Antibodies that compete with one or more single domain antibody or antigen binding fragment thereof having an amino acid sequence as disclosed herein are provided. In some embodiments, the antibody is a single domain antibody that competes with one or more single domain antibody or antigen binding fragment thereof having an amino acid sequence as disclosed herein.

In an exemplary embodiment the binding agent may comprises one or more antigen binding domains of an antibody that compete with one or more single domain antibody or antigen binding fragment thereof having an amino acid sequence as disclosed herein.

Competing binding agents of the present disclosure may comprise an amino acid sequence having at least 75% identity or more with the amino acid sequence set forth in any one of Table 3, Table 4, Table 5, Table 6 and/or Table 7 or combination thereof.

Alternatively, competing binding agents may comprise an amino acid sequence that has less than 75% identity with the amino acid sequence set forth in any one of Table 3, Table 4, Table 5, Table 6 and/or Table 7 or having different CDR1, CDR2 and/or CDR3 but still be able to bind to the same target (CD36, PD-1 and/or CD47) and remaining functional. Such competing binding agent may be identified by competition assays using target and a pre-identified binding agent (e.g., Table 6 and/or Table 7). Functionality of the competing binding agent is confirmed by testing its activity including for example and without limitation its ability to reduce fatty acid oxidation, lipid metabolism and/or fatty acid uptake by cells.

In an exemplary embodiment, competition between binding agents may be determined based on any type of binding assays (FACS, ELISA, SPR etc.) using a recombinant CD36 or portion thereof or cells expressing CD36 or portion thereof.

In exemplary embodiments, the competing binding agent is capable of blocking CD36 or comprises one or more antigen binding domains that is capable of blocking CD36.

In some embodiments, the binding agent comprises one or more antigen binding domains of an antibody that is capable of competing (competing antibody) with a single domain antibody comprising an antigen binding domain 1 (ABD1) as set forth herein. In other embodiments, the binding agent comprises one or more antigen binding domains of an antibody that is capable of competing with an anti-CD36 single domain antibody comprising an antigen binding domain having a CDRH1, CDRH2 and/or CDRH3 amino acid sequence as disclosed herein. More particularly, the binding agent comprises one or more antigen binding domains of an antibody that is capable of competing with an anti-CD36 single domain antibody comprising an antigen binding domain having a CDRH1, CDRH2 and CDRH3 amino acid sequence as disclosed herein.

In some embodiments, at least one of the one or more antigen binding domains of the competing antibody comprises the CDRH1, CDRH2 and/or CDRH3 amino acid sequence of the anti-CD36 single domain antibody of Table 3.

In some embodiments, at least one of the one or more antigen binding domains of the competing antibody comprises:
a. an amino acid sequence at least 75%, 80% 85%, 90%, 95% identical to the amino acid sequence set forth in SEQ ID NO:7 or having from one to ten amino acid substitutions, additions and/or deletions in SEQ ID NO:7;
b. an amino acid sequence at least 75%, 80% 85%, 90%, 95% identical to the amino acid sequence set forth in SEQ ID NO:14 or having from one to ten amino acid substitutions, additions and/or deletions in SEQ ID NO:14;
c. an amino acid sequence at least 75%, 80% 85%, 90%, 95% identical to the amino acid sequence set forth in SEQ ID NO:21 or having from one to ten amino acid substitutions, additions and/or deletions in SEQ ID NO:21;
d. an amino acid sequence at least 75%, 80% 85%, 90%, 95% identical to the amino acid sequence set forth in SEQ ID NO:28 or having from one to ten amino acid substitutions, additions and/or deletions in SEQ ID NO:28;
e. an amino acid sequence at least 75%, 80% 85%, 90%, 95% identical to the amino acid sequence set forth in SEQ ID NO:35 or having from one to ten amino acid substitutions, additions and/or deletions in SEQ ID NO:35;
f. an amino acid sequence at least 75%, 80% 85%, 90%, 95% identical to the amino acid sequence set forth in SEQ ID NO:42 or having from one to ten amino acid substitutions, additions and/or deletions in SEQ ID NO:42;
g. an amino acid sequence at least 75%, 80% 85%, 90%, 95% identical to the amino acid sequence set forth in SEQ ID NO:49 or having from one to ten amino acid substitutions, additions and/or deletions in SEQ ID NO:49;
h. an amino acid sequence at least 75%, 80% 85%, 90%, 95% identical to the amino acid sequence set forth in SEQ ID NO:56 or having from one to ten amino acid substitutions, additions and/or deletions in SEQ ID NO:56;
i. an amino acid sequence at least 75%, 80% 85%, 90%, 95% identical to the amino acid sequence set forth in SEQ ID NO:77 or having from one to ten amino acid substitutions, additions and/or deletions in SEQ ID NO:77;
j. an amino acid sequence at least 75%, 80% 85%, 90%, 95% identical to the amino acid sequence set forth in SEQ ID NO:84 or having from one to ten amino acid substitutions, additions and/or deletions in SEQ ID NO:84;
k. an amino acid sequence at least 75%, 80% 85%, 90%, 95% identical to the amino acid sequence set forth in SEQ ID NO:91 or having from one to ten amino acid substitutions, additions and/or deletions in SEQ ID NO:91;
l. an amino acid sequence at least 75%, 80% 85%, 90%, 95% identical to the amino acid sequence set forth in SEQ ID NO:98 or having from one to ten amino acid substitutions, additions and/or deletions in SEQ ID NO:98;
m. an amino acid sequence at least 75%, 80% 85%, 90%, 95% identical to the amino acid sequence set forth in SEQ ID NO:105 or having from one to ten amino acid substitutions, additions and/or deletions in SEQ ID NO:105;
n. an amino acid sequence at least 75%, 80% 85%, 90%, 95% identical to the amino acid sequence set forth in SEQ ID NO:112 or having from one to ten amino acid substitutions, additions and/or deletions in SEQ ID NO:112;
o. an amino acid sequence at least 75%, 80% 85%, 90%, 95% identical to the amino acid sequence set forth in SEQ ID NO:119 or having from one to ten amino acid substitutions, additions and/or deletions in SEQ ID NO:119;
p. an amino acid sequence at least 75%, 80% 85%, 90%, 95% identical to the amino acid sequence set forth in SEQ ID NO:126 or having from one to ten amino acid substitutions, additions and/or deletions in SEQ ID NO:126;
q. an amino acid sequence at least 75%, 80% 85%, 90%, 95% identical to the amino acid sequence set forth in SEQ ID NO:133 or having from one to ten amino acid substitutions, additions and/or deletions in SEQ ID NO:133 or,
r. an amino acid sequence at least 75%, 80% 85%, 90%, 95% identical to the amino acid sequence set forth in SEQ ID NO:140 or having from one to ten amino acid substitutions, additions and/or deletions in SEQ ID NO:140.

The competing antibody may be a competing single domain antibody or an antigen binding fragment thereof.

In some embodiments, the binding agent may also comprise one or more antigen binding domain of an anti-PD-1 single domain antibody disclosed herein.

In some embodiments, at least one of the one or more antigen binding domains is from an anti-PD-1 antibody capable of competing (competing antibody) with a single domain antibody comprising an antigen binding domain 2 (ABD2) as set forth herein.

In other embodiments, the binding agent may also comprise one or more antigen binding domains of an antibody that is capable of competing with an anti-PD-1 single domain antibody comprising an antigen binding domain having a CDRH1, CDRH2 and/or CDRH3 amino acid sequence as disclosed herein. More particularly, the binding agent comprises one or more antigen binding domains of an antibody that is capable of competing with an anti-PD-1 single domain antibody comprising an antigen binding domain having a CDRH1, CDRH2 and CDRH3 amino acid sequence as disclosed herein.

In some embodiments, the anti-PD-1 antibody comprises an amino acid sequence at least 75%, 80% 85%, 90%, 95% identical to the amino acid sequence set forth in any one of SEQ ID NO:63, 281, 288, 295, 302, 309, 316, 323, 330, 337, 344, 351, 358 and/or 365 or having from one to ten amino acid substitutions, additions and/or deletions in any one of SEQ ID NO: 281, 288, 295, 302, 309, 316, 323, 330, 337, 344, 351, 358 and/or 365.

In some embodiments, the binding agent may also comprise one or more antigen binding domain of an anti-CD47 single domain antibody disclosed herein.

In some embodiments, at least one of the one or more antigen binding domains is from an anti-CD471 antibody capable of competing (competing antibody) with a single domain antibody comprising an antigen binding domain 3 (ABD3) as set forth herein.

In other embodiments, the binding agent may also comprise one or more antigen binding domains of an antibody that is capable of competing with an anti-CD47 single domain antibody comprising an antigen binding domain having a CDRH1, CDRH2 and/or CDRH3 amino acid sequence as disclosed herein. More particularly, the binding agent comprises one or more antigen binding domains of an antibody that is capable of competing with an anti-CD47 single domain antibody comprising an antigen binding domain having a CDRH1, CDRH2 and CDRH3 amino acid sequence as disclosed herein.

In yet other embodiments, the anti-CD47 antibody comprises an amino acid sequence at least 75%, 80% 85%, 90%, 95% identical to the amino acid sequence set forth in SEQ ID NO:70 or having from one to ten amino acid substitutions, additions and/or deletions in SEQ ID NO:70.

In some embodiments, the binding agent comprises one or more antigen binding domains of an antibody that is capable of competing with the anti-PD-1 single domain antibody disclosed herein or one or more antigen binding domains of an antibody that is capable of competing with the anti-CD47 single domain antibody disclosed herein, and at least one of the one or more antigen binding domains of the antibody comprises:
a. an amino acid sequence identical to the amino acid sequence set forth in SEQ ID NO:7;
b. an amino acid sequence identical to the amino acid sequence set forth in SEQ ID NO:14;
c. an amino acid sequence identical to the amino acid sequence set forth in SEQ ID NO:21;
d. an amino acid sequence identical to the amino acid sequence set forth in SEQ ID NO:28;
e. an amino acid sequence identical to the amino acid sequence set forth in SEQ ID NO:35;
f. an amino acid sequence identical to the amino acid sequence set forth in SEQ ID NO:42;
g. an amino acid sequence identical to the amino acid sequence set forth in SEQ ID NO:49;
h. an amino acid sequence identical to the amino acid sequence set forth in SEQ ID NO:56;
i. an amino acid sequence identical to the amino acid sequence set forth in SEQ ID NO:77;
j. an amino acid sequence identical to the amino acid sequence set forth in SEQ ID NO:84;
k. an amino acid sequence identical to the amino acid sequence set forth in SEQ ID NO:91;
l. an amino acid sequence identical to the amino acid sequence set forth in SEQ ID NO:98;
m. an amino acid sequence identical to the amino acid sequence set forth in SEQ ID NO:105;
n. an amino acid sequence identical to the amino acid sequence set forth in SEQ ID NO:112;
o. an amino acid sequence identical to the amino acid sequence set forth in SEQ ID NO:119;
p. an amino acid sequence identical to the amino acid sequence set forth in SEQ ID NO:126;
q. an amino acid sequence identical to the amino acid sequence set forth in SEQ ID NO:133 or,
r. an amino acid sequence identical to the amino acid sequence set forth in SEQ ID NO:140.

### Exemplary Embodiments of Monospecific Binding Agents

As indicated herein the binding agent of the present disclosure may be monospecific.

Exemplary and non-limiting exemplary embodiments of monospecific binding agents are provided herein.

For example, the binding agent may comprise only one antigen binding domain. Alternatively, the binding agent may comprise more than one identical antigen binding domains.

Exemplary embodiments of monospecific binding agents include the antigen binding domains disclosed herein including ABD1, ABD2, ABD3 or heavy chains disclosed herein.

In exemplary embodiments, the monospecific binding agents may comprise the antigen binding domains disclosed herein and a dimerization domain.

Exemplary embodiments of dimerization domains are provided throughout the specification and include without limitation a CH3 domain (including natural CH3 domain and natural human CH3 domain, mutated CH3 domain) may also include a CH2 domain (natural CH2 domain or mutated CH2 domain).

In some embodiments the dimerization domain is a Fc region of an antibody or a portion thereof.

In some embodiments the dimerization domain may also comprise a hinge region of an antibody or a portion thereof. In some embodiments the hinge region or portion thereof is a human hinge region or a portion thereof. In some instances, linkers or additional amino acid sequences may be added between the antigen binding domains and dimerization domains or at any end thereof.

In some embodiments, the binding agent may comprise ABD1 and a dimerization domain.

In some embodiments, the binding agent may comprise ABD2 and a dimerization domain.

In some embodiments, the binding agent may comprise ABD3 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:7 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:14 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:21 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:28 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:35 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:42 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:49 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:56 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:77 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:84 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:91 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:98 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:105 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:112 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:119 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:126 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:133 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:140 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:63 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:281 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:288 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:295 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:302 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:309 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:316 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:323 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:330 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:337 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:344 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:351 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:358 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:365 and a dimerization domain.

In some exemplary embodiments, the binding agent of the present disclosure may comprise an amino acid sequence at least 75%, 80% 85%, 90%, 95% identical to the amino acid sequence set forth in SEQ ID NO:70 and a dimerization domain.

It is to be understood herein that the binding agents may optionally comprises one or more linkers or additional amino acid sequences.

Other exemplary embodiments of monospecific binding agents are provided herein and include without limitation, the single domain antibody of Table 6 or comprising the amino acid sequence of the single domain antibody of Table 6.

### Exemplary Embodiments of Multispecific Binding Agents

As indicated herein the binding agent of the present disclosure may be multispecific.

Exemplary and non-limiting exemplary embodiments of multispecific binding agents include are provided herein.

For example, in some embodiment, the binding agent may comprise more than one antigen binding domains, wherein at least one of the antigen binding domains is an antigen binding domain 1 (ABD1) as set forth herein and at least one of the antigen binding domains is an antigen binding domain that is capable of binding to an immune checkpoint protein.

In other embodiments, the binding agent may comprise more than one antigen binding domains, wherein at least one of the antigen binding domains is an antigen binding domain 1 (ABD1) as set forth herein, at least one of the antigen binding domains is an antigen binding domain that is capable of binding to an immune checkpoint protein and at least one of the antigen binding domains is an antigen binding domain that is capable of binding to a protein expressed at the surface of immune cells.

In yet other embodiments, the binding agent may comprise more than one antigen binding domains, wherein at least one of the antigen binding domains is an antigen binding domain 1 (ABD1) as set forth herein, at least one of the antigen binding domains is an antigen binding domain that is capable of binding to an immune checkpoint protein and at least one of the antigen binding domains is an antigen binding domain that is capable of binding to a protein expressed at the surface of tumors cells.

In some embodiments, the binding agent comprises at least one antigen binding domain 1 (ABD1) and at least one antigen binding domain 2 (ABD2).

In some embodiments, the binding agent comprises at least one antigen binding domain 1 (ABD1) and at least one antigen binding domain 3 (ABD3).

In some embodiments, the binding agent comprises at least one antigen binding domain 2 (ABD2) and at least one antigen binding domain 3 (ABD3).

In some embodiments, the binding agent comprises at least one antigen binding domain 1 (ABD1), at least one antigen binding domain 2 (ABD2) and at least one antigen binding domain 3 (ABD3).

In some embodiments, the binding agent is a multispecific binding agent that comprises two polypeptide chains that are capable of forming a dimer, wherein each polypeptide chain comprises an antigen binding domain 1 (ABD1) comprising an amino acid sequence at least 80%, 85%, 90%, 95% or 99% identical to the amino acid sequence set forth in SEQ ID NO:7, SEQ ID NO:14, SEQ ID NO:21, SEQ ID NO:28, SEQ ID NO:35, SEQ ID NO:42, SEQ ID NO:49, SEQ ID NO:56, SEQ ID NO:77, SEQ ID NO:84, SEQ ID NO:91, SEQ ID NO:98, SEQ ID NO:105, SEQ ID NO:112, SEQ ID NO:119, SEQ ID NO:126, SEQ ID NO:133, or SEQ ID NO:140, an antigen binding domain 2 (ABD2) comprising an amino acid sequence at least 80%, 85%, 90%, 95% or 99% identical to the amino acid sequence set forth in SEQ ID NO:63, SEQ ID NO:281, SEQ ID NO:288, SEQ ID NO:295, SEQ ID NO:302, SEQ ID NO:309, SEQ ID NO:316, SEQ ID NO:323, SEQ ID NO:330, SEQ ID NO:337, SEQ ID NO:344, SEQ ID NO:351, SEQ ID NO:358 or SEQ ID NO:365 and an antigen binding domain 3 (ABD3) comprising an amino acid sequence at least 80%, 85%, 90%, 95% or 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:7, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:14, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:21, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:28, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:35, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:42, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:49, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:56, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:77, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:84, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:91, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:98, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:105, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:112, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:119, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:126, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:133, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence at least 75% identical, at least 80% identical, at least 85% identical, at least 90% identical, at least 95% identical or at least 99% identical to the amino acid sequence set forth in SEQ ID NO:140, at least one of the antigen binding domains is an antigen binding domain 2 (ABD2) and comprises an amino acid sequence at least 75% identical, at least 80% identical, at least 85% identical, at least 90% identical, at least 95% identical or at least 99% identical to the amino acid sequence set forth in SEQ ID NO:63 and at least one of the antigen binding domains is an antigen binding domain 3 (ABD3) and comprises an amino acid sequence at least 75% identical, at least 80% identical, at least 85% identical, at least 90% identical, at least 95% identical or at least 99% identical to the amino acid sequence set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:7, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:14, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:21, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:28, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises having an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:35, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:42, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:49, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:56, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:77, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:84, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:91, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:98, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:105, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO: 112, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO: 119, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:126, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:133, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In some embodiments, the binding agent comprises one or more antigen binding domains, wherein at least one of the antigen binding domains is an antigen binding domain 1 (ABD1) and comprises an amino acid sequence as set forth in SEQ ID NO:140, at least one of the antigen binding domains is an antigen binding domain 2 (ABD2) and comprises an amino acid sequence as set forth in SEQ ID NO:63 and at least one of the antigen binding domains is an antigen binding domain 3 (ABD3) and comprises an amino acid sequence as set forth in SEQ ID NO:70.

In other exemplary embodiments of binding agents, SEQ ID NO:63 may be replaced with other anti-PD1 VHH sequences such as for example and without limitations, SEQ ID NO:281, SEQ ID NO:288, SEQ ID NO:295 or SEQ ID NO:337.

In some embodiments, the binding agent comprises one or more polypeptide chains, wherein at least one of the polypeptide chains has an amino acid sequence at least 75%, 80% 85%, 90%, 95%, 99% or 100% identical to the amino acid sequence set forth in SEQ ID NO:159. In some embodiments, the binding agent comprises one polypeptide chain that has the amino acid sequence set forth in SEQ ID NO:159. In other embodiments, the binding agent comprises two polypeptide chains, having an amino acid sequence at least 75%, 80% 85%, 90%, 95%, 99% or 100% identical to the amino acid sequence set forth in SEQ ID NO:159. In yet other embodiments, the binding agent comprises two polypeptide chains that have the amino acid sequence set forth in SEQ ID NO:159.

In some embodiments, the binding agent comprises one or more polypeptide chains, wherein at least one of the polypeptide chains has an amino acid sequence at least 75%, 80% 85%, 90%, 95%, 99% or 100% identical to the amino acid sequence set forth in any one of SEQ ID NOs: 160-177. In some embodiments, the binding agent comprises one polypeptide chain that has the amino acid sequence set forth in any one of SEQ ID NOs: 160-177. In other embodiments, the binding agent comprises two polypeptide chains, having an amino acid sequence at least 75%, 80% 85%, 90%, 95%, 99% or 100% identical to the amino acid sequence set forth in any one of SEQ ID NOs: 160-177. In yet other embodiments, the binding agent comprises two polypeptide chains that have the amino acid sequence set forth in any one of SEQ ID NOs: 160-177.

It is to be understood herein that the binding agents may optionally comprises one or more linkers or additional amino acid sequences.

Other exemplary embodiments of multispecific binding agents are provided herein including in Table 7.

In some embodiments, the sequence of the monospecific binding agent of Table 6 may be inserted at the N-terminal portion of SEQ ID NO:179 to make a hexavalent and trispecific binding agent containing anti-PD-1 VHH and anti-CD47 VHH.

In some embodiments, any of the VHH sequence of Table 3 may be inserted at the N-terminal portion of SEQ ID NO:178 to make a hexavalent and trispecific binding agent that contains anti-PD-1 VHH and anti-CD47 VHH.

### Variants

Variants of the sequences disclosed herein are also encompassed by the present disclosure.

Variants encompassed by the present disclosure include those which may comprise an insertion of one or more amino acid residues at one or more position, a deletion of one or more amino acid residues at one or more position or a substitution of one or more amino acid residues at one or more position (conservative or non-conservative substitutions).

For example, naturally occurring residues are divided into groups based on common side chain properties. Conservative substitutions may be made by exchanging an amino acid from one of the groups listed below (group 1 to 6) for another amino acid of the same group.

Non-conservative substitutions will entail exchanging a member of one of these groups for another.
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr), Asparagine (Asn), Glutamine (Gln),
(group 3) acidic: Aspartic acid (Asp), Glutamic acid (Glu)
(group 4) basic: Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)

Other exemplary embodiments of conservative substitutions are shown in Table 2 under the heading of “preferred substitutions”. If such substitutions result in an undesired property, then more substantial changes, denominated “exemplary substitutions” in Table 2, or as further described below in reference to amino acid classes, may be introduced and the products screened.

One of skill in the art will recognize that certain amino acids are less positively charged, are neutral, are negatively charged or have a reduced charge in comparison to other amino acids. Amino acids can be categorized based on net charge as indicated by an amino acid's isoelectric point. The isoelectric point is the pH at which the average net charge of the amino acid molecule is zero. When pH>pI, an amino acid has a net negative charge, and when the pH<pI, an amino acid has a net positive charge. In some embodiments, the measured pI value for an antibody is between about 3 and 9 (e.g. 3.0, 3.5, 4.0, 4.5, 5.0, 5.5, 6.0, 6.5, 7.0, 7.5, 8.0, 8.1, 8.2, 8.3, 8.4, 8.5, and 9) and any values in between. In some embodiments, the measured pI value for an antibody is between about 4 and 7 (e.g. 4.0, 4.5, 5.0, 5.5, 6.0, 6.5, 7.0), and any values in between. Exemplary isoelectric points of amino acids are shown in Table 2 below. Generally amino acids with positive electrically charged side chains include, for example, Arginine (R), Histidine (H), and Lysine (K). Amino acids with negative electrically charged side chains include, for example, Aspartic Acid (D) and Glutamic Acid ( ). Amino acids with polar properties include, for example, Serine (5), Threonine (T), Asparagine (N), Glutamine (Q), and Cysteine (C), Tyrosine (Y) and Tryptophan (W). Non-polar amino acids include, for example, Alanine (A), Valine (V), Isoleucine (I), Leucine (L), Methionine (M), Phenylalanine (F), Glycine (G) and Proline (P).

In some embodiments, the isoelectric point of an antibody is modified via amino acid substitution. See, e.g. US201 10076275. In some embodiments, modifying the isoelectric point of a polypeptide comprising an antibody results in a change in the antibody's half-life.

TABLE 2
Exemplary amino acid substitutions

| Original | | Conservative | pI (isoelectric |
|---|---|---|---|
| residue | Exemplary substitution | substitution | point) |
| Ala (A) | Val, Leu, Ile | Val | 6.0 |
| Arg (R) | Lys, Gln, Asn | Lys | 10.76 |
| Asn (N) | Gln, His, Lys, Arg, Asp | Gln | 5.41 |
| Asp (D) | Glu, Asn | Glu | 2.77 |
| Cys (C) | Ser, Ala | Ser | 5.07 |
| Gln (Q) | Asn; Glu | Asn | 5.65 |
| Glu (E) | Asp, Gln | Asp | 3.22 |
| Gly (G) | Ala | Ala | 5.97 |
| His (H) | Asn, Gln, Lys, Arg, | Arg | 7.59 |
| Ile (I) | Leu, Val, Met, Ala, | Leu | 6.02 |
| | Phe, norleucine | | |
| Leu (L) | Norleucine, Ile, Val, | Ile | 5.98 |
| | Met, Ala, Phe | | |
| Lys (K) | Arg, Gln, Asn | Arg | 9.74 |
| Met (M) | Leu, Phe, Ile | Leu | 5.74 |
| Phe (F) | Leu, Val, Ile, Ala, Tyr | Tyr | 5.48 |
| Pro (P) | Ala | Ala | 6.30 |
| Ser (S) | Thr | Thr | 5.58 |
| Thr (T) | Ser | Ser | 5.60 |
| Trp (W) | Tyr, Phe | Tyr | 5.89 |
| Tyr (Y) | Trp, Phe, Thr, Ser | Phe | 5.66 |
| Val (V) | Ile, Leu, Met, Phe, Ala, | Leu | 5.96 |
| | norleucine | | |

Generally, the degree of similarity and identity between variable chains is determined herein using the Blast2 sequence program (Tatiana A. Tatusova, Thomas L. Madden (1999), “Blast 2 sequences - a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity will therefore be indicative of amino acids which are identical in comparison with the original peptide, and which may occupy the same or similar position.

Percent similarity will be indicative of amino acids which are identical and those which are replaced with conservative amino acid substitution in comparison with the original peptide at the same or similar position.

Variants of the present disclosure may therefore comprise a sequence that is at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical with that of an original or reference sequence or a portion of an original sequence.

In some embodiments, polypeptide chains having an amino acid sequence which is at least 75%, 80% 85%, 90%, 95%, 99% identical or less than 100% identical to a given amino acid sequence, may have amino acid substitutions, additions or deletions that are generally located outside of the complementarity determining regions.

In some embodiments, a variant may have at least 80% sequence identity with a sequence disclosed herein. In other embodiments, a variant may have at least 85% sequence identity with a sequence disclosed herein. In yet embodiments, a variant may have at least 90% sequence identity with a sequence disclosed herein. In further embodiments, a variant may have at least 95% sequence identity with a sequence disclosed herein. In other embodiments, a variant may have at least 99% sequence identity with a sequence disclosed herein.

It is to be understood herein that the expression “from one to ten amino acid substitutions, additions and/or deletions” encompass from “one to ten, from one to nine, from one to eight, from one to seven, from one to six, from one to five, from one to four, from one to three, from one to two as well as one, two, three, four, five, six, seven, eight, nine or ten amino acid substitutions”, “one to ten, from one to nine, from one to eight, from one to seven, from one to six, from one to five, from one to four, from one to three, from one to two as well as one, two, three, four, five, six, seven, eight, nine or ten amino acid additions” or “one to ten, from one to nine, from one to eight, from one to seven, from one to six, from one to five, from one to four, from one to three, from one to two as well as one, two, three, four, five, six, seven, eight, nine or ten amino acid deletions”.

In some embodiments, the amino acid substitutions may be conservative. In other embodiments, the amino acid substitutions may be non-conservative. In yet other embodiments, the amino acid substitutions may be a combination of conservative and non-conservative substitutions.

Exemplary embodiments of variants include polypeptide chains or binding agents that comprise a hinge, Fc, CH3, CH2/CH3 region that is derived from a natural antibody but that comprise one, two, three, four, five, six, seven, eight, nine, ten or more amino acid difference (including amino acid substitutions, insertions or deletions).

In some embodiments, the polypeptide chain of the present disclosure may thus comprise a mutated hinge region that is at least 80% identical, at least 85% identical, at least 90% identical, at least 95% identical, at least 99% identical to a hinge region of a natural antibody.

In some embodiments, the polypeptide chain of the present disclosure may thus comprise a mutated Fc portion that is at least 80% identical to a Fc of a natural antibody.

In some embodiments, the polypeptide chain of the present disclosure may thus comprise a mutated CH2 domain that is at least 80% identical, at least 85% identical, at least 90% identical, at least 95% identical, at least 99% identical to the CH2 domain of a natural antibody.

In some embodiments, the polypeptide chain of the present disclosure may thus comprise a mutated CH3 domain that is at least 80% identical, at least 85% identical, at least 90% identical, at least 95% identical, at least 99% identical to the CH3 domain of a natural antibody.

In some embodiments, the polypeptide chain of the present disclosure may thus comprise a mutated CH2/CH3 domain that is at least 80% identical, at least 85% identical, at least 90% identical, at least 95% identical, at least 99% identical to the CH2/CH3 domain of a natural antibody.

Nucleic acids, vectors, kits, cells and method of making polypeptides chains Nucleic acid molecules of the present disclosure may be single-stranded or double-stranded. The nucleic acid molecules disclosed herein may comprises deoxyribonucleotides, ribonucleotides, modified deoxyribonucleotides or modified ribonucleotides. The nucleic acid molecules of the present disclosure may comprise for example DNA.

DNA segments and vectors encoding one or more modules or entire polypeptide chains are particularly provided.

The DNA segments and/or vectors may be provided in separate vials and sold as a kit. Particularly contemplated are sets of DNA segments that comprise sequence allowing directional assembly of the modules and cloning vectors that incorporate the DNA segments or entire polypeptide chains.

The DNA segments and vectors may be provided as part of a kit for assembling DNA constructs capable of expressing the polypeptide chains or binding agents disclosed herein.

The kit may at least comprise one or more DNA segment or vectors that allow a user to generate a polypeptide chain comprising the mutated dimerization domain having amino acid substitutions at position 356, 357, 370, 399 and/or 439 (in accordance with EU numbering system) as disclosed herein.

More particularly, the present disclosure relates to nucleic acid molecules encoding an antigen binding domain comprising the amino acid sequence set forth in any one of SEQ ID NOs: 7, 14, 21, 28, 35, 42, 49, 56, 63, 70, 77, 84, 91, 98, 105, 112, 119, 126, 133, 140, 281, 288, 295, 302, 309, 316, 323, 330, 337, 344, 351, 358 and/or 365.

In some embodiments, the nucleic acid molecule of the present disclosure may encode a binding agent comprising the amino acid sequence set forth in any one of SEQ ID NOs: 7, 14, 21, 28, 35, 42, 49, 56, 63, 70, 77, 84, 91, 98, 105, 112, 119, 126, 133, 140, 281, 288, 295, 302, 309, 316, 323, 330, 337, 344, 351, 358 and/or 365.

In other embodiments, the nucleic acid molecule of the present disclosure may encode a binding agent comprising the amino acid sequence set forth in any one of SEQ ID NOs:141-158.

In yet other embodiments, the nucleic acid molecule of the present disclosure may encode a binding agent comprising the amino acid sequence set forth in any one of SEQ ID NOs:159-177.

In exemplary embodiments, the nucleic acid molecule may comprise a sequence at least 50% identical to any one of SEQ ID NOs:228-267 or to any one of SEQ ID NOs: 366-378.

The present disclosure particularly relates to nucleic acid molecule having a codon-optimized sequence. In some embodiments, the codon-optimized sequence may improve expression of the polypeptide chains in mammalian cells (e.g., including without limitations human cells).

In some embodiments, the nucleic acid molecule is codon-optimized sequence and encodes a polypeptide chain comprising an amino acid sequence as set forth in any one of ID NO: 7, 14, 21, 28, 35, 42, 49, 56, 63, 70, 77, 84. 91, 98, 105, 112, 119, 126, 133, 140, 281, 288, 295, 302, 309, 316, 323, 330, 337, 344, 351, 358 and/or 365.

In exemplary embodiments, the nucleic acid molecule may comprise a sequence at least 50% identical to any one of SEQ ID NOs: SEQ ID NOs:228-267 or to any one of SEQ ID NOs: 366-378 and wherein the sequence is codon-optimized.

The present disclosure also relates to a vector comprising the nucleic acid molecule disclosed herein. In some embodiments, the vector comprises a codon-optimized sequence disclosed herein.

The present disclosure also relates to a method of making a binding agent. The method comprises transforming cells with an expression vector comprising the nucleic acid molecule disclosed herein. In some embodiments, the method is for making a binding agent by transforming cells with an expression vector comprising a codon-optimized sequence that allow expression of a polypeptide chain comprising an amino acid sequence as set forth in any one of ID NO: 7, 14, 21, 28, 35, 42, 49, 56, 63, 70, 77, 84. 91, 98, 105, 112, 119, 126, 133, 140, 141-177, 281, 288, 295, 302, 309, 316, 323, 330, 337, 344, 351, 358 and/or 365.

Due to the inherent degeneracy of the genetic code, DNA sequences that encode the same, substantially the same or a functionally equivalent amino acid sequence may be produced and used. The nucleotide sequences of the present disclosure may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth. Codon-optimized nucleic acids encoding the polypeptide chains described herein are encompassed by the present disclosure.

Exemplary embodiments of nucleic acid sequence encoding the antigen binding domain and/or binding agent of the present disclosure are provided in Table 8.

The polypeptide chains and binding agents disclosed herein may be made by a variety of methods familiar to those skilled in the art, including by recombinant DNA methods or by in vitro transcription/translation.

Generally, the polypeptide chains described herein are expressed from nucleic acid sequences inserted into an expression vector, i.e., a vector that contains the elements for transcriptional and translational control of the inserted coding sequence in a particular host.

These elements may include regulatory sequences, such as enhancers, constitutive and inducible promoters, and 5′ and 3′ un-translated regions.

A variety of expression vector/host cell systems known to those of skill in the art may be used to express the polypeptide chains described herein. In the event, that the binding agent is composed of distinct polypeptide chains, each of such polypeptide chain may be provided by separate expression vectors or by a unique expression vector. In accordance with the present disclosure, the two chains of a binding agent may be encoded by a single vector or by separate vectors (vector set).

Polypeptides are often expressed in mammalian cells. For long-term production of recombinant proteins, a stable expression system may be used in which the DNA segment is incorporated into the host cell genome or maintained in an episomal form by the use of selectable markers. A host cell type may be chosen for its ability to modulate expression of the inserted sequences or to process the expressed polypeptide in the desired fashion. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Other types of expression system can be used. These include, for example, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems.

The present disclosure therefore relates to isolated cells transformed or transfected with a vector, nucleic acid, sets of vectors or sets of nucleic acids encoding at least one of the polypeptide chains described herein. The present disclosure therefore also relates to isolated cells capable or expressing the polypeptide chains or binding agents disclosed herein.

The present disclosure also relates to a method of making binding agents. The method may comprise providing a cell (e.g., a mammalian cell) with a vector or sets of vectors encoding one or more of the polypeptide chains disclosed herein and allowing expression.

The method may also comprises purifying the binding agent from cells or cell debris.

In some embodiments, the method of manufacture allows to reach a purity level of at least 80%, of at least 85%, at least 90%, at least 99% (of dimers).

In some embodiments, the purity level of the binding agent is between 80.0% to 99.9%.

In some embodiments, the purity level of the binding agent is between 95.0% to 99.9%.

In some embodiments, the purity level of the binding agent is at least 90.0+/−5.0%.

In some embodiments, the purity level of the binding agent is at least 95.0+/−5.0%.

In some embodiments, the purity level of the binding agent equal to or higher than 95 In some embodiments, the purity level of the binding agent is 97.0+/−1.0%.

In some embodiments, the purity level of the binding agent equal to or higher than 97 In some embodiments, the purity level of the binding agent is 99.0+.- 1.0%.

In some embodiments, the purity level of the binding agent equal to or higher than 99%.

In some embodiments, the titer of the binding agents produced by cells may be 0.1 g/L or more. In some instances, the titer of the binding agents produced by cells may be 0.5 g/L or more. In some instances, the titer of the binding agent produced by cells may be 1 g/L or more.

In some instances, the titer of the binding agent produced by cells may be 2 g/L or more. In some instances, the titer of the binding agent produced by cells may be 3 g/L or more. In some instances, the titer of the binding agent produced by cells may be 4 g/L or more. In some instances, the titer of the binding agent produced by cells may be 5 g/L or more. In some instances, the titer of the binding agent produced by cells may be 6 g/L or more. Usually, homodimers are made by transfection of cells with a vector comprising a nucleic acid sequence encoding one of the polypeptide chains disclosed herein. The collected supernatant may contain homodimers or a mixture of monomers and/or homodimers.

Generally, heterodimers are made by co-transfection of cells with at least two types of vectors (a vector set) each comprising a nucleic acid sequence encoding two distinct polypeptide chains. The proper ratio of Chain A over Chain B is generally dependent on the level of protein expression obtained from each individual plasmid and may vary for example from about 1:10 to about 10:1. A DNA ratio of approximately 1:1 is particularly preferred for some of the constructs disclosed herein.

Heterodimers can also be made by transfecting cells with a single vector encoding both polypeptide chains. The collected supernatant may contain heterodimers or a mixture of monomers, heterodimers and/or homodimers.

The method of making polypeptide chains of the present disclosure may further comprise a step of separating or isolating monomers, homodimers and heterodimers from a mixture that comprises. Homodimers or heterodimers may be purified and isolated, for example, by size exclusion chromatography or with the help of tags or by other methods known to a person of skill in the art.

The method may also comprise a step of isolating and/or purifying the binding agent from impurities.

The method of the present disclosure will therefore result in compositions comprising homodimers, heterodimers or a mixture of monomers heterodimers and/or homodimers.

In some exemplary embodiments, the composition may mainly comprise homodimers.

In an exemplary embodiment, the composition may comprise a proportion of at least about 80%, at least 85%, at least 90%, at least 99% or 100% of homodimers. In other exemplary embodiments, the composition may mainly comprise heterodimers.

In an exemplary embodiment, the composition may comprise a proportion of at least about 80%, at least 85%, at least 90%, at least 99% or 100% of heterodimers.

Conjugates The binding agent of the present disclosure may be conjugated, for example, with a therapeutic moiety (for therapeutic purposes) or with a detectable moiety (i.e., for detection or diagnostic purposes) or to a protein allowing an extended half-life or is attached to nanoparticle.

In some instances, therapeutic or detectable moieties may be linked to at least one amino acid residues of the polypeptide chain.

In an exemplary embodiment, the binding agent of the present disclosure is conjugated with a therapeutic moiety such as for example and without limitation, a chemotherapeutic, a cytokine, a cytotoxic agent, an anti-cancer drug (e.g., small molecule), and the like.

Therapeutic moiety may include, for example and without limitation, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹), copper (e.g., Cu⁶⁷)), 5-fluorouracil, adriamycin, irinotecan, taxanes, pseudomonas endotoxin, ricin, auristatins (e.g., monomethyl auristatin E, monomethyl auristatin F), maytansinoids (e.g., mertansine) and other toxins.

In another exemplary embodiment, the binding agent of the present disclosure is conjugated with a detectable moiety including for example and without limitation, a moiety detectable by spectroscopic, photochemical, biochemical, immunochemical, chemical and/or other physical means. A detectable moiety may be coupled either directly and/or indirectly (for example via a linkage, such as, without limitation, a DOTA or NHS linkage) to the polypeptide chain or binding agent using methods well known in the art. A wide variety of detectable moieties may be used, with the choice depending on the sensitivity required, ease of conjugation, stability requirements and available instrumentation. A suitable detectable moiety includes, but is not limited to, a fluorescent label, a radioactive label (for example, without limitation, ¹²⁵I, In¹¹¹, Tc⁹⁹, I¹³¹ and including positron emitting isotopes for PET scanner etc), a nuclear magnetic resonance active label, a luminescent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

### Pharmaceutical Compositions

Pharmaceutical compositions comprising the binding agents of the present disclosure are also encompassed by the present disclosure. The pharmaceutical composition may also comprise a pharmaceutically acceptable carrier.

In some embodiments, the pharmaceutical composition comprises conjugated binding agent as disclosed herein. In some embodiments, the pharmaceutical composition comprises binding agents conjugated with a therapeutic moiety. In some embodiments, the pharmaceutical composition comprises binding agent is conjugated with a detectable label.

In addition to the active ingredients, a pharmaceutical composition may contain pharmaceutically acceptable carriers comprising water, PBS, salt solutions, gelatins, oils, alcohols, and other excipients and auxiliaries that facilitate processing of the active compounds into preparations that may be used pharmaceutically. In other instances, such preparations may be sterilized.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also encompassed by the disclosure are particulate compositions coated with polymers (e.g., poloxamers or poloxamines). Other embodiments of the compositions of the disclosure incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1M or 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's orfixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the EDso (the dose therapeutically effective in 50% of the population) and LDso (the dose lethal to 50% of the population) statistics. Any of the pharmaceutical compositions described above may be applied to any subject in need of therapy, including, but not limited to, mammals such as dogs, cats, cows, horses, rabbits, monkeys, and especially humans.

The pharmaceutical compositions described herein may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In some embodiments, the pharmaceutical composition is made of binding agents that have a purity level of between 80.0% to 99.9%. Accordingly, in some embodiments the pharmaceutical composition comprises binding agents that are substantially free of impurities.

For example, pharmaceutical composition comprises binding agents that are at least 90.0+/−5.0% pure. In other examples, the pharmaceutical composition comprises binding agents that are at least 95.0+/−5.0% pure. In other embodiments the pharmaceutical composition comprises binding agent that are at least 99.0+/−1.0% pure.

In some embodiments, the pharmaceutical composition comprises binding agents that are stable in solution under one or more stress conditions as described herein.

Exemplary antigens In some instances, antibodies against a specific target are obtained by immunizing a host with an antigen.

In other instances, antibodies against a specific target are obtained by screening a library with the full protein or with an antigen as described herein. For example, several in vitro methods rely on the binding of candidate antibodies or antigen binding fragments thereof to an antigen. As such antibodies or antigen binding fragments thereof may be identified by methods involving binding to the antigen disclosed herein and/or using binding assays such as ELISA, flow cytometry, surface plasmon resonance (SPR). Binding agents that bind to an antigen may be identified by in silico, in vitro and/or in vivo methods.

### CD36 Antigen

Anti-CD36 antibodies or antigen binding fragments of the present disclosure may be obtained, for example, by immunizing an animal with a CD36 antigen.

Alternatively, the antibodies or antigen binding fragments of the present disclosure may be obtained from a library of anti-CD36 antibodies such as for example anti-CD36 single domain antibody library.

In an embodiment, the CD36 antigen is a human CD36 protein (e.g., Uniprot Accession No. P16671-1 which represents the canonical sequence), a CD36 homologue or a variant having a sequence at least 80% identical to the human CD36 protein thereof or a portion of human CD36 protein, of a homologue or of a variant thereof.

In an exemplary embodiment, the CD36 homologue is cynomolgus monkey CD36.

In another exemplary embodiment, the CD36 homologue is Rhesus monkey CD36.

In another exemplary embodiment, the CD36 homologue is mouse CD36.

In another exemplary embodiment, the CD36 homologue is rat CD36.

Exemplary embodiments of CD36 antigens include an extracellular domain of CD36 or a portion thereof. An exemplary embodiment of a CD36 antigen is provided in SEQ ID NO:224.

In accordance with the present disclosure the CD36 antigen comprises between 10 to 470 amino acid residues, between 10 to 450 amino acid residues, between 10 to 410 amino acid residues, between 10 to 400 amino acid residues, between 10 to 350 amino acid residues, between 10 to 300 amino acid residues, between 10 to 250 amino acid residues, between 10 to 200 amino acid residues, between 10 to 165 amino acid residues, including for example between to 160 amino acid residues, between 10 to 150 amino acid residues, between 10 to 140 amino acid residues, between 10 to 130 amino acid residues, between 10 to 120 amino acid residues, between 10 to 110 amino acid residues, between 10 to 100 amino acid residues, between 10 to 90 amino acid residues, between 10 to 80 amino acid residues, between 10 to 70 amino acid residues, between 10 to 60 amino acid residues, between 10 to 50 amino acid residues, between to 40 amino acid residues, between 10 to 30 amino acid residues, 10 to 29 amino acid residues, between 10 to 28 amino acid residues, between 10 to 27 amino acid residues, between to 26 amino acid residues, between 10 to 25 amino acid residues, between 10 to 24 amino acid residues, between 10 to 23 amino acid residues, between 10 to 22 amino acid residues, between 10 to 21 amino acid residues, between 10 to 20 amino acid residues, between 10 to 19 amino acid residues, between 10 to 18 amino acid residues, between 10 to 17 amino acid residues, between 10 to 16 amino acid residues, between 10 to 15 amino acid residues, between to 14 amino acid residues, between 10 to 13 amino acid residues, between 10 to 12 amino acid residues between 10 to 11 amino acid residues of a human cD36 amino acid sequence.

### PD-1 Antigen

Anti-PD-1 antibodies or antigen binding fragments of the present disclosure may be obtained, for example, by immunizing an animal with a PD-1 antigen.

Alternatively, the antibodies or antigen binding fragments of the present disclosure may be obtained from a library of anti-PD-1 antibodies such as anti-PD-Isingle domain antibody library.

In an embodiment, the PD-1 antigen is a human PD-1 protein (e.g., Uniprot Accession No. Q15116), a PD-1 homologue or a variant having a sequence at least 80% identical to the human PD-1 protein thereof or a portion of human PD-1 protein, of a homologue or of a variant thereof. An exemplary embodiment of a PD-1 antigen is provided in SEQ ID NO:225.

Exemplary embodiments of PD-1 antigens include an extracellular domain of PD-1 or a portion thereof.

In accordance with the present disclosure the PD-1 antigen comprises between 10 to 250 amino acid residues, between 10 to 200 amino acid residues, between 10 to 165 amino acid residues, including for example between 10 to 160 amino acid residues, between 10 to 150 amino acid residues, between 10 to 140 amino acid residues, between 10 to 130 amino acid residues, between 10 to 120 amino acid residues, between 10 to 110 amino acid residues, between 10 to 100 amino acid residues, between 10 to 90 amino acid residues, between 10 to 80 amino acid residues, between 10 to 70 amino acid residues, between 10 to 60 amino acid residues, between 10 to 50 amino acid residues, between 10 to 40 amino acid residues, between to 30 amino acid residues, 10 to 29 amino acid residues, between 10 to 28 amino acid residues, between 10 to 27 amino acid residues, between 10 to 26 amino acid residues, between to 25 amino acid residues, between 10 to 24 amino acid residues, between 10 to 23 amino acid residues, between 10 to 22 amino acid residues, between 10 to 21 amino acid residues, between 10 to 20 amino acid residues, between 10 to 19 amino acid residues, between 10 to 18 amino acid residues, between 10 to 17 amino acid residues, between 10 to 16 amino acid residues, between 10 to 15 amino acid residues, between 10 to 14 amino acid residues, between to 13 amino acid residues, between 10 to 12 amino acid residues between 10 to 11 amino acid residues of a human PD-1 amino acid sequence.

### CD47 Antigen

Anti-CD47 antibodies or antigen binding fragments of the present disclosure may be obtained, for example, by immunizing an animal with a CD47 antigen.

Alternatively, the antibodies or antigen binding fragments of the present disclosure may be obtained from a library of anti-CD47 antibodies such as anti-CD47 single domain antibody library.

In an embodiment, the CD47 antigen is a human CD47 protein (e.g., Uniprot accession. No. Q08722-1 which represents the canonical sequence), a CD47 homologue or a variant having a sequence at least 80% identical to the human CD47 protein thereof or a portion of human CD47 protein, of a homologue or of a variant thereof.

Exemplary embodiments of CD47 antigens include an extracellular domain of CD47 or a portion thereof. Exemplary embodiments of CD47 antigen include a soluble form of a CD47 extracellular domain. An exemplary embodiment of a CD47 antigen is provided in SEQ ID NO:226.

In accordance with the present disclosure the CD47 antigen comprises between 10 to 140 amino acid residues, between 10 to 130 amino acid residues, between 10 to 120 amino acid residues, between 10 to 110 amino acid residues, between 10 to 100 amino acid residues, between 10 to 90 amino acid residues, between 10 to 80 amino acid residues, between 10 to 70 amino acid residues, between 10 to 60 amino acid residues, between 10 to 50 amino acid residues, between 10 to 40 amino acid residues, between 10 to 30 amino acid residues, 10 to 29 amino acid residues, between 10 to 28 amino acid residues, between 10 to 27 amino acid residues, between 10 to 26 amino acid residues, between 10 to 25 amino acid residues, between to 24 amino acid residues, between 10 to 23 amino acid residues, between 10 to 22 amino acid residues, between 10 to 21 amino acid residues, between 10 to 20 amino acid residues, between 10 to 19 amino acid residues, between 10 to 18 amino acid residues, between 10 to 17 amino acid residues, between 10 to 16 amino acid residues, between 10 to 15 amino acid residues, between 10 to 14 amino acid residues, between 10 to 13 amino acid residues, between to 12 amino acid residues between 10 to 11 amino acid residues of a human CD47 amino acid sequence.

### Method of Use

The binding agents, compositions or pharmaceutical compositions of the present disclosure may be used for treatment of cancer.

In some embodiments, the binding agents, compositions or pharmaceutical compositions of the present disclosure may be used for inhibiting tumor growth.

In some embodiments, the binding agents, compositions or pharmaceutical compositions of the present disclosure may be used for delaying progression of tumors.

Accordingly, the present disclosure relates to a method of treating cancer comprising administering the binding agent, composition and/or pharmaceutical composition disclosed herein to a subject in need.

In an embodiment, the method of the present disclosure comprises administering a pharmaceutical composition comprising a means for inhibiting tumor growth and a pharmaceutically acceptable carrier, wherein the means for inhibiting tumor growth comprises a means for binding to CD36 and/or a means for blocking the interaction between PD-1 and PD-L1 and/or a means for blocking the interaction between CD47 and SIRPQ carrier.

The means for inhibiting tumor growth may be achieved with a binding agent that comprises one or more antigen binding domains that specifically bind to CD36 and/or one or more antigen binding domains that specifically bind to Programmed Cell Death Protein 1 (PD-1) and/or one or more antigen binding domains that specifically bind to Cluster of Differentiation 47 (CD47).

The amino acid sequence of the antigen binding domain may correspond to that of an antibody or an antigen binding fragment thereof (including a single domain antibody or an antigen binding fragment thereof) that binds to a desired antigen and/or inhibit the interaction between the antigen and one of its ligands.

The amino acid sequence of the antigen binding domain may be identical to that of an original antibody isolated from non-human animals (such as a mouse, rat, rabbit, camelid) such as a transgenic animal able to express human, humanized or camelized antibodies. The amino acid sequence of the antigen binding domain may also be modified by design to render it more human-like or animal-like. As such, the amino acid sequence of the antigen binding domain may comprise non-humanized antibody, human antibody, humanized antibody or camelid antibody amino acid sequences.

In some embodiments, the amino acid sequence of the one or more antigen binding domains of the binding agent is in a single chain format.

In some embodiments, the antigen binding domain may be from a single domain antibody or antigen binding fragment thereof that binds CD36 and is capable of inhibiting one or more function or mechanism of action of CD36.

In some embodiments, the antigen binding domain may be from a single domain antibody or antigen binding fragment thereof that binds CD36 and is capable of reducing fatty acid oxidation, lipid metabolism and/or fatty acid uptake by cells.

In some embodiments, the antigen binding domain may be from a single domain antibody or antigen binding fragment thereof that binds CD36 and is capable of blocking interaction of CD36 and one or more CD36-ligands.

In some embodiments, the antigen binding domain may be from a single domain antibody or antigen binding fragment thereof that is capable of blocking the interaction of PD-1 with PD-L1. The antigen binding domain may thus be from a single domain antibody or antigen binding fragment thereof that binds to PD-1. Alternatively, the antigen binding domain may thus be from a single domain antibody or antigen binding fragment thereof that binds to PD-L1. PD-1/PD-L1 blockade assays may be performed with a reporter system such as, for example and without limitations, the luminescent reporter system described herein.

In some embodiments, the antigen binding domain may be from a single domain antibody or antigen binding fragment thereof that is capable of blocking the interaction of CD47 with SIRPD. The antigen binding domain may thus be from a single domain antibody or antigen binding fragment thereof that binds to CD47. Alternatively, the antigen binding domain may thus be from a single domain antibody or antigen binding fragment thereof that binds to SIRPD. CD47/SIRPu blockade assay may be performed with a reporter system such as, for example and without limitations, the luminescent reporter system described herein.

Conventional binding assay includes, for example and without limitations, ELISA, flow cytometry, surface plasmon resonance, electro-chemiluminescence method, mesoscale discovery, radioimmunoassay, fluorescence immunoassay, LC-MS detection, thermal shift assay, bio-layer interferometry, and the like. A binding assay may be performed between recombinant proteins (or portions), with a cell line expressing the protein(s) at its surface or with liposomes or nanoparticles comprising the protein. Generally, the binding of the binding agent to its target results in the emission or reduction of a detectable signal.

Binding may be assessed by competition assays, in which a candidate binding agent that is able to inhibit the interaction of a previously-identified binding agent (having a desired binding and/or activity) to its target (CD36, PD-1 or CD47) may be identified. Alternatively, binding may be assessed in silico, using the three-dimensional structure of the antibody/antigen complex (Zhao J. et al., Antibodies 2018, 7, 22; doi 10.3390/antib7030022).

Exemplary embodiments of cancer includes without limitations, endometrial cancer, breast cancer (e.g., triple negative breast cancer), liver cancer (e.g., hepatocellular carcinoma), lung cancer, prostate cancer, renal cancer e.g., (renal clear cell cancer), ovarian cancer, cervical cancer, pancreatic cancer, gastric cancer, bladder cancer (e.g., bladder urothelial carcinoma), oral cancer, head and neck cancer, glioblastoma (e.g., glioblastoma multiform), adrenocortical carcinoma or colorectal cancer.

Accordingly, in exemplary embodiments, the binding agent, composition and/or pharmaceutical composition may be administered to a subject having cancer selected from leukemia (e.g., acute myeloid leukemia), endometrial cancer, breast cancer (e.g., triple negative breast cancer), liver cancer (e.g., hepatocellular carcinoma), lung cancer, prostate cancer, renal cancer e.g., (renal clear cell cancer), ovarian cancer, cervical cancer, pancreatic cancer, gastric cancer, bladder cancer (e.g., bladder urothelial carcinoma), oral cancer, head and neck cancer, glioblastoma (e.g., glioblastoma multiform), adrenocortical carcinoma or colorectal cancer.

In particular embodiments, the binding agent, composition and/or pharmaceutical composition may be used for treating leukemia, including without limitation, acute myeloid leukemia. Accordingly, the binding agent, composition and/or pharmaceutical composition of the present disclosure may be administered to a subject having leukemia, such as acute myeloid leukemia.

In other particular embodiments, the binding agent, composition and/or pharmaceutical composition may be used for treating cancer originating from fat-rich (adipose) tissue.

Accordingly, the binding agent, composition and/or pharmaceutical composition of the present disclosure may be administered to a subject having a tumor originating from fat-rich (adipose) tissue.

In other particular embodiments, the binding agent, composition and/or pharmaceutical composition may be used for treating cancer associated wih increased lipid metabolism.

Accordingly, the binding agent, composition and/or pharmaceutical composition of the present disclosure may be administered to a subject having tumor showing evidence of increased lipid metabolism.

In other particular embodiments, the binding agent, composition and/or pharmaceutical composition may be used for treating cancer associated with increased fatty acid uptake and/or increased fatty acid oxidation. Accordingly, the binding agent, composition and/or pharmaceutical composition of the present disclosure may be administered to a subject having tumor showing evidence of increased fatty acid uptake and/or increased fatty acid oxidation.

In other particular embodiments the binding agent, composition and/or pharmaceutical composition may be used to treat metastatic cancer or for preventing metastasis in a subject in need. Accordingly, the binding agent, composition and/or pharmaceutical composition of the present disclosure may be administered to a subject having metastatic cancer or having a cancer thas has the potential of becoming metastatic.

In other particular embodiments, the binding agent, composition and/or pharmaceutical composition may be used to treat cancer that is resistant to chemotherapy (chemotherapeutics) and/or immune checkpoint therapy. The binding agent, composition and/or pharmaceutical composition may be used, for example, for reducing resistance to chemotherapy and/or immune checkpoint inhibitor therapy. Accordingly, the binding agent, composition and/or pharmaceutical composition of the present disclosure may be administered to a subject that has failed prior treatment with chemotherapy (chemotherapeutics) and/or immune checkpoint therapy.

In other particular embodiments, the binding agent, composition and/or pharmaceutical composition may be used to treat recurrent leukemia. Accordingly, the binding agent, composition and/or pharmaceutical composition of the present disclosure may be administered to a subject that has relapsed leukemia, such as for example, relapsed acute myeloid leukemia.

In some embodiments, the binding agent, composition and/or pharmaceutical composition may be used for inhibiting CD36 function in vivo.

The binding agent, composition and/or pharmaceutical composition may be used, for example, to inhibit the growth tumor cells in a subject in need thereof. Accordingly, the binding agent, composition and/or pharmaceutical composition may be administered to a subject having cancer with the goal of reducing and/or preventing tumor growth.

In other exemplary embodiments, the binding agent, composition and/or pharmaceutical composition may be used to inhibit metastasis of tumor cells in a subject in need thereof. Accordingly, the binding agent, composition and/or pharmaceutical composition may be administered to a subject having cancer with the goal of reducing and/or preventing tumor metastasis.

In yet other exemplary embodiments, the binding agent, composition and/or pharmaceutical composition may be used to modulate lipid metabolism of tumors in a subject in need thereof.

In other exemplary embodiments, the binding agent, composition and/or pharmaceutical composition may be used to inhibit fatty acid oxidation in a subject in need thereof.

In yet other exemplary embodiments, the binding agent, composition and/or pharmaceutical composition may be used to inhibit fatty acid uptake by tumors in a subject in need thereof.

In some embodiments, the binding agent, composition and/or pharmaceutical composition may be used to target therapeutics and/or diagnostics to a target cell, circulating protein or tissue.

In some embodiments, the binding agent, composition and/or pharmaceutical composition may be conjugated with a therapeutic moiety and used for therapeutic purposes.

In some embodiments, the binding agent, composition and/or pharmaceutical composition may be conjugated with a detectable moiety and used for detection or diagnostic purposes.

In some embodiments, the binding agent, composition and/or pharmaceutical composition of the present disclosure may be used for targeting tumors in vivo.

In some embodiments, the binding agent, composition and/or pharmaceutical composition amay be administered in combination with a chemotherapeutic.

In accordance with the present disclosure, the subject in need may be a human. Further in accordance with the present disclosure, the subject in need may be an animal.

In some embodiment, the binding agent is administered intravenously.

In some embodiment, the binding agent is administered by infusion.

Detection of a particular target may be performed in vitro by contacting a sample, containing or suspected of containing the target with a binding agent comprising an antigen binding domain for such target and quantifying a signal associated with positive or negative binding using a detection apparatus.

The sample may originate from a mammal (e.g., a human). The sample may be a tissue sample obtained from the mammal or a cell culture supernatant.

In some embodiments, the sample may be a serum sample, a plasma sample, a blood sample, semen or ascitic fluid obtained from the mammal.

Detection of a particular target may be performed in vivo by administering a binding agent comprising an antigen binding domain for such target to an individual and quantifying a signal associated with positive or negative binding using a detection apparatus.

Upon detecting the presence of the target in the sample or in the individual, a drug (e.g., antibody, small molecule, a binding agent disclosed herein) may be administered to the individual.

In addition to the embodiments described and provided in this disclosure, the following non-limiting embodiments are particularly contemplated.

Table 3, Table 4, Table 5 and Table 6 illustrate VHHs or sdAbs having biological activity on their own (e.g., as fusion with dimerization domain) and as part of the multimeric, multivalent and/or multispecific binding agents as exemplified in Table 7. It is to be understood herein that the polypeptide chains of Table 6 may associate to form dimers, thereby providing a binding agent comprising at least two antigen binding domains. It is to be understood herein that the polypeptide chains of Table 7 may associate to form dimers, thereby providing a binding agent comprising at least six antigen binding domains.

In some embodiments, the method of treating diseases or disorders involved administering a binding agent that is capable of inhibiting fatty acid oxidation. In some instances, KF039, KF040 or binding agents having the exact same CDR or VHH sequence may be used for purposes that does not require inhibition of fatty acid oxidation.

### Examples

### Example 1- Animal Immunization and Immune Library Construction

Anti-CD36 single-domain antibodies were generated by immunizing alpaca with 10 μg of the CD36 recombinant proteins and cDNA plasmid encoding the human CD36 antigens. The sequences of single-domain antibodies were uncovered clone picking after phage display panning of reconstituted immune libraries against the CD36 antigens. An exemplary embodiment of CD36 antigen is provided in SEQ ID NO:224.

Anti-PD-1 single domain antibodies were generated by immunizing alpacas (Vicugna pacos) with PD-1 antigens including the human PD-1 protein, human PD-1 cDNA plasmid and a human PD-1 overexpressing cell line; CHO-PD1 cells (see PCT/CA2022/050442 filed on Mar. 31, 2022, the entire content of which is incorporated herein by reference). An exemplary embodiment of PD-1 antigen is provided in SEQ ID NO:225.

Anti-CD47, single domain antibodies were generated by immunizing llamas (llama glama) with CD47 antigens including the human CD47 protein, human CD47 cDNA, and human PBMCs (activated) (see PCT/CA2022/050442 filed on Mar. 31, 2022, the entire content of which is incorporated herein by reference). An exemplary embodiment of CD47 antigen is provided in SEQ ID NO:226.

DNA fragments encoding the VHHs (Table 3) were generally cloned into constructs comprising a constant region of the human antibody thereby generating a single domain antibody comprising a camelid variable domain and a human Fc region (e.g., SEQ ID NO:227).

The anti-CD36 single domain antibodies were tested for expression and for assessing in vitro and in vivo functions.

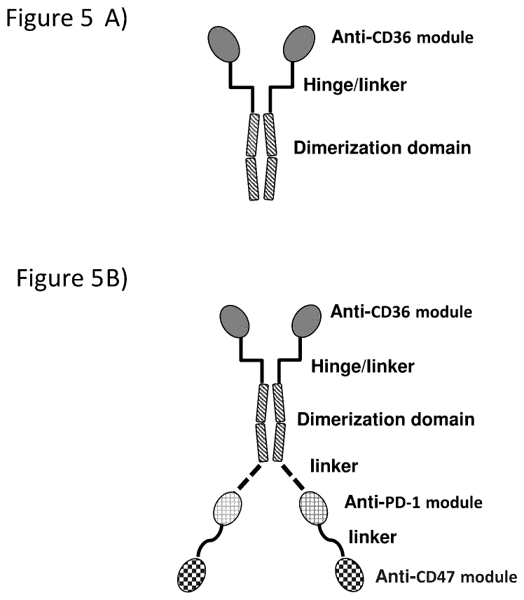

Multivalent and multispecific binding agent comprising VHH targeting CD36, VHH targeting PD-1 and VHH targeting CD47 are constructed as described in PCT/CA2020/051753 filed on Dec. 18, 2020 (published on Jun. 24, 2021, under No. WO2021/119832. The multivalent and multispecific binding agent has a format as illustrated in FIG. 5.

### Example 2- Production and Purification of CD36 sdAbs

sdAbs were expressed in cells and isolated as protein dimers.

Protein dimers (e.g., homodimers) were expressed in 2.5 mL or 400 mL culture volume from the DNA construct using the ExpiCHO™ Expression System (Thermo Fisher, Cat. no. A29133) or the Expi293™ Expression System (Thermo Fisher, Cat. no. A14635).

Freshly thawed ExpiCHO-s cells were allowed to recover in culture for two or more passages before transfection. Cells were then passaged every 3-4 days until they reach 4×10⁶-6×10⁶ cells/mL at which time they were diluted to 2×10⁵-3×10⁵ cells/mL in ExpiCHO™ Expression Medium pre-warmed to 37° C. The day prior to transfection, cells were diluted to 3×10⁶- 4×10⁶ cells/mL and on the day of transfection, cells were further diluted to 6×10⁶ cells/mL. 1 μg of DNA/mL of culture volume was diluted with cold OptiPRO™ medium (100 μL for 2.5 mL of culture volume; 16 mL for 400 mL of culture volume). ExpiFectamine™ CHO Reagent (8 μL for 2.5 mL of culture volume; 1280 μL for 400 mL of culture volume) was added to medium containing DNA and incubated with ExpiFectamine™/DNA complexes at room temperature for 1-5 min. Then the DNA complex was transferred to culture (at 6×10⁶ cells/mL) while swirling. The cells were incubated at 37° C. under 8% CO₂ and 80% humidity with shaking (INFORS HT shaker, 125 rpm). 18-22h after onset of transfection, ExpiCHO™ feed (0.6 mL for 2.5 mL of culture volume; 96 mL for 400 mL of culture volume) and ExpiCHO™ enhancer (15 μL for 2.5 mL of culture volume; 2.4 mL for 400 mL of culture volume) were added to the cells. The cells were returned to INFORS HT incubator set at 37° C. under 8% CO₂ and 80% humidity with shaking at 125 rpm (25 mm orbit). 8 days post-transfection, supernatants were clarified by centrifugation at 4000×g for 30 min. Supernatants were filter-sterilized using a Nalgene™ Rapid-Flow™ Sterile Disposable Filter Units 1000 mL filter unit (Thermo Scientific, Cat. no. 567-0020) and were stored at 4° C. or frozen for later analysis.

Freshly thawed Expi293F cells were allowed to recover in culture for two or more passages before transfection. Cells were then passaged every 3-4 days until they reach 3×10⁶-5×10⁶ cells/mL at which time they were diluted to 3×10⁵-5×10⁵ cells/mL in Expi293^{T}M Expression Medium pre-warmed to 37° C. The day prior to transfection, cells were diluted to 2.5×10⁶-3×10⁶ and on the day of transfection, cells were further diluted to 3×10⁶ viable cells/mL. 1 μg of DNA/mL of culture volume was diluted with Opti-MEM™ I Reduced Serum medium to get a final volume of 150 μL for 2.5 mL of culture volume and 24 mL for 400 mL of culture volume. ExpiFectamine™ 293 Reagent (8 μL for 2.5 mL of culture volume; 1.3 mL for 400 mL of culture volume) was added to medium Opti-MEM™ I Reduced Serum medium (140 μL for 2.5 mL of culture volume; 22.5 mL for 400 mL of culture volume) to incubate at room temperature for 5 minutes. Diluted ExpiFectamine™ was added to diluted DNA and incubate for 15 minutes at room temperature. ExpiFectamine™/DNA solution was transferred to culture drop by drop (at 3×10⁶ cells/mL) while swirling. The cells were incubated at 37° C. under 8% CO₂ and 80% humidity with overnight shaking (INFORS HT shaker, 125 rpm). 18-22h after onset of transfection, ExpiFectamine™ 293 Transfection Enhancer 1 (15 μL for 2.5 mL of culture volume; 2.4 mL for 400 mL of culture volume) and ExpiFectamine™ 293 Transfection Enhancer 2 (50 μL for 2.5 mL of culture volume; 24 mL for 400 mL of culture volume) were added to the cells. The cells were returned to INFORS HT incubator set at 37° C. under 8% CO₂ and 80% humidity with shaking at 125 rpm (25 mm orbit). 5 days post-transfection, supernatants were clarified by centrifugation at 4000×g for 30 min. Supernatants were filter-sterilized using a Nalgene™ Rapid-Flow™ Sterile Disposable Filter Units 1000 mL filter unit (Thermo Scientific, Cat. no. 567-0020) and were stored at 4° C. or frozen for later analysis.

Proteins are purified using 3-mL MabSelect SuRe resin (GE Healthcare, Cat. No. 17-5438-02) with gravity columns or 40-mL MabSelect SuRe resin with AKTA PURE (GE Healthcare, Piscataway, NJ) depending on the supernatant volume. Resin was incubated with 0.5 NaOH for one hour and equilibrated with Tris-base buffer pH 7.4 (50 mM Tris-HCl, 150 mM NaCl, pH 7.4) prior injection. Supernatant was applied on gravity columns or the at 5 mL/min on 40-mL column. Resin column was washed with 3 CV (column volume) with Tris-base buffer pH 7.4 at flow rate of 10 mL/min. Protein was eluted with 3 CV of 0.1M citrate acid pH 3 at 10 mL/min. Fractions identified with protein from the visual output of the chromatogram (absorbance at 280 nm) were pooled together. Pooled fractions were neutralized with 1M Tris-HCl pH 9 to achieve the pH - 5-6 before transferring into PBS (Phosphate-buffered saline) pH 6 buffer prepared from PBS 10 × pH 7.2 (15 mM Potassium Phosphate monobasic 1552 mM Sodium Chloride 27 mM Sodium Phosphate dibasic, ThermoFisher, Cat. no. 70013073) or 20 mM Histidine 7% Sucrose 0.02% Polysorbate80 pH 5.5.

Buffer exchange was carried out by sample concentrators for proteins purified from gravity columns or either by dialysis or by desalting column for proteins purified from AKTA PURE. Proteins purified from gravity columns were concentrated with sample concentrator VivaSpin 2, 50 kDa MWCO (GE Healthcare, Cat. no. 28932257) by centrifugation at 3,500-4,000×g at 4° C. then, diluted with PBS pH 6 to achieve 4-fold and repeated until sample reached 200-fold. Dialysis was carried out in 4L of PBS pH 6 overnight at 4° C. using 7 kDa molecular weight cut-off dialysis tubing (ThermoFisher, Cat. no. 68799). On the other hand, desalting column was incubated with 0.5 NaOH overnight and equilibrated with PBS pH 6. Volume of 15 mL of neutralized protein sample was loaded into the HiPrep 26/10 desalting column (GE Healthcare, Cat. no. 17-5087-02) at 0.5 mL/min then, protein was eluted with 2 CV of PBS pH 6. Loading and elution steps were repeated until no neutralized protein sample from elution of affinity column remained. Fractions identified with protein from the visual output of the chromatogram (absorbance at 280 nm) were pooled together.

Sample was filter-sterilized using a Nalgene™ Rapid-Flow™ Sterile Disposable Filter Units 150 mL filter unit (Thermo Scientific, Cat. no. 565-0010). Final protein sample was quantified by optical density at 280 nanometer and tested for endotoxin level with Endosafe® LAL Reagent cartridges (Charles River Cat. no. PTS2005). Final protein sample was analyzed on SDS-PAGE gels under reducing or non-reducing conditions (see section SDS PAGE and Western Blotting).

### Example 3- Binding assay

ELISA Binding Assay The 96-well plates were coated with 1 μg/ml recombinant CD36 (SinoBiological, Cat No. 10752-H08H) protein and incubated at 4° C. overnight. The next day, plate was washed three times and blocked with blocking buffer for 1 hour at 37° C. At the end of the incubation, test molecules were diluted to 10 μg/ml, and distributed into corresponding wells for incubation at 37° C. After 1 hour of incubation, wells were washed for three times. Anti-human IgG Fc-HRP 2^{nd} Ab (Sigma-Aldrich, AP113P) was diluted in 1:5000 dilution and distributed into corresponding wells for 1 hour incubation at 37° C. At the end of the incubation, the wells were washed 3 times, then 100 ul SuperSignal ELISA Pico Chemiluminescent Substrate (ThermoFisher, Cat No. 37069) was added into the wells to detect signals. Plates were read on a plate reader after the addition of the chemiluminescent substrate. Raw Data was collected as Chemiluminescence value for each well (RLUs). Data was plotted and represented as Mean of two replicates ±SD.

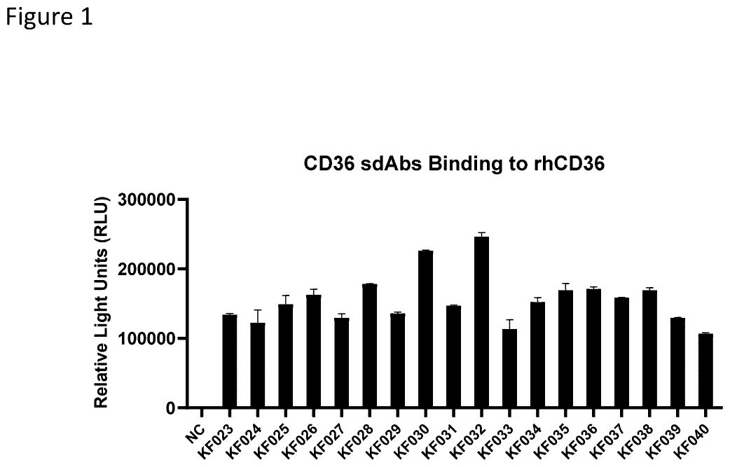

Results of FIG. 1 show that all tested CD36 antibodies bind to recombinant human CD36 protein.

### Cell Binding Assay

Cells were harvested and first blocked with Fc block (Biolegend, Cat No. 422302) on ice for 10 mins. The cells were then incubated with test molecules on ice for 30 mins. To detect the binding of test molecules, cells were washed once and further incubated with anti-human Fc-APC (Biolegend, Cat No. 410712) for 30 minutes incubation on ice. At the end of incubation, cells were washed and resuspended in FACS buffer with 1:100 7-AAD (BD Bioscience, Cat No. 559925) added. Data was acquired on BD Canto II flow cytometry machine. Raw data was collected as Mean Fluorescence Intensity (MFI). Data was plotted and represented as Mean of two replicates ±SD.

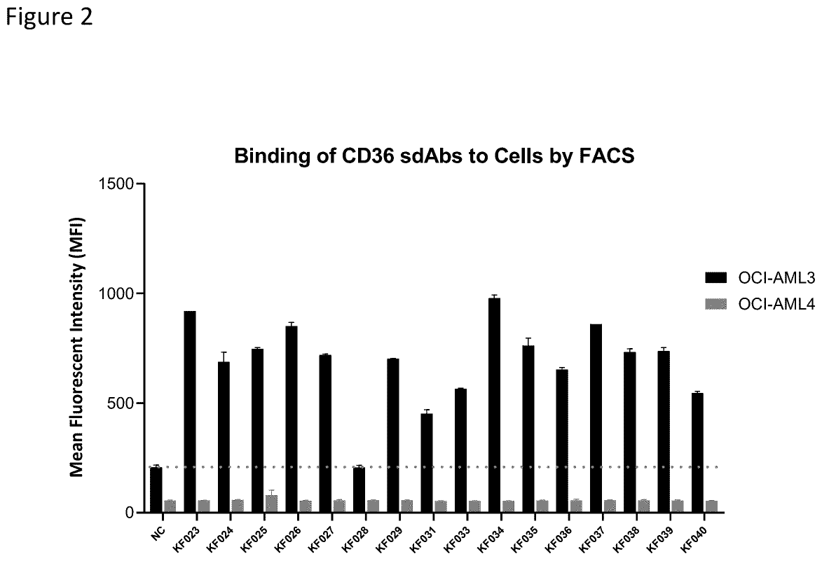

Results of FIG. 2 show that, with the exception of KC028, all tested CD36 antibodies bind to CD36 positive OCI-AML3 cells and not to the CD36 negative OCI-AML4 cells.

### Example 4- Fatty Acid Oxidation Assay

The effect of anti-CD36 sdAbs on fatty acid oxidation (FAO) levels was assessed in vitro by measuring fluorescent extracellular oxygen consumption. Briefly, OCI-AML3 cells were plated in 96-well plates at 200,000 cells per well and incubated overnight at 37° C., 5% CO2. The following day, cells were washed twice with base glucose deprivation media (Glucose free DMEM containing 1 mM glucose, 1 mM L-glutamine, 1% FBS, and 1% Pencilin- Streptomycin). To increase the metabolic dependence of cells on FAO, glucose deprivation media was added to cells and incubated overnight at 37° C., 5% CO2. Glucose deprivation media consists of base glucose deprivation media containing 0.5 Mm L-carntine. On the third day, cells were washed twice with pre-warmed fatty acid (FA)-free media (FAO tablet dissolved in double distilled water, pH 7.4 to which 0.5 mM L-cartinine and 2.5 mM glucose were added). Cells were then resuspended in FA measurement media (FA-free media containing 150 μM FAO conjugate) except for the wells used as FA-free control where FA-free media containing BSA control was added instead. Extracellular oxygen consumption reagent was added at 10 μL per well, excluding the blank control wells. Antibodies were diluted to 20× concentration in FA measurement media. Cells were treated with 350 nM sdAb for 1 hour at 37° C., 5% CO2. Thirty minutes before the end of incubation, 10 μL Etomoxir (Sigma-Aldrich, E1905-5MG) and 1 μL FCCP (Abcam, ab120081) were added to the corresponding wells. At the end of incubation period, wells were sealed with 100 μL of pre-warmed high sensitivity mineral oil. Fluorsecence was measured using a plate reader, utilizing an excitation wavelength of 380 nm and emission wavelength of 650 nm.

Raw data were collected using the Plate Reader Spectra Max i3× machine. Effects on FAO levels was assessed by calculating the extracellular oxygen consumption intensity per well which is the average relative fluorescence units (RFU) obtained from technical duplicates.

All samples described in this report were tested in duplicate. Results represented the mean+/−standard deviation.

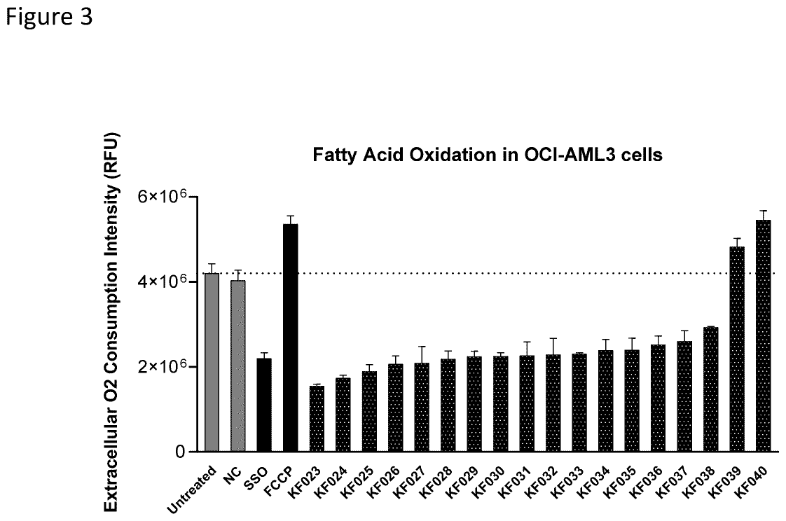

As shown in the FIG. 3, 16 CD36 sdAbs (KF023 to KF038) showed significant fatty acid oxidation inhibition when treated with the OCI-AML3 cells. This may suggest the anti-tumor activity by inhibiting the metabolic activity of the tumor cells. In addition, two of the 16 CD36 sdAbs, KF039 and KF040, showed increased fatty acid oxidation.

### Example 5- Platelet activation assay

The platelet activation capability of anti-CD36 sdAbs was assessed in vitro using platelets isolated from healthy donors using the FACS based platelet activation assay. Platelet activation markers CD62 was measured by FITC-anti-CD62 antibody (Abcam, ab239255) after treatment with the anti-CD36 sdAbs.

Briefly, blood was collected by venipuncture from four healthy volunteer after initial discard of samples containing activated platelets. Collected blood was centrifuged at 250×g for 8 minutes to collect platelet containing plasma. Platelet rich plasma was further isolated by 700× g centrifugation for 17 minutes. Titrate the volume of platelet suspension to 10 ml with PBS (containing 10 nM HEPEs and 1% BSA).

Titrated platelet suspension was then transferred into 96 well plates and incubated with 50 μg/ml anti-CD36 sdAbs for 10 minutes at 37° C.

Cells were then fixed with 1% PFA and stained with anti-CD41 (BioRad, Cat No. MCA467A647T) and anti-CD62P (Abcam, Cat No. Ab23925). Data was acquired on BD Canto II flow cytometry machine. Data was collected as CD41 and CD62P dual positive population, which was plotted and represented as Mean of two replicates ±SD.

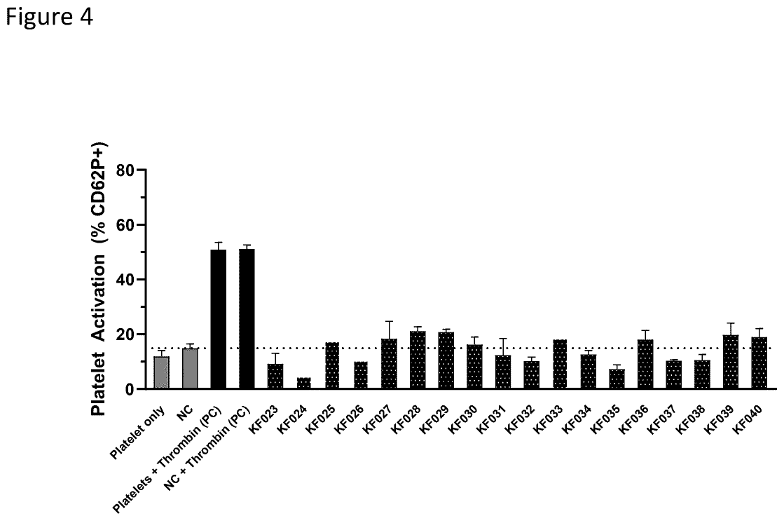

As shown in the FIG. 4, testing anti-CD36 sdAbs did not lead to platelet activation, thus displaying a good safety profile.

### Example 7- In Vivo Testing

The anti-tumor effect of the CD36 binding agent of the present disclosure was tested in the PBMC pre-engrafted model in the NCG mice (5-6 weeks old, female), which were purchased from Charles River Laboratories (St. Constant, QC). Mice were housed in a pathogen-free environment at the animal facility in UHN. The animal work was conducted according to the guidelines of the Canadian Council on Animal Care (CCAC) and the Animal Use Protocol approved by the Animal Care Committee at UHN.

Briefly, NCG mice were implanted with 10 million human PBMC (StemCell, Cat #70025) via intravenous tail injection. Four days later, 2 million OCI-AML3 tumor cells were subcutaneously injected into the mice in 100 ul PBS. Animals were randomized before starting the treatment. Treatment started one week after the tumor cell implantation and the dosing frequency was twice a week at 26 mg/kg. No signs of toxicity were observed throughout the studies. Tumor volumes were measured using vernier calipers and the mice were weighed one or two times weekly. Tumor volume was calculated using the formula: ½ (Length×Width2). For calculation of percentage tumor growth inhibition (TGI), KJ061 treated group was compared with its respective vehicle control. TGI was calculated by the following formula:

$$TGI [ \% ] = 1 \; 00 - \frac{mean ( {TV ( treated )}_{day z} - {TV ( treated )}_{day x} )}{mean ( {TV ( resp . control )}_{day z} - {TV ( resp . control )}_{day x} )} \times 100$$

TV day z represents the tumor volume of an individual animal at a defined study day (day z) and TV day x represents the tumor volume of an individual animal at the staging day (day x).

Statistical tests were performed by a student t-test (two-tailed).

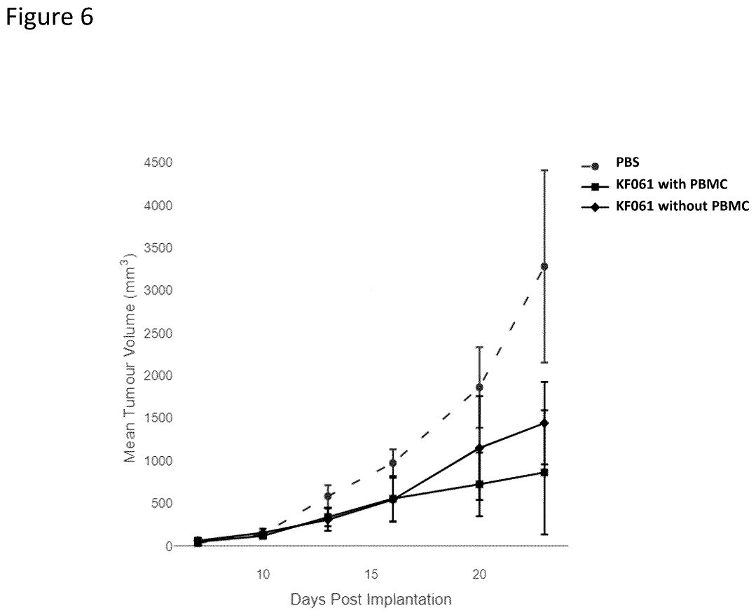

As shown in FIG. 6, KF061 treated group with or without PBMC injection showed a significant tumor growth inhibition (TGI) than the PBS group, implying the anti-tumor function of the CD36 trispecific antibody containing anti-CD36, anti-PD-1, and anti-CD47 module. Since PBMC implanted group showed slightly better efficacy than the group without PBMC, implying a role of the PD-1 module in the anti-tumor function by the human T cells.

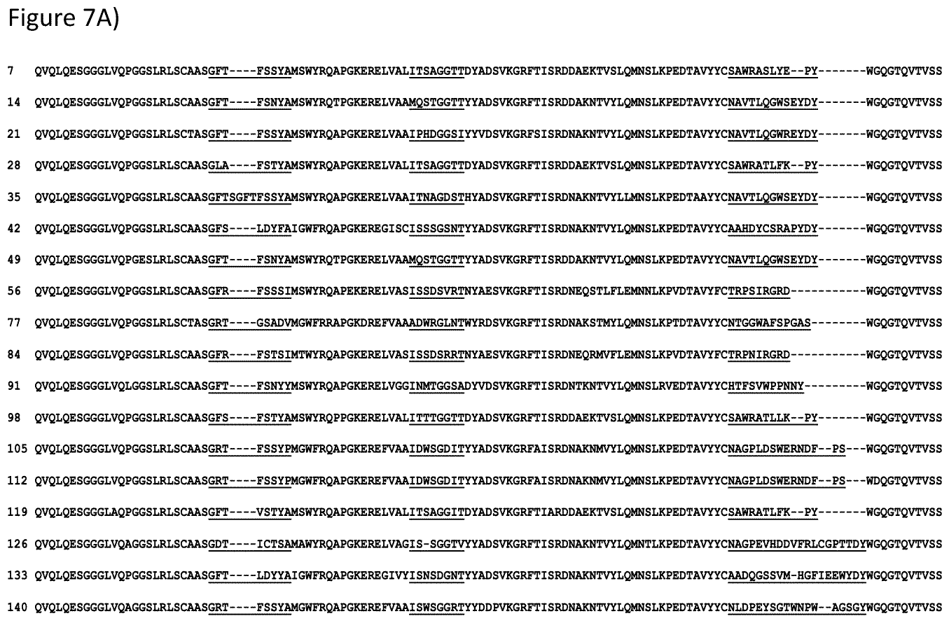

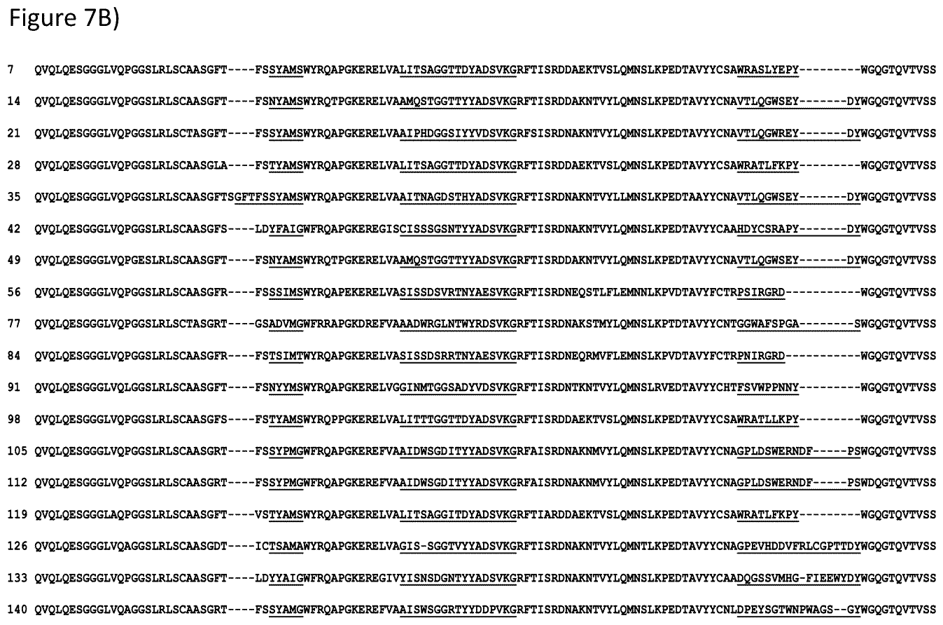

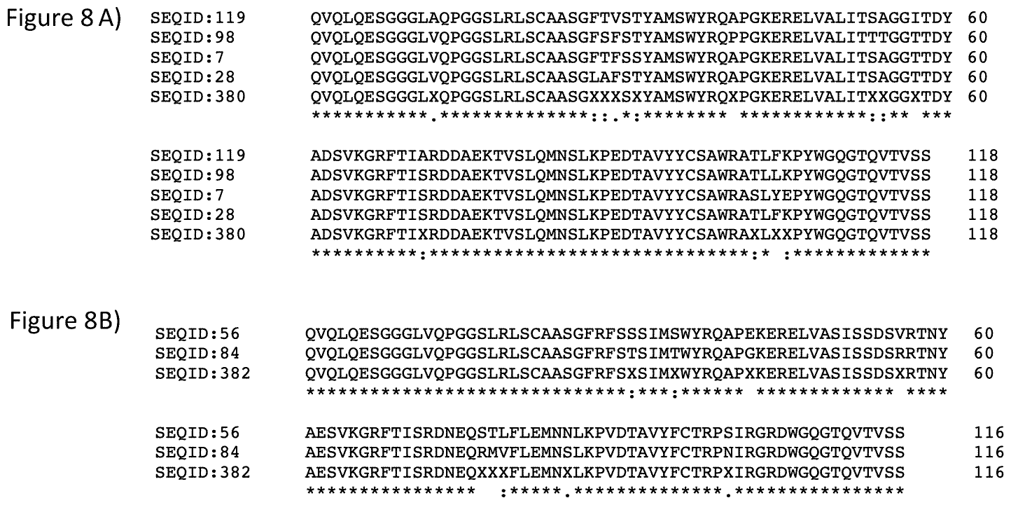

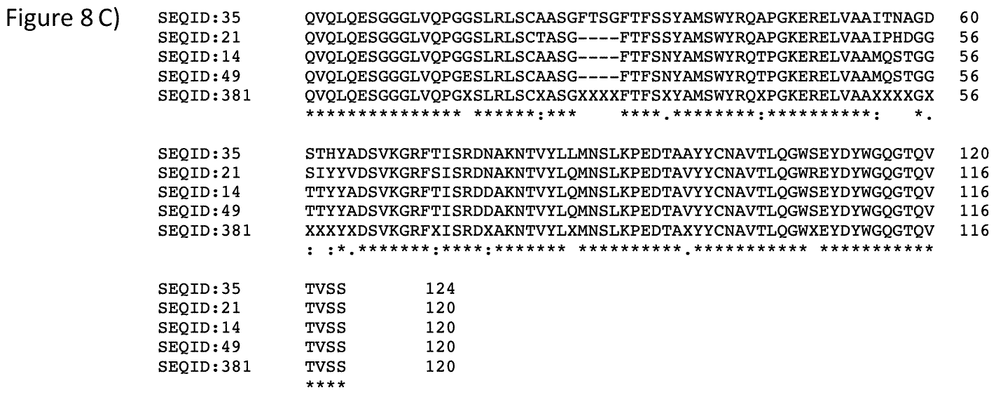

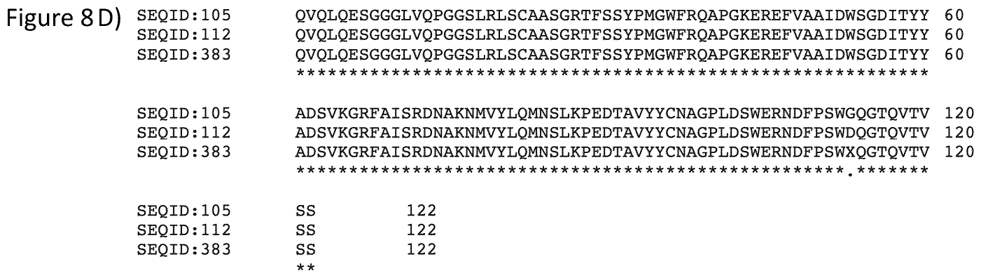

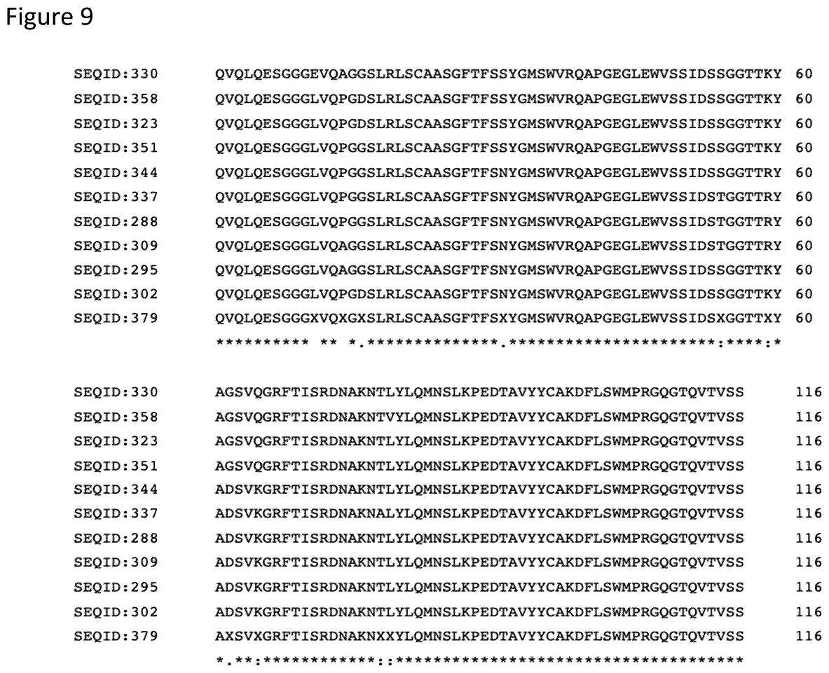

As can be seen from the sequence alignment of FIGS. 7A, 7B, and 8A-8D some of anti-CD36 VHH share a high level of identity (80% identity or more over the entire length of a given sequence) while having variations in their CDRs (including CDR3). Additional amino acid variation (substitution, addition and/or deletion) may thus be introduced in the framework regions (e.g., for humanization or other purposes) while preserving an activity similar to that of the original binding agent of Table 3. For example, an antibody having the consensus sequence set forth in SEQ ID NO:380, 381, 382 or 383 may sustain further amino acid variation such as amino acid substitutions. Moreover, it appears that further anti-CD36 VHH having slightly different CDRs as those of Table 3 may remain functional. As such, the present disclosure also encompasses binding agents with one, two, three, four or five amino acid substitutions in the CDRs of Table 3.

The embodiments and examples described herein are illustrative and are not meant to limit the scope of the claims. Variations of the foregoing embodiments, including alternatives, modifications and equivalents, are intended by the inventors to be encompassed by the claims. Citations listed in the present application are incorporated herein by reference.

### REFERENCES

All patents, patent applications and publications referred to throughout the application are incorporated herein by reference.
Angal, S. et al., A Single Amino Acid Substitution Abolishes the Heterogeneity of Chimeric Mouse/human IgG4 Antibody, Mol Immunol 30, 105-108, 1993
Chen X et al., Fusion Protein Linkers: Properties, Design and Functionality, Adv Drug Deliv Rev. 2013; 65(10):1357-1369.
Conrath, K. E. et al., Camel Single-Domain Antibodies as Modular Building Units in Bispecific and Bivalent Antibody Constructs. J. Biol. Chem. 2001, 276, 7346-7350.
Coppieters, K. et al., Formatted Anti-Tumor Necrosis Factor alpha VHH Proteins Derived from Camelids Show Superior Potency and Targeting to Inflamed Joints in a Murine Model of Collagen-induced Arthritis. Arthritis Rheum. 2006, 54, 1856-1866.
Detallea L, et al., Generation and Characterisation of ALX-0171, a Potent Novel Therapeutic Nanobody for the Treatment of Respiratory Syncytial Virus infection. Antimicrobial Agents and Chemotherapy 60(1) 2015
De Vlieger et al., Single-domain Antibodies and their Formatting to Combat Viral Infections, Antibodies 8(1), 1-22, 2019
Duggan S. Caplacizumab: First Global Approval. Drugs (2018) 78:1639-1642
Fleischmann R, et al., A Novel Individualized Treatment Approach in Open-Label Extension Study of Ozoralizumab (ATN-103) in Subjects with Rheumatoid Arthritis on a Background of Methotrexate. Abstract 1311, ACR/ARHP Annual Meeting (2012).
Fridy P C, et al., A Robust Pipeline for Rapid Production of Versatile Nanobody Repertoires. Nat Methods. 2014 December; 11(12): 1253-1260.
Godar M et al. Therapeutic Bispecific Antibody Formats: a Patent Applications Review (1994-2017) Expert Opinion on Therapeutic patents, 2018; 28(3):251-276
Ha, J-H et al. Immunoglobulin Fc Heterodimer Platform Technology: From Design to Applications in Therapeutic Antibodies and Proteins, Front Immunol, 2016; 7:394.
Hamers-Casterman C, et al., Naturally-occurring Antibodies Devoid of Light-chains. Nature 1993, 363:446-448.
Hmila, I. et al., VHH, Bivalent Domains and Chimeric Heavy Chain-only Antibodies with High Neutralizing Efficacy for Scorpion Toxin AahI. Mol. Immunol. 2008, 45, 3847-3856.
Hultberg, A et al., Llama-derived Single Domain Antibodies to Build Multivalent, Super Potent and Broadened Neutralizing Anti-viral Molecules. PLoS One 2011, doi: 10.1371/journal.pone. 0017665.
Jähnichen S, et al, CXCR4 Nanobodies (VHH-based single variable domains) Potently Inhibit Chemotaxis and HIV-1 Replication and Mobilize Stem Cells. Proc Natl Acad Sci USA. (2010) 107: 20565-20570.
Kovalenko OV et al. Atypical Antigen Recognition Mode of a Shark Immunoglobulin New Antigen Receptor (IgNAR) Variable Domain Characterized by Humanization and Structural Analysis, J Biol Chem. 2013, 288:17408-17419
Muyldermans,S and Smider, 2016. Distinct Antibody Species: Structural Differences Creating Therapeutic Opportunities. Current Opinion in Immunology 2016, 40:7-13
Muyldermans, S et al., 1994. Sequence and Structure of VH Domain From Naturally Occurring Camel Heavy Chain Immunoglobulins Lacking Light Chains. Protein Eng. 7: 1129-1135.
Saunders K.O., Conceptual Approach to Modulating Antibody Effector Functions and Circuation Half-Life, Front. Immunol. 10:1296, 2019.
Schoen P, et al., Anti-RANKL Nanobody® ALX-0141 Shows Sustained Biomarker Inhibition in a Phase I Study in Healthy Postmenopausal Women. DOI: 10.1530/boneabs. 1.PP135 (2013)
Simmons, D. P et al., Dimerisation Strategies for Shark IgNAR Single Domain Antibody Fragments. J. Immunol. Methods 2006, 315, 171-184.
Svecova D, et al., A Randomized, Double-Blind, Placebo-Controlled Phase 1 Study of Multiple Ascending Doses of Subcutaneous M1095, an Anti-Interleukin-17A/F Nanobody®, in Moderate-to-Severe Psoriasis. J Am Acad Dermatol. 2019 Mar 26.
Tatusova, T. A, et al. (1999), BLAST 2 Sequences, a New Tool for Comparing Protein and Nucleotide Sequences, FEMS Microbiol Lett. 174:247-250)
Van Roy M, et al., The Preclinical Pharmacology of the High Affinity Anti-IL-6R Nanobody® ALX-0061 Supports its Clinical Development in Rheumatoid Arthritis. Arthritis Research & Therapy (2015) 17:135
Vincke C. et al. General Strategy to Humanize a Camelid Single-Domain Antibody and Identification of a Universal Humanized Nanobody Scaffold, J.Biol Chem. 2009, 284(5):3273-3284
Vu, K. B., et al., 1997. Comparison of Llama VH Sequences from Conventional and Heavy Chain Antibodies. Mol. Immunol. 34: 1121-1131.

TABLE 3
SEQUENCE TABLES
anti-CD36 VHHs sequences

| | CDR1 | CDR2 | CDR3 |
|---|---|---|---|
| KF001 (Kabat | SYAMS | LITSAGGTTDYADS | WRASLYEPY |
| CDRs) | (SEQ ID NO: 1) | VKG | (SEQ ID NO: 3) |
| | | (SEQ ID NO: 2) | |
| KF001 (IMGT | GFTFSSYA | ITSAGGTT | SAWRASLYEPY |
| CDRs) | (SEQ ID NO: 4) | (SEQ ID NO: 5) | (SEQ ID NO: 6) |
| KF001 (VHH) | QVQLQESGGGLVQPGGSLRLSCAASGFTFSSYAMSWYRQAPGKE | | |
| (SEQ ID NO: 7) | RELVALITSAGGTTDYADSVKGRFTISRDDAEKTVSLQMNSLKPE | | |
| | DTAVYYCSAWRASLYEPYWGQGTQVTVSS | | |
| KF002 (Kabat | NYAMS | AMQSTGGTTYYAD | VTLQGWSEYDY |
| CDRs) | (SEQ ID NO: 8) | SVKG | (SEQ ID NO: 10) |
| | | (SEQ ID NO: 9) | |
| KF002 (IMGT | GFTFSNYA | MQSTGGTT | NAVTLQGWSEYDY |
| CDRs) | (SEQ ID NO: 11) | (SEQ ID NO: 12) | (SEQ ID NO: 13) |
| KF002 (VHH) | QVQLQESGGGLVQPGGSLRLSCAASGFTFSNYAMSWYRQTPGKE | | |
| (SEQ ID NO: 14) | RELVAAMQSTGGTTYYADSVKGRFTISRDDAKNTVYLQMNSLKP | | |
| | EDTAVYYCNAVTLQGWSEYDYWGQGTQVTVSS | | |
| KF003 (Kabat | SYAMS | AIPHDGGSIYYVDS | VTLQGWREYDY |
| CDRs) | (SEQ ID NO: 15) | VKG | (SEQ ID NO: 17) |
| | | (SEQ ID NO: 16) | |
| KF003 (IMGT | GFTFSSYA | IPHDGGSI | NAVTLQGWREYDY |
| CDRs) | (SEQ ID NO: 18) | (SEQ ID NO: 19) | (SEQ ID NO: 20) |
| KF003 (VHH) | QVQLQESGGGLVQPGGSLRLSCTASGFTFSSYAMSWYRQAPGKE | | |
| (SEQ ID NO: 21) | RELVAAIPHDGGSIYYVDSVKGRFSISRDNAKNTVYLQMNSLKPE | | |
| | DTAVYYCNAVTLQGWREYDYWGQGTQVTVSS | | |
| KF004 (Kabat | TYAMS | LITSAGGTTDYADS | WRATLFKPY |
| CDRs) | (SEQ ID NO: 22) | VKG | (SEQ ID NO: 24) |
| | | (SEQ ID NO: 23) | |
| KF004 (IMGT | GLAFSTYA | ITSAGGTT | SAWRATLFKPY |
| CDRs) | (SEQ ID NO: 25) | (SEQ ID NO: 26) | (SEQ ID NO: 27) |
| KF004 (VHH) | QVQLQESGGGLVQPGGSLRLSCAASGLAFSTYAMSWYRQAPGKE | | |
| (SEQ ID NO: 28) | RELVALITSAGGTTDYADSVKGRFTISRDDAEKTVSLQMNSLKPE | | |
| | DTAVYYCSAWRATLFKPYWGQGTQVTVSS | | |
| KF005 (Kabat | FTFSSYAMS | AITNAGDSTHYADS | VTLQGWSEYDY |
| CDRs) | (SEQ ID NO: 29) | VKG | (SEQ ID NO: 31) |
| | | (SEQ ID NO: 30) | |
| KF005 (IMGT | GFTSGFTFSSYA | ITNAGDST | NAVTLQGWSEYDY |
| CDRs) | (SEQ ID NO: 32) | (SEQ ID NO: 33) | (SEQ ID NO: 34) |
| KF005 (VHH) | QVQLQESGGGLVQPGGSLRLSCAASGFTSGFTFSSYAMSWYRQAP | | |
| (SEQ ID NO: 35) | GKERELVAAITNAGDSTHYADSVKGRFTISRDNAKNTVYLLMNSL | | |
| | KPEDTAAYYCNAVTLQGWSEYDYWGQGTQVTVSS | | |
| KF006 (Kabat | YFAIG | CISSSGSNTYYADS | HDYCSRAPYDY |
| CDRs) | (SEQ ID NO: 36) | VKG | (SEQ ID NO: 38) |
| | | (SEQ ID NO: 37) | |
| KF006 (IMGT | GFSLDYFA | ISSSGSNT | AAHDYCSRAPYDY |
| CDRs) | (SEQ ID NO: 39) | (SEQ ID NO: 40) | (SEQ ID NO: 41) |
| KF006 (VHH) | QVQLQESGGGLVQPGGSLRLSCAASGFSLDYFAIGWFRQAPGKER | | |
| (SEQ ID NO: 42) | EGISCISSSGSNTYYADSVKGRFTISRDNAKNTVYLQMNSLKPEDT | | |
| | AVYYCAAHDYCSRAPYDYWGQGTQVTVSS | | |
| KF007 (Kabat | NYAMS | AMQSTGGTTYYAD | VTLQGWSEYDY |
| CDRs) | (SEQ ID NO: 43) | SVKG | (SEQ ID NO: 45) |
| | | (SEQ ID NO: 44) | |
| KF007 (IMGT | GFTFSNYA | MQSTGGTT | NAVTLQGWSEYDY |
| CDRs) | (SEQ ID NO: 46) | (SEQ ID NO: 47) | (SEQ ID NO: 48) |
| KF007 (VHH) | QVQLQESGGGLVQPGESLRLSCAASGFTFSNYAMSWYRQTPGKE | | |
| (SEQ ID NO: 49) | RELVAAMQSTGGTTYYADSVKGRFTISRDDAKNTVYLQMNSLKP | | |
| | EDTAVYYCNAVTLQGWSEYDYWGQGTQVTVSS | | |
| KF008 (Kabat | SSIMS | SISSDSVRTNYAESV | PSIRGRD |
| CDRs) | (SEQ ID NO: 50) | KG | (SEQ ID NO: 52) |
| | | (SEQ ID NO: 51) | |
| KF008 (IMGT | GFRFSSSI | ISSDSVRT | TRPSIRGRD |
| CDRs) | (SEQ ID NO: 53) | (SEQ ID NO: 54) | (SEQ ID NO: 55) |
| KF008 (VHH) | QVQLQESGGGLVQPGGSLRLSCAASGFRFSSSIMSWYRQAPEKER | | |
| (SEQ ID NO: 56) | ELVASISSDSVRTNYAESVKGRFTISRDNEQSTLFLEMNNLKPVDT | | |
| | AVYFCTRPSIRGRDWGQGTQVTVSS | | |
| KF009 (Kabat | ADVMG | AADWRGLNTWYR | (GGWAFSPGAS |
| CDRs) | (SEQ ID NO: 71) | DSVKG | SEQ ID NO: 73) |
| | | (SEQ ID NO: 72) | |
| KF009 (IMGT | GRTGSADV | ADWRGLNT | NTGGWAFSPGAS |
| CDRs) | (SEQ ID NO: 74) | (SEQ ID NO: 75) | (SEQ ID NO: 76) |
| KF009 (VHH) | QVQLQESGGGLVQPGGSLRLSCTASGRTGSADVMGWFRRAPGKD | | |
| (SEQ ID NO: 77) | REFVAAADWRGLNTWYRDSVKGRFTISRDNAKSTMYLQMNSLK | | |
| | PTDTAVYYCNTGGWAFSPGASWGQGTQVTVSS | | |
| KF010 (Kabat | TSIMT | SISSDSRRTNYAESV | PNIRGRD |
| CDRs) | (SEQ ID NO: 78) | KG | (SEQ ID NO: 80) |
| | | (SEQ ID NO: 79) | |
| KF010 (IMGT | GFRFSTSI | ISSDSRRT | TRPNIRGRD |
| CDRs) | (SEQ ID NO: 81) | (SEQ ID NO: 82) | (SEQ ID NO: 83) |
| KF010 (VHH) | QVQLQESGGGLVQPGGSLRLSCAASGFRFSTSIMTWYRQAPGKER | | |
| (SEQ ID NO: 84) | ELVASISSDSRRTNYAESVKGRFTISRDNEQRMVFLEMNSLKPVDT | | |
| | AVYFCTRPNIRGRDWGQGTQVTVSS | | |
| KF011 (Kabat | NYYMS | GINMTGGSADYVD | FSVWPPNNY |
| CDRs) | (SEQ ID NO: 85) | SVKG | (SEQ ID NO: 87) |
| | | (SEQ ID NO: 86) | |
| KF011 (IMGT | GFTFSNYY | INMTGGSA | HTFSVWPPNNY |
| CDRs) | (SEQ ID NO: 88) | (SEQ ID NO: 89) | (SEQ ID NO: 90) |
| KF011 (VHH) | QVQLQESGGGLVQLGGSLRLSCAASGFTFSNYYMSWYRQAPGKE | | |
| (SEQ ID NO: 91) | RELVGGINMTGGSADYVDSVKGRFTISRDNTKNTVYLQMNSLRV | | |
| | EDTAVYYCHTFSVWPPNNYWGQGTQVTVSS | | |
| KF012 (Kabat | TYAMS | LITTTGGTTDYADS | WRATLLKPY |
| CDRs) | (SEQ ID NO: 92) | VKG | (SEQ ID NO: 94) |
| | | (SEQ ID NO: 93) | |
| KF012 (IMGT | GFSFSTYA | ITTTGGTT | SAWRATLLKPY |
| CDRs) | (SEQ ID NO: 95) | (SEQ ID NO: 96) | (SEQ ID NO: 97) |
| KF012 (VHH) | QVQLQESGGGLVQPGGSLRLSCAASGFSFSTYAMSWYRQPPGKE | | |
| (SEQ ID NO: 98) | RELVALITTTGGTTDYADSVKGRFTISRDDAEKTVSLQMNSLKPE | | |
| | DTAVYYCSAWRATLLKPYWGQGTQVTVSS | | |
| KF013 (Kabat | SYPMG | AIDWSGDITYYADS | GPLDSWERNDFPS |
| CDRs) | (SEQ ID NO: 99) | VKG | (SEQ ID NO: 101) |
| | | (SEQ ID NO: 100) | |
| KF013 (IMGT | GRTFSSYP | IDWSGDIT | NAGPLDSWERNDF |
| CDRs) | (SEQ ID NO: 102) | (SEQ ID NO: 103) | PS |
| | | | (SEQ ID NO: 104) |
| KF013 (VHH) | QVQLQESGGGLVQPGGSLRLSCAASGRTFSSYPMGWFRQAPGKE | | |
| (SEQ ID NO: 105) | REFVAAIDWSGDITYYADSVKGRFAISRDNAKNMVYLQMNSLKP | | |
| | EDTAVYYCNAGPLDSWERNDFPSWGQGTQVTVSS | | |
| KF014 (Kabat | SYPMG | AIDWSGDITYYADS | GPLDSWERNDFPS |
| CDRs) | (SEQ ID NO: 106) | VKG | (SEQ ID NO: 108) |
| | | (SEQ ID NO: 107) | |
| KF014 (IMGT | GRTFSSYP | IDWSGDIT | NAGPLDSWERNDF |
| CDRs) | (SEQ ID NO: 109) | (SEQ ID NO: 110) | PS |
| | | | (SEQ ID NO: 111) |
| KF014 (VHH) | QVQLQESGGGLVQPGGSLRLSCAASGRTFSSYPMGWFRQAPGKE | | |
| (SEQ ID NO: 112) | REFVAAIDWSGDITYYADSVKGRFAISRDNAKNMVYLQMNSLKP | | |
| | EDTAVYYCNAGPLDSWERNDFPSWDQGTQVTVSS | | |
| KF015 (Kabat | TYAMS | LITSAGGITDYADS | WRATLFKPY |
| CDRs) | (SEQ ID NO: 113) | VKG | (SEQ ID NO: 115) |
| | | (SEQ ID NO: 114) | |
| KF015 (IMGT | GFTVSTYA | ITSAGGIT | SAWRATLFKPY |
| CDRs) | (SEQ ID NO: 116) | (SEQ ID NO: 117) | (SEQ ID NO: 118) |
| KF015 (VHH) | QVQLQESGGGLAQPGGSLRLSCAASGFTVSTYAMSWYRQAPGKE | | |
| (SEQ ID NO: 119) | RELVALITSAGGITDYADSVKGRFTIARDDAEKTVSLQMNSLKPE | | |
| | DTAVYYCSAWRATLFKPYWGQGTQVTVSS | | |
| KF016 (Kabat | TSAMA | GISSGGTVYYADSV | GPEVHDDVFRLCGP |
| CDRs) | (SEQ ID NO: 120) | KG | TTDY |
| | | (SEQ ID NO: 121) | (SEQ ID NO: 122) |
| KF016 (IMGT | GDTICTSA | ISSGGTV | NAGPEVHDDVFRL |
| CDRs) | (SEQ ID NO: 123) | (SEQ ID NO: 124) | CGPTTDY |
| | | | (SEQ ID NO: 125) |
| KF016 (VHH) | QVQLQESGGGLVQAGGSLRLSCAASGDTICTSAMAWYRQAPGKE | | |
| (SEQ ID NO: 126) | RELVAGISSGGTVYYADSVKGRFTISRDNAKNTVYLQMNTLKPED | | |
| | TAVYYCNAGPEVHDDVFRLCGPTTDYWGQGTQVTVSS | | |
| KF017 (Kabat | YYAIG | YISNSDGNTYYADS | DQGSSVMHGFIEE |
| CDRs) | (SEQ ID NO: 127) | VKG | WYDY |
| | | (SEQ ID NO: 128) | (SEQ ID NO: 129) |
| KF017 (IMGT | GFTLDYYA | ISNSDGNT | AADQGSSVMHGFIE |
| CDRs) | (SEQ ID NO: 130) | (SEQ ID NO: 131) | EWYDY |
| | | | (SEQ ID NO: 132) |
| KF017 (VHH) | QVQLQESGGGLVQPGGSLRLSCAASGFTLDYYAIGWFRQAPGKE | | |
| (SEQ ID NO: 133) | REGIVYISNSDGNTYYADSVKGRFTISRDNAKNTVYLQMNSLKPE | | |
| | DTAVYYCAADQGSSVMHGFIEEWYDYWGQGTQVTVSS | | |
| KF018 (Kabat | SYAMG | AISWSGGRTYYDDP | DPEYSGTWNPWAG |
| CDRs) | (SEQ ID NO: 134) | VKG | SGY |
| | | (SEQ ID NO: 135) | (SEQ ID NO: 136) |
| KF018 (IMGT | GRTFSSYA | ISWSGGRT | NLDPEYSGTWNPW |
| CDRs) | (SEQ ID NO: 137) | (SEQ ID NO: 138) | AGSGY |
| | | | (SEQ ID NO: 139) |

| | |
|---|---|
| KF018 (VHH) | QVQLQESGGGLVQAGGSLRLSCAASGRTFSSYAMGWFRQAPGKE |
| (SEQ ID NO: 140) | REFVAAISWSGGRTYYDDPVKGRFTISRDNAKNTVYLQMNSLKPE |
| | DTAVYYCNLDPEYSGTWNPWAGSGYWGQGTQVTVSS |

TABLE 4
anti-PD-1 VHHs

| Anti-PD-1 VHHs | CDR1 | CDR2 | CDR3 |
|---|---|---|---|
| KF021 (Kabat | RSSMS | IISSSGIITHYSDSVK | VLDDDSGDY |
| CDRs) | (SEQ ID NO: 57) | G | (SEQ ID NO: 59) |
| | | (SEQ ID NO: 58) | |
| KF021 | GFTFSRSS | ISSSGIIT | NAVLDDDSGDY |
| (IMGT CDRs) | (SEQ ID NO: 60) | (SEQ ID NO: 61) | (SEQ ID NO: 62) |
| KF021 (VHH) | QVQLQESGGGWVQPGGSLRLSCVASGFTFSRSSMSWHRQAPGKE | | |
| (SEQ ID NO: 63) | RDLVAIISSSGIITHYSDSVKGRFTISRDNAKDTVYLQMNSLKPEDT | | |
| | AVYVCNAVLDDDSGDYWGQGTQVTVSS | | |
| KF062 | FENVA | IIAQYITHYNDAVKG | DQY |
| (Kabat CDRs) | (SEQ ID NO: 275) | (SEQ ID NO: 276) | (SEQ ID NO: 277) |
| KF062 | TSMHSFEN | IAQYIT | NVDQY |
| (IMGT CDRs) | (SEQ ID NO: 278) | (SEQ ID NO: 279) | (SEQ ID NO: 280) |
| KF062 (VHH) | QVQLQESGGGLVQPGGSLRLSCATSTSMHSFENVAWYRQAPGKQ | | |
| (SEQ ID NO: 281) | RELVAIIAQYITHYNDAVKGRFTISRDDSKNTAYLQMNNLKPEDT | | |
| | AVYYCNVDQYWGQGTQVTVSS | | |
| KF063 | NYGMS | SIDSTGGTTRYADSV | DFLSWMP |
| (Kabat CDRs) | (SEQ ID NO: 282) | KG | (SEQ ID NO: 284) |
| | | (SEQ ID NO: 283) | |
| KF063 | GFTFSNYG | IDSTGGTT | AKDFLSWMP |
| (IMGT CDRs) | (SEQ ID NO: 285) | (SEQ ID NO: 286) | (SEQ ID NO: 287) |
| KF063 (VHH) | QVQLQESGGGLVQPGGSLRLSCAASGFTFSNYGMSWVRQAPGEG | | |
| (SEQ ID NO: 288) | LEWVSSIDSTGGTTRYADSVKGRFTISRDNAKNTLYLQMNSLKPE | | |
| | DTAVYYCAKDFLSWMPRGQGTQVTVSS | | |
| KF064 | NYGMS | SIDSSGGTTKYADSV | DFLSWMP |
| (Kabat CDRs) | (SEQ ID NO: 289) | KG | (SEQ ID NO: 291) |
| | | (SEQ ID NO: 290) | |
| KF064 | GFTFSNYG | IDSSGGTT | AKDFLSWMP |
| (IMGT CDRs) | (SEQ ID NO: 292) | (SEQ ID NO: 293) | (SEQ ID NO: 294) |
| KF064 (VHH) | QVQLQESGGGLVQAGGSLRLSCAASGFTFSNYGMSWVRQAPGEG | | |
| (SEQ ID NO: 295) | LEWVSSIDSSGGTTKYADSVKGRFTISRDNAKNTLYLQMNSLKPE | | |
| | DTAVYYCAKDFLSWMPRGQGTQVTVSS | | |
| KF065 | NYGMS | SIDSSGGTTKYADSV | DFLSWMP |
| (Kabat CDRs) | (SEQ ID NO: 296) | KG | (SEQ ID NO: 298) |
| | | (SEQ ID NO: 297) | |
| KF065 | GFTFSNYG | IDSSGGTT | AKDFLSWMP |
| (IMGT CDRs) | (SEQ ID NO: 299) | (SEQ ID NO: 300) | (SEQ ID NO: 301) |
| KF065 (VHH) | QVQLQESGGGLVQPGDSLRLSCAASGFTFSNYGMSWVRQAPGEG | | |
| (SEQ ID NO: 302) | LEWVSSIDSSGGTTKYADSVKGRFTISRDNAKNTLYLQMNSLKPE | | |
| | DTAVYYCAKDFLSWMPRGQGTQVTVSS | | |
| KF066 | NYGMS | SIDSTGGTTRYADSV | DFLSWMP |
| (Kabat CDRs) | (SEQ ID NO: 303) | KG | (SEQ ID NO: 305) |
| | | (SEQ ID NO: 304) | |
| KF066 | GFTFSNYG | IDSTGGTT | AKDFLSWMP |
| (IMGT CDRs) | (SEQ ID NO: 306) | (SEQ ID NO: 307) | (SEQ ID NO: 308) |
| KF066 (VHH) | QVQLQESGGGLVQAGGSLRLSCAASGFTFSNYGMSWVRQAPGEG | | |
| (SEQ ID NO: 309) | LEWVSSIDSTGGTTRYADSVKGRFTISRDNAKNTLYLQMNSLKPE | | |
| | DTAVYYCAKDFLSWMPRGQGTQVTVSS | | |
| KF067 | FENVA | IIAQYITHYNDAVKG | DQY |
| (Kabat CDRs) | (SEQ ID NO: 310) | (SEQ ID NO: 311) | (SEQ ID NO: 312) |
| KF067 | TSMHSFEN | IAQYIT | NVDQY |
| (IMGT CDRs) | (SEQ ID NO: 313) | (SEQ ID NO: 314) | (SEQ ID NO: 315) |
| KF067 VHH) | QVQLQESGGGLVQPGGSLRLSCATSTSMHSFENVAWYRQAPGKQ | | |
| (SEQ ID NO: 316) | RELAAIIAQYITHYNDAVKGRFTISRDDSKNTAYLQMNNLKPEDT | | |
| | AVYYCNVDQYWGQGTQVTVSS | | |
| KF068 | SYGMS | SIDSSGGTTKYAGSV | DFLSWMP |
| (Kabat CDRs) | (SEQ ID NO: 317) | QG | (SEQ ID NO: 319) |
| | | (SEQ ID NO: 318) | |
| KF068 | GFTFSSYG | IDSSGGTT | AKDFLSWMP |
| (IMGT CDRs) | (SEQ ID NO: 320) | (SEQ ID NO: 321) | (SEQ ID NO: 322) |
| KF068 (VHH) | QVQLQESGGGLVQPGDSLRLSCAASGFTFSSYGMSWVRQAPGEG | | |
| (SEQ ID NO: 323) | LEWVSSIDSSGGTTKYAGSVQGRFTISRDNAKNTLYLQMNSLKPE | | |
| | DTAVYYCAKDFLSWMPRGQGTQVTVSS | | |
| KF069 | SYGMS | SIDSSGGTTKYAGSV | DFLSWMP |
| (Kabat CDRs) | (SEQ ID NO: 324) | QG | (SEQ ID NO: 326) |
| | | (SEQ ID NO: 325) | |
| KF069 | GFTFSSYG | IDSSGGTT | AKDFLSWMP |
| (IMGT CDRs) | (SEQ ID NO: 327) | (SEQ ID NO: 328) | (SEQ ID NO: 329) |
| KF069 (VHH) | QVQLQESGGGEVQAGGSLRLSCAASGFTFSSYGMSWVRQAPGEG | | |
| (SEQ ID NO: 330) | LEWVSSIDSSGGTTKYAGSVQGRFTISRDNAKNTLYLQMNSLKPE | | |
| | DTAVYYCAKDFLSWMPRGQGTQVTVSS | | |
| KF070 | NYGMS | SIDSTGGTTRYADSV | DFLSWMP |
| (Kabat CDRs) | (SEQ ID NO: 331) | KG | (SEQ ID NO: 333) |
| | | (SEQ ID NO: 332) | |
| KF070 | GFTFSNYG | IDSTGGTT | AKDFLSWMP |
| (IMGT CDRs) | (SEQ ID NO: 334) | (SEQ ID NO: 335) | (SEQ ID NO: 336) |
| KF070 (VHH) | QVQLQESGGGLVQPGGSLRLSCAASGFTFSNYGMSWVRQAPGEG | | |
| (SEQ ID NO: 337) | LEWVSSIDSTGGTTRYADSVKGRFTISRDNAKNALYLQMNSLKPE | | |
| | DTAVYYCAKDFLSWMPRGQGTQVTVSS | | |
| KF071 | NYGMS | SIDSSGGTTRYADSV | DFLSWMP |
| (Kabat CDRs) | (SEQ ID NO: 338) | KG | (SEQ ID NO: 340) |
| | | (SEQ ID NO: 339) | |
| KF071 | GFTFSNYG | IDSSGGTT | AKDFLSWMP |
| (IMGT CDRs) | (SEQ ID NO: 341) | (SEQ ID NO: 342) | (SEQ ID NO: 343) |
| KF071 (VHH) | QVQLQESGGGLVQPGGSLRLSCAASGFTFSNYGMSWVRQAPGEG | | |
| (SEQ ID NO: 344) | LEWVSSIDSSGGTTRYADSVKGRFTISRDNAKNTLYLQMNSLKPE | | |
| | DTAVYYCAKDFLSWMPRGQGTQVTVSS | | |
| KF072 | SYGMS | SIDSSGGTTKYAGSV | DFLSWMP |
| (Kabat CDRs) | (SEQ ID NO: 345) | QG | (SEQ ID NO: 347) |
| | | (SEQ ID NO: 346) | |
| KF072 | GFTFSSYG | IDSSGGTT | AKDFLSWMP |
| (IMGT CDRs) | (SEQ ID NO: 348) | (SEQ ID NO: 349) | (SEQ ID NO: 350) |
| KF072 (VHH) | QVQLQESGGGLVQPGGSLRLSCAASGFTFSSYGMSWVRQAPGEG | | |
| (SEQ ID NO: 351) | LEWVSSIDSSGGTTKYAGSVQGRFTISRDNAKNTLYLQMNSLKPE | | |
| | DTAVYYCAKDFLSWMPRGQGTQVTVSS | | |
| KF073 | SYGMS | SIDSSGGTTKYAGSV | DFLSWMP |
| (Kabat CDRs) | (SEQ ID NO: 352) | QG | (SEQ ID NO: 354) |
| | | (SEQ ID NO: 353) | |
| KF073 | GFTFSSYG | IDSSGGTT | AKDFLSWMP |
| (IMGT CDRs) | (SEQ ID NO: 355) | (SEQ ID NO: 356) | (SEQ ID NO: 357) |
| KF073 (VHH) | QVQLQESGGGLVQPGDSLRLSCAASGFTFSSYGMSWVRQAPGEG | | |
| (SEQ ID NO: 358) | LEWVSSIDSSGGTTKYAGSVQGRFTISRDNAKNTVYLQMNSLKPE | | |
| | DTAVYYCAKDFLSWMPRGQGTQVTVSS | | |
| KF074 | SDTMG | AIQRRGGSIYYADSV | SPVNGDNYGLLPR |
| (Kabat CDRs) | (SEQ ID NO: 359) | KG | HYDY |
| | | (SEQ ID NO: 360) | (SEQ ID NO: 361) |
| KF074 | SVTFSSDT | IQRRGGSI | AASPVNGDNYGLL |
| (IMGT CDRs) | (SEQ ID NO: 362) | (SEQ ID NO: 363) | PRHYDY |
| | | | (SEQ ID NO: 364) |

| | |
|---|---|
| KF074 (VHH) | QVQLQESGGGLVQAGGSLRLSCTASSVTFSSDTMGWFRQAPGKE |
| (SEQ ID NO: 365) | REFVAAIQRRGGSIYYADSVKGRFTISRDNPKSTVYLQMNSLKPED |
| | TAVYYCAASPVNGDNYGLLPRHYDYWGQGTQVTVSS |

TABLE 5
anti-CD47 VHHs

| Anti-CD47 VHHs | CDR1 | CDR2 | CDR3 |
|---|---|---|---|
| KF022 (Kabat | VNDIR | RITGGGRTDYADSV | WGRGY |
| CDRs) | (SEQ ID NO: 64) | KG | (SEQ ID NO: 66) |
| | | (SEQ ID NO: 65) | |
| KF022 | RFDFSVND | ITGGGRT | WGRGY |
| (IMGT CDRs) | (SEQ ID NO: 67) | (SEQ ID NO: 68) | (SEQ ID NO: 69) |

| | |
|---|---|
| KF022 (VHH) | QVQLQESGGGLVQPGGSLRLSCAASRFDFSVNDIRWYRQAPGNE |
| (SEQ ID NO: 70) | RELVARITGGGRTDYADSVKGRFTISRDNAKNTVYLQMNNLKPE |
| | DTAVYYCWGRGYWGQGTQVTVSS |

TABLE 6
Exemplary embodiments of polypeptide chains comprising one antigen binding
domain (VHHs of Table 3 fused with human hinge-CH2-CH3 (SEQ ID NO: 227))
(may comprise a His-tag or not)

| SEQ ID NO: | |
|---|---|
| (specificity) | SEQUENCE |
| KF023 | QVQLQESGGGLVQPGGSLRLSCAASGFTFSSYAMSWYRQAPGKER |
| SEQ ID NO: 141 | ELVALITSAGGTTDYADSVKGRFTISRDDAEKTVSLQMNSLKPEDT |
| (CD36) | AVYYCSAWRASLYEPYWGQGTQVTVSSEPKSCDKTHTCPPCPAPE |
| | LLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKV |
| | SNKALPAPIEKTISKAKGQPREPQVYTLPPSREEMTKNQVSLTCLVK |
| | GFYPSDIAVEWESNGQPENNYKTTPPVLDSDGSFFLYSKLTVDKSR |
| | WQQGNVFSCSVMHEALHNHYTQKSLSLSPGK |
| KF024 | QVQLQESGGGLVQPGGSLRLSCAASGFTFSNYAMSWYRQTPGKER |
| SEQ ID NO: 142 | ELVAAMQSTGGTTYYADSVKGRFTISRDDAKNTVYLQMNSLKPED |
| (CD36) | TAVYYCNAVTLQGWSEYDYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPGK |
| KF025 | QVQLQESGGGLVQPGGSLRLSCTASGFTFSSYAMSWYRQAPGKER |
| SEQ ID NO: 143 | ELVAAIPHDGGSIYYVDSVKGRFSISRDNAKNTVYLQMNSLKPEDT |
| (CD36) | AVYYCNAVTLQGWREYDYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPGK |
| KF026 | QVQLQESGGGLVQPGGSLRLSCAASGLAFSTYAMSWYRQAPGKER |
| SEQ ID NO: 144 | ELVALITSAGGTTDYADSVKGRFTISRDDAEKTVSLQMNSLKPEDT |
| (CD36) | AVYYCSAWRATLFKPYWGQGTQVTVSSEPKSCDKTHTCPPCPAPE |
| | LLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKV |
| | SNKALPAPIEKTISKAKGQPREPQVYTLPPSREEMTKNQVSLTCLVK |
| | GFYPSDIAVEWESNGQPENNYKTTPPVLDSDGSFFLYSKLTVDKSR |
| | WQQGNVFSCSVMHEALHNHYTQKSLSLSPGK |
| KF027 | QVQLQESGGGLVQPGGSLRLSCAASGFTSGFTFSSYAMSWYRQAP |
| SEQ ID NO: 145 | GKERELVAAITNAGDSTHYADSVKGRFTISRDNAKNTVYLLMNSL |
| (CD36) | KPEDTAAYYCNAVTLQGWSEYDYWGQGTQVTVSSEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTKN |
| | QVSLTCLVKGFYPSDIAVEWESNGQPENNYKTTPPVLDSDGSFFLY |
| | SKLTVDKSRWQQGNVFSCSVMHEALHNHYTQKSLSLSPGK |
| KF028 | QVQLQESGGGLVQPGGSLRLSCAASGFSLDYFAIGWFRQAPGKERE |
| SEQ ID NO: 146 | GISCISSSGSNTYYADSVKGRFTISRDNAKNTVYLQMNSLKPEDTAV |
| (CD36) | YYCAAHDYCSRAPYDYWGQGTQVTVSSEPKSCDKTHTCPPCPAPE |
| | LLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKV |
| | SNKALPAPIEKTISKAKGQPREPQVYTLPPSREEMTKNQVSLTCLVK |
| | GFYPSDIAVEWESNGQPENNYKTTPPVLDSDGSFFLYSKLTVDKSR |
| | WQQGNVFSCSVMHEALHNHYTQKSLSLSPGK |
| KF029 | QVQLQESGGGLVQPGESLRLSCAASGFTFSNYAMSWYRQTPGKER |
| SEQ ID NO: 147 | ELVAAMQSTGGTTYYADSVKGRFTISRDDAKNTVYLQMNSLKPED |
| (CD36) | TAVYYCNAVTLQGWSEYDYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPGK |
| KF030 | QVQLQESGGGLVQPGGSLRLSCAASGFRFSSSIMSWYRQAPEKERE |
| SEQ ID NO: 148 | LVASISSDSVRTNYAESVKGRFTISRDNEQSTLFLEMNNLKPVDTAV |
| (CD36) | YFCTRPSIRGRDWGQGTQVTVSSEPKSCDKTHTCPPCPAPELLGGPS |
| | VFLFPPKPKDTLMISRTPEVTCVVVDVSHEDPEVKFNWYVDGVEV |
| | HNAKTKPREEQYNSTYRVVSVLTVLHQDWLNGKEYKCKVSNKAL |
| | PAPIEKTISKAKGQPREPQVYTLPPSREEMTKNQVSLTCLVKGFYPS |
| | DIAVEWESNGQPENNYKTTPPVLDSDGSFFLYSKLTVDKSRWQQG |
| | NVFSCSVMHEALHNHYTQKSLSLSPGK |
| KF031 | KSTMYLQMNSLKPTDTAVYYCNTGGWAFSPGASWGQGTQVTVSS |
| SEQ ID NO: 149 | EPKSCDKTHTCPPCPAPELLGGPSVFLFPPKPKDTLMISRTPEVTCVV |
| (CD36) | VDVSHEDPEVKFNWYVDGVEVHNAKTKPREEQYNSTYRVVSVLT |
| | VLHQDWLNGKEYKCKVSNKALPAPIEKTISKAKGQPREPQVYTLPP |
| | SREEMTKNQVSLTCLVKGFYPSDIAVEWESNGQPENNYKTTPPVLD |
| | SDGSFFLYSKLTVDKSRWQQGNVFSCSVMHEALHNHYTQKSLSLS |
| | PGK |
| KF032 | QVQLQESGGGLVQPGGSLRLSCAASGFRFSTSIMTWYRQAPGKERE |
| SEQ ID NO: 150 | LVASISSDSRRTNYAESVKGRFTISRDNEQRMVFLEMNSLKPVDTA |
| (CD36) | VYFCTRPNIRGRDWGQGTQVTVSSEPKSCDKTHTCPPCPAPELLGG |
| | PSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKA |
| | LPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQG |
| | NVFSCSVMHEALHNHYTQKSLSLSPGK |
| KF033 | QVQLQESGGGLVQLGGSLRLSCAASGFTFSNYYMSWYRQAPGKER |
| SEQ ID NO: 151 | ELVGGINMTGGSADYVDSVKGRFTISRDNTKNTVYLQMNSLRVED |
| (CD36) | TAVYYCHTFSVWPPNNYWGQGTQVTVSSEPKSCDKTHTCPPCPAP |
| | ELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCK |
| | VSNKALPAPIEKTISKAKGQPREPQVYTLPPSREEMTKNQVSLTCLV |
| | KGFYPSDIAVEWESNGQPENNYKTTPPVLDSDGSFFLYSKLTVDKS |
| | RWQQGNVFSCSVMHEALHNHYTQKSLSLSPGK |
| KF034 | QVQLQESGGGLVQPGGSLRLSCAASGFSFSTYAMSWYRQPPGKER |
| SEQ ID NO: 152 | ELVALITTTGGTTDYADSVKGRFTISRDDAEKTVSLQMNSLKPEDT |
| (CD36) | AVYYCSAWRATLLKPYWGQGTQVTVSSEPKSCDKTHTCPPCPAPE |
| | LLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKV |
| | SNKALPAPIEKTISKAKGQPREPQVYTLPPSREEMTKNQVSLTCLVK |
| | GFYPSDIAVEWESNGQPENNYKTTPPVLDSDGSFFLYSKLTVDKSR |
| | WQQGNVFSCSVMHEALHNHYTQKSLSLSPGK |
| KF035 | QVQLQESGGGLVQPGGSLRLSCAASGRTFSSYPMGWFRQAPGKER |
| SEQ ID NO: 153 | EFVAAIDWSGDITYYADSVKGRFAISRDNAKNMVYLQMNSLKPED |
| (CD36) | TAVYYCNAGPLDSWERNDFPSWGQGTQVTVSSEPKSCDKTHTCPP |
| | CPAPELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGK |
| KF036 | QVQLQESGGGLVQPGGSLRLSCAASGRTFSSYPMGWFRQAPGKER |
| SEQ ID NO: 154 | EFVAAIDWSGDITYYADSVKGRFAISRDNAKNMVYLQMNSLKPED |
| (CD36) | TAVYYCNAGPLDSWERNDFPSWDQGTQVTVSSEPKSCDKTHTCPP |
| | CPAPELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGK |
| KF037 | QVQLQESGGGLAQPGGSLRLSCAASGFTVSTYAMSWYRQAPGKER |
| SEQ ID NO: 155 | ELVALITSAGGITDYADSVKGRFTIARDDAEKTVSLQMNSLKPEDT |
| (CD36) | AVYYCSAWRATLFKPYWGQGTQVTVSSEPKSCDKTHTCPPCPAPE |
| | LLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKV |
| | SNKALPAPIEKTISKAKGQPREPQVYTLPPSREEMTKNQVSLTCLVK |
| | GFYPSDIAVEWESNGQPENNYKTTPPVLDSDGSFFLYSKLTVDKSR |
| | WQQGNVFSCSVMHEALHNHYTQKSLSLSPGK |
| KF038 | QVQLQESGGGLVQAGGSLRLSCAASGDTICTSAMAWYRQAPGKER |
| SEQ ID NO: 156 | ELVAGISSGGTVYYADSVKGRFTISRDNAKNTVYLQMNTLKPEDTA |
| (CD36) | VYYCNAGPEVHDDVFRLCGPTTDYWGQGTQVTVSSEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTKN |
| | QVSLTCLVKGFYPSDIAVEWESNGQPENNYKTTPPVLDSDGSFFLY |
| | SKLTVDKSRWQQGNVFSCSVMHEALHNHYTQKSLSLSPGK |
| KF039 | QVQLQESGGGLVQPGGSLRLSCAASGFTLDYYAIGWFRQAPGKER |
| SEQ ID NO: 157 | EGIVYISNSDGNTYYADSVKGRFTISRDNAKNTVYLQMNSLKPEDT |
| (CD36) | AVYYCAADQGSSVMHGFIEEWYDYWGQGTQVTVSSEPKSCDKTH |
| | TCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTKN |
| | QVSLTCLVKGFYPSDIAVEWESNGQPENNYKTTPPVLDSDGSFFLY |
| | SKLTVDKSRWQQGNVFSCSVMHEALHNHYTQKSLSLSPGK |
| KF040 | QVQLQESGGGLVQAGGSLRLSCAASGRTFSSYAMGWFRQAPGKER |
| SEQ ID NO: 158 | EFVAAISWSGGRTYYDDPVKGRFTISRDNAKNTVYLQMNSLKPED |
| (CD36) | TAVYYCNLDPEYSGTWNPWAGSGYWGQGTQVTVSSEPKSCDKTH |
| | TCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTKN |
| | QVSLTCLVKGFYPSDIAVEWESNGQPENNYKTTPPVLDSDGSFFLY |
| | SKLTVDKSRWQQGNVFSCSVMHEALHNHYTQKSLSLSPGK |

TABLE 7
Exemplary embodiments of polypeptide chains comprising three antigen
binding domains (VHH-linker-CH2-CH3-linker-VHH-linker-VHH)
(may comprise a His-tag or not)

| Code | |
|---|---|
| SEQ ID NO: | |
| (specificity) | SEQUENCE |
| KF061 | QVQLQESGGGLVQPGGSLRLSCAASGFTFSSYAMSWYRQAPGKE |
| SEQ ID NO: 159 | RELVALITSAGGTTDYADSVKGRFTISRDDAEKTVSLQMNSLKPE |
| (CD36/PD1/CD47) | DTAVYYCSAWRASLYEPYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGKAPAPAPAP |
| | APKAQVQLQESGGGWVQPGGSLRLSCVASGFTFSRSSMSWHRQA |
| | PGKERDLVAIISSSGIITHYSDSVKGRFTISRDNAKDTVYLQMNSLK |
| | PEDTAVYVCNAVLDDDSGDYWGQGTQVTVSSGGGGSGGGGSGG |
| | GGSQVQLQESGGGLVQPGGSLRLSCAASRFDFSVNDIRWYRQAP |
| | GNERELVARITGGGRTDYADSVKGRFTISRDNAKNTVYLQMNNL |
| | KPEDTAVYYCWGRGYWGQGTQVTVSS |
| KF042 | QVQLQESGGGLVQPGGSLRLSCAASGFTFSSYAMSWYRQAPGKE |
| SEQ ID NO: 160 | RELVALITSAGGTTDYADSVKGRFTISRDDAEKTVSLQMNSLKPE |
| (CD36/HEWL/HEWL) | DTAVYYCSAWRASLYEPYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGKAPAPAPAP |
| | APKAQVQLVESGGGSVQAGGSLRLSCAASGSTDSIEYMTWFRQA |
| | PGKAREGVAALYTHTGNTYYTDSVKGRFTISQDKAKNMAYLRM |
| | DSVKSEDTAIYTCGATRKYVPVRFALDQSSYDYWGQGTQVTVSS |
| | GGGGSGGGGSGGGGSQVQLVESGGGSVQAGGSLRLSCAASGSTD |
| | SIEYMTWFRQAPGKAREGVAALYTHTGNTYYTDSVKGRFTISQD |
| | KAKNMAYLRMDSVKSEDTAIYTCGATRKYVPVRFALDQSSYDY |
| | WGQGTQVTVSS |
| KF043 | QVQLQESGGGLVQPGGSLRLSCAASGFTFSNYAMSWYRQTPGKE |
| SEQ ID NO: 161 | RELVAAMQSTGGTTYYADSVKGRFTISRDDAKNTVYLQMNSLKP |
| (CD36/HEWL/HEWL) | EDTAVYYCNAVTLQGWSEYDYWGQGTQVTVSSEPKSCDKTHTC |
| | PPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGKAP |
| | APAPAPAPKAQVQLVESGGGSVQAGGSLRLSCAASGSTDSIEYMT |
| | WFRQAPGKAREGVAALYTHTGNTYYTDSVKGRFTISQDKAKNM |
| | AYLRMDSVKSEDTAIYTCGATRKYVPVRFALDQSSYDYWGQGTQ |
| | VTVSSGGGGSGGGGSGGGGSQVQLVESGGGSVQAGGSLRLSCAA |
| | SGSTDSIEYMTWFRQAPGKAREGVAALYTHTGNTYYTDSVKGRF |
| | TISQDKAKNMAYLRMDSVKSEDTAIYTCGATRKYVPVRFALDQS |
| | SYDYWGQGTQVTVSS |
| KF044 | QVQLQESGGGLVQPGGSLRLSCTASGFTFSSYAMSWYRQAPGKE |
| SEQ ID NO: 162 | RELVAAIPHDGGSIYYVDSVKGRFSISRDNAKNTVYLQMNSLKPE |
| (CD36/HEWL/HEWL) | DTAVYYCNAVTLQGWREYDYWGQGTQVTVSSEPKSCDKTHTCP |
| | PCPAPELLGGPSVFLFPPKPKDTLMISRTPEVTCVVVDVSHEDPEV |
| | KFNWYVDGVEVHNAKTKPREEQYNSTYRVVSVLTVLHQDWLNG |
| | KEYKCKVSNKALPAPIEKTISKAKGQPREPQVYTLPPSREEMTKN |
| | QVSLTCLVKGFYPSDIAVEWESNGQPENNYKTTPPVLDSDGSFFL |
| | YSKLTVDKSRWQQGNVFSCSVMHEALHNHYTQKSLSLSPGKAPA |
| | PAPAPAPKAQVQLVESGGGSVQAGGSLRLSCAASGSTDSIEYMTW |
| | FRQAPGKAREGVAALYTHTGNTYYTDSVKGRFTISQDKAKNMAY |
| | LRMDSVKSEDTAIYTCGATRKYVPVRFALDQSSYDYWGQGTQVT |
| | VSSGGGGSGGGGSGGGGSQVQLVESGGGSVQAGGSLRLSCAASG |
| | STDSIEYMTWFRQAPGKAREGVAALYTHTGNTYYTDSVKGRFTIS |
| | QDKAKNMAYLRMDSVKSEDTAIYTCGATRKYVPVRFALDQSSY |
| | DYWGQGTQVTVSS |
| KF045 | QVQLQESGGGLVQPGGSLRLSCAASGLAFSTYAMSWYRQAPGKE |
| SEQ ID NO: 163 | RELVALITSAGGTTDYADSVKGRFTISRDDAEKTVSLQMNSLKPE |
| (CD36/HEWL/HEWL) | DTAVYYCSAWRATLFKPYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGKAPAPAPAP |
| | APKAQVQLVESGGGSVQAGGSLRLSCAASGSTDSIEYMTWFRQA |
| | PGKAREGVAALYTHTGNTYYTDSVKGRFTISQDKAKNMAYLRM |
| | DSVKSEDTAIYTCGATRKYVPVRFALDQSSYDYWGQGTQVTVSS |
| | GGGGSGGGGSGGGGSQVQLVESGGGSVQAGGSLRLSCAASGSTD |
| | SIEYMTWFRQAPGKAREGVAALYTHTGNTYYTDSVKGRFTISQD |
| | KAKNMAYLRMDSVKSEDTAIYTCGATRKYVPVRFALDQSSYDY |
| | WGQGTQVTVSS |
| KF046 | QVQLQESGGGLVQPGGSLRLSCAASGFTSGFTFSSYAMSWYRQAP |
| SEQ ID NO: 164 | GKERELVAAITNAGDSTHYADSVKGRFTISRDNAKNTVYLLMNSL |
| (CD36/HEWL/HEWL) | KPEDTAAYYCNAVTLQGWSEYDYWGQGTQVTVSSEPKSCDKTH |
| | TCPPCPAPELLGGPSVFLFPPKPKDTLMISRTPEVTCVVVDVSHEDP |
| | EVKFNWYVDGVEVHNAKTKPREEQYNSTYRVVSVLTVLHQDWL |
| | NGKEYKCKVSNKALPAPIEKTISKAKGQPREPQVYTLPPSREEMT |
| | KNQVSLTCLVKGFYPSDIAVEWESNGQPENNYKTTPPVLDSDGSF |
| | FLYSKLTVDKSRWQQGNVFSCSVMHEALHNHYTQKSLSLSPGKA |
| | PAPAPAPAPKAQVQLVESGGGSVQAGGSLRLSCAASGSTDSIEYM |
| | TWFRQAPGKAREGVAALYTHTGNTYYTDSVKGRFTISQDKAKN |
| | MAYLRMDSVKSEDTAIYTCGATRKYVPVRFALDQSSYDYWGQG |
| | TQVTVSSGGGGSGGGGSGGGGSQVQLVESGGGSVQAGGSLRLSC |
| | AASGSTDSIEYMTWFRQAPGKAREGVAALYTHTGNTYYTDSVKG |
| | RFTISQDKAKNMAYLRMDSVKSEDTAIYTCGATRKYVPVRFALD |
| | QSSYDYWGQGTQVTVSS |
| KF047 | QVQLQESGGGLVQPGGSLRLSCAASGFSLDYFAIGWFRQAPGKER |
| SEQ ID NO: 165 | EGISCISSSGSNTYYADSVKGRFTISRDNAKNTVYLQMNSLKPEDT |
| (CD36/HEWL/HEWL) | AVYYCAAHDYCSRAPYDYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGKAPAPAPAP |
| | APKAQVQLVESGGGSVQAGGSLRLSCAASGSTDSIEYMTWFRQA |
| | PGKAREGVAALYTHTGNTYYTDSVKGRFTISQDKAKNMAYLRM |
| | DSVKSEDTAIYTCGATRKYVPVRFALDQSSYDYWGQGTQVTVSS |
| | GGGGSGGGGSGGGGSQVQLVESGGGSVQAGGSLRLSCAASGSTD |
| | SIEYMTWFRQAPGKAREGVAALYTHTGNTYYTDSVKGRFTISQD |
| | KAKNMAYLRMDSVKSEDTAIYTCGATRKYVPVRFALDQSSYDY |
| | WGQGTQVTVSS |
| KF048 | QVQLQESGGGLVQPGESLRLSCAASGFTFSNYAMSWYRQTPGKE |
| SEQ ID NO: 166 | RELVAAMQSTGGTTYYADSVKGRFTISRDDAKNTVYLQMNSLKP |
| (CD36/HEWL/HEWL) | EDTAVYYCNAVTLQGWSEYDYWGQGTQVTVSSEPKSCDKTHTC |
| | PPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGKAP |
| | APAPAPAPKAQVQLVESGGGSVQAGGSLRLSCAASGSTDSIEYMT |
| | WFRQAPGKAREGVAALYTHTGNTYYTDSVKGRFTISQDKAKNM |
| | AYLRMDSVKSEDTAIYTCGATRKYVPVRFALDQSSYDYWGQGTQ |
| | VTVSSGGGGSGGGGSGGGGSQVQLVESGGGSVQAGGSLRLSCAA |
| | SGSTDSIEYMTWFRQAPGKAREGVAALYTHTGNTYYTDSVKGRF |
| | TISQDKAKNMAYLRMDSVKSEDTAIYTCGATRKYVPVRFALDQS |
| | SYDYWGQGTQVTVSS |
| KF049 | QVQLQESGGGLVQPGGSLRLSCAASGFRFSSSIMSWYRQAPEKER |
| SEQ ID NO: 167 | ELVASISSDSVRTNYAESVKGRFTISRDNEQSTLFLEMNNLKPVDT |
| (CD36/HEWL/HEWL) | AVYFCTRPSIRGRDWGQGTQVTVSSEPKSCDKTHTCPPCPAPELL |
| | GGPSVFLFPPKPKDTLMISRTPEVTCVVVDVSHEDPEVKFNWYVD |
| | GVEVHNAKTKPREEQYNSTYRVVSVLTVLHQDWLNGKEYKCKV |
| | SNKALPAPIEKTISKAKGQPREPQVYTLPPSREEMTKNQVSLTCLV |
| | KGFYPSDIAVEWESNGQPENNYKTTPPVLDSDGSFFLYSKLTVDK |
| | SRWQQGNVFSCSVMHEALHNHYTQKSLSLSPGKAPAPAPAPAPK |
| | AQVQLVESGGGSVQAGGSLRLSCAASGSTDSIEYMTWFRQAPGK |
| | AREGVAALYTHTGNTYYTDSVKGRFTISQDKAKNMAYLRMDSV |
| | KSEDTAIYTCGATRKYVPVRFALDQSSYDYWGQGTQVTVSSGGG |
| | GSGGGGSGGGGSQVQLVESGGGSVQAGGSLRLSCAASGSTDSIEY |
| | MTWFRQAPGKAREGVAALYTHTGNTYYTDSVKGRFTISQDKAK |
| | NMAYLRMDSVKSEDTAIYTCGATRKYVPVRFALDQSSYDYWGQ |
| | GTQVTVSS |
| KF050 | QVQLQESGGGLVQPGGSLRLSCTASGRTGSADVMGWFRRAPGKD |
| SEQ ID NO: 168 | REFVAAADWRGLNTWYRDSVKGRFTISRDNAKSTMYLQMNSLK |
| (CD36/HEWL/HEWL) | PTDTAVYYCNTGGWAFSPGASWGQGTQVTVSSEPKSCDKTHTCP |
| | PCPAPELLGGPSVFLFPPKPKDTLMISRTPEVTCVVVDVSHEDPEV |
| | KFNWYVDGVEVHNAKTKPREEQYNSTYRVVSVLTVLHQDWLNG |
| | KEYKCKVSNKALPAPIEKTISKAKGQPREPQVYTLPPSREEMTKN |
| | QVSLTCLVKGFYPSDIAVEWESNGQPENNYKTTPPVLDSDGSFFL |
| | YSKLTVDKSRWQQGNVFSCSVMHEALHNHYTQKSLSLSPGKAPA |
| | PAPAPAPKAQVQLVESGGGSVQAGGSLRLSCAASGSTDSIEYMTW |
| | FRQAPGKAREGVAALYTHTGNTYYTDSVKGRFTISQDKAKNMAY |
| | LRMDSVKSEDTAIYTCGATRKYVPVRFALDQSSYDYWGQGTQVT |
| | VSSGGGGSGGGGSGGGGSQVQLVESGGGSVQAGGSLRLSCAASG |
| | STDSIEYMTWFRQAPGKAREGVAALYTHTGNTYYTDSVKGRFTIS |
| | QDKAKNMAYLRMDSVKSEDTAIYTCGATRKYVPVRFALDQSSY |
| | DYWGQGTQVTVSS |
| KF051 | QVQLQESGGGLVQPGGSLRLSCAASGFRFSTSIMTWYRQAPGKER |
| SEQ ID NO: 169 | ELVASISSDSRRTNYAESVKGRFTISRDNEQRMVFLEMNSLKPVDT |
| (CD36/HEWL/HEWL) | AVYFCTRPNIRGRDWGQGTQVTVSSEPKSCDKTHTCPPCPAPELL |
| | GGPSVFLFPPKPKDTLMISRTPEVTCVVVDVSHEDPEVKFNWYVD |
| | GVEVHNAKTKPREEQYNSTYRVVSVLTVLHQDWLNGKEYKCKV |
| | SNKALPAPIEKTISKAKGQPREPQVYTLPPSREEMTKNQVSLTCLV |
| | KGFYPSDIAVEWESNGQPENNYKTTPPVLDSDGSFFLYSKLTVDK |
| | SRWQQGNVFSCSVMHEALHNHYTQKSLSLSPGKAPAPAPAPAPK |
| | AQVQLVESGGGSVQAGGSLRLSCAASGSTDSIEYMTWFRQAPGK |
| | AREGVAALYTHTGNTYYTDSVKGRFTISQDKAKNMAYLRMDSV |
| | KSEDTAIYTCGATRKYVPVRFALDQSSYDYWGQGTQVTVSSGGG |
| | GSGGGGSGGGGSQVQLVESGGGSVQAGGSLRLSCAASGSTDSIEY |
| | MTWFRQAPGKAREGVAALYTHTGNTYYTDSVKGRFTISQDKAK |
| | NMAYLRMDSVKSEDTAIYTCGATRKYVPVRFALDQSSYDYWGQ |
| | GTQVTVSS |
| KF052 | QVQLQESGGGLVQLGGSLRLSCAASGFTFSNYYMSWYRQAPGKE |
| SEQ ID NO: 170 | RELVGGINMTGGSADYVDSVKGRFTISRDNTKNTVYLQMNSLRV |
| (CD36/HEWL/HEWL) | EDTAVYYCHTFSVWPPNNYWGQGTQVTVSSEPKSCDKTHTCPPC |
| | PAPELLGGPSVFLFPPKPKDTLMISRTPEVTCVVVDVSHEDPEVKF |
| | NWYVDGVEVHNAKTKPREEQYNSTYRVVSVLTVLHQDWLNGKE |
| | YKCKVSNKALPAPIEKTISKAKGQPREPQVYTLPPSREEMTKNQVS |
| | LTCLVKGFYPSDIAVEWESNGQPENNYKTTPPVLDSDGSFFLYSKL |
| | TVDKSRWQQGNVFSCSVMHEALHNHYTQKSLSLSPGKAPAPAPA |
| | PAPKAQVQLVESGGGSVQAGGSLRLSCAASGSTDSIEYMTWFRQ |
| | APGKAREGVAALYTHTGNTYYTDSVKGRFTISQDKAKNMAYLR |
| | MDSVKSEDTAIYTCGATRKYVPVRFALDQSSYDYWGQGTQVTVS |
| | SGGGGSGGGGSGGGGSQVQLVESGGGSVQAGGSLRLSCAASGST |
| | DSIEYMTWFRQAPGKAREGVAALYTHTGNTYYTDSVKGRFTISQ |
| | DKAKNMAYLRMDSVKSEDTAIYTCGATRKYVPVRFALDQSSYD |
| | YWGQGTQVTVSS |
| KF053 | QVQLQESGGGLVQPGGSLRLSCAASGFSFSTYAMSWYRQPPGKE |
| SEQ ID NO: 171 | RELVALITTTGGTTDYADSVKGRFTISRDDAEKTVSLQMNSLKPE |
| (CD36/HEWL/HEWL) | DTAVYYCSAWRATLLKPYWGQGTQVTVSSEPKSCDKTHTCPPCP |
| | APELLGGPSVFLFPPKPKDTLMISRTPEVTCVVVDVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGKAPAPAPAP |
| | APKAQVQLVESGGGSVQAGGSLRLSCAASGSTDSIEYMTWFRQA |
| | PGKAREGVAALYTHTGNTYYTDSVKGRFTISQDKAKNMAYLRM |
| | DSVKSEDTAIYTCGATRKYVPVRFALDQSSYDYWGQGTQVTVSS |
| | GGGGSGGGGSGGGGSQVQLVESGGGSVQAGGSLRLSCAASGSTD |
| | SIEYMTWFRQAPGKAREGVAALYTHTGNTYYTDSVKGRFTISQD |
| | KAKNMAYLRMDSVKSEDTAIYTCGATRKYVPVRFALDQSSYDY |
| | WGQGTQVTVSS |
| KF054 | QVQLQESGGGLVQPGGSLRLSCAASGRTFSSYPMGWFRQAPGKE |
| SEQ ID NO: 172 | REFVAAIDWSGDITYYADSVKGRFAISRDNAKNMVYLQMNSLKP |
| (CD36/HEWL/HEWL) | EDTAVYYCNAGPLDSWERNDFPSWGQGTQVTVSSEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGKAP |
| | APAPAPAPKAQVQLVESGGGSVQAGGSLRLSCAASGSTDSIEYMT |
| | WFRQAPGKAREGVAALYTHTGNTYYTDSVKGRFTISQDKAKNM |
| | AYLRMDSVKSEDTAIYTCGATRKYVPVRFALDQSSYDYWGQGTQ |
| | VTVSSGGGGSGGGGSGGGGSQVQLVESGGGSVQAGGSLRLSCAA |
| | SGSTDSIEYMTWFRQAPGKAREGVAALYTHTGNTYYTDSVKGRF |
| | TISQDKAKNMAYLRMDSVKSEDTAIYTCGATRKYVPVRFALDQS |
| | SYDYWGQGTQVTVSS |
| KF055 | QVQLQESGGGLVQPGGSLRLSCAASGRTFSSYPMGWFRQAPGKE |
| SEQ ID NO: 173 | REFVAAIDWSGDITYYADSVKGRFAISRDNAKNMVYLQMNSLKP |
| (CD36/HEWL/HEWL) | EDTAVYYCNAGPLDSWERNDFPSWDQGTQVTVSSEPKSCDKTHT |
| | CPPCPAPELLGGPSVFLFPPKPKDTLMISRTPEVTCVVVDVSHEDPE |
| | VKFNWYVDGVEVHNAKTKPREEQYNSTYRVVSVLTVLHQDWLN |
| | GKEYKCKVSNKALPAPIEKTISKAKGQPREPQVYTLPPSREEMTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGKAP |
| | APAPAPAPKAQVQLVESGGGSVQAGGSLRLSCAASGSTDSIEYMT |
| | WFRQAPGKAREGVAALYTHTGNTYYTDSVKGRFTISQDKAKNM |
| | AYLRMDSVKSEDTAIYTCGATRKYVPVRFALDQSSYDYWGQGTQ |
| | VTVSSGGGGSGGGGSGGGGSQVQLVESGGGSVQAGGSLRLSCAA |
| | SGSTDSIEYMTWFRQAPGKAREGVAALYTHTGNTYYTDSVKGRF |
| | TISQDKAKNMAYLRMDSVKSEDTAIYTCGATRKYVPVRFALDQS |
| | SYDYWGQGTQVTVSS |
| KF056 | QVQLQESGGGLAQPGGSLRLSCAASGFTVSTYAMSWYRQAPGKE |
| SEQ ID NO: 174 | RELVALITSAGGITDYADSVKGRFTIARDDAEKTVSLQMNSLKPE |
| (CD36/HEWL/HEWL) | DTAVYYCSAWRATLFKPYWGQGTQVTVSSEPKSCDKTHTCPPCP |
| | APELLGGPSVFLFPPKPKDTLMISRTPEVTCVVVDVSHEDPEVKEN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | TCLVKGFYPSDIAVEWESNGQPENNYKTTPPVLDSDGSFFLYSKLT |
| | VDKSRWQQGNVFSCSVMHEALHNHYTQKSLSLSPGKAPAPAPAP |
| | APKAQVQLVESGGGSVQAGGSLRLSCAASGSTDSIEYMTWFRQA |
| | PGKAREGVAALYTHTGNTYYTDSVKGRFTISQDKAKNMAYLRM |
| | DSVKSEDTAIYTCGATRKYVPVRFALDQSSYDYWGQGTQVTVSS |
| | GGGGSGGGGSGGGGSQVQLVESGGGSVQAGGSLRLSCAASGSTD |
| | SIEYMTWFRQAPGKAREGVAALYTHTGNTYYTDSVKGRFTISQD |
| | KAKNMAYLRMDSVKSEDTAIYTCGATRKYVPVRFALDQSSYDY |
| | WGQGTQVTVSS |
| KF057 | QVQLQESGGGLVQAGGSLRLSCAASGDTICTSAMAWYRQAPGKE |
| SEQ ID NO: 175 | RELVAGISSGGTVYYADSVKGRFTISRDNAKNTVYLQMNTLKPED |
| (CD36/HEWL/HEWL) | TAVYYCNAGPEVHDDVFRLCGPTTDYWGQGTQVTVSSEPKSCDK |
| | THTCPPCPAPELLGGPSVFLFPPKPKDTLMISRTPEVTCVVVDVSH |
| | EDPEVKFNWYVDGVEVHNAKTKPREEQYNSTYRVVSVLTVLHQ |
| | DWLNGKEYKCKVSNKALPAPIEKTISKAKGQPREPQVYTLPPSRE |
| | EMTKNQVSLTCLVKGFYPSDIAVEWESNGQPENNYKTTPPVLDSD |
| | GSFFLYSKLTVDKSRWQQGNVFSCSVMHEALHNHYTQKSLSLSP |
| | GKAPAPAPAPAPKAQVQLVESGGGSVQAGGSLRLSCAASGSTDSI |
| | EYMTWFRQAPGKAREGVAALYTHTGNTYYTDSVKGRFTISQDKA |
| | KNMAYLRMDSVKSEDTAIYTCGATRKYVPVRFALDQSSYDYWG |
| | QGTQVTVSSGGGGSGGGGSGGGGSQVQLVESGGGSVQAGGSLRL |
| | SCAASGSTDSIEYMTWFRQAPGKAREGVAALYTHTGNTYYTDSV |
| | KGRFTISQDKAKNMAYLRMDSVKSEDTAIYTCGATRKYVPVRFA |
| | LDQSSYDYWGQGTQVTVSS |
| KF058 | QVQLQESGGGLVQPGGSLRLSCAASGFTLDYYAIGWFRQAPGKE |
| SEQ ID NO: 176 | REGIVYISNSDGNTYYADSVKGRFTISRDNAKNTVYLQMNSLKPE |
| (CD36/HEWL/HEWL) | DTAVYYCAADQGSSVMHGFIEEWYDYWGQGTQVTVSSEPKSCD |
| | KTHTCPPCPAPELLGGPSVFLFPPKPKDTLMISRTPEVTCVVVDVS |
| | HEDPEVKFNWYVDGVEVHNAKTKPREEQYNSTYRVVSVLTVLH |
| | QDWLNGKEYKCKVSNKALPAPIEKTISKAKGQPREPQVYTLPPSR |
| | EEMTKNQVSLTCLVKGFYPSDIAVEWESNGQPENNYKTTPPVLDS |
| | DGSFFLYSKLTVDKSRWQQGNVFSCSVMHEALHNHYTQKSLSLS |
| | PGKAPAPAPAPAPKAQVQLVESGGGSVQAGGSLRLSCAASGSTDS |
| | IEYMTWFRQAPGKAREGVAALYTHTGNTYYTDSVKGRFTISQDK |
| | AKNMAYLRMDSVKSEDTAIYTCGATRKYVPVRFALDQSSYDYW |
| | GQGTQVTVSSGGGGSGGGGSGGGGSQVQLVESGGGSVQAGGSL |
| | RLSCAASGSTDSIEYMTWFRQAPGKAREGVAALYTHTGNTYYTD |
| | SVKGRFTISQDKAKNMAYLRMDSVKSEDTAIYTCGATRKYVPVR |
| | FALDQSSYDYWGQGTQVTVSS |
| KF059 | QVQLQESGGGLVQAGGSLRLSCAASGRTESSYAMGWFRQAPGKE |
| SEQ ID NO: 177 | REFVAAISWSGGRTYYDDPVKGRFTISRDNAKNTVYLQMNSLKPE |
| (CD36/HEWL/HEWL) | DTAVYYCNLDPEYSGTWNPWAGSGYWGQGTQVTVSSEPKSCDK |
| | THTCPPCPAPELLGGPSVFLFPPKPKDTLMISRTPEVTCVVVDVSH |
| | EDPEVKFNWYVDGVEVHNAKTKPREEQYNSTYRVVSVLTVLHQ |
| | DWLNGKEYKCKVSNKALPAPIEKTISKAKGQPREPQVYTLPPSRE |
| | EMTKNQVSLTCLVKGFYPSDIAVEWESNGQPENNYKTTPPVLDSD |
| | GSFFLYSKLTVDKSRWQQGNVFSCSVMHEALHNHYTQKSLSLSP |
| | GKAPAPAPAPAPKAQVQLVESGGGSVQAGGSLRLSCAASGSTDSI |
| | EYMTWFRQAPGKAREGVAALYTHTGNTYYTDSVKGRFTISQDKA |
| | KNMAYLRMDSVKSEDTAIYTCGATRKYVPVRFALDQSSYDYWG |
| | QGTQVTVSSGGGGSGGGGSGGGGSQVQLVESGGGSVQAGGSLRL |
| | SCAASGSTDSIEYMTWFRQAPGKAREGVAALYTHTGNTYYTDSV |
| | KGRFTISQDKAKNMAYLRMDSVKSEDTAIYTCGATRKYVPVRFA |
| | LDQSSYDYWGQGTQVTVSS |

SEQ ID NO: 178
EPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSL
SLSPGKAPAPAPAPAPKAQVQLQESGGGWVQPGGSLRLSCVASGFTFSRSSMSWHR
QAPGKERDLVAIISSSGIITHYSDSVKGRFTISRDNAKDTVYLQMNSLKPEDTAVYVC
NAVLDDDSGDYWGQGTQVTVSSGGGGSGGGGSGGGGSQVQLQESGGGLVQPGGS
LRLSCAASRFDFSVNDIRWYRQAPGNERELVARITGGGRTDYADSVKGRFTISRDNA
KNTVYLQMNNLKPEDTAVYYCWGRGYWGQGTQVTVSS
SEQ ID NO: 179
APAPAPAPAPKAQVQLQESGGGWVQPGGSLRLSCVASGFTFSRSSMSWHRQAPGKE
RDLVAIISSSGIITHYSDSVKGRFTISRDNAKDTVYLQMNSLKPEDTAVYVCNAVLDD
DSGDYWGQGTQVTVSSGGGGSGGGGSGGGGSQVQLQESGGGLVQPGGSLRLSCAA
SRFDFSVNDIRWYRQAPGNERELVARITGGGRTDYADSVKGRFTISRDNAKNTVYLQ
MNNLKPEDTAVYYCWGRGYWGQGTQVTVSS
SEQ ID NO: 180 (natural human IgG1 hinge region)
EPKSCDKTHTCPPCP
SEQ ID NO: 181 (Linker-HL1)
EPKIPQPQPKPQPQPQPGGSGSAEAAAKAPKAP
SEQ ID NO: 182 (flexible linker-FL2)
GGGGSGGGGS
SEQ ID NO: 183 (flexible linker-FL18)
GGGGSGGGGSGGGGS
SEQ ID NO: 184 (flexible linker-FL4)
GGGGSGGGGSGGGGSGGGGSGGGGS
SEQ ID NO: 185 (flexible linker-FL5)
GGGGSGGGGSGGGGSGGGGSGGGGSGGGGS
SEQ ID NO: 186 (flexible linker)
(GGGGS)ₙ wherein n is an integer selected from 1 to 10
SEQ ID NO: 187 (rigid linker-RL5)
PAPAPKA
SEQ ID NO: 188 (rigid linker-RL7)
APAPAPAPAPKA
SEQ ID NO: 189 (rigid linker-RL12)
APAPAPAPAPAPAPAPAPAPKA
SEQ ID NO: 190 (rigid linker)
(X(PAPAP))ₙKA wherein n is an integer selected from 1 to 10, wherein X is present or
absent and is A
SEQ ID NO: 191 (helical linker-RL1)
AEAAAKEAAAKA
SEQ ID NO: 192 (helical linker-RL2)
AEAAAKEAAAKEAAAKA
SEQ ID NO: 193 (helical linker-RL4)
AEAAAKEAAAKEAAAKEAAAKEAAAKA
SEQ ID NO: 194 (helical linker
X(EAAAK)ₙY wherein n is an integer selected from 1 to 10, more preferably 2-5 wherein X
and Y are independently present or absent and is preferably A
SEQ ID NO: 195 Anti-HEWL VHH
XVQLVESGGGSVQAGGSLRLSCAASGSTDSIEYMTWFRQAPGKAREGVAALYTHTG
NTYYTDSVKGRFTISQDKAKNMAYLRMDSVKSEDTAIYTCGATRKYVPVRFALDQS
SYDYWGQGTQVTVSS
Wherein X is E or Q
SEQ ID NO: 196 Anti-4HEM VHH
XVQLVESGGGLVQAGGSLRLSCAASESTESNYAMGWFRQAPGPEREFVATISQTGSH
TYYRNSVKGRFTISRDNAKNTVYLQMNNMKPEDTAVYYCAAGDNYYYTRTYEYD
YWGQGTQVTVSS
Wherein X is E or Q
SEQ ID NO: 197 Anti-CD3 VHH
XVQLVESGGGLVQPGGSLRLSCAASGDIYKSFDMGWYRQAPGKQRDLVAVIGSRG
NNRGRTNYADSVKGRFTISRDGTGNTVYLLMNKLRPEDTAIYYCNTAPLVAGRPWG
RGTLVTVSS
Wherein X is E or D
SEQ ID NO: 198 natural human IgG1 CH3
GQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 199 natural human IgG1 CH2
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAK
SEQ ID NO: 200 mutated human IgG1 hinge region
EPKSSDKTHTCPPCP
SEQ ID NO: 201 mutated human IgG1 hinge region
EPKSSDKTHTSPPSP
SEQ ID NO: 202 mutated human IgG1 hinge region
DKTHTCPPC
SEQ ID NO: 203 natural human IgG2 hinge region
ERKCCVECPPCP
SEQ ID NO: 204 mutated human IgG2 hinge region
ERKSSVECPPCP
SEQ ID NO: 205 mutated human IgG2 hinge region
ERKSSVESPPCP
SEQ ID NO: 206 mutated human IgG2 hinge region
ERKSSVESPPSP
SEQ ID NO: 207 natural human IgG3 hinge region
ELKTPLGDTTHTCPRCPEPKSCDTPPPCPRCPEPKSCDTPPPCPRCPEPKSCDTPPPCPR
CP
SEQ ID NO: 208 mutated human IgG3 hinge region
EPKSSDTPPPCPRCP
SEQ ID NO: 209 mutated human IgG3 hinge region
EPKSSDTPPPSPRCP
SEQ ID NO: 210 mutated human IgG3 hinge region
EPKSSDTPPPSPRSP
SEQ ID NO: 211 natural human IgG4 hinge region
ESKYGPPCPSCP
SEQ ID NO: 212 mutated human IgG4 hinge region
ESKYGPPCPPCP
SEQ ID NO: 213 mutated human IgG4 hinge region
ESKYGPPSPSCP
SEQ ID NO: 214 mutated human IgG4 hinge region)
ESKYGPPSPSSP
SEQ ID NO: 215 Signal Peptide
MEWSWVFLFFLSVTTGVHS
SEQ ID NO: 216 Epitope Tag
HHHHHH
SEQ ID NO: 217 Natural human IgG1 constant region
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 218 Mutated CH3 domain (Chain A-mutations D399N; K370E; E356Q)
GQPREPQVYTLPPSRQEMTKNQVSLTCLVEGFYPSDIAVEWESNGQPENNYKTTPPV
LNSDGSFFLYSKLTVDKSRWQQGNVFSCSVMHEALHNHYTQKSLSLSPGK
SEQ ID NO: 219 Mutated CH3 domain (Chain B-mutations D399N; K439E; E357Q)
GQPREPQVYTLPPSREQMTKNQVSLTCLVKGFYPSDIAVEWESNGQPENNYKTTPPV
LNSDGSFFLYSKLTVDKSRWQQGNVFSCSVMHEALHNHYTQESLSLSPGK
SEQ ID NO: 220 Mutated human IgG1 constant region (Chain A-mutations D399N; K370E;
E356Q)
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQP
REPQVYTLPPSRQEMTKNQVSLTCLVEGFYPSDIAVEWESNGQPENNYKTTPPVLNS
DGSFFLYSKLTVDKSRWQQGNVFSCSVMHEALHNHYTQKSLSLSPGK
SEQ ID NO: 221 Mutated human IgG1 constant region (Chain B- mutations D399N; K439E; E357Q)
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQP
REPQVYTLPPSREQMTKNQVSLTCLVKGFYPSDIAVEWESNGQPENNYKTTPPVLNS
DGSFFLYSKLTVDKSRWQQGNVFSCSVMHEALHNHYTQESLSLSPGK
SEQ ID NO: 222 Mutated human IgG4 constant region
APEAAGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 223 (anti-HEW VHH)
QVQLVESGGGSVQAGGSLRLSCAASGSTDSIEYMTWFRQAPGKAREGVAALYTHTG
NTYYTDSVKGRFTISQDKAKNMAYLRMDSVKSEDTAIYTCGATRKYVPVRFALDQS
SYDYWGQGTQVTVSS
SEQ ID NO: 224 (CD36 antigen)
GDLLIQKTIKKQVVLEEGTIAFKNWVKTGTEVYRQFWIFDVQNPQEVMMNSSNIQV
KQRGPYTYRVRFLAKENVTQDAEDNTVSFLQPNGAIFEPSLSVGTEADNFTVLNLAV
AAASHIYQNQFVQMILNSLINKSKSSMFQVRTLRELLWGYRDPFLSLVPYPVTTTVG
LFYPYNNTADGVYKVFNGKDNISKVAIIDTYKGKRNLSYWESHCDMINGTDAASFPP
FVEKSQVLQFFSSDICRSIYAVFESDVNLKGIPVYRFVLPSKAFASPVENPDNYCFCTE
KIISKNCTSYGVLDISKCKEGRPVYISLPHFLYASPDVSEPIDGLNPNEEEHRTYLDIEPI
TGFTLQFAKRLQVNLLVKPSEKIQVLKNLKRNYIVPILWLNETGTIGDEKANMFRSQ
VTGKIN
SEQ ID NO: 268 (human CD36 protein: Uniprot Accession No. P16671-1)
MGCDRNCGLIAGAVIGAVLAVFGGILMPVGDLLIQKTIKKQVVLEEGTIAFKNWVKT
GTEVYRQFWIFDVQNPQEVMMNSSNIQVKQRGPYTYRVRFLAKENVTQDAEDNTV
SFLQPNGAIFEPSLSVGTEADNFTVLNLAVAAASHIYQNQFVQMILNSLINKSKSSMF
QVRTLRELLWGYRDPFLSLVPYPVTTTVGLFYPYNNTADGVYKVENGKDNISKVAII
DTYKGKRNLSYWESHCDMINGTDAASFPPFVEKSQVLQFFSSDICRSIYAVFESDVNL
KGIPVYRFVLPSKAFASPVENPDNYCFCTEKIISKNCTSYGVLDISKCKEGRPVYISLP
HFLYASPDVSEPIDGLNPNEEEHRTYLDIEPITGFTLQFAKRLQVNLLVKPSEKIQVLK
NLKRNYIVPILWLNETGTIGDEKANMFRSQVTGKINLLGLIEMILLSVGVVMFVAFMI
SYCACRSKTIK
SEQ ID NO: 225 (PD-1 antigen)
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHPSPSPRPAGQFQ
SEQ ID NO: 269 (human PD-1 protein: Uniprot Accession No. Q15116).
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHPSPSPRPAGQFQTLV
VGVVGGLLGSLVLLVWVLAVICSRAARGTIGARRTGQPLKEDPSAVPVFSVDYGEL
DFQWREKTPEPPVPCVPEQTEYATIVFPSGMGTSSPARRGSADGPRSAQPLRPEDGHC
SWPL
SEQ ID NO: 226 (CD47 antigen)
MWPLVAALLLGSACCGSAQLLFNKTKSVEFTFCNDTVVIPCFVTNMEAQNTTEVYV
KWKFKGRDIYTFDGALNKSTVPTDFSSAKIEVSQLLKGDASLKMDKSDAVSHTGNY
TCEVTELTREGETIIELKYRVVSWFSP
SEQ ID NO: 270 (human CD47 protein: Uniprot Accession No. Q08722-1).
MWPLVAALLLGSACCGSAQLLFNKTKSVEFTFCNDTVVIPCFVTNMEAQNTTEVYV
KWKFKGRDIYTFDGALNKSTVPTDFSSAKIEVSQLLKGDASLKMDKSDAVSHTGNY
TCEVTELTREGETIIELKYRVVSWFSPNENILIVIFPIFAILLFWGQFGIKTLKYRSGGM
DEKTIALLVAGLVITVIVIVGAILFVPGEYSLKNATGLGLIVTSTGILILLHYYVFSTAIG
LTSFVIAILVIQVIAYILAVVGLSLCIAACIPMHGPLLISGLSILALAQLLGLVYMKFVA
SNQKTIQPPRKAVEEPLNAFKESKGMMNDE
SEQ ID NO: 227 constant region of sdAbs of Table 6
EPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSL
SLSPGK
SEQ ID NO: 379 consensus of selected anti-PD-1 VHH sequences
QVQLQESGGGX₁ₐVQX_{1b}GX_{1c}SLRLSCAASGFTFSX_{1d}YGMSWVRQAPGEGLEWVSSI
DSX₁ₑGGTTX_{1f}YAX_{1g}SVX₁ₕGRFTISRDNAKNX₁ⱼX₁ₖYLQMNSLKPEDTAVYYCAKDF
LSWMPRGQGTQVTVSS
Wherein X₁ₐ is L or E; Wherein X_{1b} is A or P; Wherein X_{1c} is D or G; Wherein X_{1d} is N or S;
Wherein X₁ₑ is S or T; Wherein X_{1f} is K or R; Wherein X_{1g} is D or G; Wherein X1h is K or Q;
Wherein X1j is T or A and/or Wherein X1k is L or V.
SEQ ID NO: 380 consensus of selected anti-CD36 VHH sequences
QVQLQESGGGLX₂ₐQPGGSLRLSCAASGX_{2b}X_{2c}X_{2d}SX₂ₑYAMSWYRQX_{2f}PGKERELVA
LITX_{2g}X₂ₕGGX₂ᵢTDYADSVKGRFTI_{X2j}RDDAEKTVSLQMNSLKPEDTAVYYCSAWRA
X2kLX2lX2mPYWGQGTQVTVSS
wherein X₂ₐ is A or V, wherein X_{2b} is L or F, wherein X_{2c} is A, T or S, wherein X_{2d} is For V,
wherein X₂ₑ is T or S, wherein X_{2f} is A or P, wherein X_{2g} is S or T, wherein X₂ₕ is A or T,
wherein X₂ᵢ is I or T, wherein X₂ⱼ is A or S, wherein X₂ₖ is S or T, wherein X₂ₗ is F, Y or L
and/or wherein X₂ₘ is E or K.
SEQ ID NO: 381 consensus of selected anti-CD36 VHH sequences
QVQLQESGGGLVQPGX₃ₐSLRLSCX_{3b}ASGX_{3c}X_{3d}X₃ₑX_{3f}FTFSX_{3g}YAMSWYRQX₃ₕPGKE
RELVAAX₃ᵢX₃ⱼX₃ₖX₃ₗGX₃ₘX₃ₙX₃ₒX₃ₚYX_{3q}DSVKGRFX₃ᵣISRDX₃ₛAKNTVYLX₃ₜMNSLKP
EDTAX₃ᵤYYCNAVTLQGWX₃ᵥEYDYWGQGTQVTVSS
wherein X₃ₐ is G or E, wherein X_{3b} is A or T, wherein X_{3c} is absent or F, wherein X_{3d} is
absent or T, wherein X₃ₑ is absent or S, wherein X_{3f} is absent or G, wherein X_{3g} is S or N,
wherein X₃ₕ is A or T, wherein X₃ᵢ is M or I, wherein X₃ⱼ is Q, P or T, wherein X₃ₖ is S, H or
N, wherein X₃ₗ is T, A or D, wherein X₃ₘ is D or G, wherein X₃ₙ is S or T, wherein X₃ₒ is T
or I, wherein X₃ₚ is Y or H, wherein X_{3q} is A or V, wherein X₃ᵣ is S or T, wherein X₃ₛ is D or
N, wherein X₃ₜ is Q or L, wherein X₃ᵤ is V or A and/or wherein X₃ᵥ is S or R.
SEQ ID NO: 382 consensus of selected anti-CD36 VHH sequences
QVQLQESGGGLVQPGGSLRLSCAASGFRFSX₄ₐSIMX_{4b}WYRQAPX_{4c}KERELVASISSD
S
X_{4d}RTNYAESVKGRFTISRDNEQX₄ₑX_{4f}X_{4g}FLEMNX₄ₕLKPVDTAVYFCTRPX₄ᵢIRGRDW
GQGTQVTVSS
wherein X₄ₐ is S or T, wherein X_{4b} is S or T, wherein X_{4c} is E or G, wherein X_{4d} is V or R,
wherein X₄ₑ is R or S, wherein X_{4f} is M or T, wherein X_{4g} is V or L, wherein X₄ₕ is S or N,
wherein X₄ᵢ is S or N.
SEQ ID NO: 383 consensus of selected anti-CD36 VHH sequences
QVQLQESGGGLVQPGGSLRLSCAASGRTFSSYPMGWFRQAPGKEREFVAAIDWSGD
ITYYADSVKGRFAISRDNAKNMVYLQMNSLKPEDTAVYYCNAGPLDSWERNDFPS
WX₅ₐQGTQVTVSS
wherein X₅ₐ is G or D.

TABLE 8
Exemplary nucleic acid sequences

| Code | SEQUENCE |
|---|---|
| KF001 | CAGGTGCAATTGCAAGAATCTGGCGGCGGACTGGTTCAGCCTGGCGGATCTCTG |
| (SEQ ID | AGACTGTCTTGTGCCGCCTCCGGCTTCACCTTCTCCTCTTACGCCATGTCCTGGT |
| NO: 228) | ACAGGCAGGCCCCTGGCAAAGAGAGAGAGCTGGTCGCTCTGATCACCTCTGCT |
| | GGCGGCACCACCGATTACGCCGATTCTGTGAAGGGCAGATTCACCATCTCTCGG |
| | GACGACGCCGAAAAGACCGTGTCTCTCCAGATGAACTCCCTGAAGCCTGAGGA |
| | CACCGCCGTGTACTACTGCTCCGCTTGGAGAGCCTCTCTGTACGAGCCTTATTG |
| | GGGCCAGGGAACCCAGGTCACCGTGTCCTCC |
| KF002 | CAGGTGCAATTGCAAGAATCTGGCGGCGGACTGGTTCAGCCTGGCGGATCTCTG |
| (SEQ ID | AGACTGTCTTGTGCCGCCTCTGGCTTCACCTTCTCCAACTACGCCATGTCCTGGT |
| NO: 229) | ACAGACAGACCCCTGGCAAAGAGAGAGAACTGGTCGCCGCCATGCAGTCTACC |
| | GGCGGCACCACCTACTACGCCGATTCTGTGAAGGGCAGATTCACCATCTCTCGG |
| | GACGACGCCAAGAACACCGTGTACCTCCAGATGAACTCCCTGAAGCCTGAGGA |
| | CACCGCCGTGTACTACTGCAACGCTGTGACCCTTCAAGGCTGGAGCGAGTACGA |
| | TTATTGGGGCCAGGGAACCCAGGTCACCGTGTCCTCC |
| KF003 | CAGGTGCAATTGCAAGAATCTGGCGGCGGACTGGTTCAGCCTGGCGGATCTCTG |
| (SEQ ID | AGACTGTCTTGTACCGCCTCCGGCTTCACCTTCTCCAGCTACGCCATGTCCTGGT |
| NO: 230) | ACAGGCAGGCCCCTGGCAAAGAGAGAGAGCTGGTTGCTGCTATCCCTCACGAC |
| | GGCGGCTCCATCTACTACGTGGACTCTGTGAAGGGCAGATTCTCCATCAGCCGG |
| | GACAACGCCAAGAACACCGTGTACCTCCAGATGAACTCCCTGAAGCCTGAGGA |
| | CACCGCCGTGTACTACTGCAACGCTGTGACCCTTCAAGGCTGGCGCGAGTACGA |
| | TTATTGGGGCCAGGGAACACAGGTCACCGTGTCCTCC |
| KF004 | CAGGTGCAATTGCAAGAATCTGGCGGCGGACTGGTTCAGCCTGGCGGATCTCTG |
| (SEQ ID | AGACTGTCTTGTGCTGCTTCTGGCCTGGCCTTCTCCACCTACGCCATGTCCTGGT |
| NO: 231) | ATAGACAGGCCCCTGGCAAAGAGAGAGAGCTGGTCGCTCTGATCACCTCTGCT |
| | GGCGGCACCACCGATTACGCCGATTCTGTGAAGGGCAGATTCACCATCTCTCGG |
| | GACGACGCCGAAAAGACCGTGTCTCTCCAGATGAACTCCCTGAAGCCTGAGGA |
| | CACCGCCGTGTACTACTGCTCCGCTTGGAGAGCTACCCTGTTCAAGCCTTATTG |
| | GGGCCAGGGCACACAGGTCACCGTGTCCTCC |
| KF005 | CAGGTGCAATTGCAAGAATCTGGCGGCGGACTGGTTCAGCCTGGCGGATCTCTG |
| (SEQ ID | AGACTGTCTTGTGCCGCCTCCGGCTTCACCAGCGGCTTTACCTTTTCCTCCTACG |
| NO: 232) | CCATGTCCTGGTACAGGCAGGCCCCTGGCAAAGAGAGAGAGCTGGTCGCCGCT |
| | ATCACCAACGCTGGCGATTCTACCCACTACGCCGACTCCGTGAAGGGCAGATTC |
| | ACCATCTCCAGAGACAACGCCAAGAACACCGTGTACCTGCTGATGAACTCCCTG |
| | AAGCCTGAGGACACCGCCGCCTACTACTGTAACGCTGTGACCCTTCAAGGCTGG |
| | AGCGAGTACGATTATTGGGGCCAGGGAACCCAGGTCACCGTGTCCTCC |
| KF006 | CAGGTGCAATTGCAAGAATCTGGCGGCGGACTGGTTCAGCCTGGCGGATCTCTG |
| (SEQ ID | AGACTGTCTTGTGCCGCCTCTGGCTTCTCCCTGGACTACTTTGCCATCGGCTGGT |
| NO: 233) | TCAGACAGGCCCCTGGCAAAGAGAGAGAGGGAATCTCCTGCATCTCCTCCTCCG |
| | GCTCCAACACCTACTACGCCGACTCTGTGAAGGGCAGATTCACCATCTCTCGGG |
| | ACAACGCCAAGAACACCGTGTACCTCCAGATGAACTCCCTGAAGCCTGAGGAC |
| | ACCGCCGTGTACTACTGTGCCGCTCACGACTACTGTTCTCGGGCCCCTTACGATT |
| | ATTGGGGCCAGGGAACACAGGTCACCGTGTCCTCC |
| KF007 | CAGGTGCAATTGCAAGAATCTGGCGGCGGACTGGTGCAGCCTGGCGAATCTCT |
| (SEQ ID | GAGACTGTCTTGTGCCGCCTCCGGCTTCACCTTCTCCAACTACGCCATGTCCTGG |
| NO: 234) | TACAGACAGACCCCTGGCAAAGAGAGAGAACTGGTCGCCGCCATGCAGTCTAC |
| | CGGCGGCACCACCTACTACGCCGATTCTGTGAAGGGCAGATTCACCATCTCTCG |
| | GGACGACGCCAAGAACACCGTGTACCTCCAGATGAACTCCCTGAAGCCTGAGG |
| | ACACCGCCGTGTACTACTGCAACGCTGTGACCCTTCAAGGCTGGAGCGAGTACG |
| | ATTATTGGGGCCAGGGAACCCAGGTCACCGTGTCCTCC |
| KF008 | CAGGTGCAATTGCAAGAATCTGGCGGCGGACTGGTTCAGCCTGGCGGATCTCTG |
| (SEQ ID | AGACTGTCTTGTGCCGCCTCCGGCTTCAGATTCTCCTCCTCCATCATGTCCTGGT |
| NO: 235) | ACAGGCAGGCCCCTGAGAAAGAGAGAGAGCTGGTCGCCTCCATCTCCTCCGAT |
| | TCTGTGCGGACCAACTACGCCGAGTCCGTGAAGGGCAGATTCACCATCTCTCGG |
| | GACAACGAGCAGAGCACCCTGTTTCTGGAAATGAACAACCTGAAGCCTGTGGA |
| | TACCGCCGTGTACTTCTGCACCCGGCCTTCCATCAGAGGCAGAGATTGGGGACA |
| | GGGCACACAGGTCACCGTGTCCTCC |
| KF009 | CAGGTGCAATTGCAAGAATCTGGCGGCGGACTGGTTCAGCCTGGCGGATCTCTG |
| (SEQ ID | AGACTGTCTTGTACCGCCTCTGGCAGAACCGGCTCTGCTGATGTGATGGGCTGG |
| NO: 236) | TTTCGGAGAGCCCCTGGCAAGGACAGAGAATTTGTGGCCGCTGCCGATTGGAG |
| | AGGCCTGAACACCTGGTACAGAGACTCCGTGAAGGGCAGATTCACCATCAGCC |
| | GGGACAACGCCAAGTCCACCATGTACCTCCAGATGAACTCCCTGAAGCCTACCG |
| | ACACCGCCGTGTACTACTGTAATACCGGCGGCTGGGCCTTTTCTCCTGGCGCTTC |
| | TTGGGGACAGGGAACCCAGGTCACCGTGTCCTCC |
| KF010 | CAGGTGCAATTGCAAGAATCTGGCGGCGGACTGGTTCAGCCTGGCGGATCTCTG |
| (SEQ ID | AGACTGTCTTGTGCCGCCTCCGGCTTCAGATTCTCCACCTCCATCATGACCTGGT |
| NO: 237) | ACAGGCAGGCCCCTGGCAAAGAGAGAGAGCTGGTCGCCTCCATCTCCTCCGAC |
| | TCTAGAAGGACCAACTACGCCGAGTCCGTGAAGGGCAGATTCACCATCTCTCGG |
| | GACAACGAGCAGCGGATGGTGTTTCTGGAAATGAACTCCCTGAAGCCTGTGGA |
| | TACCGCCGTGTACTTCTGCACCCGGCCTAACATCAGAGGCAGAGATTGGGGCCA |
| | GGGCACACAGGTCACCGTGTCCTCC |
| KF011 | CAGGTGCAATTGCAAGAATCTGGCGGCGGACTGGTGCAGCTCGGAGGATCTCT |
| (SEQ ID | GAGACTGTCTTGTGCCGCCTCCGGCTTCACCTTCTCCAACTACTACATGTCCTGG |
| NO: 238) | TACAGGCAGGCCCCTGGCAAAGAGAGAGAACTCGTCGGCGGCATCAATATGAC |
| | CGGCGGCTCTGCTGACTACGTGGACTCTGTGAAGGGCAGATTCACCATCAGCCG |
| | GGACAACACCAAGAACACCGTGTACCTCCAGATGAACTCCCTGAGAGTGGAAG |
| | ATACCGCCGTGTACTACTGCCACACCTTCAGCGTGTGGCCTCCAAACAACTATT |
| | GGGGCCAGGGCACACAGGTCACCGTGTCCTCC |
| KF012 | CAGGTGCAATTGCAAGAATCTGGCGGCGGACTGGTTCAGCCTGGCGGATCTCTG |
| (SEQ ID | AGACTGTCTTGTGCCGCCTCCGGCTTCTCCTTCTCTACCTACGCCATGTCCTGGT |
| NO: 239) | ACAGACAGCCTCCTGGCAAAGAGAGAGAGCTGGTCGCTCTGATCACCACCACA |
| | GGCGGCACCACCGATTACGCCGATTCTGTGAAGGGCAGATTCACCATCTCTCGG |
| | GACGACGCCGAAAAGACCGTGTCTCTCCAGATGAACTCCCTGAAGCCTGAGGA |
| | CACCGCCGTGTACTACTGCTCCGCTTGGAGAGCTACCCTGCTGAAGCCTTATTG |
| | GGGCCAGGGAACACAGGTCACCGTGTCCTCC |
| KF013 | CAGGTGCAATTGCAAGAATCTGGCGGCGGACTGGTTCAGCCTGGCGGATCTCTG |
| (SEQ ID | AGACTGTCTTGTGCCGCTTCCGGCAGAACCTTCAGCAGCTATCCTATGGGCTGG |
| NO: 240) | TTCAGACAGGCCCCTGGCAAAGAGAGAGAGTTCGTCGCCGCTATCGATTGGAG |
| | CGGCGACATCACCTACTACGCCGACTCTGTGAAGGGCAGATTCGCCATCTCCAG |
| | AGACAACGCCAAGAACATGGTGTACCTCCAGATGAACTCCCTGAAGCCTGAGG |
| | ACACCGCCGTGTACTACTGTAACGCTGGCCCTCTGGACTCCTGGGAGAGAAACG |
| | ATTTCCCTAGCTGGGGCCAGGGCACACAGGTCACCGTGTCCTCC |
| KF014 | CAGGTGCAATTGCAAGAATCTGGCGGCGGACTGGTTCAGCCTGGCGGATCTCTG |
| (SEQ ID | AGACTGTCTTGTGCCGCTTCCGGCAGAACCTTCAGCAGCTATCCTATGGGCTGG |
| NO: 241) | TTCAGACAGGCCCCTGGCAAAGAGAGAGAGTTCGTCGCCGCTATCGATTGGAG |
| | CGGCGACATCACCTACTACGCCGACTCTGTGAAGGGCAGATTCGCCATCTCCAG |
| | AGACAACGCCAAGAACATGGTGTACCTCCAGATGAACTCCCTGAAGCCTGAGG |
| | ACACCGCCGTGTACTACTGTAACGCTGGCCCTCTGGACTCCTGGGAGAGAAACG |
| | ATTTCCCTAGCTGGGACCAGGGCACCCAGGTCACCGTGTCCTCC |
| KF015 | CAGGTGCAATTGCAAGAATCTGGCGGCGGATTGGCTCAGCCTGGCGGATCTCTG |
| (SEQ ID | AGACTGTCTTGTGCCGCCTCTGGCTTCACCGTGTCTACCTACGCCATGTCCTGGT |
| NO: 242) | ACAGGCAGGCCCCTGGCAAAGAGAGAGAGCTGGTCGCTCTGATCACCTCCGCT |
| | GGCGGCATCACCGATTACGCCGATTCTGTGAAGGGCAGATTCACAATCGCCCGG |
| | GACGACGCCGAAAAGACCGTGTCTCTCCAGATGAACTCCCTGAAGCCTGAGGA |
| | CACCGCCGTGTACTACTGCTCCGCTTGGAGAGCTACCCTGTTCAAGCCTTATTG |
| | GGGCCAGGGCACACAGGTCACCGTGTCCTCC |
| KF016 | CAGGTGCAATTGCAAGAATCTGGCGGCGGACTGGTTCAGGCTGGCGGATCTCTG |
| (SEQ ID | AGACTGTCTTGTGCCGCTTCTGGCGACACCATCTGCACCTCTGCCATGGCCTGGT |
| NO: 243) | ATAGACAGGCCCCTGGCAAAGAGAGAGAGCTGGTCGCTGGAATCTCCTCTGGC |
| | GGCACCGTGTACTACGCCGACTCTGTGAAGGGCAGATTCACCATCTCTCGGGAC |
| | AACGCCAAGAATACCGTGTACCTCCAGATGAACACCCTGAAGCCTGAGGACAC |
| | CGCTGTGTACTACTGCAACGCTGGACCTGAGGTGCACGACGACGTGTTCAGACT |
| | GTGTGGCCCTACCACCGATTATTGGGGCCAGGGAACACAGGTCACCGTGTCCTC |
| | C |
| KF017 | CAGGTGCAATTGCAAGAATCTGGCGGCGGACTGGTTCAGCCTGGCGGATCTCTG |
| (SEQ ID | AGACTGTCTTGTGCCGCCTCTGGCTTCACCCTGGACTACTATGCCATCGGCTGGT |
| NO: 244) | TCAGACAGGCCCCTGGCAAAGAAAGAGAGGGCATCGTCTACATCTCCAACTCC |
| | GACGGCAACACCTACTACGCCGACTCCGTGAAGGGCAGATTCACCATCTCCAG |
| | AGACAACGCCAAGAACACCGTGTACCTCCAGATGAACTCCCTGAAGCCTGAGG |
| | ACACCGCCGTGTACTACTGTGCTGCCGACCAGGGATCCTCTGTGATGCACGGCT |
| | TCATCGAAGAGTGGTACGACTACTGGGGCCAGGGCACACAGGTCACCGTGTCC |
| | TCC |
| KF018 | CAGGTGCAATTGCAAGAATCTGGCGGCGGACTGGTTCAGGCTGGCGGATCTCTG |
| (SEQ ID | AGACTGTCTTGTGCCGCTTCCGGCAGAACCTTCTCCTCTTACGCCATGGGCTGGT |
| NO: 245) | TCAGACAGGCCCCTGGCAAAGAGAGAGAGTTCGTCGCCGCTATCTCTTGGAGC |
| | GGCGGCAGGACCTACTACGACGATCCTGTGAAGGGCAGATTCACCATCAGCCG |
| | GGACAACGCCAAGAACACCGTGTACCTCCAGATGAACTCCCTGAAGCCTGAGG |
| | ACACCGCCGTGTACTACTGCAACCTGGATCCTGAGTACTCCGGCACCTGGAATC |
| | CTTGGGCTGGCTCTGGATATTGGGGCCAGGGAACACAGGTCACCGTGTCCTCC |
| KF021 | CAGGTGCAATTGCAAGAGTCTGGCGGAGGCTGGGTTCAGCCTGGTGGATCTCTG |
| (SEQ ID | AGACTGTCCTGTGTGGCCTCCGGCTTCACCTTCTCCAGATCCTCCATGTCTTGGC |
| NO: 246) | ACAGACAGGCCCCTGGCAAAGAGAGAGATCTGGTCGCCATCATCTCCTCCAGC |
| | GGCATCATCACCCACTACTCCGACTCCGTGAAGGGCAGATTCACCATCTCTCGG |
| | GACAACGCCAAGGACACCGTGTACCTCCAGATGAACTCCCTGAAGCCTGAGGA |
| | CACCGCCGTGTACGTGTGTAACGCCGTGCTGGACGACGACTCTGGCGATTATTG |
| | GGGCCAGGGAACACAGGTCACCGTGTCCTCC |
| KF022 | CAATTGCAAGAGTCTGGCGGAGGACTGGTTCAGCCTGGCGGATCTCTGAGACTG |
| (SEQ ID | TCTTGCGCCGCCTCCAGATTCGACTTCTCCGTGAACGACATCCGGTGGTACAGA |
| NO: 247) | CAGGCCCCTGGCAATGAGAGAGAGCTGGTCGCTAGAATCACCGGCGGAGGCAG |
| | AACCGACTACGCCGATTCTGTGAAGGGCAGATTCACCATCAGCCGGGACAACG |
| | CCAAGAACACCGTGTACCTCCAGATGAACAACCTGAAGCCTGAGGACACCGCC |
| | GTGTACTACTGTTGGGGCAGAGGCTATTGGGGCCAGGGAACTCAGGTCACCGT |
| | GTCTAGT |
| KF023 | CAGGTGCAATTGCAAGAATCTGGCGGCGGACTGGTTCAGCCTGGCGGATCTCTG |
| (SEQ ID | AGACTGTCTTGTGCCGCCTCCGGCTTCACCTTCTCCTCTTACGCCATGTCCTGGT |
| NO: 248) | ACAGGCAGGCCCCTGGCAAAGAGAGAGAGCTGGTCGCTCTGATCACCTCTGCT |
| | GGCGGCACCACCGATTACGCCGATTCTGTGAAGGGCAGATTCACCATCTCTCGG |
| | GACGACGCCGAAAAGACCGTGTCTCTCCAGATGAACTCCCTGAAGCCTGAGGA |
| | CACCGCCGTGTACTACTGCTCCGCTTGGAGAGCCTCTCTGTACGAGCCTTATTG |
| | GGGCCAGGGAACCCAGGTCACCGTGTCCTCCGAGCCTAAGTCTTGCGACAAGA |
| | CCCACACCTGTCCTCCATGTCCTGCTCCAGAATTGCTCGGCGGACCCTCCGTGTT |
| | TCTGTTCCCTCCAAAGCCTAAGGACACCCTGATGATCTCTCGGACCCCTGAAGT |
| | GACCTGCGTGGTGGTGGATGTGTCCCACGAAGATCCCGAAGTGAAGTTTAATTG |
| | GTACGTGGACGGCGTCGAGGTGCACAACGCTAAGACCAAGCCTAGAGAGGAAC |
| | AGTACAACTCCACCTACAGAGTGGTGTCCGTGCTGACCGTGCTGCACCAGGATT |
| | GGCTGAACGGCAAAGAGTACAAGTGCAAGGTGTCCAACAAGGCCCTGCCTGCT |
| | CCTATCGAAAAGACCATCTCCAAGGCCAAGGGCCAGCCTAGGGAACCCCAGGT |
| | TTACACCCTGCCTCCAAGCCGGGAAGAGATGACCAAGAACCAGGTGTCCCTGA |
| | CCTGCCTGGTCAAGGGCTTCTACCCTTCCGATATCGCCGTGGAATGGGAGAGCA |
| | ATGGCCAGCCTGAGAACAACTACAAGACAACCCCTCCTGTGCTGGACTCCGAC |
| | GGCTCATTCTTTCTGTACTCCAAGCTGACAGTGGACAAGTCCAGATGGCAGCAG |
| | GGCAACGTGTTCTCCTGCTCTGTGATGCACGAGGCCCTGCACAACCACTACACC |
| | CAGAAGTCCCTGTCTCTGTCCCCTGGCAAA |
| KF024 | CAGGTGCAATTGCAAGAATCTGGCGGCGGACTGGTTCAGCCTGGCGGATCTCTG |
| (SEQ ID | AGACTGTCTTGTGCCGCCTCTGGCTTCACCTTCTCCAACTACGCCATGTCCTGGT |
| NO: 249) | ACAGACAGACCCCTGGCAAAGAGAGAGAACTGGTCGCCGCCATGCAGTCTACC |
| | GGCGGCACCACCTACTACGCCGATTCTGTGAAGGGCAGATTCACCATCTCTCGG |
| | GACGACGCCAAGAACACCGTGTACCTCCAGATGAACTCCCTGAAGCCTGAGGA |
| | CACCGCCGTGTACTACTGCAACGCTGTGACCCTTCAAGGCTGGAGCGAGTACGA |
| | TTATTGGGGCCAGGGAACCCAGGTCACCGTGTCCTCCGAGCCTAAGTCTTGCGA |
| | CAAGACCCACACCTGTCCTCCATGTCCTGCTCCAGAATTGCTCGGCGGACCCTC |
| | CGTGTTTCTGTTCCCTCCAAAGCCTAAGGACACCCTGATGATCTCTCGGACCCCT |
| | GAAGTGACCTGCGTGGTGGTGGATGTGTCCCACGAAGATCCCGAAGTGAAGTTT |
| | AATTGGTACGTGGACGGCGTCGAGGTGCACAACGCTAAGACCAAGCCTAGAGA |
| | GGAACAGTACAACTCCACCTACAGAGTGGTGTCCGTGCTGACCGTGCTGCACCA |
| | GGATTGGCTGAACGGCAAAGAGTACAAGTGCAAGGTGTCCAACAAGGCCCTGC |
| | CTGCTCCTATCGAAAAGACCATCTCCAAGGCCAAGGGCCAGCCTAGGGAACCC |
| | CAGGTTTACACCCTGCCTCCAAGCCGGGAAGAGATGACCAAGAACCAGGTGTC |
| | CCTGACCTGCCTGGTCAAGGGCTTCTACCCTTCCGATATCGCCGTGGAATGGGA |
| | GAGCAATGGCCAGCCTGAGAACAACTACAAGACAACCCCTCCTGTGCTGGACT |
| | CCGACGGCTCATTCTTTCTGTACTCCAAGCTGACAGTGGACAAGTCCAGATGGC |
| | AGCAGGGCAACGTGTTCTCCTGCTCTGTGATGCACGAGGCCCTGCACAACCACT |
| | ACACCCAGAAGTCCCTGTCTCTGTCCCCTGGCAAA |
| KF025 | CAGGTGCAATTGCAAGAATCTGGCGGCGGACTGGTTCAGCCTGGCGGATCTCTG |
| (SEQ ID | AGACTGTCTTGTACCGCCTCCGGCTTCACCTTCTCCAGCTACGCCATGTCCTGGT |
| NO: 250) | ACAGGCAGGCCCCTGGCAAAGAGAGAGAGCTGGTTGCTGCTATCCCTCACGAC |
| | GGCGGCTCCATCTACTACGTGGACTCTGTGAAGGGCAGATTCTCCATCAGCCGG |
| | GACAACGCCAAGAACACCGTGTACCTCCAGATGAACTCCCTGAAGCCTGAGGA |
| | CACCGCCGTGTACTACTGCAACGCTGTGACCCTTCAAGGCTGGCGCGAGTACGA |
| | TTATTGGGGCCAGGGAACACAGGTCACCGTGTCCTCCGAGCCTAAGTCTTGCGA |
| | CAAGACCCACACCTGTCCTCCATGTCCTGCTCCAGAATTGCTCGGCGGACCCTC |
| | CGTGTTTCTGTTCCCTCCAAAGCCTAAGGACACCCTGATGATCTCTCGGACCCCT |
| | GAAGTGACCTGCGTGGTGGTGGATGTGTCCCACGAAGATCCCGAAGTGAAGTTT |
| | AATTGGTACGTGGACGGCGTCGAGGTGCACAACGCTAAGACCAAGCCTAGAGA |
| | GGAACAGTACAACTCCACCTACAGAGTGGTGTCCGTGCTGACCGTGCTGCACCA |
| | GGATTGGCTGAACGGCAAAGAGTACAAGTGCAAGGTGTCCAACAAGGCCCTGC |
| | CTGCTCCTATCGAAAAGACCATCTCCAAGGCCAAGGGCCAGCCTAGGGAACCC |
| | CAGGTTTACACCCTGCCTCCAAGCCGGGAAGAGATGACCAAGAACCAGGTGTC |
| | CCTGACCTGCCTGGTCAAGGGCTTCTACCCTTCCGATATCGCCGTGGAATGGGA |
| | GAGCAATGGCCAGCCTGAGAACAACTACAAGACAACCCCTCCTGTGCTGGACT |
| | CCGACGGCTCATTCTTTCTGTACTCCAAGCTGACAGTGGACAAGTCCAGATGGC |
| | AGCAGGGCAACGTGTTCTCCTGCTCTGTGATGCACGAGGCCCTGCACAACCACT |
| | ACACCCAGAAGTCCCTGTCTCTGTCCCCTGGCAAA |
| KF026 | CAGGTGCAATTGCAAGAATCTGGCGGCGGACTGGTTCAGCCTGGCGGATCTCTG |
| (SEQ ID | AGACTGTCTTGTGCTGCTTCTGGCCTGGCCTTCTCCACCTACGCCATGTCCTGGT |
| NO: 251) | ATAGACAGGCCCCTGGCAAAGAGAGAGAGCTGGTCGCTCTGATCACCTCTGCT |
| | GGCGGCACCACCGATTACGCCGATTCTGTGAAGGGCAGATTCACCATCTCTCGG |
| | GACGACGCCGAAAAGACCGTGTCTCTCCAGATGAACTCCCTGAAGCCTGAGGA |
| | CACCGCCGTGTACTACTGCTCCGCTTGGAGAGCTACCCTGTTCAAGCCTTATTG |
| | GGGCCAGGGCACACAGGTCACCGTGTCCTCCGAGCCTAAGTCTTGCGACAAGA |
| | CCCACACCTGTCCTCCATGTCCTGCTCCAGAATTGCTCGGCGGACCCTCCGTGTT |
| | TCTGTTCCCTCCAAAGCCTAAGGACACCCTGATGATCTCTCGGACCCCTGAAGT |
| | GACCTGCGTGGTGGTGGATGTGTCCCACGAAGATCCCGAAGTGAAGTTTAATTG |
| | GTACGTGGACGGCGTCGAGGTGCACAACGCTAAGACCAAGCCTAGAGAGGAAC |
| | AGTACAACTCCACCTACAGAGTGGTGTCCGTGCTGACCGTGCTGCACCAGGATT |
| | GGCTGAACGGCAAAGAGTACAAGTGCAAGGTGTCCAACAAGGCCCTGCCTGCT |
| | CCTATCGAAAAGACCATCTCCAAGGCCAAGGGCCAGCCTAGGGAACCCCAGGT |
| | TTACACCCTGCCTCCAAGCCGGGAAGAGATGACCAAGAACCAGGTGTCCCTGA |
| | CCTGCCTGGTCAAGGGCTTCTACCCTTCCGATATCGCCGTGGAATGGGAGAGCA |
| | ATGGCCAGCCTGAGAACAACTACAAGACAACCCCTCCTGTGCTGGACTCCGAC |
| | GGCTCATTCTTTCTGTACTCCAAGCTGACAGTGGACAAGTCCAGATGGCAGCAG |
| | GGCAACGTGTTCTCCTGCTCTGTGATGCACGAGGCCCTGCACAACCACTACACC |
| | CAGAAGTCCCTGTCTCTGTCCCCTGGCAAA |
| KF027 | CAGGTGCAATTGCAAGAATCTGGCGGCGGACTGGTTCAGCCTGGCGGATCTCTG |
| (SEQ ID | AGACTGTCTTGTGCCGCCTCCGGCTTCACCAGCGGCTTTACCTTTTCCTCCTACG |
| NO: 252) | CCATGTCCTGGTACAGGCAGGCCCCTGGCAAAGAGAGAGAGCTGGTCGCCGCT |
| | ATCACCAACGCTGGCGATTCTACCCACTACGCCGACTCCGTGAAGGGCAGATTC |
| | ACCATCTCCAGAGACAACGCCAAGAACACCGTGTACCTGCTGATGAACTCCCTG |
| | AAGCCTGAGGACACCGCCGCCTACTACTGTAACGCTGTGACCCTTCAAGGCTGG |
| | AGCGAGTACGATTATTGGGGCCAGGGAACCCAGGTCACCGTGTCCTCCGAGCCT |
| | AAGTCTTGCGACAAGACCCACACCTGTCCTCCATGTCCTGCTCCAGAATTGCTC |
| | GGCGGACCCTCCGTGTTTCTGTTCCCTCCAAAGCCTAAGGACACCCTGATGATC |
| | TCTCGGACCCCTGAAGTGACCTGCGTGGTGGTGGATGTGTCCCACGAAGATCCC |
| | GAAGTGAAGTTTAATTGGTACGTGGACGGCGTCGAGGTGCACAACGCTAAGAC |
| | CAAGCCTAGAGAGGAACAGTACAACTCCACCTACAGAGTGGTGTCCGTGCTGA |
| | CCGTGCTGCACCAGGATTGGCTGAACGGCAAAGAGTACAAGTGCAAGGTGTCC |
| | AACAAGGCCCTGCCTGCTCCTATCGAAAAGACCATCTCCAAGGCCAAGGGCCA |
| | GCCTAGGGAACCCCAGGTTTACACCCTGCCTCCAAGCCGGGAAGAGATGACCA |
| | AGAACCAGGTGTCCCTGACCTGCCTGGTCAAGGGCTTCTACCCTTCCGATATCG |
| | CCGTGGAATGGGAGAGCAATGGCCAGCCTGAGAACAACTACAAGACAACCCCT |
| | CCTGTGCTGGACTCCGACGGCTCATTCTTTCTGTACTCCAAGCTGACAGTGGAC |
| | AAGTCCAGATGGCAGCAGGGCAACGTGTTCTCCTGCTCTGTGATGCACGAGGCC |
| | CTGCACAACCACTACACCCAGAAGTCCCTGTCTCTGTCCCCTGGCAAA |
| KF028 | CAGGTGCAATTGCAAGAATCTGGCGGCGGACTGGTTCAGCCTGGCGGATCTCTG |
| (SEQ ID | AGACTGTCTTGTGCCGCCTCTGGCTTCTCCCTGGACTACTTTGCCATCGGCTGGT |
| NO: 253) | TCAGACAGGCCCCTGGCAAAGAGAGAGAGGGAATCTCCTGCATCTCCTCCTCCG |
| | GCTCCAACACCTACTACGCCGACTCTGTGAAGGGCAGATTCACCATCTCTCGGG |
| | ACAACGCCAAGAACACCGTGTACCTCCAGATGAACTCCCTGAAGCCTGAGGAC |
| | ACCGCCGTGTACTACTGTGCCGCTCACGACTACTGTTCTCGGGCCCCTTACGATT |
| | ATTGGGGCCAGGGAACACAGGTCACCGTGTCCTCCGAGCCTAAGTCTTGCGACA |
| | AGACCCACACCTGTCCTCCATGTCCTGCTCCAGAATTGCTCGGCGGACCCTCCG |
| | TGTTTCTGTTCCCTCCAAAGCCTAAGGACACCCTGATGATCTCTCGGACCCCTGA |
| | AGTGACCTGCGTGGTGGTGGATGTGTCCCACGAAGATCCCGAAGTGAAGTTTAA |
| | TTGGTACGTGGACGGCGTCGAGGTGCACAACGCTAAGACCAAGCCTAGAGAGG |
| | AACAGTACAACTCCACCTACAGAGTGGTGTCCGTGCTGACCGTGCTGCACCAGG |
| | ATTGGCTGAACGGCAAAGAGTACAAGTGCAAGGTGTCCAACAAGGCCCTGCCT |
| | GCTCCTATCGAAAAGACCATCTCCAAGGCCAAGGGCCAGCCTAGGGAACCCCA |
| | GGTTTACACCCTGCCTCCAAGCCGGGAAGAGATGACCAAGAACCAGGTGTCCC |
| | TGACCTGCCTGGTCAAGGGCTTCTACCCTTCCGATATCGCCGTGGAATGGGAGA |
| | GCAATGGCCAGCCTGAGAACAACTACAAGACAACCCCTCCTGTGCTGGACTCC |
| | GACGGCTCATTCTTTCTGTACTCCAAGCTGACAGTGGACAAGTCCAGATGGCAG |
| | CAGGGCAACGTGTTCTCCTGCTCTGTGATGCACGAGGCCCTGCACAACCACTAC |
| | ACCCAGAAGTCCCTGTCTCTGTCCCCTGGCAAA |
| KF029 | CAGGTGCAATTGCAAGAATCTGGCGGCGGACTGGTGCAGCCTGGCGAATCTCT |
| (SEQ ID | GAGACTGTCTTGTGCCGCCTCCGGCTTCACCTTCTCCAACTACGCCATGTCCTGG |
| NO: 254) | TACAGACAGACCCCTGGCAAAGAGAGAGAACTGGTCGCCGCCATGCAGTCTAC |
| | CGGCGGCACCACCTACTACGCCGATTCTGTGAAGGGCAGATTCACCATCTCTCG |
| | GGACGACGCCAAGAACACCGTGTACCTCCAGATGAACTCCCTGAAGCCTGAGG |
| | ACACCGCCGTGTACTACTGCAACGCTGTGACCCTTCAAGGCTGGAGCGAGTACG |
| | ATTATTGGGGCCAGGGAACCCAGGTCACCGTGTCCTCCGAGCCTAAGTCTTGCG |
| | ACAAGACCCACACCTGTCCTCCATGTCCTGCTCCAGAATTGCTCGGCGGACCCT |
| | CCGTGTTTCTGTTCCCTCCAAAGCCTAAGGACACCCTGATGATCTCTCGGACCCC |
| | TGAAGTGACCTGCGTGGTGGTGGATGTGTCCCACGAAGATCCCGAAGTGAAGTT |
| | TAATTGGTACGTGGACGGCGTCGAGGTGCACAACGCTAAGACCAAGCCTAGAG |
| | AGGAACAGTACAACTCCACCTACAGAGTGGTGTCCGTGCTGACCGTGCTGCACC |
| | AGGATTGGCTGAACGGCAAAGAGTACAAGTGCAAGGTGTCCAACAAGGCCCTG |
| | CCTGCTCCTATCGAAAAGACCATCTCCAAGGCCAAGGGCCAGCCTAGGGAACC |
| | CCAGGTTTACACCCTGCCTCCAAGCCGGGAAGAGATGACCAAGAACCAGGTGT |
| | CCCTGACCTGCCTGGTCAAGGGCTTCTACCCTTCCGATATCGCCGTGGAATGGG |
| | AGAGCAATGGCCAGCCTGAGAACAACTACAAGACAACCCCTCCTGTGCTGGAC |
| | TCCGACGGCTCATTCTTTCTGTACTCCAAGCTGACAGTGGACAAGTCCAGATGG |
| | CAGCAGGGCAACGTGTTCTCCTGCTCTGTGATGCACGAGGCCCTGCACAACCAC |
| | TACACCCAGAAGTCCCTGTCTCTGTCCCCTGGCAAA |
| KF030 | CAGGTGCAATTGCAAGAATCTGGCGGCGGACTGGTTCAGCCTGGCGGATCTCTG |
| (SEQ ID | AGACTGTCTTGTGCCGCCTCCGGCTTCAGATTCTCCTCCTCCATCATGTCCTGGT |
| NO: 255) | ACAGGCAGGCCCCTGAGAAAGAGAGAGAGCTGGTCGCCTCCATCTCCTCCGAT |
| | TCTGTGCGGACCAACTACGCCGAGTCCGTGAAGGGCAGATTCACCATCTCTCGG |
| | GACAACGAGCAGAGCACCCTGTTTCTGGAAATGAACAACCTGAAGCCTGTGGA |
| | TACCGCCGTGTACTTCTGCACCCGGCCTTCCATCAGAGGCAGAGATTGGGGACA |
| | GGGCACACAGGTCACCGTGTCCTCCGAGCCTAAGTCTTGCGACAAGACCCACAC |
| | CTGTCCTCCATGTCCTGCTCCAGAATTGCTCGGCGGACCCTCCGTGTTTCTGTTC |
| | CCTCCAAAGCCTAAGGACACCCTGATGATCTCTCGGACCCCTGAAGTGACCTGC |
| | GTGGTGGTGGATGTGTCCCACGAAGATCCCGAAGTGAAGTTTAATTGGTACGTG |
| | GACGGCGTCGAGGTGCACAACGCTAAGACCAAGCCTAGAGAGGAACAGTACAA |
| | CTCCACCTACAGAGTGGTGTCCGTGCTGACCGTGCTGCACCAGGATTGGCTGAA |
| | CGGCAAAGAGTACAAGTGCAAGGTGTCCAACAAGGCCCTGCCTGCTCCTATCG |
| | AAAAGACCATCTCCAAGGCCAAGGGCCAGCCTAGGGAACCCCAGGTTTACACC |
| | CTGCCTCCAAGCCGGGAAGAGATGACCAAGAACCAGGTGTCCCTGACCTGCCT |
| | GGTCAAGGGCTTCTACCCTTCCGATATCGCCGTGGAATGGGAGAGCAATGGCCA |
| | GCCTGAGAACAACTACAAGACAACCCCTCCTGTGCTGGACTCCGACGGCTCATT |
| | CTTTCTGTACTCCAAGCTGACAGTGGACAAGTCCAGATGGCAGCAGGGCAACGT |
| | GTTCTCCTGCTCTGTGATGCACGAGGCCCTGCACAACCACTACACCCAGAAGTC |
| | CCTGTCTCTGTCCCCTGGCAAA |
| KF031 | CAGGTGCAATTGCAAGAATCTGGCGGCGGACTGGTTCAGCCTGGCGGATCTCTG |
| (SEQ ID | AGACTGTCTTGTACCGCCTCTGGCAGAACCGGCTCTGCTGATGTGATGGGCTGG |
| NO: 256) | TTTCGGAGAGCCCCTGGCAAGGACAGAGAATTTGTGGCCGCTGCCGATTGGAG |
| | AGGCCTGAACACCTGGTACAGAGACTCCGTGAAGGGCAGATTCACCATCAGCC |
| | GGGACAACGCCAAGTCCACCATGTACCTCCAGATGAACTCCCTGAAGCCTACCG |
| | ACACCGCCGTGTACTACTGTAATACCGGCGGCTGGGCCTTTTCTCCTGGCGCTTC |
| | TTGGGGACAGGGAACCCAGGTCACCGTGTCCTCCGAGCCTAAGTCTTGCGACAA |
| | GACCCACACCTGTCCTCCATGTCCTGCTCCAGAATTGCTCGGCGGACCCTCCGT |
| | GTTTCTGTTCCCTCCAAAGCCTAAGGACACCCTGATGATCTCTCGGACCCCTGA |
| | AGTGACCTGCGTGGTGGTGGATGTGTCCCACGAAGATCCCGAAGTGAAGTTTAA |
| | TTGGTACGTGGACGGCGTCGAGGTGCACAACGCTAAGACCAAGCCTAGAGAGG |
| | AACAGTACAACTCCACCTACAGAGTGGTGTCCGTGCTGACCGTGCTGCACCAGG |
| | ATTGGCTGAACGGCAAAGAGTACAAGTGCAAGGTGTCCAACAAGGCCCTGCCT |
| | GCTCCTATCGAAAAGACCATCTCCAAGGCCAAGGGCCAGCCTAGGGAACCCCA |
| | GGTTTACACCCTGCCTCCAAGCCGGGAAGAGATGACCAAGAACCAGGTGTCCC |
| | TGACCTGCCTGGTCAAGGGCTTCTACCCTTCCGATATCGCCGTGGAATGGGAGA |
| | GCAATGGCCAGCCTGAGAACAACTACAAGACAACCCCTCCTGTGCTGGACTCC |
| | GACGGCTCATTCTTTCTGTACTCCAAGCTGACAGTGGACAAGTCCAGATGGCAG |
| | CAGGGCAACGTGTTCTCCTGCTCTGTGATGCACGAGGCCCTGCACAACCACTAC |
| | ACCCAGAAGTCCCTGTCTCTGTCCCCTGGCAAA |
| KF032 | CAGGTGCAATTGCAAGAATCTGGCGGCGGACTGGTTCAGCCTGGCGGATCTCTG |
| (SEQ ID | AGACTGTCTTGTGCCGCCTCCGGCTTCAGATTCTCCACCTCCATCATGACCTGGT |
| NO: 257) | ACAGGCAGGCCCCTGGCAAAGAGAGAGAGCTGGTCGCCTCCATCTCCTCCGAC |
| | TCTAGAAGGACCAACTACGCCGAGTCCGTGAAGGGCAGATTCACCATCTCTCGG |
| | GACAACGAGCAGCGGATGGTGTTTCTGGAAATGAACTCCCTGAAGCCTGTGGA |
| | TACCGCCGTGTACTTCTGCACCCGGCCTAACATCAGAGGCAGAGATTGGGGCCA |
| | GGGCACACAGGTCACCGTGTCCTCCGAGCCTAAGTCTTGCGACAAGACCCACAC |
| | CTGTCCTCCATGTCCTGCTCCAGAATTGCTCGGCGGACCCTCCGTGTTTCTGTTC |
| | CCTCCAAAGCCTAAGGACACCCTGATGATCTCTCGGACCCCTGAAGTGACCTGC |
| | GTGGTGGTGGATGTGTCCCACGAAGATCCCGAAGTGAAGTTTAATTGGTACGTG |
| | GACGGCGTCGAGGTGCACAACGCTAAGACCAAGCCTAGAGAGGAACAGTACAA |
| | CTCCACCTACAGAGTGGTGTCCGTGCTGACCGTGCTGCACCAGGATTGGCTGAA |
| | CGGCAAAGAGTACAAGTGCAAGGTGTCCAACAAGGCCCTGCCTGCTCCTATCG |
| | AAAAGACCATCTCCAAGGCCAAGGGCCAGCCTAGGGAACCCCAGGTTTACACC |
| | CTGCCTCCAAGCCGGGAAGAGATGACCAAGAACCAGGTGTCCCTGACCTGCCT |
| | GGTCAAGGGCTTCTACCCTTCCGATATCGCCGTGGAATGGGAGAGCAATGGCCA |
| | GCCTGAGAACAACTACAAGACAACCCCTCCTGTGCTGGACTCCGACGGCTCATT |
| | CTTTCTGTACTCCAAGCTGACAGTGGACAAGTCCAGATGGCAGCAGGGCAACGT |
| | GTTCTCCTGCTCTGTGATGCACGAGGCCCTGCACAACCACTACACCCAGAAGTC |
| | CCTGTCTCTGTCCCCTGGCAAA |
| KF033 | CAGGTGCAATTGCAAGAATCTGGCGGCGGACTGGTGCAGCTCGGAGGATCTCT |
| (SEQ ID | GAGACTGTCTTGTGCCGCCTCCGGCTTCACCTTCTCCAACTACTACATGTCCTGG |
| NO: 258) | TACAGGCAGGCCCCTGGCAAAGAGAGAGAACTCGTCGGCGGCATCAATATGAC |
| | CGGCGGCTCTGCTGACTACGTGGACTCTGTGAAGGGCAGATTCACCATCAGCCG |
| | GGACAACACCAAGAACACCGTGTACCTCCAGATGAACTCCCTGAGAGTGGAAG |
| | ATACCGCCGTGTACTACTGCCACACCTTCAGCGTGTGGCCTCCAAACAACTATT |
| | GGGGCCAGGGCACACAGGTCACCGTGTCCTCCGAGCCTAAGTCTTGCGACAAG |
| | ACCCACACCTGTCCTCCATGTCCTGCTCCAGAATTGCTCGGCGGACCCTCCGTGT |
| | TTCTGTTCCCTCCAAAGCCTAAGGACACCCTGATGATCTCTCGGACCCCTGAAG |
| | TGACCTGCGTGGTGGTGGATGTGTCCCACGAAGATCCCGAAGTGAAGTTTAATT |
| | GGTACGTGGACGGCGTCGAGGTGCACAACGCTAAGACCAAGCCTAGAGAGGAA |
| | CAGTACAACTCCACCTACAGAGTGGTGTCCGTGCTGACCGTGCTGCACCAGGAT |
| | TGGCTGAACGGCAAAGAGTACAAGTGCAAGGTGTCCAACAAGGCCCTGCCTGC |
| | TCCTATCGAAAAGACCATCTCCAAGGCCAAGGGCCAGCCTAGGGAACCCCAGG |
| | TTTACACCCTGCCTCCAAGCCGGGAAGAGATGACCAAGAACCAGGTGTCCCTG |
| | ACCTGCCTGGTCAAGGGCTTCTACCCTTCCGATATCGCCGTGGAATGGGAGAGC |
| | AATGGCCAGCCTGAGAACAACTACAAGACAACCCCTCCTGTGCTGGACTCCGA |
| | CGGCTCATTCTTTCTGTACTCCAAGCTGACAGTGGACAAGTCCAGATGGCAGCA |
| | GGGCAACGTGTTCTCCTGCTCTGTGATGCACGAGGCCCTGCACAACCACTACAC |
| | CCAGAAGTCCCTGTCTCTGTCCCCTGGCAAA |
| KF034 | CAGGTGCAATTGCAAGAATCTGGCGGCGGACTGGTTCAGCCTGGCGGATCTCTG |
| (SEQ ID | AGACTGTCTTGTGCCGCCTCCGGCTTCTCCTTCTCTACCTACGCCATGTCCTGGT |
| NO: 259) | ACAGACAGCCTCCTGGCAAAGAGAGAGAGCTGGTCGCTCTGATCACCACCACA |
| | GGCGGCACCACCGATTACGCCGATTCTGTGAAGGGCAGATTCACCATCTCTCGG |
| | GACGACGCCGAAAAGACCGTGTCTCTCCAGATGAACTCCCTGAAGCCTGAGGA |
| | CACCGCCGTGTACTACTGCTCCGCTTGGAGAGCTACCCTGCTGAAGCCTTATTG |
| | GGGCCAGGGAACACAGGTCACCGTGTCCTCCGAGCCTAAGTCTTGCGACAAGA |
| | CCCACACCTGTCCTCCATGTCCTGCTCCAGAATTGCTCGGCGGACCCTCCGTGTT |
| | TCTGTTCCCTCCAAAGCCTAAGGACACCCTGATGATCTCTCGGACCCCTGAAGT |
| | GACCTGCGTGGTGGTGGATGTGTCCCACGAAGATCCCGAAGTGAAGTTTAATTG |
| | GTACGTGGACGGCGTCGAGGTGCACAACGCTAAGACCAAGCCTAGAGAGGAAC |
| | AGTACAACTCCACCTACAGAGTGGTGTCCGTGCTGACCGTGCTGCACCAGGATT |
| | GGCTGAACGGCAAAGAGTACAAGTGCAAGGTGTCCAACAAGGCCCTGCCTGCT |
| | CCTATCGAAAAGACCATCTCCAAGGCCAAGGGCCAGCCTAGGGAACCCCAGGT |
| | TTACACCCTGCCTCCAAGCCGGGAAGAGATGACCAAGAACCAGGTGTCCCTGA |
| | CCTGCCTGGTCAAGGGCTTCTACCCTTCCGATATCGCCGTGGAATGGGAGAGCA |
| | ATGGCCAGCCTGAGAACAACTACAAGACAACCCCTCCTGTGCTGGACTCCGAC |
| | GGCTCATTCTTTCTGTACTCCAAGCTGACAGTGGACAAGTCCAGATGGCAGCAG |
| | GGCAACGTGTTCTCCTGCTCTGTGATGCACGAGGCCCTGCACAACCACTACACC |
| | CAGAAGTCCCTGTCTCTGTCCCCTGGCAAA |
| KF035 | CAGGTGCAATTGCAAGAATCTGGCGGCGGACTGGTTCAGCCTGGCGGATCTCTG |
| (SEQ ID | AGACTGTCTTGTGCCGCTTCCGGCAGAACCTTCAGCAGCTATCCTATGGGCTGG |
| NO: 260) | TTCAGACAGGCCCCTGGCAAAGAGAGAGAGTTCGTCGCCGCTATCGATTGGAG |
| | CGGCGACATCACCTACTACGCCGACTCTGTGAAGGGCAGATTCGCCATCTCCAG |
| | AGACAACGCCAAGAACATGGTGTACCTCCAGATGAACTCCCTGAAGCCTGAGG |
| | ACACCGCCGTGTACTACTGTAACGCTGGCCCTCTGGACTCCTGGGAGAGAAACG |
| | ATTTCCCTAGCTGGGGCCAGGGCACACAGGTCACCGTGTCCTCCGAGCCTAAGT |
| | CTTGCGACAAGACCCACACCTGTCCTCCATGTCCTGCTCCAGAATTGCTCGGCG |
| | GACCCTCCGTGTTTCTGTTCCCTCCAAAGCCTAAGGACACCCTGATGATCTCTCG |
| | GACCCCTGAAGTGACCTGCGTGGTGGTGGATGTGTCCCACGAAGATCCCGAAGT |
| | GAAGTTTAATTGGTACGTGGACGGCGTCGAGGTGCACAACGCTAAGACCAAGC |
| | CTAGAGAGGAACAGTACAACTCCACCTACAGAGTGGTGTCCGTGCTGACCGTG |
| | CTGCACCAGGATTGGCTGAACGGCAAAGAGTACAAGTGCAAGGTGTCCAACAA |
| | GGCCCTGCCTGCTCCTATCGAAAAGACCATCTCCAAGGCCAAGGGCCAGCCTAG |
| | GGAACCCCAGGTTTACACCCTGCCTCCAAGCCGGGAAGAGATGACCAAGAACC |
| | AGGTGTCCCTGACCTGCCTGGTCAAGGGCTTCTACCCTTCCGATATCGCCGTGG |
| | AATGGGAGAGCAATGGCCAGCCTGAGAACAACTACAAGACAACCCCTCCTGTG |
| | CTGGACTCCGACGGCTCATTCTTTCTGTACTCCAAGCTGACAGTGGACAAGTCC |
| | AGATGGCAGCAGGGCAACGTGTTCTCCTGCTCTGTGATGCACGAGGCCCTGCAC |
| | AACCACTACACCCAGAAGTCCCTGTCTCTGTCCCCTGGCAAA |
| KF036 | CAGGTGCAATTGCAAGAATCTGGCGGCGGACTGGTTCAGCCTGGCGGATCTCTG |
| (SEQ ID | AGACTGTCTTGTGCCGCTTCCGGCAGAACCTTCAGCAGCTATCCTATGGGCTGG |
| NO: 261) | TTCAGACAGGCCCCTGGCAAAGAGAGAGAGTTCGTCGCCGCTATCGATTGGAG |
| | CGGCGACATCACCTACTACGCCGACTCTGTGAAGGGCAGATTCGCCATCTCCAG |
| | AGACAACGCCAAGAACATGGTGTACCTCCAGATGAACTCCCTGAAGCCTGAGG |
| | ACACCGCCGTGTACTACTGTAACGCTGGCCCTCTGGACTCCTGGGAGAGAAACG |
| | ATTTCCCTAGCTGGGACCAGGGCACCCAGGTCACCGTGTCCTCCGAGCCTAAGT |
| | CTTGCGACAAGACCCACACCTGTCCTCCATGTCCTGCTCCAGAATTGCTCGGCG |
| | GACCCTCCGTGTTTCTGTTCCCTCCAAAGCCTAAGGACACCCTGATGATCTCTCG |
| | GACCCCTGAAGTGACCTGCGTGGTGGTGGATGTGTCCCACGAAGATCCCGAAGT |
| | GAAGTTTAATTGGTACGTGGACGGCGTCGAGGTGCACAACGCTAAGACCAAGC |
| | CTAGAGAGGAACAGTACAACTCCACCTACAGAGTGGTGTCCGTGCTGACCGTG |
| | CTGCACCAGGATTGGCTGAACGGCAAAGAGTACAAGTGCAAGGTGTCCAACAA |
| | GGCCCTGCCTGCTCCTATCGAAAAGACCATCTCCAAGGCCAAGGGCCAGCCTAG |
| | GGAACCCCAGGTTTACACCCTGCCTCCAAGCCGGGAAGAGATGACCAAGAACC |
| | AGGTGTCCCTGACCTGCCTGGTCAAGGGCTTCTACCCTTCCGATATCGCCGTGG |
| | AATGGGAGAGCAATGGCCAGCCTGAGAACAACTACAAGACAACCCCTCCTGTG |
| | CTGGACTCCGACGGCTCATTCTTTCTGTACTCCAAGCTGACAGTGGACAAGTCC |
| | AGATGGCAGCAGGGCAACGTGTTCTCCTGCTCTGTGATGCACGAGGCCCTGCAC |
| | AACCACTACACCCAGAAGTCCCTGTCTCTGTCCCCTGGCAAA |
| KF037 | CAGGTGCAATTGCAAGAATCTGGCGGCGGATTGGCTCAGCCTGGCGGATCTCTG |
| (SEQ ID | AGACTGTCTTGTGCCGCCTCTGGCTTCACCGTGTCTACCTACGCCATGTCCTGGT |
| NO: 262) | ACAGGCAGGCCCCTGGCAAAGAGAGAGAGCTGGTCGCTCTGATCACCTCCGCT |
| | GGCGGCATCACCGATTACGCCGATTCTGTGAAGGGCAGATTCACAATCGCCCGG |
| | GACGACGCCGAAAAGACCGTGTCTCTCCAGATGAACTCCCTGAAGCCTGAGGA |
| | CACCGCCGTGTACTACTGCTCCGCTTGGAGAGCTACCCTGTTCAAGCCTTATTG |
| | GGGCCAGGGCACACAGGTCACCGTGTCCTCCGAGCCTAAGTCTTGCGACAAGA |
| | CCCACACCTGTCCTCCATGTCCTGCTCCAGAATTGCTCGGCGGACCCTCCGTGTT |
| | TCTGTTCCCTCCAAAGCCTAAGGACACCCTGATGATCTCTCGGACCCCTGAAGT |
| | GACCTGCGTGGTGGTGGATGTGTCCCACGAAGATCCCGAAGTGAAGTTTAATTG |
| | GTACGTGGACGGCGTCGAGGTGCACAACGCTAAGACCAAGCCTAGAGAGGAAC |
| | AGTACAACTCCACCTACAGAGTGGTGTCCGTGCTGACCGTGCTGCACCAGGATT |
| | GGCTGAACGGCAAAGAGTACAAGTGCAAGGTGTCCAACAAGGCCCTGCCTGCT |
| | CCTATCGAAAAGACCATCTCCAAGGCCAAGGGCCAGCCTAGGGAACCCCAGGT |
| | TTACACCCTGCCTCCAAGCCGGGAAGAGATGACCAAGAACCAGGTGTCCCTGA |
| | CCTGCCTGGTCAAGGGCTTCTACCCTTCCGATATCGCCGTGGAATGGGAGAGCA |
| | ATGGCCAGCCTGAGAACAACTACAAGACAACCCCTCCTGTGCTGGACTCCGAC |
| | GGCTCATTCTTTCTGTACTCCAAGCTGACAGTGGACAAGTCCAGATGGCAGCAG |
| | GGCAACGTGTTCTCCTGCTCTGTGATGCACGAGGCCCTGCACAACCACTACACC |
| | CAGAAGTCCCTGTCTCTGTCCCCTGGCAAA |
| KF038 | CAGGTGCAATTGCAAGAATCTGGCGGCGGACTGGTTCAGGCTGGCGGATCTCTG |
| (SEQ ID | AGACTGTCTTGTGCCGCTTCTGGCGACACCATCTGCACCTCTGCCATGGCCTGGT |
| NO: 263) | ATAGACAGGCCCCTGGCAAAGAGAGAGAGCTGGTCGCTGGAATCTCCTCTGGC |
| | GGCACCGTGTACTACGCCGACTCTGTGAAGGGCAGATTCACCATCTCTCGGGAC |
| | AACGCCAAGAATACCGTGTACCTCCAGATGAACACCCTGAAGCCTGAGGACAC |
| | CGCTGTGTACTACTGCAACGCTGGACCTGAGGTGCACGACGACGTGTTCAGACT |
| | GTGTGGCCCTACCACCGATTATTGGGGCCAGGGAACACAGGTCACCGTGTCCTC |
| | CGAGCCTAAGTCTTGCGACAAGACCCACACCTGTCCTCCATGTCCTGCTCCAGA |
| | ATTGCTCGGCGGACCCTCCGTGTTTCTGTTCCCTCCAAAGCCTAAGGACACCCT |
| | GATGATCTCTCGGACCCCTGAAGTGACCTGCGTGGTGGTGGATGTGTCCCACGA |
| | AGATCCCGAAGTGAAGTTTAATTGGTACGTGGACGGCGTCGAGGTGCACAACG |
| | CTAAGACCAAGCCTAGAGAGGAACAGTACAACTCCACCTACAGAGTGGTGTCC |
| | GTGCTGACCGTGCTGCACCAGGATTGGCTGAACGGCAAAGAGTACAAGTGCAA |
| | GGTGTCCAACAAGGCCCTGCCTGCTCCTATCGAAAAGACCATCTCCAAGGCCAA |
| | GGGCCAGCCTAGGGAACCCCAGGTTTACACCCTGCCTCCAAGCCGGGAAGAGA |
| | TGACCAAGAACCAGGTGTCCCTGACCTGCCTGGTCAAGGGCTTCTACCCTTCCG |
| | ATATCGCCGTGGAATGGGAGAGCAATGGCCAGCCTGAGAACAACTACAAGACA |
| | ACCCCTCCTGTGCTGGACTCCGACGGCTCATTCTTTCTGTACTCCAAGCTGACAG |
| | TGGACAAGTCCAGATGGCAGCAGGGCAACGTGTTCTCCTGCTCTGTGATGCACG |
| | AGGCCCTGCACAACCACTACACCCAGAAGTCCCTGTCTCTGTCCCCTGGCAAA |
| KF039 | CAGGTGCAATTGCAAGAATCTGGCGGCGGACTGGTTCAGCCTGGCGGATCTCTG |
| (SEQ ID | AGACTGTCTTGTGCCGCCTCTGGCTTCACCCTGGACTACTATGCCATCGGCTGGT |
| NO: 264) | TCAGACAGGCCCCTGGCAAAGAAAGAGAGGGCATCGTCTACATCTCCAACTCC |
| | GACGGCAACACCTACTACGCCGACTCCGTGAAGGGCAGATTCACCATCTCCAG |
| | AGACAACGCCAAGAACACCGTGTACCTCCAGATGAACTCCCTGAAGCCTGAGG |
| | ACACCGCCGTGTACTACTGTGCTGCCGACCAGGGATCCTCTGTGATGCACGGCT |
| | TCATCGAAGAGTGGTACGACTACTGGGGCCAGGGCACACAGGTCACCGTGTCC |
| | TCCGAGCCTAAGTCTTGCGACAAGACCCACACCTGTCCTCCATGTCCTGCTCCA |
| | GAATTGCTCGGCGGACCCTCCGTGTTTCTGTTCCCTCCAAAGCCTAAGGACACC |
| | CTGATGATCTCTCGGACCCCTGAAGTGACCTGCGTGGTGGTGGATGTGTCCCAC |
| | GAAGATCCCGAAGTGAAGTTTAATTGGTACGTGGACGGCGTCGAGGTGCACAA |
| | CGCTAAGACCAAGCCTAGAGAGGAACAGTACAACTCCACCTACAGAGTGGTGT |
| | CCGTGCTGACCGTGCTGCACCAGGATTGGCTGAACGGCAAAGAGTACAAGTGC |
| | AAGGTGTCCAACAAGGCCCTGCCTGCTCCTATCGAAAAGACCATCTCCAAGGCC |
| | AAGGGCCAGCCTAGGGAACCCCAGGTTTACACCCTGCCTCCAAGCCGGGAAGA |
| | GATGACCAAGAACCAGGTGTCCCTGACCTGCCTGGTCAAGGGCTTCTACCCTTC |
| | CGATATCGCCGTGGAATGGGAGAGCAATGGCCAGCCTGAGAACAACTACAAGA |
| | CAACCCCTCCTGTGCTGGACTCCGACGGCTCATTCTTTCTGTACTCCAAGCTGAC |
| | AGTGGACAAGTCCAGATGGCAGCAGGGCAACGTGTTCTCCTGCTCTGTGATGCA |
| | CGAGGCCCTGCACAACCACTACACCCAGAAGTCCCTGTCTCTGTCCCCTGGCAA |
| | A |
| KF040 | CAGGTGCAATTGCAAGAATCTGGCGGCGGACTGGTTCAGGCTGGCGGATCTCTG |
| (SEQ ID | AGACTGTCTTGTGCCGCTTCCGGCAGAACCTTCTCCTCTTACGCCATGGGCTGGT |
| NO: 265) | TCAGACAGGCCCCTGGCAAAGAGAGAGAGTTCGTCGCCGCTATCTCTTGGAGC |
| | GGCGGCAGGACCTACTACGACGATCCTGTGAAGGGCAGATTCACCATCAGCCG |
| | GGACAACGCCAAGAACACCGTGTACCTCCAGATGAACTCCCTGAAGCCTGAGG |
| | ACACCGCCGTGTACTACTGCAACCTGGATCCTGAGTACTCCGGCACCTGGAATC |
| | CTTGGGCTGGCTCTGGATATTGGGGCCAGGGAACACAGGTCACCGTGTCCTCCG |
| | AGCCTAAGTCTTGCGACAAGACCCACACCTGTCCTCCATGTCCTGCTCCAGAAT |
| | TGCTCGGCGGACCCTCCGTGTTTCTGTTCCCTCCAAAGCCTAAGGACACCCTGA |
| | TGATCTCTCGGACCCCTGAAGTGACCTGCGTGGTGGTGGATGTGTCCCACGAAG |
| | ATCCCGAAGTGAAGTTTAATTGGTACGTGGACGGCGTCGAGGTGCACAACGCT |
| | AAGACCAAGCCTAGAGAGGAACAGTACAACTCCACCTACAGAGTGGTGTCCGT |
| | GCTGACCGTGCTGCACCAGGATTGGCTGAACGGCAAAGAGTACAAGTGCAAGG |
| | TGTCCAACAAGGCCCTGCCTGCTCCTATCGAAAAGACCATCTCCAAGGCCAAGG |
| | GCCAGCCTAGGGAACCCCAGGTTTACACCCTGCCTCCAAGCCGGGAAGAGATG |
| | ACCAAGAACCAGGTGTCCCTGACCTGCCTGGTCAAGGGCTTCTACCCTTCCGAT |
| | ATCGCCGTGGAATGGGAGAGCAATGGCCAGCCTGAGAACAACTACAAGACAAC |
| | CCCTCCTGTGCTGGACTCCGACGGCTCATTCTTTCTGTACTCCAAGCTGACAGTG |
| | GACAAGTCCAGATGGCAGCAGGGCAACGTGTTCTCCTGCTCTGTGATGCACGAG |
| | GCCCTGCACAACCACTACACCCAGAAGTCCCTGTCTCTGTCCCCTGGCAAA |
| KF042 | CAGGTCCAATTGCAAGAATCTGGCGGCGGACTGGTTCAGCCTGGCGGATCTCTG |
| (SEQ ID | AGACTGTCTTGTGCCGCCTCCGGCTTCACCTTCTCCTCTTACGCCATGTCCTGGT |
| NO: 266) | ACAGGCAGGCCCCTGGCAAAGAGAGAGAGCTGGTCGCTCTGATCACCTCTGCT |
| | GGCGGCACCACCGATTACGCCGATTCTGTGAAGGGCAGATTCACCATCTCTCGG |
| | GACGACGCCGAAAAGACCGTGTCTCTGCAGATGAACTCCCTGAAGCCTGAGGA |
| | CACCGCCGTGTACTACTGCTCCGCTTGGAGAGCCTCTCTGTACGAGCCTTATTG |
| | GGGCCAGGGAACCCAGGTCACCGTGTCTAGTGAACCCAAGTCCTGCGACAAGA |
| | CCCACACCTGTCCTCCATGTCCTGCTCCAGAACTGCTCGGCGGACCTTCCGTGTT |
| | CCTGTTTCCTCCAAAGCCTAAGGACACCCTGATGATCTCTCGGACCCCTGAAGT |
| | GACCTGCGTGGTGGTGGATGTGTCTCACGAGGATCCCGAAGTGAAGTTCAATTG |
| | GTACGTGGACGGCGTGGAAGTGCACAACGCCAAGACCAAGCCTAGAGAGGAAC |
| | AGTACAACTCCACCTACAGAGTGGTGTCCGTGCTGACCGTGCTGCACCAGGATT |
| | GGCTGAACGGCAAAGAGTACAAGTGCAAGGTGTCCAACAAGGCCCTGCCTGCT |
| | CCTATCGAAAAGACCATCTCCAAGGCCAAGGGCCAGCCTAGGGAACCCCAGGT |
| | TTACACCCTGCCTCCAAGCCGGGAAGAGATGACCAAGAACCAGGTGTCCCTGA |
| | CCTGCCTGGTCAAGGGCTTCTACCCTTCCGATATCGCCGTGGAATGGGAGAGCA |
| | ATGGCCAGCCTGAGAACAACTACAAGACAACCCCTCCTGTGCTGGACTCCGAC |
| | GGCTCATTCTTCCTGTACTCCAAGCTGACAGTGGACAAGTCCAGATGGCAGCAG |
| | GGCAACGTGTTCTCCTGCTCCGTGATGCACGAGGCCCTGCACAATCACTACACC |
| | CAGAAGTCCCTGTCTCTGTCCCCTGGCAAAGCCCCAGCCCCTGCCCCTGCACCC |
| | GCGCCTAAAGCCCAGGTTCAATTGGTGGAATCTGGCGGCGGATCTGTACAAGCT |
| | GGCGGCTCTCTGAGACTGTCTTGTGCCGCTTCTGGCTCCACCGACTCCATCGAGT |
| | ACATGACCTGGTTCAGACAGGCCCCTGGCAAGGCTAGAGAAGGTGTCGCTGCT |
| | CTGTATACCCACACCGGCAACACCTACTACACCGACAGCGTGAAGGGCAGATT |
| | CACCATCAGCCAGGACAAGGCCAAGAACATGGCCTACCTGCGGATGGACTCCG |
| | TGAAGTCTGAGGACACCGCCATCTACACCTGTGGCGCTACCCGGAAATACGTGC |
| | CCGTCAGATTTGCCCTGGACCAGTCCTCCTACGACTACTGGGGACAAGGGACCC |
| | AGGTCACCGTGTCATCGGGAGGCGGAGGATCTGGCGGAGGTGGAAGTGGCGGA |
| | GGCGGAAGCCAGGTTCAATTGGTGGAATCTGGCGGCGGATCTGTACAAGCTGG |
| | CGGCTCTCTGAGACTGTCTTGTGCCGCTTCTGGCTCCACCGACTCCATCGAGTAC |
| | ATGACCTGGTTCAGACAGGCCCCTGGCAAGGCTAGAGAAGGTGTCGCTGCTCTG |
| | TATACCCACACCGGCAACACCTACTACACCGACAGCGTGAAGGGCAGATTCAC |
| | CATCAGCCAGGACAAGGCCAAGAACATGGCCTACCTGCGGATGGACTCCGTGA |
| | AGTCTGAGGACACCGCCATCTACACCTGTGGCGCTACCCGGAAATACGTGCCCG |
| | TCAGATTTGCCCTGGACCAGTCCTCCTACGACTACTGGGGACAAGGGACCCAGG |
| | TCACCGTGAGTTCT |
| KF061 | CAGGTCCAATTGCAAGAATCTGGCGGCGGACTGGTTCAGCCTGGCGGATCTCTG |
| (SEQ ID | AGACTGTCTTGTGCCGCCTCCGGCTTCACCTTCTCCTCTTACGCCATGTCCTGGT |
| NO: 267) | ACAGGCAGGCCCCTGGCAAAGAGAGAGAGCTGGTCGCTCTGATCACCTCTGCT |
| | GGCGGCACCACCGATTACGCCGATTCTGTGAAGGGCAGATTCACCATCTCTCGG |
| | GACGACGCCGAAAAGACCGTGTCTCTGCAGATGAACTCCCTGAAGCCTGAGGA |
| | CACCGCCGTGTACTACTGCTCCGCTTGGAGAGCCTCTCTGTACGAGCCTTATTG |
| | GGGCCAGGGAACCCAGGTCACCGTGTCTAGTGAACCCAAGTCCTGCGACAAGA |
| | CCCACACCTGTCCTCCATGTCCTGCTCCAGAACTGCTCGGCGGACCTTCCGTGTT |
| | CCTGTTTCCTCCAAAGCCTAAGGACACCCTGATGATCTCTCGGACCCCTGAAGT |
| | GACCTGCGTGGTGGTGGATGTGTCTCACGAGGATCCCGAAGTGAAGTTCAATTG |
| | GTACGTGGACGGCGTGGAAGTGCACAACGCCAAGACCAAGCCTAGAGAGGAAC |
| | AGTACAACTCCACCTACAGAGTGGTGTCCGTGCTGACCGTGCTGCACCAGGATT |
| | GGCTGAACGGCAAAGAGTACAAGTGCAAGGTGTCCAACAAGGCCCTGCCTGCT |
| | CCTATCGAAAAGACCATCTCCAAGGCCAAGGGCCAGCCTAGGGAACCCCAGGT |
| | TTACACCCTGCCTCCAAGCCGGGAAGAGATGACCAAGAACCAGGTGTCCCTGA |
| | CCTGCCTGGTCAAGGGCTTCTACCCTTCCGATATCGCCGTGGAATGGGAGAGCA |
| | ATGGCCAGCCTGAGAACAACTACAAGACAACCCCTCCTGTGCTGGACTCCGAC |
| | GGCTCATTCTTCCTGTACTCCAAGCTGACAGTGGACAAGTCCAGATGGCAGCAG |
| | GGCAACGTGTTCTCCTGCTCCGTGATGCACGAGGCCCTGCACAATCACTACACC |
| | CAGAAGTCCCTGTCTCTGTCCCCTGGCAAAGCCCCAGCCCCTGCCCCTGCACCC |
| | GCGCCTAAAGCCCAAGTCCAATTGCAAGAGTCTGGCGGAGGCTGGGTTCAGCC |
| | TGGTGGATCTCTGAGACTGTCCTGTGTGGCCTCCGGCTTCACCTTCTCCAGATCC |
| | TCCATGTCTTGGCACAGACAGGCCCCTGGCAAAGAGAGAGATCTGGTCGCCATC |
| | ATCTCCTCCAGCGGCATCATCACCCACTACTCCGACTCCGTGAAGGGCAGATTC |
| | ACCATCTCTCGGGACAACGCCAAGGACACCGTGTACCTCCAGATGAACTCCCTG |
| | AAGCCTGAGGACACCGCCGTGTACGTGTGTAACGCCGTGCTGGACGACGACTCT |
| | GGCGATTATTGGGGCCAGGGAACACAGGTCACCGTGTCATCGGGAGGCGGAGG |
| | ATCTGGCGGAGGTGGAAGTGGCGGAGGCGGAAGCCAGGTTCAATTGCAAGAGT |
| | CTGGCGGAGGACTGGTTCAGCCTGGCGGATCTCTGAGACTGTCTTGCGCCGCCT |
| | CCAGATTCGACTTCTCCGTGAACGACATCCGGTGGTACAGACAGGCCCCTGGCA |
| | ATGAGAGAGAGCTGGTCGCTAGAATCACCGGCGGAGGCAGAACCGACTACGCC |
| | GATTCTGTGAAGGGCAGATTCACCATCAGCCGGGACAACGCCAAGAACACCGT |
| | GTACCTCCAGATGAACAACCTGAAGCCTGAGGACACCGCCGTGTACTACTGTTG |
| | GGGCAGAGGCTATTGGGGCCAGGGAACTCAGGTCACCGTGAGTTCT |
| KF062 | CAAGTCCAATTGCAAGAGTCTGGCGGAGGCCTGGTTCAGCCTGGTGGAT |
| (SEQ ID | CTCTGAGACTGTCCTGCGCCACCTCTACCTCCATGCACAGCTTCGAGAAC |
| NO: 366) | GTGGCCTGGTACAGACAGGCTCCTGGCAAGCAGAGAGAGCTGGTCGCCA |
| | TCATTGCCCAGTACATCACCCACTACAACGACGCCGTGAAGGGCAGATT |
| | CACCATCTCTCGGGACGACTCCAAGAACACCGCCTACCTCCAGATGAAC |
| | AACCTGAAGCCTGAGGACACCGCCGTGTACTACTGCAACGTGGACCAGT |
| | ATTGGGGCCAGGGCACACAGGTCACCGTTTCATCG |
| KF063 | CAAGTCCAATTGCAAGAGTCTGGCGGAGGCCTGGTTCAGCCTGGTGGAT |
| (SEQ ID | CTCTGAGACTGTCTTGTGCCGCCTCCGGCTTCACCTTCTCCAACTACGGA |
| NO: 367) | ATGTCCTGGGTCCGACAGGCTCCTGGCGAAGGATTGGAATGGGTGTCCT |
| | CCATCGATTCCACCGGCGGCACCACCAGATACGCCGATTCTGTGAAGGG |
| | CAGATTCACCATCAGCCGGGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF064 | CAAGTCCAATTGCAAGAGTCTGGCGGAGGCCTGGTTCAGGCTGGTGGAT |
| (SEQ ID | CTCTGAGACTGTCTTGTGCCGCCTCCGGCTTCACCTTCTCCAACTACGGA |
| NO: 368) | ATGTCCTGGGTCCGACAGGCTCCTGGCGAAGGATTGGAATGGGTGTCCT |
| | CCATCGATTCCTCCGGCGGCACCACTAAGTACGCCGACTCTGTGAAGGG |
| | CAGATTCACCATCTCTCGGGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF065 | CAAGTCCAATTGCAAGAGTCTGGCGGAGGCCTGGTTCAGCCTGGCGATT |
| (SEQ ID | CTCTGAGACTGTCTTGTGCCGCCTCCGGCTTCACCTTCTCCAACTACGGA |
| NO: 369) | ATGTCCTGGGTCCGACAGGCTCCTGGCGAAGGATTGGAATGGGTGTCCT |
| | CCATCGATTCCTCCGGCGGCACCACTAAGTACGCCGACTCTGTGAAGGG |
| | CAGATTCACCATCTCTCGGGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF066 | CAAGTCCAATTGCAAGAGTCTGGCGGAGGCCTGGTTCAGGCTGGTGGAT |
| (SEQ ID | CTCTGAGACTGTCTTGTGCCGCCTCCGGCTTCACCTTCTCCAACTACGGA |
| NO: 370) | ATGTCCTGGGTCCGACAGGCTCCTGGCGAAGGATTGGAATGGGTGTCCT |
| | CCATCGATTCCACCGGCGGCACCACCAGATACGCCGATTCTGTGAAGGG |
| | CAGATTCACCATCAGCCGGGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF067 | CAAGTCCAATTGCAAGAGTCTGGCGGAGGCCTGGTTCAGCCTGGTGGAT |
| (SEQ ID | CTCTGAGACTGTCCTGCGCCACCTCTACCTCCATGCACAGCTTCGAGAAC |
| NO: 371) | GTGGCCTGGTACAGACAGGCTCCCGGAAAGCAGAGAGAGCTGGCCGCT |
| | ATCATTGCCCAGTACATCACCCACTACAACGACGCCGTGAAGGGCAGAT |
| | TCACCATCTCTCGGGACGACTCCAAGAACACCGCCTACCTCCAGATGAA |
| | CAACCTGAAGCCTGAGGACACCGCCGTGTACTACTGCAACGTGGACCAG |
| | TATTGGGGCCAGGGCACACAGGTCACCGTTTCATCG |
| KF068 | CAAGTCCAATTGCAAGAGTCTGGCGGAGGCCTGGTTCAGCCTGGCGATT |
| (SEQ ID | CTCTGAGACTGTCTTGTGCCGCCTCCGGCTTCACCTTCTCCTCTTACGGA |
| NO: 372) | ATGTCCTGGGTCCGACAGGCTCCTGGCGAAGGATTGGAATGGGTGTCCT |
| | CCATCGATTCCTCCGGCGGCACCACTAAGTACGCCGGATCTGTGCAGGG |
| | CAGATTCACCATCTCCAGAGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF069 | CAAGTCCAATTGCAAGAATCTGGCGGCGGAGAGGTTCAGGCTGGCGGAT |
| (SEQ ID | CTCTGAGACTGTCTTGTGCCGCCTCCGGCTTCACCTTCTCCTCTTACGGA |
| NO: 373) | ATGTCCTGGGTCCGACAGGCTCCTGGCGAAGGATTGGAATGGGTGTCCT |
| | CCATCGATTCCTCCGGCGGCACCACTAAGTACGCCGGATCTGTGCAGGG |
| | CAGATTCACCATCTCCAGAGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF070 | CAAGTCCAATTGCAAGAGTCTGGCGGAGGCCTGGTTCAGCCTGGTGGAT |
| (SEQ ID | CTCTGAGACTGTCTTGTGCCGCCTCCGGCTTCACCTTCTCCAACTACGGA |
| NO: 374) | ATGTCCTGGGTCCGACAGGCTCCTGGCGAAGGATTGGAATGGGTGTCCT |
| | CCATCGATTCCACCGGCGGCACCACCAGATACGCCGATTCTGTGAAGGG |
| | CAGATTCACCATCAGCCGGGACAACGCCAAGAACGCCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF071 | CAGGTCCAATTGCAAGAGTCTGGCGGCGGATTGGTTCAGCCTGGCGGAT |
| (SEQ ID | CTCTGAGACTGTCTTGTGCCGCCTCTGGCTTCACCTTCTCCAACTACGGC |
| NO: 375) | ATGTCCTGGGTCCGACAGGCTCCTGGCGAAGGATTGGAATGGGTGTCCT |
| | CCATCGATTCCTCCGGCGGCACCACCAGATACGCCGATTCTGTGAAGGG |
| | CAGATTCACCATCAGCCGGGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF072 | CAGGTCCAATTGCAAGAGTCTGGCGGCGGATTGGTTCAGCCTGGCGGAT |
| (SEQ ID | CTCTGAGACTGTCTTGTGCCGCCTCCGGCTTCACCTTCTCCTCTTACGGA |
| NO: 376) | ATGTCCTGGGTCCGACAGGCTCCTGGCGAAGGATTGGAATGGGTGTCCT |
| | CCATCGATTCCTCCGGCGGCACCACTAAGTACGCCGGATCTGTGCAGGG |
| | CAGATTCACCATCTCCAGAGACAACGCCAAGAACACCCTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF073 | CAGGTCCAATTGCAAGAGTCTGGCGGCGGATTGGTGCAGCCTGGCGATT |
| (SEQ ID | CTCTGAGACTGTCTTGTGCCGCCTCCGGCTTCACCTTCTCCTCTTACGGA |
| NO: 377) | ATGTCCTGGGTCCGACAGGCTCCTGGCGAAGGATTGGAATGGGTGTCCT |
| | CCATCGATTCCTCCGGCGGCACCACTAAGTACGCCGGATCTGTGCAGGG |
| | CAGATTCACCATCTCCAGAGACAACGCCAAGAACACCGTGTACCTCCAG |
| | ATGAACTCCCTGAAGCCTGAGGACACCGCCGTGTACTACTGCGCCAAGG |
| | ACTTCCTGTCCTGGATGCCTAGAGGCCAGGGAACACAGGTCACCGTGTC |
| | ATCG |
| KF074 | CAGGTCCAATTGCAAGAGTCTGGCGGCGGATTGGTTCAGGCTGGCGGAT |
| (SEQ ID | CTCTGAGACTGTCCTGTACCGCCTCCTCCGTGACCTTCTCCTCTGACACA |
| NO: 378) | ATGGGCTGGTTCAGACAGGCCCCTGGCAAAGAGAGAGAGTTCGTCGCTG |
| | CCATCCAGCGGAGAGGCGGCTCTATCTACTACGCCGACTCTGTGAAGGG |
| | CAGATTCACCATCAGCCGGGACAACCCCAAGTCCACCGTGTACCTCCAG |
| | ATGAACTCCCTGAAGCCTGAGGACACCGCCGTGTACTACTGTGCCGCTTC |
| | TCCTGTGAACGGCGACAACTATGGCCTGCTGCCTCGGCACTACGATTATT |
| | GGGGACAGGGCACACAGGTCACCGTGTCATCG |

1. A binding agent comprising one or more antigen binding domains, wherein at least one of the one or more antigen binding domains is an antigen binding domain that is capable of specifically binding to Cluster of Differentiation 36 (CD36), and comprises:
a. a heavy chain complementarity determining region 1 (CDRH1) having the amino acid sequence set forth in SEQ ID NO:1, a heavy chain complementarity determining region 2 (CDRH2) having the amino acid sequence set forth in SEQ ID NO:2 and a heavy chain complementarity determining region 3 (CDRH3) having the amino acid sequence set forth in SEQ ID NO:3;
b. a CDRH1 having the amino acid sequence set forth in SEQ ID NO:4, a CDRH2 having the amino acid sequence set forth in SEQ ID NO:5 and a CDRH3 having the amino acid sequence set forth in SEQ ID NO:6;
c. a CDRH1 having the amino acid sequence set forth in SEQ ID NO:8, a CDRH2 having the amino acid sequence set forth in SEQ ID NO:9 and a CDRH3 having the amino acid sequence set forth in SEQ ID NO:10;
d. a CDRH1 having the amino acid sequence set forth in SEQ ID NO:11, a CDRH2 having the amino acid sequence set forth in SEQ ID NO:12 and a CDRH3 having the amino acid sequence set forth in SEQ ID NO:13;
e. a CDRH1 having the amino acid sequence set forth in SEQ ID NO:15, a CDRH2 having the amino acid sequence set forth in SEQ ID NO:16 and a CDRH3 having the amino acid sequence set forth in SEQ ID NO:17;
f. a CDRH1 having the amino acid sequence set forth in SEQ ID NO:18, a CDRH2 having the amino acid sequence set forth in SEQ ID NO:19 and a CDRH3 having the amino acid sequence set forth in SEQ ID NO:20;
g. a CDRH1 having the amino acid sequence set forth in SEQ ID NO:22, a CDRH2 having the amino acid sequence set forth in SEQ ID NO:23 and a CDRH3 having the amino acid sequence set forth in SEQ ID NO:24;
h. a CDRH1 having the amino acid sequence set forth in SEQ ID NO:25, a CDRH2 having the amino acid sequence set forth in SEQ ID NO:26 and a CDRH3 having the amino acid sequence set forth in SEQ ID NO:27;
i. a CDRH1 having the amino acid sequence set forth in SEQ ID NO:29, a CDRH2 having the amino acid sequence set forth in SEQ ID NO:30 and a CDRH3 having the amino acid sequence set forth in SEQ ID NO:31;
j. a CDRH1 having the amino acid sequence set forth in SEQ ID NO:32, a CDRH2 having the amino acid sequence set forth in SEQ ID NO:33 and a CDRH3 having the amino acid sequence set forth in SEQ ID NO:34;
k. a CDRH1 having the amino acid sequence set forth in SEQ ID NO:36, a CDRH2 having the amino acid sequence set forth in SEQ ID NO:37 and a CDRH3 having the amino acid sequence set forth in SEQ ID NO:38;
l. a CDRH1 having the amino acid sequence set forth in SEQ ID NO:39, a CDRH2 having the amino acid sequence set forth in SEQ ID NO:40 and a CDRH3 having the amino acid sequence set forth in SEQ ID NO:41;
m. a CDRH1 having the amino acid sequence set forth in SEQ ID NO:43, a CDRH2 having the amino acid sequence set forth in SEQ ID NO:44 and a CDRH3 having the amino acid sequence set forth in SEQ ID NO:45;
n. a CDRH1 having the amino acid sequence set forth in SEQ ID NO:46, a CDRH2 having the amino acid sequence set forth in SEQ ID NO:47 and a CDRH3 having the amino acid sequence set forth in SEQ ID NO:48;
o. a CDRH1 having the amino acid sequence set forth in SEQ ID NO:50, a CDRH2 having the amino acid sequence set forth in SEQ ID NO:51 and a CDRH3 having the amino acid sequence set forth in SEQ ID NO:52;
p. a CDRH1 having the amino acid sequence set forth in SEQ ID NO:53, a CDRH2 having the amino acid sequence set forth in SEQ ID NO:54 and a CDRH3 having the amino acid sequence set forth in SEQ ID NO:55;
q. a CDRH1 having the amino acid sequence set forth in SEQ ID NO:71, a CDRH2 having the amino acid sequence set forth in SEQ ID NO:72 and a CDRH3 having the amino acid sequence set forth in SEQ ID NO:73;
r. a CDRH1 having the amino acid sequence set forth in SEQ ID NO:74, a CDRH2 having the amino acid sequence set forth in SEQ ID NO:75 and a CDRH3 having the amino acid sequence set forth in SEQ ID NO:76;
s. a CDRH1 having the amino acid sequence set forth in SEQ ID NO:78, a CDRH2 having the amino acid sequence set forth in SEQ ID NO:79 and a CDRH3 having the amino acid sequence set forth in SEQ ID NO:80;
t. a CDRH1 having the amino acid sequence set forth in SEQ ID NO:81, a CDRH2 having the amino acid sequence set forth in SEQ ID NO:82 and a CDRH3 having the amino acid sequence set forth in SEQ ID NO:83;
u. a CDRH1 having the amino acid sequence set forth in SEQ ID NO:85, a CDRH2 having the amino acid sequence set forth in SEQ ID NO:86 and a CDRH3 having the amino acid sequence set forth in SEQ ID NO:87;
v. a CDRH1 having the amino acid sequence set forth in SEQ ID NO:88, a CDRH2 having the amino acid sequence set forth in SEQ ID NO:89 and a CDRH3 having the amino acid sequence set forth in SEQ ID NO:90;
w. a CDRH1 having the amino acid sequence set forth in SEQ ID NO:92, a CDRH2 having the amino acid sequence set forth in SEQ ID NO:93 and a CDRH3 having the amino acid sequence set forth in SEQ ID NO:94;
x. a CDRH1 having the amino acid sequence set forth in SEQ ID NO:95, a CDRH2 having the amino acid sequence set forth in SEQ ID NO:96 and a CDRH3 having the amino acid sequence set forth in SEQ ID NO:97;
y. a CDRH1 having the amino acid sequence set forth in SEQ ID NO:99, a CDRH2 having the amino acid sequence set forth in SEQ ID NO:100 and a CDRH3 having the amino acid sequence set forth in SEQ ID NO:101;
z. a CDRH1 having the amino acid sequence set forth in SEQ ID NO:102, a CDRH2 having the amino acid sequence set forth in SEQ ID NO:103 and a CDRH3 having the amino acid sequence set forth in SEQ ID NO:104;
aa. a CDRH1 having the amino acid sequence set forth in SEQ ID NO:106, a CDRH2 having the amino acid sequence set forth in SEQ ID NO:107 and a CDRH3 having the amino acid sequence set forth in SEQ ID NO:108;
bb. a CDRH1 having the amino acid sequence set forth in SEQ ID NO:109, a CDRH2 having the amino acid sequence set forth in SEQ ID NO:110 and a CDRH3 having the amino acid sequence set forth in SEQ ID NO:111;
cc. a CDRH1 having the amino acid sequence set forth in SEQ ID NO:113, a CDRH2 having the amino acid sequence set forth in SEQ ID NO:114 and a CDRH3 having the amino acid sequence set forth in SEQ ID NO:115;
dd. a CDRH1 having the amino acid sequence set forth in SEQ ID NO:116, a CDRH2 having the amino acid sequence set forth in SEQ ID NO:117 and a CDRH3 having the amino acid sequence set forth in SEQ ID NO:118;
ee. a CDRH1 having the amino acid sequence set forth in SEQ ID NO:120, a CDRH2 having the amino acid sequence set forth in SEQ ID NO:121 and a CDRH3 having the amino acid sequence set forth in SEQ ID NO:122;
ff. a CDRH1 having the amino acid sequence set forth in SEQ ID NO:123, a CDRH2 having the amino acid sequence set forth in SEQ ID NO:124 and a CDRH3 having the amino acid sequence set forth in SEQ ID NO:125;
gg. a CDRH1 having the amino acid sequence set forth in SEQ ID NO:127, a CDRH2 having the amino acid sequence set forth in SEQ ID NO:128 and a CDRH3 having the amino acid sequence set forth in SEQ ID NO:129;
hh. a CDRH1 having the amino acid sequence set forth in SEQ ID NO:130, a CDRH2 having the amino acid sequence set forth in SEQ ID NO:131 and a CDRH3 having the amino acid sequence set forth in SEQ ID NO:132;
ii. a CDRH1 having the amino acid sequence set forth in SEQ ID NO:134, a CDRH2 having the amino acid sequence set forth in SEQ ID NO:135 and a CDRH3 having the amino acid sequence set forth in SEQ ID NO:136; or
jj. a CDRH1 having the amino acid sequence set forth in SEQ ID NO:137, a CDRH2 having the amino acid sequence set forth in SEQ ID NO:138 and a CDRH3 having the amino acid sequence set forth in SEQ ID NO:139.

2.-5. (canceled)

6. The binding agent of claim 1, wherein the antigen binding domain that is capable of specifically binding to CD36 is an antigen binding domain 1 (ABD1) and comprises:
g. an amino acid sequence at least 80% identical to the amino acid sequence set forth in SEQ ID NO:28;
h. an amino acid sequence at least 80%, identical to the amino acid sequence set forth in SEQ ID NO:35;
i. an amino acid sequence at least 80% identical to the amino acid sequence set forth in SEQ ID NO:42;
j. an amino acid sequence at least 80% identical to the amino acid sequence set forth in SEQ ID NO:49;
k. an amino acid sequence at least 80% identical to the amino acid sequence set forth in SEQ ID NO:56;
l. an amino acid sequence at least 80% identical to the amino acid sequence set forth in SEQ ID NO:77;
m. an amino acid sequence at least 80% identical to the amino acid sequence set forth in SEQ ID NO:84;
n. an amino acid sequence at least 80% identical to the amino acid sequence set forth in SEQ ID NO:91;
o. an amino acid sequence at least 80% identical to the amino acid sequence set forth in SEQ ID NO:98;
p. an amino acid sequence at least 80% identical to the amino acid sequence set forth in SEQ ID NO:105;
q. an amino acid sequence at least 80% identical to the amino acid sequence set forth in SEQ ID NO:112;
r. an amino acid sequence at least 80% identical to the amino acid sequence set forth in SEQ ID NO:119;
s. an amino acid sequence at least 80% identical to the amino acid sequence set forth in SEQ ID NO:126;
t. an amino acid sequence at least 80% identical to the amino acid sequence set forth in SEQ ID NO:133, or
u. an amino acid sequence at least 80% identical to the amino acid sequence set forth in SEQ ID NO:140.

7. The binding agent of claim 1, wherein the binding agent comprises more than one antigen binding domain and wherein at least one of the one or more antigen binding domains is an antigen binding domain 2 (ABD2) that is capable of specifically binding to PD-1 and comprises:
a. a heavy chain complementarity determining region 1 (CDRH1) having the amino acid sequence set forth in SEQ ID NO:57, a heavy chain complementarity determining region 2 (CDRH2) having the amino acid sequence set forth in SEQ ID NO:58 and a heavy chain complementarity determining region 3 (CDRH3) having the amino acid sequence set forth in SEQ ID NO:59;
b. a CDRH1 having the amino acid sequence set forth in SEQ ID NO:60, a CDRH2 having the amino acid sequence set forth in SEQ ID NO:61 and a CDRH3 having the amino acid sequence set forth in SEQ ID NO:62;
c. a CDRH1 having the amino acid sequence set forth in SEQ ID NO:275, a CDRH2 having the amino acid sequence set forth in SEQ ID NO:276 and a CDRH3 having the amino acid sequence set forth in SEQ ID NO:277;
d. a CDRH1 having the amino acid sequence set forth in SEQ ID NO:278, a CDRH2 having the amino acid sequence set forth in SEQ ID NO:279 and a CDRH3 having the amino acid sequence set forth in SEQ ID NO:280;
e. a CDRH1 having the amino acid sequence set forth in SEQ ID NO:282, a CDRH2 having the amino acid sequence set forth in SEQ ID NO:283 and a CDRH3 having the amino acid sequence set forth in SEQ ID NO:284;
f. a CDRH1 having the amino acid sequence set forth in SEQ ID NO:285, a CDRH2 having the amino acid sequence set forth in SEQ ID NO:286 and a CDRH3 having the amino acid sequence set forth in SEQ ID NO:287;
g. a CDRH1 having the amino acid sequence set forth in SEQ ID NO:289, a CDRH2 having the amino acid sequence set forth in SEQ ID NO:290 and a CDRH3 having the amino acid sequence set forth in SEQ ID NO:291;
h. a CDRH1 having the amino acid sequence set forth in SEQ ID NO:292, a CDRH2 having the amino acid sequence set forth in SEQ ID NO:293 and a CDRH3 having the amino acid sequence set forth in SEQ ID NO:294;
i. a CDRH1 having the amino acid sequence set forth in SEQ ID NO:296, a CDRH2 having the amino acid sequence set forth in SEQ ID NO:297 and a CDRH3 having the amino acid sequence set forth in SEQ ID NO:298;
j. a CDRH1 having the amino acid sequence set forth in SEQ ID NO:299, a CDRH2 having the amino acid sequence set forth in SEQ ID NO:300 and a CDRH3 having the amino acid sequence set forth in SEQ ID NO:301;
k. a CDRH1 having the amino acid sequence set forth in SEQ ID NO:303, a CDRH2 having the amino acid sequence set forth in SEQ ID NO:304 and a CDRH3 having the amino acid sequence set forth in SEQ ID NO:305;
l. a CDRH1 having the amino acid sequence set forth in SEQ ID NO:306, a CDRH2 having the amino acid sequence set forth in SEQ ID NO:307 and a CDRH3 having the amino acid sequence set forth in SEQ ID NO:308;
m. a CDRH1 having the amino acid sequence set forth in SEQ ID NO:310, a CDRH2 having the amino acid sequence set forth in SEQ ID NO:311 and a CDRH3 having the amino acid sequence set forth in SEQ ID NO:312;
n. a CDRH1 having the amino acid sequence set forth in SEQ ID NO:313, a CDRH2 having the amino acid sequence set forth in SEQ ID NO:314 and a CDRH3 having the amino acid sequence set forth in SEQ ID NO:315;
o. a CDRH1 having the amino acid sequence set forth in SEQ ID NO:317, a CDRH2 having the amino acid sequence set forth in SEQ ID NO:318 and a CDRH3 having the amino acid sequence set forth in SEQ ID NO:319;
p. a CDRH1 having the amino acid sequence set forth in SEQ ID NO:320, a CDRH2 having the amino acid sequence set forth in SEQ ID NO:321 and a CDRH3 having the amino acid sequence set forth in SEQ ID NO:322;
q. a CDRH1 having the amino acid sequence set forth in SEQ ID NO:324, a CDRH2 having the amino acid sequence set forth in SEQ ID NO:325 and a CDRH3 having the amino acid sequence set forth in SEQ ID NO:326;
r. a CDRH1 having the amino acid sequence set forth in SEQ ID NO:327, a CDRH2 having the amino acid sequence set forth in SEQ ID NO:328 and a CDRH3 having the amino acid sequence set forth in SEQ ID NO:329;
s. a CDRH1 having the amino acid sequence set forth in SEQ ID NO:331, a CDRH2 having the amino acid sequence set forth in SEQ ID NO:332 and a CDRH3 having the amino acid sequence set forth in SEQ ID NO:333;
t. a CDRH1 having the amino acid sequence set forth in SEQ ID NO:334, a CDRH2 having the amino acid sequence set forth in SEQ ID NO:335 and a CDRH3 having the amino acid sequence set forth in SEQ ID NO:336;
u. a CDRH1 having the amino acid sequence set forth in SEQ ID NO:338, a CDRH2 having the amino acid sequence set forth in SEQ ID NO:339 and a CDRH3 having the amino acid sequence set forth in SEQ ID NO:340;
v. a CDRH1 having the amino acid sequence set forth in SEQ ID NO:341, a CDRH2 having the amino acid sequence set forth in SEQ ID NO:342 and a CDRH3 having the amino acid sequence set forth in SEQ ID NO:343;
w. a CDRH1 having the amino acid sequence set forth in SEQ ID NO:345, a CDRH2 having the amino acid sequence set forth in SEQ ID NO:346 and a CDRH3 having the amino acid sequence set forth in SEQ ID NO:347;
x. a CDRH1 having the amino acid sequence set forth in SEQ ID NO:348, a CDRH2 having the amino acid sequence set forth in SEQ ID NO:349 and a CDRH3 having the amino acid sequence set forth in SEQ ID NO:350;
y. a CDRH1 having the amino acid sequence set forth in SEQ ID NO:352, a CDRH2 having the amino acid sequence set forth in SEQ ID NO:353 and a CDRH3 having the amino acid sequence set forth in SEQ ID NO:354;
z. a CDRH1 having the amino acid sequence set forth in SEQ ID NO:355, a CDRH2 having the amino acid sequence set forth in SEQ ID NO:356 and a CDRH3 having the amino acid sequence set forth in SEQ ID NO:357;
aa. a CDRH1 having the amino acid sequence set forth in SEQ ID NO:359, a CDRH2 having the amino acid sequence set forth in SEQ ID NO:360 and a CDRH3 having the amino acid sequence set forth in SEQ ID NO:361, or
bb. a CDRH1 having the amino acid sequence set forth in SEQ ID NO:362, a CDRH2 having the amino acid sequence set forth in SEQ ID NO:363 and a CDRH3 having the amino acid sequence set forth in SEQ ID NO:364 and/or wherein at least one of the one or more antigen binding domains is an antigen binding domain 3 (ABD3) that is capable of specifically binding to CD47 and comprises:
a. a heavy chain complementarity determining region 1 (CDRH1) having the amino acid sequence set forth in SEO ID NO:64, a heavy chain complementarity determining region 2 (CDRH2) having the amino acid sequence set forth in SEO ID NO:65 and a heavy chain complementarity determining region 3 (CDRH3) having the amino acid sequence set forth in SEO ID NO:66: or
b. a CDRH1 having the amino acid sequence set forth in SEO ID NO:67, a CDRH2 having the amino acid sequence set forth in SEO ID NO:68 and a CDRH3 having the amino acid sequence set forth in SEO ID NO:69.

8. (canceled)

9. The binding agent of claim 7, wherein the binding agent ABD2 comprises:
a. an amino acid sequence at least 80% identical to the amino acid sequence set forth in SEQ ID NO:63;
b. an amino acid sequence at least 80% identical to the amino acid sequence set forth in SEQ ID NO:281;
c. an amino acid sequence at least 80% identical to the amino acid sequence set forth in SEQ ID NO:288;
d. an amino acid sequence at least 80% identical to the amino acid sequence set forth in SEQ ID NO:295;
e. an amino acid sequence at least 80% identical to the amino acid sequence set forth in SEQ ID NO:302;
f. an amino acid sequence at least 80% identical to the amino acid sequence set forth in SEQ ID NO:309;
g. an amino acid sequence at least 80% identical to the amino acid sequence set forth in SEQ ID NO:316;
h. an amino acid sequence at least 80% identical to the amino acid sequence set forth in SEQ ID NO:323;
i. an amino acid sequence at least 80% identical to the amino acid sequence set forth in SEQ ID NO:330 or;
j. an amino acid sequence as set forth in SEQ ID NO:379.

10. The binding agent of claim 7, wherein ABD3 comprises an amino acid sequence at least 80% identical to the amino acid sequence set forth in SEQ ID NO:70.

11. (canceled)

12. The binding agent of claim 7, wherein the binding agent comprises a) an antigen binding domain 1 (ABD1) comprising an amino acid sequence at least 85% identical to the amino acid sequence set forth in SEQ ID NO:7, SEQ ID NO:14, SEQ ID NO:21, SEQ ID NO:28, SEQ ID NO:35, SEQ ID NO:42, SEQ ID NO:49, SEQ ID NO:56, SEQ ID NO:77, SEQ ID NO:84, SEQ ID NO:91, SEQ ID NO:98, SEQ ID NO:105, SEQ ID NO:112, SEQ ID NO:119, SEQ ID NO:126, SEQ ID NO:133, or SEQ ID NO:140, b) an antigen binding domain 2 (ABD2) comprising an amino acid sequence at least 85% identical to the amino acid sequence set forth in SEQ ID NO:63, SEQ ID NO:281, SEQ ID NO:288, SEQ ID NO:295, SEQ ID NO:302, SEQ ID NO:309, SEQ ID NO:316, SEQ ID NO:323, SEQ ID NO:330, SEQ ID NO:337, SEQ ID NO:344, SEQ ID NO:351, SEQ ID NO:358 or SEQ ID NO:365 and c) an antigen binding domain 3 (ABD3) comprising an amino acid sequence at least 85% identical to the amino acid sequence set forth in SEQ ID NO:70.

13. The binding agent of claim 7, wherein the binding agent comprises:
a. at least one an antigen binding domain 1 (ABD1) comprising an amino acid sequence at least 95% identical to the amino acid sequence set forth in SEQ ID NO:7, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
b. at least one antigen binding domain 1 (ABD1) comprising an amino acid sequence at least 95% identical to the amino acid sequence set forth in SEQ ID NO:14, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
c. at least one antigen binding domain 1 (ABD1) comprising an amino acid sequence at least 95% identical to the amino acid sequence set forth in SEQ ID NO:21, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
d. at least one antigen binding domain 1 (ABD1) comprising an amino acid sequence at least 95% identical to the amino acid sequence set forth in SEQ ID NO:28, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
e. at least one antigen binding domain 1 (ABD1) comprising an amino acid sequence at least 95% identical to the amino acid sequence set forth in SEQ ID NO:35, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
f. at least one antigen binding domain 1 (ABD1) comprising an amino acid sequence at least 95% identical to the amino acid sequence set forth in SEQ ID NO:42, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
g. at least one antigen binding domain 1 (ABD1) comprising an amino acid sequence at least 95% identical to the amino acid sequence set forth in SEQ ID NO:49, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
h. at least one antigen binding domain 1 (ABD1) comprising an amino acid sequence at least 95% identical to the amino acid sequence set forth in SEQ ID NO:56, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
i. at least one antigen binding domain 1 (ABD1) comprising an amino acid sequence at least 95% identical to the amino acid sequence set forth in SEQ ID NO:77, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
j. at least one antigen binding domain 1 (ABD1) comprising an amino acid sequence at least 95% identical to the amino acid sequence set forth in SEQ ID NO:84, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
k. at least one antigen binding domain 1 (ABD1) comprising an amino acid sequence at least 95% identical to the amino acid sequence set forth in SEQ ID NO:91, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
l. at least one antigen binding domain 1 (ABD1) comprising an amino acid sequence at least 95% identical to the amino acid sequence set forth in SEQ ID NO:98, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
m. at least one antigen binding domain 1 (ABD1) comprising an amino acid sequence at least 95% identical to the amino acid sequence set forth in SEQ ID NO:105, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
n. at least one antigen binding domain 1 (ABD1) comprising an amino acid sequence at least 95% identical to the amino acid sequence set forth in SEQ ID NO:112, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
o. at least one antigen binding domain 1 (ABD1) comprising an amino acid sequence at least 95% identical to the amino acid sequence set forth in SEQ ID NO:119, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
p. at least one antigen binding domain 1 (ABD1) comprising an amino acid sequence at least 95% identical to the amino acid sequence set forth in SEQ ID NO:126, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70;
q. at least one antigen binding domain 1 (ABD1) comprising an amino acid sequence at least 95% identical to the amino acid sequence set forth in SEQ ID NO:133, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70; or
r. at least one antigen binding domain 1 (ABD1) comprising an amino acid sequence at least 95% identical to the amino acid sequence set forth in SEQ ID NO:140, at least one antigen binding domain 2 (ABD2) comprising an amino acid sequence at least 95% identical to the amino acid sequence set forth in SEQ ID NO:63 and at least one antigen binding domain 3 (ABD3) comprising an amino acid sequence at least 95% identical to the amino acid sequence set forth in SEQ ID NO:70.

14.-18. (canceled)

19. The binding agent of claim 13, wherein the binding agent comprises two polypeptide chains that are capable of assembling to form a dimer and wherein each polypeptide chain are the same.

20.-38. (canceled)

39. The binding agent of claim 19, wherein each of the polypeptide chains independently comprises in a N- to C-terminal fashion an amino acid sequence of formula III 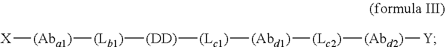
wherein one of Abₐ₁, Ab_{d1} or Ab_{d2} represents the antigen binding domain 1 (ABD1), one of Abₐ₁, Ab_{d1} or Ab_{d2} represents the antigen binding domain 2 (ABD2) and one of Abₐ₁, Ab_{d1} or Ab_{d2} represents the antigen binding domain 3 (ABD3);
wherein X or Y are independently present or absent and comprises an amino acid sequence;
wherein L_{b1} comprises a linker or a hinge region and wherein, L_{c1}, L_{c2}, each independently comprises one or more linkers; and
wherein DD represents a dimerization domain.

40. The binding agent of claim 39, wherein each of the polypeptide chains independently comprises in a N- to C-terminal fashion an amino acid sequence of formula IIIa: 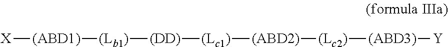
of formula IIIb:

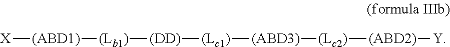

41.-43. (canceled)

44. The binding agent of claim 40, wherein the dimerization is a CH2-CH3 domain of a natural human antibody heavy chain.

45.-56. (canceled)

57. The binding agent of claim 7, wherein the antigen binding domain is a VHH.

58.-60. (canceled)

61. The binding agent of claim 1, wherein the binding agent is or comprises a single domain antibody.

62.-67. (canceled)

68. The binding agent of claim 1, wherein the binding agent comprises:
a. an amino acid sequence at least 80% identical to the amino acid sequence set forth in SEQ ID NO:141;
b. an amino acid sequence at least 80% identical to the amino acid sequence set forth in SEQ ID NO:142;
c. an amino acid sequence at least 80% identical to the amino acid sequence set forth in SEQ ID NO:143;
d. an amino acid sequence at least 80% identical to the amino acid sequence set forth in SEQ ID NO:144;
e. an amino acid sequence at least 80% identical to the amino acid sequence set forth in SEQ ID NO:145;
f. an amino acid sequence at least 80% identical to the amino acid sequence set forth in SEQ ID NO:146;
g. an amino acid sequence at least 80% identical to the amino acid sequence set forth in SEQ ID NO:147;
h. an amino acid sequence at least 80% identical to the amino acid sequence set forth in SEQ ID NO:148;
i. an amino acid sequence at least 80% identical to the amino acid sequence set forth in SEQ ID NO:149;
j. an amino acid sequence at least 80% identical to the amino acid sequence set forth in SEQ ID NO:150;
k. an amino acid sequence at least 80% identical to the amino acid sequence set forth in SEQ ID NO:151;
l. an amino acid sequence at least 80% identical to the amino acid sequence set forth in SEQ ID NO:152;
m. an amino acid sequence at least 80% identical to the amino acid sequence set forth in SEQ ID NO:153;
n. an amino acid sequence at least 80% identical to the amino acid sequence set forth in SEQ ID NO:154;
o. an amino acid sequence at least 80% identical to the amino acid sequence set forth in SEQ ID NO:155;
p. an amino acid sequence at least 80% identical to the amino acid sequence set forth in SEQ ID NO:156;
q. an amino acid sequence at least 80% identical to the amino acid sequence set forth in SEQ ID NO:157 or,
r. an amino acid sequence at least 80% identical to the amino acid sequence set forth in SEQ ID NO:158.

69.-70. (canceled)

71. The binding agent of claim 7, wherein the binding agent comprises an amino acid sequence at least 80% identical to the amino acid sequence set forth in SEQ ID NO:159 or as set forth in SEQ ID NO:159.

72. The binding agent of claim 71, wherein the binding agent comprises two polypeptide chains each having an amino acid sequence at least 95% identical to the amino acid sequence set forth in SEQ ID NO:159.

73.-79. (canceled)

80. The binding agent of claim 1, wherein the binding agent is conjugated to a therapeutic moiety.

81.-91. (canceled)

92. A pharmaceutical composition comprising the binding agent of any claim 72 and a pharmaceutically acceptable carrier.

93. (canceled)

94. A nucleic acid or set of nucleic acids encoding the binding agent of claim 1 or a vector comprising same.

95.-99. (canceled)

100. A method of treating or preventing a disease or disorder associated with CD36, CD36 expression, CD36 overexpression or CD36 upregulation in a subject, comprising administering to the subject the binding agent of claim 72.

101. A method of treating a cancer comprising administering the binding agent of claim 72 to a subject in need.

102.-105. (canceled)

106. The method of claim 101, wherein the cancer is leukemia, acute myeloid leukemia, endometrial cancer, breast cancer (e.g., triple negative breast cancer), liver cancer (e.g., hepatocellular carcinoma), lung cancer, prostate cancer, renal cancer e.g., (renal clear cell cancer), ovarian cancer, cervical cancer, pancreatic cancer, gastric cancer, bladder cancer (e.g., bladder urothelial carcinoma), brain cancer, oral cancer, head and neck cancer, glioblastoma (e.g., glioblastoma multiform), adrenocortical carcinoma, colon cancer or colorectal cancer.

107.-114. (canceled)


SEQUENCE LISTING (383 sequences)

SEQ ID NO: 1 - protein, 5 aa, synthetic construct
SYAMS

SEQ ID NO: 2 - protein, 17 aa, synthetic construct
LITSAGGTTDYADSVKG

SEQ ID NO: 3 - protein, 9 aa, synthetic construct
WRASLYEPY

SEQ ID NO: 4 - protein, 8 aa, synthetic construct
GFTFSSYA

SEQ ID NO: 5 - protein, 8 aa, synthetic construct
ITSAGGTT

SEQ ID NO: 6 - protein, 11 aa, synthetic construct
SAWRASLYEPY

SEQ ID NO: 7 - protein, 118 aa, synthetic construct
QVQLQESGGGLVQPGGSLRLSCAASGFTFSSYAMSWYRQAPGKERELVALITSAGGTTDYADSVKGRFTISRDDAEKTVSLQMNSLKPEDTAVYYCSAWRASLYEPYWGQGTQVTVSS

SEQ ID NO: 8 - protein, 5 aa, synthetic construct
NYAMS

SEQ ID NO: 9 - protein, 17 aa, synthetic construct
AMQSTGGTTYYADSVKG

SEQ ID NO: 10 - protein, 11 aa, synthetic construct
VTLQGWSEYDY

SEQ ID NO: 11 - protein, 8 aa, synthetic construct
GFTFSNYA

SEQ ID NO: 12 - protein, 8 aa, synthetic construct
MQSTGGTT

SEQ ID NO: 13 - protein, 13 aa, synthetic construct
NAVTLQGWSEYDY

SEQ ID NO: 14 - protein, 120 aa, synthetic construct
QVQLQESGGGLVQPGGSLRLSCAASGFTFSNYAMSWYRQTPGKERELVAAMQSTGGTTYYADSVKGRFTISRDDAKNTVYLQMNSLKPEDTAVYYCNAVTLQGWSEYDYWGQGTQVTVSS

SEQ ID NO: 15 - protein, 5 aa, synthetic construct
SYAMS

SEQ ID NO: 16 - protein, 17 aa, synthetic construct
AIPHDGGSIYYVDSVKG

SEQ ID NO: 17 - protein, 11 aa, synthetic construct
VTLQGWREYDY

SEQ ID NO: 18 - protein, 8 aa, synthetic construct
GFTFSSYA

SEQ ID NO: 19 - protein, 8 aa, synthetic construct
IPHDGGSI

SEQ ID NO: 20 - protein, 13 aa, synthetic construct
NAVTLQGWREYDY

SEQ ID NO: 21 - protein, 120 aa, synthetic construct
QVQLQESGGGLVQPGGSLRLSCTASGFTFSSYAMSWYRQAPGKERELVAAIPHDGGSIYYVDSVKGRFSISRDNAKNTVYLQMNSLKPEDTAVYYCNAVTLQGWREYDYWGQGTQVTVSS

SEQ ID NO: 22 - protein, 5 aa, synthetic construct
TYAMS

SEQ ID NO: 23 - protein, 17 aa, synthetic construct
LITSAGGTTDYADSVKG

SEQ ID NO: 24 - protein, 9 aa, synthetic construct
WRATLFKPY

SEQ ID NO: 25 - protein, 8 aa, synthetic construct
GLAFSTYA

SEQ ID NO: 26 - protein, 8 aa, synthetic construct
ITSAGGTT

SEQ ID NO: 27 - protein, 11 aa, synthetic construct
SAWRATLFKPY

SEQ ID NO: 28 - protein, 118 aa, synthetic construct
QVQLQESGGGLVQPGGSLRLSCAASGLAFSTYAMSWYRQAPGKERELVALITSAGGTTDYADSVKGRFTISRDDAEKTVSLQMNSLKPEDTAVYYCSAWRATLFKPYWGQGTQVTVSS

SEQ ID NO: 29 - protein, 9 aa, synthetic construct
FTFSSYAMS

SEQ ID NO: 30 - protein, 17 aa, synthetic construct
AITNAGDSTHYADSVKG

SEQ ID NO: 31 - protein, 11 aa, synthetic construct
VTLQGWSEYDY

SEQ ID NO: 32 - protein, 12 aa, synthetic construct
GFTSGFTFSSYA

SEQ ID NO: 33 - protein, 8 aa, synthetic construct
ITNAGDST

SEQ ID NO: 34 - protein, 13 aa, synthetic construct
NAVTLQGWSEYDY

SEQ ID NO: 35 - protein, 124 aa, synthetic construct
QVQLQESGGGLVQPGGSLRLSCAASGFTSGFTFSSYAMSWYRQAPGKERELVAAITNAGDSTHYADSVKGRFTISRDNAKNTVYLLMNSLKPEDTAAYYCNAVTLQGWSEYDYWGQGTQVTVSS

SEQ ID NO: 36 - protein, 5 aa, synthetic construct
YFAIG

SEQ ID NO: 37 - protein, 17 aa, synthetic construct
CISSSGSNTYYADSVKG

SEQ ID NO: 38 - protein, 11 aa, synthetic construct
HDYCSRAPYDY

SEQ ID NO: 39 - protein, 8 aa, synthetic construct
GFSLDYFA

SEQ ID NO: 40 - protein, 8 aa, synthetic construct
ISSSGSNT

SEQ ID NO: 41 - protein, 13 aa, synthetic construct
AAHDYCSRAPYDY

SEQ ID NO: 42 - protein, 120 aa, synthetic construct
QVQLQESGGGLVQPGGSLRLSCAASGFSLDYFAIGWFRQAPGKEREGISCISSSGSNTYYADSVKGRFTISRDNAKNTVYLQMNSLKPEDTAVYYCAAHDYCSRAPYDYWGQGTQVTVSS

SEQ ID NO: 43 - protein, 5 aa, synthetic construct
NYAMS

SEQ ID NO: 44 - protein, 17 aa, synthetic construct
AMQSTGGTTYYADSVKG

SEQ ID NO: 45 - protein, 11 aa, synthetic construct
VTLQGWSEYDY

SEQ ID NO: 46 - protein, 8 aa, synthetic construct
GFTFSNYA

SEQ ID NO: 47 - protein, 8 aa, synthetic construct
MQSTGGTT

SEQ ID NO: 48 - protein, 13 aa, synthetic construct
NAVTLQGWSEYDY

SEQ ID NO: 49 - protein, 120 aa, synthetic construct
QVQLQESGGGLVQPGESLRLSCAASGFTFSNYAMSWYRQTPGKERELVAAMQSTGGTTYYADSVKGRFTISRDDAKNTVYLQMNSLKPEDTAVYYCNAVTLQGWSEYDYWGQGTQVTVSS

SEQ ID NO: 50 - protein, 5 aa, synthetic construct
SSIMS

SEQ ID NO: 51 - protein, 17 aa, synthetic construct
SISSDSVRTNYAESVKG

SEQ ID NO: 52 - protein, 7 aa, synthetic construct
PSIRGRD

SEQ ID NO: 53 - protein, 8 aa, synthetic construct
GFRFSSSI

SEQ ID NO: 54 - protein, 8 aa, synthetic construct
ISSDSVRT

SEQ ID NO: 55 - protein, 9 aa, synthetic construct
TRPSIRGRD

SEQ ID NO: 56 - protein, 116 aa, synthetic construct
QVQLQESGGGLVQPGGSLRLSCAASGFRFSSSIMSWYRQAPEKERELVASISSDSVRTNYAESVKGRFTISRDNEQSTLFLEMNNLKPVDTAVYFCTRPSIRGRDWGQGTQVTVSS

SEQ ID NO: 57 - protein, 5 aa, synthetic construct
RSSMS

SEQ ID NO: 58 - protein, 17 aa, synthetic construct
IISSSGIITHYSDSVKG

SEQ ID NO: 59 - protein, 9 aa, synthetic construct
VLDDDSGDY

SEQ ID NO: 60 - protein, 8 aa, synthetic construct
GFTFSRSS

SEQ ID NO: 61 - protein, 8 aa, synthetic construct
ISSSGIIT

SEQ ID NO: 62 - protein, 11 aa, synthetic construct
NAVLDDDSGDY

SEQ ID NO: 63 - protein, 118 aa, synthetic construct
QVQLQESGGGWVQPGGSLRLSCVASGFTFSRSSMSWHRQAPGKERDLVAIISSSGIITHYSDSVKGRFTISRDNAKDTVYLQMNSLKPEDTAVYVCNAVLDDDSGDYWGQGTQVTVSS

SEQ ID NO: 64 - protein, 5 aa, synthetic construct
VNDIR

SEQ ID NO: 65 - protein, 16 aa, synthetic construct
RITGGGRTDYADSVKG

SEQ ID NO: 66 - protein, 5 aa, synthetic construct
WGRGY

SEQ ID NO: 67 - protein, 8 aa, synthetic construct
RFDFSVND

SEQ ID NO: 68 - protein, 7 aa, synthetic construct
ITGGGRT

SEQ ID NO: 69 - protein, 5 aa, synthetic construct
WGRGY

SEQ ID NO: 70 - protein, 111 aa, synthetic construct
QVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSS

SEQ ID NO: 71 - protein, 5 aa, synthetic construct
ADVMG

SEQ ID NO: 72 - protein, 17 aa, synthetic construct
AADWRGLNTWYRDSVKG

SEQ ID NO: 73 - protein, 10 aa, synthetic construct
GGWAFSPGAS

SEQ ID NO: 74 - protein, 8 aa, synthetic construct
GRTGSADV

SEQ ID NO: 75 - protein, 8 aa, synthetic construct
ADWRGLNT

SEQ ID NO: 76 - protein, 12 aa, synthetic construct
NTGGWAFSPGAS

SEQ ID NO: 77 - protein, 119 aa, synthetic construct
QVQLQESGGGLVQPGGSLRLSCTASGRTGSADVMGWFRRAPGKDREFVAAADWRGLNTWYRDSVKGRFTISRDNAKSTMYLQMNSLKPTDTAVYYCNTGGWAFSPGASWGQGTQVTVSS

SEQ ID NO: 78 - protein, 5 aa, synthetic construct
TSIMT

SEQ ID NO: 79 - protein, 17 aa, synthetic construct
SISSDSRRTNYAESVKG

SEQ ID NO: 80 - protein, 7 aa, synthetic construct
PNIRGRD

SEQ ID NO: 81 - protein, 8 aa, synthetic construct
GFRFSTSI

SEQ ID NO: 82 - protein, 8 aa, synthetic construct
ISSDSRRT

SEQ ID NO: 83 - protein, 9 aa, synthetic construct
TRPNIRGRD

SEQ ID NO: 84 - protein, 116 aa, synthetic construct
QVQLQESGGGLVQPGGSLRLSCAASGFRFSTSIMTWYRQAPGKERELVASISSDSRRTNYAESVKGRFTISRDNEQRMVFLEMNSLKPVDTAVYFCTRPNIRGRDWGQGTQVTVSS

SEQ ID NO: 85 - protein, 5 aa, synthetic construct
NYYMS

SEQ ID NO: 86 - protein, 17 aa, synthetic construct
GINMTGGSADYVDSVKG

SEQ ID NO: 87 - protein, 9 aa, synthetic construct
FSVWPPNNY

SEQ ID NO: 88 - protein, 8 aa, synthetic construct
GFTFSNYY

SEQ ID NO: 89 - protein, 8 aa, synthetic construct
INMTGGSA

SEQ ID NO: 90 - protein, 11 aa, synthetic construct
HTFSVWPPNNY

SEQ ID NO: 91 - protein, 118 aa, synthetic construct
QVQLQESGGGLVQLGGSLRLSCAASGFTFSNYYMSWYRQAPGKERELVGGINMTGGSADYVDSVKGRFTISRDNTKNTVYLQMNSLRVEDTAVYYCHTFSVWPPNNYWGQGTQVTVSS

SEQ ID NO: 92 - protein, 5 aa, synthetic construct
TYAMS

SEQ ID NO: 93 - protein, 17 aa, synthetic construct
LITTTGGTTDYADSVKG

SEQ ID NO: 94 - protein, 9 aa, synthetic construct
WRATLLKPY

SEQ ID NO: 95 - protein, 8 aa, synthetic construct
GFSFSTYA

SEQ ID NO: 96 - protein, 8 aa, synthetic construct
ITTTGGTT

SEQ ID NO: 97 - protein, 11 aa, synthetic construct
SAWRATLLKPY

SEQ ID NO: 98 - protein, 118 aa, synthetic construct
QVQLQESGGGLVQPGGSLRLSCAASGFSFSTYAMSWYRQPPGKERELVALITTTGGTTDYADSVKGRFTISRDDAEKTVSLQMNSLKPEDTAVYYCSAWRATLLKPYWGQGTQVTVSS

SEQ ID NO: 99 - protein, 5 aa, synthetic construct
SYPMG

SEQ ID NO: 100 - protein, 17 aa, synthetic construct
AIDWSGDITYYADSVKG

SEQ ID NO: 101 - protein, 13 aa, synthetic construct
GPLDSWERNDFPS

SEQ ID NO: 102 - protein, 8 aa, synthetic construct
GRTFSSYP

SEQ ID NO: 103 - protein, 8 aa, synthetic construct
IDWSGDIT

SEQ ID NO: 104 - protein, 15 aa, synthetic construct
NAGPLDSWERNDFPS

SEQ ID NO: 105 - protein, 122 aa, synthetic construct
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWGQGTQVTVSS

SEQ ID NO: 106 - protein, 5 aa, synthetic construct
SYPMG

SEQ ID NO: 107 - protein, 17 aa, synthetic construct
AIDWSGDITYYADSVKG

SEQ ID NO: 108 - protein, 13 aa, synthetic construct
GPLDSWERNDFPS

SEQ ID NO: 109 - protein, 8 aa, synthetic construct
GRTFSSYP

SEQ ID NO: 110 - protein, 8 aa, synthetic construct
IDWSGDIT

SEQ ID NO: 111 - protein, 15 aa, synthetic construct
NAGPLDSWERNDFPS

SEQ ID NO: 112 - protein, 122 aa, synthetic construct
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWDQGTQVTVSS

SEQ ID NO: 113 - protein, 5 aa, synthetic construct
TYAMS

SEQ ID NO: 114 - protein, 17 aa, synthetic construct
LITSAGGITDYADSVKG

SEQ ID NO: 115 - protein, 9 aa, synthetic construct
WRATLFKPY

SEQ ID NO: 116 - protein, 8 aa, synthetic construct
GFTVSTYA

SEQ ID NO: 117 - protein, 8 aa, synthetic construct
ITSAGGIT

SEQ ID NO: 118 - protein, 11 aa, synthetic construct
SAWRATLFKPY

SEQ ID NO: 119 - protein, 118 aa, synthetic construct
QVQLQESGGGLAQPGGSLRLSCAASGFTVSTYAMSWYRQAPGKERELVALITSAGGITDYADSVKGRFTIARDDAEKTVSLQMNSLKPEDTAVYYCSAWRATLFKPYWGQGTQVTVSS

SEQ ID NO: 120 - protein, 5 aa, synthetic construct
TSAMA

SEQ ID NO: 121 - protein, 16 aa, synthetic construct
GISSGGTVYYADSVKG

SEQ ID NO: 122 - protein, 18 aa, synthetic construct
GPEVHDDVFRLCGPTTDY

SEQ ID NO: 123 - protein, 8 aa, synthetic construct
GDTICTSA

SEQ ID NO: 124 - protein, 7 aa, synthetic construct
ISSGGTV

SEQ ID NO: 125 - protein, 20 aa, synthetic construct
NAGPEVHDDVFRLCGPTTDY

SEQ ID NO: 126 - protein, 126 aa, synthetic construct
QVQLQESGGGLVQAGGSLRLSCAASGDTICTSAMAWYRQAPGKERELVAGISSGGTVYYADSVKGRFTISRDNAKNTVYLQMNTLKPEDTAVYYCNAGPEVHDDVFRLCGPTTDYWGQGTQVTVSS

SEQ ID NO: 127 - protein, 5 aa, synthetic construct
YYAIG

SEQ ID NO: 128 - protein, 17 aa, synthetic construct
YISNSDGNTYYADSVKG

SEQ ID NO: 129 - protein, 17 aa, synthetic construct
DQGSSVMHGFIEEWYDY

SEQ ID NO: 130 - protein, 8 aa, synthetic construct
GFTLDYYA

SEQ ID NO: 131 - protein, 8 aa, synthetic construct
ISNSDGNT

SEQ ID NO: 132 - protein, 19 aa, synthetic construct
AADQGSSVMHGFIEEWYDY

SEQ ID NO: 133 - protein, 126 aa, synthetic construct
QVQLQESGGGLVQPGGSLRLSCAASGFTLDYYAIGWFRQAPGKEREGIVYISNSDGNTYYADSVKGRFTISRDNAKNTVYLQMNSLKPEDTAVYYCAADQGSSVMHGFIEEWYDYWGQGTQVTVSS

SEQ ID NO: 134 - protein, 5 aa, synthetic construct
SYAMG

SEQ ID NO: 135 - protein, 17 aa, synthetic construct
AISWSGGRTYYDDPVKG

SEQ ID NO: 136 - protein, 16 aa, synthetic construct
DPEYSGTWNPWAGSGY

SEQ ID NO: 137 - protein, 8 aa, synthetic construct
GRTFSSYA

SEQ ID NO: 138 - protein, 8 aa, synthetic construct
ISWSGGRT

SEQ ID NO: 139 - protein, 18 aa, synthetic construct
NLDPEYSGTWNPWAGSGY

SEQ ID NO: 140 - protein, 125 aa, synthetic construct
QVQLQESGGGLVQAGGSLRLSCAASGRTFSSYAMGWFRQAPGKEREFVAAISWSGGRTYYDDPVKGRFTISRDNAKNTVYLQMNSLKPEDTAVYYCNLDPEYSGTWNPWAGSGYWGQGTQVTVSS

SEQ ID NO: 141 - protein, 350 aa, synthetic construct
QVQLQESGGGLVQPGGSLRLSCAASGFTFSSYAMSWYRQAPGKERELVALITSAGGTTDYADSVKGRFTISRDDAEKTVSLQMNSLKPEDTAVYYCSAWRASLYEPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 352 aa, synthetic construct
QVQLQESGGGLVQPGGSLRLSCAASGFTFSNYAMSWYRQTPGKERELVAAMQSTGGTTYYADSVKGRFTISRDDAKNTVYLQMNSLKPEDTAVYYCNAVTLQGWSEYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 352 aa, synthetic construct
QVQLQESGGGLVQPGGSLRLSCTASGFTFSSYAMSWYRQAPGKERELVAAIPHDGGSIYYVDSVKGRFSISRDNAKNTVYLQMNSLKPEDTAVYYCNAVTLQGWREYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 350 aa, synthetic construct
QVQLQESGGGLVQPGGSLRLSCAASGLAFSTYAMSWYRQAPGKERELVALITSAGGTTDYADSVKGRFTISRDDAEKTVSLQMNSLKPEDTAVYYCSAWRATLFKPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 356 aa, synthetic construct
QVQLQESGGGLVQPGGSLRLSCAASGFTSGFTFSSYAMSWYRQAPGKERELVAAITNAGDSTHYADSVKGRFTISRDNAKNTVYLLMNSLKPEDTAAYYCNAVTLQGWSEYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 352 aa, synthetic construct
QVQLQESGGGLVQPGGSLRLSCAASGFSLDYFAIGWFRQAPGKEREGISCISSSGSNTYYADSVKGRFTISRDNAKNTVYLQMNSLKPEDTAVYYCAAHDYCSRAPYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 352 aa, synthetic construct
QVQLQESGGGLVQPGESLRLSCAASGFTFSNYAMSWYRQTPGKERELVAAMQSTGGTTYYADSVKGRFTISRDDAKNTVYLQMNSLKPEDTAVYYCNAVTLQGWSEYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 348 aa, synthetic construct
QVQLQESGGGLVQPGGSLRLSCAASGFRFSSSIMSWYRQAPEKERELVASISSDSVRTNYAESVKGRFTISRDNEQSTLFLEMNNLKPVDTAVYFCTRPSIRGRDWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 276 aa, synthetic construct
KSTMYLQMNSLKPTDTAVYYCNTGGWAFSPGASWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 348 aa, synthetic construct
QVQLQESGGGLVQPGGSLRLSCAASGFRFSTSIMTWYRQAPGKERELVASISSDSRRTNYAESVKGRFTISRDNEQRMVFLEMNSLKPVDTAVYFCTRPNIRGRDWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 350 aa, synthetic construct
QVQLQESGGGLVQLGGSLRLSCAASGFTFSNYYMSWYRQAPGKERELVGGINMTGGSADYVDSVKGRFTISRDNTKNTVYLQMNSLRVEDTAVYYCHTFSVWPPNNYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 350 aa, synthetic construct
QVQLQESGGGLVQPGGSLRLSCAASGFSFSTYAMSWYRQPPGKERELVALITTTGGTTDYADSVKGRFTISRDDAEKTVSLQMNSLKPEDTAVYYCSAWRATLLKPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 354 aa, synthetic construct
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 354 aa, synthetic construct
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWDQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 350 aa, synthetic construct
QVQLQESGGGLAQPGGSLRLSCAASGFTVSTYAMSWYRQAPGKERELVALITSAGGITDYADSVKGRFTIARDDAEKTVSLQMNSLKPEDTAVYYCSAWRATLFKPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 358 aa, synthetic construct
QVQLQESGGGLVQAGGSLRLSCAASGDTICTSAMAWYRQAPGKERELVAGISSGGTVYYADSVKGRFTISRDNAKNTVYLQMNTLKPEDTAVYYCNAGPEVHDDVFRLCGPTTDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 358 aa, synthetic construct
QVQLQESGGGLVQPGGSLRLSCAASGFTLDYYAIGWFRQAPGKEREGIVYISNSDGNTYYADSVKGRFTISRDNAKNTVYLQMNSLKPEDTAVYYCAADQGSSVMHGFIEEWYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 357 aa, synthetic construct
QVQLQESGGGLVQAGGSLRLSCAASGRTFSSYAMGWFRQAPGKEREFVAAISWSGGRTYYDDPVKGRFTISRDNAKNTVYLQMNSLKPEDTAVYYCNLDPEYSGTWNPWAGSGYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 606 aa, synthetic construct
QVQLQESGGGLVQPGGSLRLSCAASGFTFSSYAMSWYRQAPGKERELVALITSAGGTTDYADSVKGRFTISRDDAEKTVSLQMNSLKPEDTAVYYCSAWRASLYEPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLQESGGGWVQPGGSLRLSCVASGFTFSRSSMSWHRQAPGKERDLVAIISSSGIITHYSDSVKGRFTISRDNAKDTVYLQMNSLKPEDTAVYVCNAVLDDDSGDYWGQGTQVTVSSGGGGSGGGGSGGGGSQVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSS

SEQ ID NO: 160 - protein, 631 aa, synthetic construct
QVQLQESGGGLVQPGGSLRLSCAASGFTFSSYAMSWYRQAPGKERELVALITSAGGTTDYADSVKGRFTISRDDAEKTVSLQMNSLKPEDTAVYYCSAWRASLYEPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 161 - protein, 633 aa, synthetic construct
QVQLQESGGGLVQPGGSLRLSCAASGFTFSNYAMSWYRQTPGKERELVAAMQSTGGTTYYADSVKGRFTISRDDAKNTVYLQMNSLKPEDTAVYYCNAVTLQGWSEYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 162 - protein, 633 aa, synthetic construct
QVQLQESGGGLVQPGGSLRLSCTASGFTFSSYAMSWYRQAPGKERELVAAIPHDGGSIYYVDSVKGRFSISRDNAKNTVYLQMNSLKPEDTAVYYCNAVTLQGWREYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 163 - protein, 631 aa, synthetic construct
QVQLQESGGGLVQPGGSLRLSCAASGLAFSTYAMSWYRQAPGKERELVALITSAGGTTDYADSVKGRFTISRDDAEKTVSLQMNSLKPEDTAVYYCSAWRATLFKPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 164 - protein, 637 aa, synthetic construct
QVQLQESGGGLVQPGGSLRLSCAASGFTSGFTFSSYAMSWYRQAPGKERELVAAITNAGDSTHYADSVKGRFTISRDNAKNTVYLLMNSLKPEDTAAYYCNAVTLQGWSEYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 165 - protein, 633 aa, synthetic construct
QVQLQESGGGLVQPGGSLRLSCAASGFSLDYFAIGWFRQAPGKEREGISCISSSGSNTYYADSVKGRFTISRDNAKNTVYLQMNSLKPEDTAVYYCAAHDYCSRAPYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 166 - protein, 633 aa, synthetic construct
QVQLQESGGGLVQPGESLRLSCAASGFTFSNYAMSWYRQTPGKERELVAAMQSTGGTTYYADSVKGRFTISRDDAKNTVYLQMNSLKPEDTAVYYCNAVTLQGWSEYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 167 - protein, 629 aa, synthetic construct
QVQLQESGGGLVQPGGSLRLSCAASGFRFSSSIMSWYRQAPEKERELVASISSDSVRTNYAESVKGRFTISRDNEQSTLFLEMNNLKPVDTAVYFCTRPSIRGRDWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 168 - protein, 632 aa, synthetic construct
QVQLQESGGGLVQPGGSLRLSCTASGRTGSADVMGWFRRAPGKDREFVAAADWRGLNTWYRDSVKGRFTISRDNAKSTMYLQMNSLKPTDTAVYYCNTGGWAFSPGASWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 169 - protein, 629 aa, synthetic construct
QVQLQESGGGLVQPGGSLRLSCAASGFRFSTSIMTWYRQAPGKERELVASISSDSRRTNYAESVKGRFTISRDNEQRMVFLEMNSLKPVDTAVYFCTRPNIRGRDWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 170 - protein, 631 aa, synthetic construct
QVQLQESGGGLVQLGGSLRLSCAASGFTFSNYYMSWYRQAPGKERELVGGINMTGGSADYVDSVKGRFTISRDNTKNTVYLQMNSLRVEDTAVYYCHTFSVWPPNNYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 171 - protein, 631 aa, synthetic construct
QVQLQESGGGLVQPGGSLRLSCAASGFSFSTYAMSWYRQPPGKERELVALITTTGGTTDYADSVKGRFTISRDDAEKTVSLQMNSLKPEDTAVYYCSAWRATLLKPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 172 - protein, 635 aa, synthetic construct
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 173 - protein, 635 aa, synthetic construct
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWDQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 174 - protein, 631 aa, synthetic construct
QVQLQESGGGLAQPGGSLRLSCAASGFTVSTYAMSWYRQAPGKERELVALITSAGGITDYADSVKGRFTIARDDAEKTVSLQMNSLKPEDTAVYYCSAWRATLFKPYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 175 - protein, 639 aa, synthetic construct
QVQLQESGGGLVQAGGSLRLSCAASGDTICTSAMAWYRQAPGKERELVAGISSGGTVYYADSVKGRFTISRDNAKNTVYLQMNTLKPEDTAVYYCNAGPEVHDDVFRLCGPTTDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 176 - protein, 639 aa, synthetic construct
QVQLQESGGGLVQPGGSLRLSCAASGFTLDYYAIGWFRQAPGKEREGIVYISNSDGNTYYADSVKGRFTISRDNAKNTVYLQMNSLKPEDTAVYYCAADQGSSVMHGFIEEWYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 177 - protein, 638 aa, synthetic construct
QVQLQESGGGLVQAGGSLRLSCAASGRTFSSYAMGWFRQAPGKEREFVAAISWSGGRTYYDDPVKGRFTISRDNAKNTVYLQMNSLKPEDTAVYYCNLDPEYSGTWNPWAGSGYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSQVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 178 - protein, 488 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAPKAQVQLQESGGGWVQPGGSLRLSCVASGFTFSRSSMSWHRQAPGKERDLVAIISSSGIITHYSDSVKGRFTISRDNAKDTVYLQMNSLKPEDTAVYVCNAVLDDDSGDYWGQGTQVTVSSGGGGSGGGGSGGGGSQVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSS

SEQ ID NO: 179 - protein, 256 aa, synthetic construct
APAPAPAPAPKAQVQLQESGGGWVQPGGSLRLSCVASGFTFSRSSMSWHRQAPGKERDLVAIISSSGIITHYSDSVKGRFTISRDNAKDTVYLQMNSLKPEDTAVYVCNAVLDDDSGDYWGQGTQVTVSSGGGGSGGGGSGGGGSQVQLQESGGGLVQPGGSLRLSCAASRFDFSVNDIRWYRQAPGNERELVARITGGGRTDYADSVKGRFTISRDNAKNTVYLQMNNLKPEDTAVYYCWGRGYWGQGTQVTVSS

SEQ ID NO: 180 - protein, 15 aa, synthetic construct
EPKSCDKTHTCPPCP

SEQ ID NO: 181 - protein, 33 aa, synthetic construct
EPKIPQPQPKPQPQPQPGGSGSAEAAAKAPKAP

SEQ ID NO: 182 - protein, 10 aa, synthetic construct
GGGGSGGGGS

SEQ ID NO: 183 - protein, 15 aa, synthetic construct
GGGGSGGGGSGGGGS

SEQ ID NO: 184 - protein, 25 aa, synthetic construct
GGGGSGGGGSGGGGSGGGGSGGGGS

SEQ ID NO: 185 - protein, 30 aa, synthetic construct
GGGGSGGGGSGGGGSGGGGSGGGGSGGGGS

SEQ ID NO: 186 - protein, 6 aa, synthetic construct
  VARIANT 6: note = Xaa is absent or, if present is from 1 to 9 repeats of amino acid residues 1 to 5
GGGGSX

SEQ ID NO: 187 - protein, 7 aa, synthetic construct
PAPAPKA

SEQ ID NO: 188 - protein, 12 aa, synthetic construct
APAPAPAPAPKA

SEQ ID NO: 189 - protein, 22 aa, synthetic construct
APAPAPAPAPAPAPAPAPAPKA

SEQ ID NO: 190 - protein, 9 aa, synthetic construct
  VARIANT 1: note = Xaa is present or absent and, if present , is A
  VARIANT 7: note = Xaa is absent or, if present, is from 1 to 9 repeats of amino acid residues 1 to 6
XPAPAPXKA

SEQ ID NO: 191 - protein, 12 aa, synthetic construct
AEAAAKEAAAKA

SEQ ID NO: 192 - protein, 17 aa, synthetic construct
AEAAAKEAAAKEAAAKA

SEQ ID NO: 193 - protein, 27 aa, synthetic construct
AEAAAKEAAAKEAAAKEAAAKEAAAKA

SEQ ID NO: 194 - protein, 8 aa, synthetic construct
  VARIANT 1: note = Xaa is absent and, if present, is preferably A
  VARIANT 7: note = Xaa is present or absent and, if present is preferably A
  VARIANT 8: note = Xaa is present or, if present is from 1 to 9 repeats of amino acid residues 2-6
XEAAAKXX

SEQ ID NO: 195 - protein, 127 aa, synthetic construct
  VARIANT 1: note = Xaa is E or Q
XVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 196 - protein, 123 aa, synthetic construct
  VARIANT 1: note = Xaa is E or Q
XVQLVESGGGLVQAGGSLRLSCAASESTFSNYAMGWFRQAPGPEREFVATISQTGSHTYYRNSVKGRFTISRDNAKNTVYLQMNNMKPEDTAVYYCAAGDNYYYTRTYEYDYWGQGTQVTVSS

SEQ ID NO: 197 - protein, 120 aa, synthetic construct
  VARIANT 1: note = Xaa is E or D
XVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSS

SEQ ID NO: 198 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 200 - protein, 15 aa, synthetic construct
EPKSSDKTHTCPPCP

SEQ ID NO: 201 - protein, 15 aa, synthetic construct
EPKSSDKTHTSPPSP

SEQ ID NO: 202 - protein, 9 aa, synthetic construct
DKTHTCPPC

SEQ ID NO: 203 - protein, 12 aa, synthetic construct
ERKCCVECPPCP

SEQ ID NO: 204 - protein, 12 aa, synthetic construct
ERKSSVECPPCP

SEQ ID NO: 205 - protein, 12 aa, synthetic construct
ERKSSVESPPCP

SEQ ID NO: 206 - protein, 12 aa, synthetic construct
ERKSSVESPPSP

SEQ ID NO: 207 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 208 - protein, 15 aa, synthetic construct
EPKSSDTPPPCPRCP

SEQ ID NO: 209 - protein, 15 aa, synthetic construct
EPKSSDTPPPSPRCP

SEQ ID NO: 210 - protein, 15 aa, synthetic construct
EPKSSDTPPPSPRSP

SEQ ID NO: 211 - protein, 12 aa, synthetic construct
ESKYGPPCPSCP

SEQ ID NO: 212 - protein, 12 aa, synthetic construct
ESKYGPPCPPCP

SEQ ID NO: 213 - protein, 12 aa, synthetic construct
ESKYGPPSPSCP

SEQ ID NO: 214 - protein, 12 aa, synthetic construct
ESKYGPPSPSSP

SEQ ID NO: 215 - protein, 19 aa, synthetic construct
MEWSWVFLFFLSVTTGVHS

SEQ ID NO: 216 - protein, 6 aa, synthetic construct
HHHHHH

SEQ ID NO: 217 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 107 aa, synthetic construct
GQPREPQVYTLPPSRQEMTKNQVSLTCLVEGFYPSDIAVEWESNGQPENNYKTTPPVLNSDGSFFLYSKLTVDKSRWQQGNVFSCSVMHEALHNHYTQKSLSLSPGK

SEQ ID NO: 219 - protein, 107 aa, synthetic construct
GQPREPQVYTLPPSREQMTKNQVSLTCLVKGFYPSDIAVEWESNGQPENNYKTTPPVLNSDGSFFLYSKLTVDKSRWQQGNVFSCSVMHEALHNHYTQESLSLSPGK

SEQ ID NO: 220 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRQEMTKNQVSLTCLVEGFYPSDIAVEWESNGQPENNYKTTPPVLNSDGSFFLYSKLTVDKSRWQQGNVFSCSVMHEALHNHYTQKSLSLSPGK

SEQ ID NO: 221 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVSLTCLVKGFYPSDIAVEWESNGQPENNYKTTPPVLNSDGSFFLYSKLTVDKSRWQQGNVFSCSVMHEALHNHYTQESLSLSPGK

SEQ ID NO: 222 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 223 - protein, 127 aa, synthetic construct
QVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 224 - protein, 410 aa, synthetic construct
GDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKIN

SEQ ID NO: 225 - protein, 167 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 226 - protein, 139 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSP

SEQ ID NO: 227 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - DNA, 354 bp, synthetic construct
  source 1..354: mol_type = other DNA
caggtgcaattgcaagaatctggcggcggactggttcagcctggcggatctctgagactgtcttgtgccgcctccggcttcaccttctcctcttacgccatgtcctggtacaggcaggcccctggcaaagagagagagctggtcgctctgatcacctctgctggcggcaccaccgattacgccgattctgtgaagggcagattcaccatctctcgggacgacgccgaaaagaccgtgtctctccagatgaactccctgaagcctgaggacaccgccgtgtactactgctccgcttggagagcctctctgtacgagccttattggggccagggaacccaggtcaccgtgtcctcc

SEQ ID NO: 229 - DNA, 360 bp, synthetic construct
  source 1..360: mol_type = other DNA
caggtgcaattgcaagaatctggcggcggactggttcagcctggcggatctctgagactgtcttgtgccgcctctggcttcaccttctccaactacgccatgtcctggtacagacagacccctggcaaagagagagaactggtcgccgccatgcagtctaccggcggcaccacctactacgccgattctgtgaagggcagattcaccatctctcgggacgacgccaagaacaccgtgtacctccagatgaactccctgaagcctgaggacaccgccgtgtactactgcaacgctgtgacccttcaaggctggagcgagtacgattattggggccagggaacccaggtcaccgtgtcctcc

SEQ ID NO: 230 - DNA, 360 bp, synthetic construct
  source 1..360: mol_type = other DNA
caggtgcaattgcaagaatctggcggcggactggttcagcctggcggatctctgagactgtcttgtaccgcctccggcttcaccttctccagctacgccatgtcctggtacaggcaggcccctggcaaagagagagagctggttgctgctatccctcacgacggcggctccatctactacgtggactctgtgaagggcagattctccatcagccgggacaacgccaagaacaccgtgtacctccagatgaactccctgaagcctgaggacaccgccgtgtactactgcaacgctgtgacccttcaaggctggcgcgagtacgattattggggccagggaacacaggtcaccgtgtcctcc

SEQ ID NO: 231 - DNA, 354 bp, synthetic construct
  source 1..354: mol_type = other DNA
caggtgcaattgcaagaatctggcggcggactggttcagcctggcggatctctgagactgtcttgtgctgcttctggcctggccttctccacctacgccatgtcctggtatagacaggcccctggcaaagagagagagctggtcgctctgatcacctctgctggcggcaccaccgattacgccgattctgtgaagggcagattcaccatctctcgggacgacgccgaaaagaccgtgtctctccagatgaactccctgaagcctgaggacaccgccgtgtactactgctccgcttggagagctaccctgttcaagccttattggggccagggcacacaggtcaccgtgtcctcc

SEQ ID NO: 232 - DNA, 372 bp, synthetic construct
  source 1..372: mol_type = other DNA
caggtgcaattgcaagaatctggcggcggactggttcagcctggcggatctctgagactgtcttgtgccgcctccggcttcaccagcggctttaccttttcctcctacgccatgtcctggtacaggcaggcccctggcaaagagagagagctggtcgccgctatcaccaacgctggcgattctacccactacgccgactccgtgaagggcagattcaccatctccagagacaacgccaagaacaccgtgtacctgctgatgaactccctgaagcctgaggacaccgccgcctactactgtaacgctgtgacccttcaaggctggagcgagtacgattattggggccagggaacccaggtcaccgtgtcctcc

SEQ ID NO: 233 - DNA, 360 bp, synthetic construct
  source 1..360: mol_type = other DNA
caggtgcaattgcaagaatctggcggcggactggttcagcctggcggatctctgagactgtcttgtgccgcctctggcttctccctggactactttgccatcggctggttcagacaggcccctggcaaagagagagagggaatctcctgcatctcctcctccggctccaacacctactacgccgactctgtgaagggcagattcaccatctctcgggacaacgccaagaacaccgtgtacctccagatgaactccctgaagcctgaggacaccgccgtgtactactgtgccgctcacgactactgttctcgggccccttacgattattggggccagggaacacaggtcaccgtgtcctcc

SEQ ID NO: 234 - DNA, 360 bp, synthetic construct
  source 1..360: mol_type = other DNA
caggtgcaattgcaagaatctggcggcggactggtgcagcctggcgaatctctgagactgtcttgtgccgcctccggcttcaccttctccaactacgccatgtcctggtacagacagacccctggcaaagagagagaactggtcgccgccatgcagtctaccggcggcaccacctactacgccgattctgtgaagggcagattcaccatctctcgggacgacgccaagaacaccgtgtacctccagatgaactccctgaagcctgaggacaccgccgtgtactactgcaacgctgtgacccttcaaggctggagcgagtacgattattggggccagggaacccaggtcaccgtgtcctcc

SEQ ID NO: 235 - DNA, 348 bp, synthetic construct
  source 1..348: mol_type = other DNA
caggtgcaattgcaagaatctggcggcggactggttcagcctggcggatctctgagactgtcttgtgccgcctccggcttcagattctcctcctccatcatgtcctggtacaggcaggcccctgagaaagagagagagctggtcgcctccatctcctccgattctgtgcggaccaactacgccgagtccgtgaagggcagattcaccatctctcgggacaacgagcagagcaccctgtttctggaaatgaacaacctgaagcctgtggataccgccgtgtacttctgcacccggccttccatcagaggcagagattggggacagggcacacaggtcaccgtgtcctcc

SEQ ID NO: 236 - DNA, 357 bp, synthetic construct
  source 1..357: mol_type = other DNA
caggtgcaattgcaagaatctggcggcggactggttcagcctggcggatctctgagactgtcttgtaccgcctctggcagaaccggctctgctgatgtgatgggctggtttcggagagcccctggcaaggacagagaatttgtggccgctgccgattggagaggcctgaacacctggtacagagactccgtgaagggcagattcaccatcagccgggacaacgccaagtccaccatgtacctccagatgaactccctgaagcctaccgacaccgccgtgtactactgtaataccggcggctgggccttttctcctggcgcttcttggggacagggaacccaggtcaccgtgtcctcc

SEQ ID NO: 237 - DNA, 348 bp, synthetic construct
  source 1..348: mol_type = other DNA
caggtgcaattgcaagaatctggcggcggactggttcagcctggcggatctctgagactgtcttgtgccgcctccggcttcagattctccacctccatcatgacctggtacaggcaggcccctggcaaagagagagagctggtcgcctccatctcctccgactctagaaggaccaactacgccgagtccgtgaagggcagattcaccatctctcgggacaacgagcagcggatggtgtttctggaaatgaactccctgaagcctgtggataccgccgtgtacttctgcacccggcctaacatcagaggcagagattggggccagggcacacaggtcaccgtgtcctcc

SEQ ID NO: 238 - DNA, 354 bp, synthetic construct
  source 1..354: mol_type = other DNA
caggtgcaattgcaagaatctggcggcggactggtgcagctcggaggatctctgagactgtcttgtgccgcctccggcttcaccttctccaactactacatgtcctggtacaggcaggcccctggcaaagagagagaactcgtcggcggcatcaatatgaccggcggctctgctgactacgtggactctgtgaagggcagattcaccatcagccgggacaacaccaagaacaccgtgtacctccagatgaactccctgagagtggaagataccgccgtgtactactgccacaccttcagcgtgtggcctccaaacaactattggggccagggcacacaggtcaccgtgtcctcc

SEQ ID NO: 239 - DNA, 354 bp, synthetic construct
  source 1..354: mol_type = other DNA
caggtgcaattgcaagaatctggcggcggactggttcagcctggcggatctctgagactgtcttgtgccgcctccggcttctccttctctacctacgccatgtcctggtacagacagcctcctggcaaagagagagagctggtcgctctgatcaccaccacaggcggcaccaccgattacgccgattctgtgaagggcagattcaccatctctcgggacgacgccgaaaagaccgtgtctctccagatgaactccctgaagcctgaggacaccgccgtgtactactgctccgcttggagagctaccctgctgaagccttattggggccagggaacacaggtcaccgtgtcctcc

SEQ ID NO: 240 - DNA, 366 bp, synthetic construct
  source 1..366: mol_type = other DNA
caggtgcaattgcaagaatctggcggcggactggttcagcctggcggatctctgagactgtcttgtgccgcttccggcagaaccttcagcagctatcctatgggctggttcagacaggcccctggcaaagagagagagttcgtcgccgctatcgattggagcggcgacatcacctactacgccgactctgtgaagggcagattcgccatctccagagacaacgccaagaacatggtgtacctccagatgaactccctgaagcctgaggacaccgccgtgtactactgtaacgctggccctctggactcctgggagagaaacgatttccctagctggggccagggcacacaggtcaccgtgtcctcc

SEQ ID NO: 241 - DNA, 366 bp, synthetic construct
  source 1..366: mol_type = other DNA
caggtgcaattgcaagaatctggcggcggactggttcagcctggcggatctctgagactgtcttgtgccgcttccggcagaaccttcagcagctatcctatgggctggttcagacaggcccctggcaaagagagagagttcgtcgccgctatcgattggagcggcgacatcacctactacgccgactctgtgaagggcagattcgccatctccagagacaacgccaagaacatggtgtacctccagatgaactccctgaagcctgaggacaccgccgtgtactactgtaacgctggccctctggactcctgggagagaaacgatttccctagctgggaccagggcacccaggtcaccgtgtcctcc

SEQ ID NO: 242 - DNA, 354 bp, synthetic construct
  source 1..354: mol_type = other DNA
caggtgcaattgcaagaatctggcggcggattggctcagcctggcggatctctgagactgtcttgtgccgcctctggcttcaccgtgtctacctacgccatgtcctggtacaggcaggcccctggcaaagagagagagctggtcgctctgatcacctccgctggcggcatcaccgattacgccgattctgtgaagggcagattcacaatcgcccgggacgacgccgaaaagaccgtgtctctccagatgaactccctgaagcctgaggacaccgccgtgtactactgctccgcttggagagctaccctgttcaagccttattggggccagggcacacaggtcaccgtgtcctcc

SEQ ID NO: 243 - DNA, 378 bp, synthetic construct
  source 1..378: mol_type = other DNA
caggtgcaattgcaagaatctggcggcggactggttcaggctggcggatctctgagactgtcttgtgccgcttctggcgacaccatctgcacctctgccatggcctggtatagacaggcccctggcaaagagagagagctggtcgctggaatctcctctggcggcaccgtgtactacgccgactctgtgaagggcagattcaccatctctcgggacaacgccaagaataccgtgtacctccagatgaacaccctgaagcctgaggacaccgctgtgtactactgcaacgctggacctgaggtgcacgacgacgtgttcagactgtgtggccctaccaccgattattggggccagggaacacaggtcaccgtgtcctcc

SEQ ID NO: 244 - DNA, 378 bp, synthetic construct
  source 1..378: mol_type = other DNA
caggtgcaattgcaagaatctggcggcggactggttcagcctggcggatctctgagactgtcttgtgccgcctctggcttcaccctggactactatgccatcggctggttcagacaggcccctggcaaagaaagagagggcatcgtctacatctccaactccgacggcaacacctactacgccgactccgtgaagggcagattcaccatctccagagacaacgccaagaacaccgtgtacctccagatgaactccctgaagcctgaggacaccgccgtgtactactgtgctgccgaccagggatcctctgtgatgcacggcttcatcgaagagtggtacgactactggggccagggcacacaggtcaccgtgtcctcc

SEQ ID NO: 245 - DNA, 375 bp, synthetic construct
  source 1..375: mol_type = other DNA
caggtgcaattgcaagaatctggcggcggactggttcaggctggcggatctctgagactgtcttgtgccgcttccggcagaaccttctcctcttacgccatgggctggttcagacaggcccctggcaaagagagagagttcgtcgccgctatctcttggagcggcggcaggacctactacgacgatcctgtgaagggcagattcaccatcagccgggacaacgccaagaacaccgtgtacctccagatgaactccctgaagcctgaggacaccgccgtgtactactgcaacctggatcctgagtactccggcacctggaatccttgggctggctctggatattggggccagggaacacaggtcaccgtgtcctcc

SEQ ID NO: 246 - DNA, 354 bp, synthetic construct
  source 1..354: mol_type = other DNA
caggtgcaattgcaagagtctggcggaggctgggttcagcctggtggatctctgagactgtcctgtgtggcctccggcttcaccttctccagatcctccatgtcttggcacagacaggcccctggcaaagagagagatctggtcgccatcatctcctccagcggcatcatcacccactactccgactccgtgaagggcagattcaccatctctcgggacaacgccaaggacaccgtgtacctccagatgaactccctgaagcctgaggacaccgccgtgtacgtgtgtaacgccgtgctggacgacgactctggcgattattggggccagggaacacaggtcaccgtgtcctcc

SEQ ID NO: 247 - DNA, 327 bp, synthetic construct
  source 1..327: mol_type = other DNA
caattgcaagagtctggcggaggactggttcagcctggcggatctctgagactgtcttgcgccgcctccagattcgacttctccgtgaacgacatccggtggtacagacaggcccctggcaatgagagagagctggtcgctagaatcaccggcggaggcagaaccgactacgccgattctgtgaagggcagattcaccatcagccgggacaacgccaagaacaccgtgtacctccagatgaacaacctgaagcctgaggacaccgccgtgtactactgttggggcagaggctattggggccagggaactcaggtcaccgtgtctagt

SEQ ID NO: 248 - DNA, 1050 bp, synthetic construct
  source 1..1050: mol_type = other DNA
caggtgcaattgcaagaatctggcggcggactggttcagcctggcggatctctgagactgtcttgtgccgcctccggcttcaccttctcctcttacgccatgtcctggtacaggcaggcccctggcaaagagagagagctggtcgctctgatcacctctgctggcggcaccaccgattacgccgattctgtgaagggcagattcaccatctctcgggacgacgccgaaaagaccgtgtctctccagatgaactccctgaagcctgaggacaccgccgtgtactactgctccgcttggagagcctctctgtacgagccttattggggccagggaacccaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 249 - DNA, 1056 bp, synthetic construct
  source 1..1056: mol_type = other DNA
caggtgcaattgcaagaatctggcggcggactggttcagcctggcggatctctgagactgtcttgtgccgcctctggcttcaccttctccaactacgccatgtcctggtacagacagacccctggcaaagagagagaactggtcgccgccatgcagtctaccggcggcaccacctactacgccgattctgtgaagggcagattcaccatctctcgggacgacgccaagaacaccgtgtacctccagatgaactccctgaagcctgaggacaccgccgtgtactactgcaacgctgtgacccttcaaggctggagcgagtacgattattggggccagggaacccaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 250 - DNA, 1056 bp, synthetic construct
  source 1..1056: mol_type = other DNA
caggtgcaattgcaagaatctggcggcggactggttcagcctggcggatctctgagactgtcttgtaccgcctccggcttcaccttctccagctacgccatgtcctggtacaggcaggcccctggcaaagagagagagctggttgctgctatccctcacgacggcggctccatctactacgtggactctgtgaagggcagattctccatcagccgggacaacgccaagaacaccgtgtacctccagatgaactccctgaagcctgaggacaccgccgtgtactactgcaacgctgtgacccttcaaggctggcgcgagtacgattattggggccagggaacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 251 - DNA, 1050 bp, synthetic construct
  source 1..1050: mol_type = other DNA
caggtgcaattgcaagaatctggcggcggactggttcagcctggcggatctctgagactgtcttgtgctgcttctggcctggccttctccacctacgccatgtcctggtatagacaggcccctggcaaagagagagagctggtcgctctgatcacctctgctggcggcaccaccgattacgccgattctgtgaagggcagattcaccatctctcgggacgacgccgaaaagaccgtgtctctccagatgaactccctgaagcctgaggacaccgccgtgtactactgctccgcttggagagctaccctgttcaagccttattggggccagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 252 - DNA, 1068 bp, synthetic construct
  source 1..1068: mol_type = other DNA
caggtgcaattgcaagaatctggcggcggactggttcagcctggcggatctctgagactgtcttgtgccgcctccggcttcaccagcggctttaccttttcctcctacgccatgtcctggtacaggcaggcccctggcaaagagagagagctggtcgccgctatcaccaacgctggcgattctacccactacgccgactccgtgaagggcagattcaccatctccagagacaacgccaagaacaccgtgtacctgctgatgaactccctgaagcctgaggacaccgccgcctactactgtaacgctgtgacccttcaaggctggagcgagtacgattattggggccagggaacccaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 253 - DNA, 1056 bp, synthetic construct
  source 1..1056: mol_type = other DNA
caggtgcaattgcaagaatctggcggcggactggttcagcctggcggatctctgagactgtcttgtgccgcctctggcttctccctggactactttgccatcggctggttcagacaggcccctggcaaagagagagagggaatctcctgcatctcctcctccggctccaacacctactacgccgactctgtgaagggcagattcaccatctctcgggacaacgccaagaacaccgtgtacctccagatgaactccctgaagcctgaggacaccgccgtgtactactgtgccgctcacgactactgttctcgggccccttacgattattggggccagggaacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 254 - DNA, 1056 bp, synthetic construct
  source 1..1056: mol_type = other DNA
caggtgcaattgcaagaatctggcggcggactggtgcagcctggcgaatctctgagactgtcttgtgccgcctccggcttcaccttctccaactacgccatgtcctggtacagacagacccctggcaaagagagagaactggtcgccgccatgcagtctaccggcggcaccacctactacgccgattctgtgaagggcagattcaccatctctcgggacgacgccaagaacaccgtgtacctccagatgaactccctgaagcctgaggacaccgccgtgtactactgcaacgctgtgacccttcaaggctggagcgagtacgattattggggccagggaacccaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 255 - DNA, 1044 bp, synthetic construct
  source 1..1044: mol_type = other DNA
caggtgcaattgcaagaatctggcggcggactggttcagcctggcggatctctgagactgtcttgtgccgcctccggcttcagattctcctcctccatcatgtcctggtacaggcaggcccctgagaaagagagagagctggtcgcctccatctcctccgattctgtgcggaccaactacgccgagtccgtgaagggcagattcaccatctctcgggacaacgagcagagcaccctgtttctggaaatgaacaacctgaagcctgtggataccgccgtgtacttctgcacccggccttccatcagaggcagagattggggacagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 256 - DNA, 1053 bp, synthetic construct
  source 1..1053: mol_type = other DNA
caggtgcaattgcaagaatctggcggcggactggttcagcctggcggatctctgagactgtcttgtaccgcctctggcagaaccggctctgctgatgtgatgggctggtttcggagagcccctggcaaggacagagaatttgtggccgctgccgattggagaggcctgaacacctggtacagagactccgtgaagggcagattcaccatcagccgggacaacgccaagtccaccatgtacctccagatgaactccctgaagcctaccgacaccgccgtgtactactgtaataccggcggctgggccttttctcctggcgcttcttggggacagggaacccaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 257 - DNA, 1044 bp, synthetic construct
  source 1..1044: mol_type = other DNA
caggtgcaattgcaagaatctggcggcggactggttcagcctggcggatctctgagactgtcttgtgccgcctccggcttcagattctccacctccatcatgacctggtacaggcaggcccctggcaaagagagagagctggtcgcctccatctcctccgactctagaaggaccaactacgccgagtccgtgaagggcagattcaccatctctcgggacaacgagcagcggatggtgtttctggaaatgaactccctgaagcctgtggataccgccgtgtacttctgcacccggcctaacatcagaggcagagattggggccagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 258 - DNA, 1050 bp, synthetic construct
  source 1..1050: mol_type = other DNA
caggtgcaattgcaagaatctggcggcggactggtgcagctcggaggatctctgagactgtcttgtgccgcctccggcttcaccttctccaactactacatgtcctggtacaggcaggcccctggcaaagagagagaactcgtcggcggcatcaatatgaccggcggctctgctgactacgtggactctgtgaagggcagattcaccatcagccgggacaacaccaagaacaccgtgtacctccagatgaactccctgagagtggaagataccgccgtgtactactgccacaccttcagcgtgtggcctccaaacaactattggggccagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 259 - DNA, 1050 bp, synthetic construct
  source 1..1050: mol_type = other DNA
caggtgcaattgcaagaatctggcggcggactggttcagcctggcggatctctgagactgtcttgtgccgcctccggcttctccttctctacctacgccatgtcctggtacagacagcctcctggcaaagagagagagctggtcgctctgatcaccaccacaggcggcaccaccgattacgccgattctgtgaagggcagattcaccatctctcgggacgacgccgaaaagaccgtgtctctccagatgaactccctgaagcctgaggacaccgccgtgtactactgctccgcttggagagctaccctgctgaagccttattggggccagggaacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 260 - DNA, 1062 bp, synthetic construct
  source 1..1062: mol_type = other DNA
caggtgcaattgcaagaatctggcggcggactggttcagcctggcggatctctgagactgtcttgtgccgcttccggcagaaccttcagcagctatcctatgggctggttcagacaggcccctggcaaagagagagagttcgtcgccgctatcgattggagcggcgacatcacctactacgccgactctgtgaagggcagattcgccatctccagagacaacgccaagaacatggtgtacctccagatgaactccctgaagcctgaggacaccgccgtgtactactgtaacgctggccctctggactcctgggagagaaacgatttccctagctggggccagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 261 - DNA, 1062 bp, synthetic construct
  source 1..1062: mol_type = other DNA
caggtgcaattgcaagaatctggcggcggactggttcagcctggcggatctctgagactgtcttgtgccgcttccggcagaaccttcagcagctatcctatgggctggttcagacaggcccctggcaaagagagagagttcgtcgccgctatcgattggagcggcgacatcacctactacgccgactctgtgaagggcagattcgccatctccagagacaacgccaagaacatggtgtacctccagatgaactccctgaagcctgaggacaccgccgtgtactactgtaacgctggccctctggactcctgggagagaaacgatttccctagctgggaccagggcacccaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 262 - DNA, 1050 bp, synthetic construct
  source 1..1050: mol_type = other DNA
caggtgcaattgcaagaatctggcggcggattggctcagcctggcggatctctgagactgtcttgtgccgcctctggcttcaccgtgtctacctacgccatgtcctggtacaggcaggcccctggcaaagagagagagctggtcgctctgatcacctccgctggcggcatcaccgattacgccgattctgtgaagggcagattcacaatcgcccgggacgacgccgaaaagaccgtgtctctccagatgaactccctgaagcctgaggacaccgccgtgtactactgctccgcttggagagctaccctgttcaagccttattggggccagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 263 - DNA, 1074 bp, synthetic construct
  source 1..1074: mol_type = other DNA
caggtgcaattgcaagaatctggcggcggactggttcaggctggcggatctctgagactgtcttgtgccgcttctggcgacaccatctgcacctctgccatggcctggtatagacaggcccctggcaaagagagagagctggtcgctggaatctcctctggcggcaccgtgtactacgccgactctgtgaagggcagattcaccatctctcgggacaacgccaagaataccgtgtacctccagatgaacaccctgaagcctgaggacaccgctgtgtactactgcaacgctggacctgaggtgcacgacgacgtgttcagactgtgtggccctaccaccgattattggggccagggaacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 264 - DNA, 1074 bp, synthetic construct
  source 1..1074: mol_type = other DNA
caggtgcaattgcaagaatctggcggcggactggttcagcctggcggatctctgagactgtcttgtgccgcctctggcttcaccctggactactatgccatcggctggttcagacaggcccctggcaaagaaagagagggcatcgtctacatctccaactccgacggcaacacctactacgccgactccgtgaagggcagattcaccatctccagagacaacgccaagaacaccgtgtacctccagatgaactccctgaagcctgaggacaccgccgtgtactactgtgctgccgaccagggatcctctgtgatgcacggcttcatcgaagagtggtacgactactggggccagggcacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 265 - DNA, 1071 bp, synthetic construct
  source 1..1071: mol_type = other DNA
caggtgcaattgcaagaatctggcggcggactggttcaggctggcggatctctgagactgtcttgtgccgcttccggcagaaccttctcctcttacgccatgggctggttcagacaggcccctggcaaagagagagagttcgtcgccgctatctcttggagcggcggcaggacctactacgacgatcctgtgaagggcagattcaccatcagccgggacaacgccaagaacaccgtgtacctccagatgaactccctgaagcctgaggacaccgccgtgtactactgcaacctggatcctgagtactccggcacctggaatccttgggctggctctggatattggggccagggaacacaggtcaccgtgtcctccgagcctaagtcttgcgacaagacccacacctgtcctccatgtcctgctccagaattgctcggcggaccctccgtgtttctgttccctccaaagcctaaggacaccctgatgatctctcggacccctgaagtgacctgcgtggtggtggatgtgtcccacgaagatcccgaagtgaagtttaattggtacgtggacggcgtcgaggtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattctttctgtactccaagctgacagtggacaagtccagatggcagcagggcaacgtgttctcctgctctgtgatgcacgaggccctgcacaaccactacacccagaagtccctgtctctgtcccctggcaaa

SEQ ID NO: 266 - DNA, 1893 bp, synthetic construct
  source 1..1893: mol_type = other DNA
caggtccaattgcaagaatctggcggcggactggttcagcctggcggatctctgagactgtcttgtgccgcctccggcttcaccttctcctcttacgccatgtcctggtacaggcaggcccctggcaaagagagagagctggtcgctctgatcacctctgctggcggcaccaccgattacgccgattctgtgaagggcagattcaccatctctcgggacgacgccgaaaagaccgtgtctctgcagatgaactccctgaagcctgaggacaccgccgtgtactactgctccgcttggagagcctctctgtacgagccttattggggccagggaacccaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaagccccagcccctgcccctgcacccgcgcctaaagcccaggttcaattggtggaatctggcggcggatctgtacaagctggcggctctctgagactgtcttgtgccgcttctggctccaccgactccatcgagtacatgacctggttcagacaggcccctggcaaggctagagaaggtgtcgctgctctgtatacccacaccggcaacacctactacaccgacagcgtgaagggcagattcaccatcagccaggacaaggccaagaacatggcctacctgcggatggactccgtgaagtctgaggacaccgccatctacacctgtggcgctacccggaaatacgtgcccgtcagatttgccctggaccagtcctcctacgactactggggacaagggacccaggtcaccgtgtcatcgggaggcggaggatctggcggaggtggaagtggcggaggcggaagccaggttcaattggtggaatctggcggcggatctgtacaagctggcggctctctgagactgtcttgtgccgcttctggctccaccgactccatcgagtacatgacctggttcagacaggcccctggcaaggctagagaaggtgtcgctgctctgtatacccacaccggcaacacctactacaccgacagcgtgaagggcagattcaccatcagccaggacaaggccaagaacatggcctacctgcggatggactccgtgaagtctgaggacaccgccatctacacctgtggcgctacccggaaatacgtgcccgtcagatttgccctggaccagtcctcctacgactactggggacaagggacccaggtcaccgtgagttct

SEQ ID NO: 267 - DNA, 1818 bp, synthetic construct
  source 1..1818: mol_type = other DNA
caggtccaattgcaagaatctggcggcggactggttcagcctggcggatctctgagactgtcttgtgccgcctccggcttcaccttctcctcttacgccatgtcctggtacaggcaggcccctggcaaagagagagagctggtcgctctgatcacctctgctggcggcaccaccgattacgccgattctgtgaagggcagattcaccatctctcgggacgacgccgaaaagaccgtgtctctgcagatgaactccctgaagcctgaggacaccgccgtgtactactgctccgcttggagagcctctctgtacgagccttattggggccagggaacccaggtcaccgtgtctagtgaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaagccccagcccctgcccctgcacccgcgcctaaagcccaagtccaattgcaagagtctggcggaggctgggttcagcctggtggatctctgagactgtcctgtgtggcctccggcttcaccttctccagatcctccatgtcttggcacagacaggcccctggcaaagagagagatctggtcgccatcatctcctccagcggcatcatcacccactactccgactccgtgaagggcagattcaccatctctcgggacaacgccaaggacaccgtgtacctccagatgaactccctgaagcctgaggacaccgccgtgtacgtgtgtaacgccgtgctggacgacgactctggcgattattggggccagggaacacaggtcaccgtgtcatcgggaggcggaggatctggcggaggtggaagtggcggaggcggaagccaggttcaattgcaagagtctggcggaggactggttcagcctggcggatctctgagactgtcttgcgccgcctccagattcgacttctccgtgaacgacatccggtggtacagacaggcccctggcaatgagagagagctggtcgctagaatcaccggcggaggcagaaccgactacgccgattctgtgaagggcagattcaccatcagccgggacaacgccaagaacaccgtgtacctccagatgaacaacctgaagcctgaggacaccgccgtgtactactgttggggcagaggctattggggccagggaactcaggtcaccgtgagttct

SEQ ID NO: 268 - protein, 472 aa, synthetic construct
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 269 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 270 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 271 - protein, 11 aa, synthetic construct
  VARIANT 5: note = Xaa is absent or, if present is from 1 to 9 repeats of amino acid residues 1 to 4
  VARIANT 11: note = Xaa is absent or, if present is from 1 to 9 repeats or amino acid residues 6 to 10
GGGSXGGGGSX

SEQ ID NO: 272 - protein, 5 aa, synthetic construct
  VARIANT 5: note = Xaa is absent or, if present is from 1 to 9 repeats of amino acid residues 1 to 4
GGGSX

SEQ ID NO: 273 - protein, 5 aa, synthetic construct
  VARIANT 5: note = Xaa is absent or, if present is from 1 to 9 repeats of amino acid residues 1 to 4
GGSGX

SEQ ID NO: 274 - protein, 6 aa, synthetic construct
  VARIANT 6: note = Xaa is absent or, if present is from 1 to 9 repeats of amino acid residues 1 to 5
GGGSSX

SEQ ID NO: 275 - protein, 5 aa, synthetic construct
FENVA

SEQ ID NO: 276 - protein, 15 aa, synthetic construct
IIAQYITHYNDAVKG

SEQ ID NO: 277 - skipped

SEQ ID NO: 278 - protein, 8 aa, synthetic construct
TSMHSFEN

SEQ ID NO: 279 - protein, 6 aa, synthetic construct
IAQYIT

SEQ ID NO: 280 - protein, 5 aa, synthetic construct
NVDQY

SEQ ID NO: 281 - protein, 110 aa, synthetic construct
QVQLQESGGGLVQPGGSLRLSCATSTSMHSFENVAWYRQAPGKQRELVAIIAQYITHYNDAVKGRFTISRDDSKNTAYLQMNNLKPEDTAVYYCNVDQYWGQGTQVTVSS

SEQ ID NO: 282 - protein, 5 aa, synthetic construct
NYGMS

SEQ ID NO: 283 - protein, 17 aa, synthetic construct
SIDSTGGTTRYADSVKG

SEQ ID NO: 284 - protein, 7 aa, synthetic construct
DFLSWMP

SEQ ID NO: 285 - protein, 8 aa, synthetic construct
GFTFSNYG

SEQ ID NO: 286 - protein, 8 aa, synthetic construct
IDSTGGTT

SEQ ID NO: 287 - protein, 9 aa, synthetic construct
AKDFLSWMP

SEQ ID NO: 288 - protein, 116 aa, synthetic construct
QVQLQESGGGLVQPGGSLRLSCAASGFTFSNYGMSWVRQAPGEGLEWVSSIDSTGGTTRYADSVKGRFTISRDNAKNTLYLQMNSLKPEDTAVYYCAKDFLSWMPRGQGTQVTVSS

SEQ ID NO: 289 - protein, 5 aa, synthetic construct
NYGMS

SEQ ID NO: 290 - protein, 17 aa, synthetic construct
SIDSSGGTTKYADSVKG

SEQ ID NO: 291 - protein, 7 aa, synthetic construct
DFLSWMP

SEQ ID NO: 292 - protein, 8 aa, synthetic construct
GFTFSNYG

SEQ ID NO: 293 - protein, 8 aa, synthetic construct
IDSSGGTT

SEQ ID NO: 294 - protein, 9 aa, synthetic construct
AKDFLSWMP

SEQ ID NO: 295 - protein, 116 aa, synthetic construct
QVQLQESGGGLVQAGGSLRLSCAASGFTFSNYGMSWVRQAPGEGLEWVSSIDSSGGTTKYADSVKGRFTISRDNAKNTLYLQMNSLKPEDTAVYYCAKDFLSWMPRGQGTQVTVSS

SEQ ID NO: 296 - protein, 5 aa, synthetic construct
NYGMS

SEQ ID NO: 297 - protein, 17 aa, synthetic construct
SIDSSGGTTKYADSVKG

SEQ ID NO: 298 - protein, 7 aa, synthetic construct
DFLSWMP

SEQ ID NO: 299 - protein, 8 aa, synthetic construct
GFTFSNYG

SEQ ID NO: 300 - protein, 8 aa, synthetic construct
IDSSGGTT

SEQ ID NO: 301 - protein, 9 aa, synthetic construct
AKDFLSWMP

SEQ ID NO: 302 - protein, 116 aa, synthetic construct
QVQLQESGGGLVQPGDSLRLSCAASGFTFSNYGMSWVRQAPGEGLEWVSSIDSSGGTTKYADSVKGRFTISRDNAKNTLYLQMNSLKPEDTAVYYCAKDFLSWMPRGQGTQVTVSS

SEQ ID NO: 303 - protein, 5 aa, synthetic construct
NYGMS

SEQ ID NO: 304 - protein, 17 aa, synthetic construct
SIDSTGGTTRYADSVKG

SEQ ID NO: 305 - protein, 7 aa, synthetic construct
DFLSWMP

SEQ ID NO: 306 - protein, 8 aa, synthetic construct
GFTFSNYG

SEQ ID NO: 307 - protein, 8 aa, synthetic construct
IDSTGGTT

SEQ ID NO: 308 - protein, 9 aa, synthetic construct
AKDFLSWMP

SEQ ID NO: 309 - protein, 116 aa, synthetic construct
QVQLQESGGGLVQAGGSLRLSCAASGFTFSNYGMSWVRQAPGEGLEWVSSIDSTGGTTRYADSVKGRFTISRDNAKNTLYLQMNSLKPEDTAVYYCAKDFLSWMPRGQGTQVTVSS

SEQ ID NO: 310 - protein, 5 aa, synthetic construct
FENVA

SEQ ID NO: 311 - protein, 15 aa, synthetic construct
IIAQYITHYNDAVKG

SEQ ID NO: 312 - skipped

SEQ ID NO: 313 - protein, 8 aa, synthetic construct
TSMHSFEN

SEQ ID NO: 314 - protein, 6 aa, synthetic construct
IAQYIT

SEQ ID NO: 315 - protein, 5 aa, synthetic construct
NVDQY

SEQ ID NO: 316 - protein, 110 aa, synthetic construct
QVQLQESGGGLVQPGGSLRLSCATSTSMHSFENVAWYRQAPGKQRELAAIIAQYITHYNDAVKGRFTISRDDSKNTAYLQMNNLKPEDTAVYYCNVDQYWGQGTQVTVSS

SEQ ID NO: 317 - protein, 5 aa, synthetic construct
SYGMS

SEQ ID NO: 318 - protein, 17 aa, synthetic construct
SIDSSGGTTKYAGSVQG

SEQ ID NO: 319 - protein, 7 aa, synthetic construct
DFLSWMP

SEQ ID NO: 320 - protein, 8 aa, synthetic construct
GFTFSSYG

SEQ ID NO: 321 - protein, 8 aa, synthetic construct
IDSSGGTT

SEQ ID NO: 322 - protein, 9 aa, synthetic construct
AKDFLSWMP

SEQ ID NO: 323 - protein, 116 aa, synthetic construct
QVQLQESGGGLVQPGDSLRLSCAASGFTFSSYGMSWVRQAPGEGLEWVSSIDSSGGTTKYAGSVQGRFTISRDNAKNTLYLQMNSLKPEDTAVYYCAKDFLSWMPRGQGTQVTVSS

SEQ ID NO: 324 - protein, 5 aa, synthetic construct
SYGMS

SEQ ID NO: 325 - protein, 17 aa, synthetic construct
SIDSSGGTTKYAGSVQG

SEQ ID NO: 326 - protein, 7 aa, synthetic construct
DFLSWMP

SEQ ID NO: 327 - protein, 8 aa, synthetic construct
GFTFSSYG

SEQ ID NO: 328 - protein, 8 aa, synthetic construct
IDSSGGTT

SEQ ID NO: 329 - protein, 9 aa, synthetic construct
AKDFLSWMP

SEQ ID NO: 330 - protein, 116 aa, synthetic construct
QVQLQESGGGEVQAGGSLRLSCAASGFTFSSYGMSWVRQAPGEGLEWVSSIDSSGGTTKYAGSVQGRFTISRDNAKNTLYLQMNSLKPEDTAVYYCAKDFLSWMPRGQGTQVTVSS

SEQ ID NO: 331 - protein, 5 aa, synthetic construct
NYGMS

SEQ ID NO: 332 - protein, 17 aa, synthetic construct
SIDSTGGTTRYADSVKG

SEQ ID NO: 333 - protein, 7 aa, synthetic construct
DFLSWMP

SEQ ID NO: 334 - protein, 8 aa, synthetic construct
GFTFSNYG

SEQ ID NO: 335 - protein, 8 aa, synthetic construct
IDSTGGTT

SEQ ID NO: 336 - protein, 9 aa, synthetic construct
AKDFLSWMP

SEQ ID NO: 337 - protein, 116 aa, synthetic construct
QVQLQESGGGLVQPGGSLRLSCAASGFTFSNYGMSWVRQAPGEGLEWVSSIDSTGGTTRYADSVKGRFTISRDNAKNALYLQMNSLKPEDTAVYYCAKDFLSWMPRGQGTQVTVSS

SEQ ID NO: 338 - protein, 5 aa, synthetic construct
NYGMS

SEQ ID NO: 339 - protein, 17 aa, synthetic construct
SIDSSGGTTRYADSVKG

SEQ ID NO: 340 - protein, 7 aa, synthetic construct
DFLSWMP

SEQ ID NO: 341 - protein, 8 aa, synthetic construct
GFTFSNYG

SEQ ID NO: 342 - protein, 8 aa, synthetic construct
IDSSGGTT

SEQ ID NO: 343 - protein, 9 aa, synthetic construct
AKDFLSWMP

SEQ ID NO: 344 - protein, 116 aa, synthetic construct
QVQLQESGGGLVQPGGSLRLSCAASGFTFSNYGMSWVRQAPGEGLEWVSSIDSSGGTTRYADSVKGRFTISRDNAKNTLYLQMNSLKPEDTAVYYCAKDFLSWMPRGQGTQVTVSS

SEQ ID NO: 345 - protein, 5 aa, synthetic construct
SYGMS

SEQ ID NO: 346 - protein, 17 aa, synthetic construct
SIDSSGGTTKYAGSVQG

SEQ ID NO: 347 - protein, 7 aa, synthetic construct
DFLSWMP

SEQ ID NO: 348 - protein, 8 aa, synthetic construct
GFTFSSYG

SEQ ID NO: 349 - protein, 8 aa, synthetic construct
IDSSGGTT

SEQ ID NO: 350 - protein, 9 aa, synthetic construct
AKDFLSWMP

SEQ ID NO: 351 - protein, 116 aa, synthetic construct
QVQLQESGGGLVQPGGSLRLSCAASGFTFSSYGMSWVRQAPGEGLEWVSSIDSSGGTTKYAGSVQGRFTISRDNAKNTLYLQMNSLKPEDTAVYYCAKDFLSWMPRGQGTQVTVSS

SEQ ID NO: 352 - protein, 5 aa, synthetic construct
SYGMS

SEQ ID NO: 353 - protein, 17 aa, synthetic construct
SIDSSGGTTKYAGSVQG

SEQ ID NO: 354 - protein, 7 aa, synthetic construct
DFLSWMP

SEQ ID NO: 355 - protein, 8 aa, synthetic construct
GFTFSSYG

SEQ ID NO: 356 - protein, 8 aa, synthetic construct
IDSSGGTT

SEQ ID NO: 357 - protein, 9 aa, synthetic construct
AKDFLSWMP

SEQ ID NO: 358 - protein, 116 aa, synthetic construct
QVQLQESGGGLVQPGDSLRLSCAASGFTFSSYGMSWVRQAPGEGLEWVSSIDSSGGTTKYAGSVQGRFTISRDNAKNTVYLQMNSLKPEDTAVYYCAKDFLSWMPRGQGTQVTVSS

SEQ ID NO: 359 - protein, 5 aa, synthetic construct
SDTMG

SEQ ID NO: 360 - protein, 17 aa, synthetic construct
AIQRRGGSIYYADSVKG

SEQ ID NO: 361 - protein, 17 aa, synthetic construct
SPVNGDNYGLLPRHYDY

SEQ ID NO: 362 - protein, 8 aa, synthetic construct
SVTFSSDT

SEQ ID NO: 363 - protein, 8 aa, synthetic construct
IQRRGGSI

SEQ ID NO: 364 - protein, 19 aa, synthetic construct
AASPVNGDNYGLLPRHYDY

SEQ ID NO: 365 - protein, 126 aa, synthetic construct
QVQLQESGGGLVQAGGSLRLSCTASSVTFSSDTMGWFRQAPGKEREFVAAIQRRGGSIYYADSVKGRFTISRDNPKSTVYLQMNSLKPEDTAVYYCAASPVNGDNYGLLPRHYDYWGQGTQVTVSS

SEQ ID NO: 366 - DNA, 330 bp, synthetic construct
  source 1..330: mol_type = other DNA
caagtccaattgcaagagtctggcggaggcctggttcagcctggtggatctctgagactgtcctgcgccacctctacctccatgcacagcttcgagaacgtggcctggtacagacaggctcctggcaagcagagagagctggtcgccatcattgcccagtacatcacccactacaacgacgccgtgaagggcagattcaccatctctcgggacgactccaagaacaccgcctacctccagatgaacaacctgaagcctgaggacaccgccgtgtactactgcaacgtggaccagtattggggccagggcacacaggtcaccgtttcatcg

SEQ ID NO: 367 - DNA, 348 bp, synthetic construct
  source 1..348: mol_type = other DNA
caagtccaattgcaagagtctggcggaggcctggttcagcctggtggatctctgagactgtcttgtgccgcctccggcttcaccttctccaactacggaatgtcctgggtccgacaggctcctggcgaaggattggaatgggtgtcctccatcgattccaccggcggcaccaccagatacgccgattctgtgaagggcagattcaccatcagccgggacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 368 - DNA, 348 bp, synthetic construct
  source 1..348: mol_type = other DNA
caagtccaattgcaagagtctggcggaggcctggttcaggctggtggatctctgagactgtcttgtgccgcctccggcttcaccttctccaactacggaatgtcctgggtccgacaggctcctggcgaaggattggaatgggtgtcctccatcgattcctccggcggcaccactaagtacgccgactctgtgaagggcagattcaccatctctcgggacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 369 - DNA, 348 bp, synthetic construct
  source 1..348: mol_type = other DNA
caagtccaattgcaagagtctggcggaggcctggttcagcctggcgattctctgagactgtcttgtgccgcctccggcttcaccttctccaactacggaatgtcctgggtccgacaggctcctggcgaaggattggaatgggtgtcctccatcgattcctccggcggcaccactaagtacgccgactctgtgaagggcagattcaccatctctcgggacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 370 - DNA, 348 bp, synthetic construct
  source 1..348: mol_type = other DNA
caagtccaattgcaagagtctggcggaggcctggttcaggctggtggatctctgagactgtcttgtgccgcctccggcttcaccttctccaactacggaatgtcctgggtccgacaggctcctggcgaaggattggaatgggtgtcctccatcgattccaccggcggcaccaccagatacgccgattctgtgaagggcagattcaccatcagccgggacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 371 - DNA, 330 bp, synthetic construct
  source 1..330: mol_type = other DNA
caagtccaattgcaagagtctggcggaggcctggttcagcctggtggatctctgagactgtcctgcgccacctctacctccatgcacagcttcgagaacgtggcctggtacagacaggctcccggaaagcagagagagctggccgctatcattgcccagtacatcacccactacaacgacgccgtgaagggcagattcaccatctctcgggacgactccaagaacaccgcctacctccagatgaacaacctgaagcctgaggacaccgccgtgtactactgcaacgtggaccagtattggggccagggcacacaggtcaccgtttcatcg

SEQ ID NO: 372 - DNA, 348 bp, synthetic construct
  source 1..348: mol_type = other DNA
caagtccaattgcaagagtctggcggaggcctggttcagcctggcgattctctgagactgtcttgtgccgcctccggcttcaccttctcctcttacggaatgtcctgggtccgacaggctcctggcgaaggattggaatgggtgtcctccatcgattcctccggcggcaccactaagtacgccggatctgtgcagggcagattcaccatctccagagacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 373 - DNA, 348 bp, synthetic construct
  source 1..348: mol_type = other DNA
caagtccaattgcaagaatctggcggcggagaggttcaggctggcggatctctgagactgtcttgtgccgcctccggcttcaccttctcctcttacggaatgtcctgggtccgacaggctcctggcgaaggattggaatgggtgtcctccatcgattcctccggcggcaccactaagtacgccggatctgtgcagggcagattcaccatctccagagacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 374 - DNA, 348 bp, synthetic construct
  source 1..348: mol_type = other DNA
caagtccaattgcaagagtctggcggaggcctggttcagcctggtggatctctgagactgtcttgtgccgcctccggcttcaccttctccaactacggaatgtcctgggtccgacaggctcctggcgaaggattggaatgggtgtcctccatcgattccaccggcggcaccaccagatacgccgattctgtgaagggcagattcaccatcagccgggacaacgccaagaacgccctgtacctccagatgaactccctgaagcctgaggacaccgccgtgtactactgcgccaaggacttcctgtcctggatgcctagaggccagggaacacaggtcaccgtgtcatcg

SEQ ID NO: 375 - DNA, 348 bp, synthetic construct
  source 1..348: mol_type = other DNA
caggtccaattgcaagagtctggcggcggattggttcagcctggcggatctctgagactgtcttgtgccgcctctggcttcaccttctccaactacggcatgtcctgggtccgacaggctcctggcgaaggattggaatgggtgtcctccatcgattcctccggcggcaccaccagatacgccgattctgtgaagggcagattcaccatcagccgggacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 376 - DNA, 348 bp, synthetic construct
  source 1..348: mol_type = other DNA
caggtccaattgcaagagtctggcggcggattggttcagcctggcggatctctgagactgtcttgtgccgcctccggcttcaccttctcctcttacggaatgtcctgggtccgacaggctcctggcgaaggattggaatgggtgtcctccatcgattcctccggcggcaccactaagtacgccggatctgtgcagggcagattcaccatctccagagacaacgccaagaacaccctgtacctccagatgaactccctgaagcctgaggacaccgccgtgtactactgcgccaaggacttcctgtcctggatgcctagaggccagggaacacaggtcaccgtgtcatcg

SEQ ID NO: 377 - DNA, 348 bp, synthetic construct
  source 1..348: mol_type = other DNA
caggtccaattgcaagagtctggcggcggattggtgcagcctggcgattctctgagactgtcttgtgccgcctccggcttcaccttctcctcttacggaatgtcctgggtccgacaggctcctggcgaaggattggaatgggtgtcctccatcgattcctccggcggcaccactaagtacgccggatctgtgcagggcagattcaccatctccagagacaacgccaagaacaccgtgtacctccagatgaactccctgaagcctgaggacaccgccgtgtactactgcgccaaggacttcctgtcctggatgcctagaggccagggaacacaggtcaccgtgtcatcg

SEQ ID NO: 378 - DNA, 378 bp, synthetic construct
  source 1..378: mol_type = other DNA
caggtccaattgcaagagtctggcggcggattggttcaggctggcggatctctgagactgtcctgtaccgcctcctccgtgaccttctcctctgacacaatgggctggttcagacaggcccctggcaaagagagagagttcgtcgctgccatccagcggagaggcggctctatctactacgccgactctgtgaagggcagattcaccatcagccgggacaaccccaagtccaccgtgtacctccagatgaactccctgaagcctgaggacaccgccgtgtactactgtgccgcttctcctgtgaacggcgacaactatggcctgctgcctcggcactacgattattggggacagggcacacaggtcaccgtgtcatcg

SEQ ID NO: 379 - protein, 116 aa, synthetic construct
  VARIANT 11: note = Xaa is L or E
  VARIANT 14: note = Xaais A or P
  VARIANT 16: note = Xaa is D or G
  VARIANT 31: note = Xaa is N or S
  VARIANT 54: note = Xaa is S or T
  VARIANT 59: note = Xaa is K or R
  VARIANT 62: note = Xaa is D or G
  VARIANT 65: note = Xaa is K or Q
  VARIANT 78: note = Xaa is T or A
  VARIANT 79: note = Xaa is L or V
QVQLQESGGGXVQXGXSLRLSCAASGFTFSXYGMSWVRQAPGEGLEWVSSIDSXGGTTXYAXSVXGRFTISRDNAKNXXYLQMNSLKPEDTAVYYCAKDFLSWMPRGQGTQVTVSS

SEQ ID NO: 380 - protein, 118 aa, synthetic construct
  VARIANT 12: note = Xaa is A or V
  VARIANT 27: note = Xaa is L or F
  VARIANT 28: note = Xaa is A, T or S
  VARIANT 29: note = Xaa is F or V
  VARIANT 31: note = Xaa is T or S
  VARIANT 40: note = Xaa is A or P
  VARIANT 53: note = Xaa is S or T
  VARIANT 54: note = Xaa is A or T
  VARIANT 57: note = Xaa is I or T
  VARIANT 71: note = Xaa is A or S
  VARIANT 102: note = Xaa is S or T
  VARIANT 104: note = Xaa is F, Y or L
  VARIANT 105: note = Xaa is E or K
QVQLQESGGGLXQPGGSLRLSCAASGXXXSXYAMSWYRQXPGKERELVALITXXGGXTDYADSVKGRFTIXRDDAEKTVSLQMNSLKPEDTAVYYCSAWRAXLXXPYWGQGTQVTVSS

SEQ ID NO: 381 - protein, 124 aa, synthetic construct
  VARIANT 16: note = Xaa is G or E
  VARIANT 23: note = Xaa is A or T
  VARIANT 27: note = Xaa is absent or F
  VARIANT 28: note = Xaa is absent of T
  VARIANT 29: note = Xaa is absent or S
  VARIANT 30: note = Xaa is absent or G
  VARIANT 35: note = Xaa is S or N
  VARIANT 44: note = Xaa is A or T
  VARIANT 55: note = Xaa is M or I
  VARIANT 56: note = Xaa is Q, P or T
  VARIANT 57: note = Xaa is S, H or N
  VARIANT 58: note = Xaa is T, A or D
  VARIANT 60: note = Xaa is D or G
  VARIANT 61: note = Xaa is S or T
  VARIANT 62: note = Xaa is T or I
  VARIANT 63: note = Xaa is Y or H
  VARIANT 65: note = Xaa is A or V
  VARIANT 73: note = Xaa is S or T
  VARIANT 78: note = Xaa is D or N
  VARIANT 86: note = Xaa is Q or L
  VARIANT 97: note = Xaa is V or A
  VARIANT 109: note = Xaa is S or R
QVQLQESGGGLVQPGXSLRLSCXASGXXXXFTFSXYAMSWYRQXPGKERELVAAXXXXGXXXXYXDSVKGRFXISRDXAKNTVYLXMNSLKPEDTAXYYCNAVTLQGWXEYDYWGQGTQVTVSS

SEQ ID NO: 382 - protein, 116 aa, synthetic construct
  VARIANT 31: note = Xaa is S or T
  VARIANT 35: note = Xaa is S or T
  VARIANT 42: note = Xaa is E or G
  VARIANT 56: note = Xaa is V or R
  VARIANT 77: note = Xaa is R or S
  VARIANT 78: note = Xaa is M or T
  VARIANT 79: note = Xaa is V or L
  VARIANT 85: note = Xaa is S or N
  VARIANT 100: note = Xaa is S or N
QVQLQESGGGLVQPGGSLRLSCAASGFRFSXSIMXWYRQAPXKERELVASISSDSXRTNYAESVKGRFTISRDNEQXXXFLEMNXLKPVDTAVYFCTRPXIRGRDWGQGTQVTVSS

SEQ ID NO: 383 - protein, 122 aa, synthetic construct
  VARIANT 113: note = Xaa is G or D
QVQLQESGGGLVQPGGSLRLSCAASGRTFSSYPMGWFRQAPGKEREFVAAIDWSGDITYYADSVKGRFAISRDNAKNMVYLQMNSLKPEDTAVYYCNAGPLDSWERNDFPSWXQGTQVTVSS